SEQUENCE LISTING (292 sequences)

SEQ ID NO: 1 - DNA, 981 bp, Ebola virus
atgaggcgggttatattacctactgctcctcctgaatatatggaggccatataccctgtcaggtcaaattcaacaattgctagaggtggcaacagcaatacaggcttcctgacgccggagtcagtcaatggggacactccatcgaatccactcaggccaattgccgatgacaccatcgaccatgccagccacacaccaggcagtgtgtcatcagcattcatccttgaagctatggtgaatgtcatatcgggccccaaagtgctaatgaagcaaattccaatttggcttcctctaggtgtcgctgatcaaaagacctacagctttgactcaactacggccgccatcatgcttgcttcatacactatcacccatttcggcaaggcaaccaatccacttgtcagagtcaatcggctgggtcctggaatcccggatcatcccctcaggctcctgcgaattggaaaccaggctttcctccaggagttcgttcttccgccagtccaactaccccagtatttcacctttgatttgacagcactcaaactgatcacccaaccactgcctgctgcaacatggaccgatgacactccaacaggatcaaatggagcgttgcgtccaggaatttcatttcatccaaaacttcgccccattcttttacccaacaaaagtgggaagaaggggaacagtgccgatctaacatctccggagaaaatccaagcaataatgacctcactccaggacttcaagatcgttccaattgatccaaccaaaaatatcatgggaatcgaagtgccagaaactctggtccacaagctgaccggtaagaaggtgacttctaaaaatggacaaccaatcatccctgttcttttgccaaagtacattgggttggacccggtggctccaggagacctcaccatggtaatcacacaggattgtgacacgtgtcattctcctgcaagtcttccagctgtgattgagaagtaa

SEQ ID NO: 2 - protein, 326 aa, Ebola virus
MRRVILPTAPPEYMEAIYPVRSNSTIARGGNSNTGFLTPESVNGDTPSNPLRPIADDTIDHASHTPGSVSSAFILEAMVNVISGPKVLMKQIPIWLPLGVADQKTYSFDSTTAAIMLASYTITHFGKATNPLVRVNRLGPGIPDHPLRLLRIGNQAFLQEFVLPPVQLPQYFTFDLTALKLITQPLPAATWTDDTPTGSNGALRPGISFHPKLRPILLPNKSGKKGNSADLTSPEKIQAIMTSLQDFKIVPIDPTKNIMGIEVPETLVHKLTGKKVTSKNGQPIIPVLLPKYIGLDPVAPGDLTMVITQDCDTCHSPASLPAVIEK

SEQ ID NO: 3 - DNA, 2220 bp, Ebola virus
atggattctcgtcctcagaaagtctggatggcgccgagtctcactgaatctgacatggattaccacaagatcttgacagcaggtctgtccgttcaacaggggattgttcggcaaagagtcatcccagtgtatcaagtaaacaatcttgaagaaatttgccaacttatcatacaggcctttgaagcaggtgttgattttcaagagagtgcggacagtttccttctcatgctttgtcttcatcatgcgtaccagggagattacaaacttttcttggaaagtggcgcagtcaagtatttggaagggcacgggttccgttttgaagtcaagaagcgtgatggagtgaagcgccttgaggaattgctgccagcagtatctagtggaaaaaacattaagagaacacttgctgccatgccggaagaggagacaactgaagctaatgccggtcagtttctctcctttgcaagtctattccttccgaaattggtagtaggagaaaaggcttgccttgagaaggttcaaaggcaaattcaagtacatgcagagcaaggactgatacaatatccaacagcttggcaatcagtaggacacatgatggtgattttccgtttgatgcgaacaaattttctgatcaaatttctcctaatacaccaagggatgcacatggttgccgggcatgatgccaacgatgctgtgatttcaaattcagtggctcaagctcgtttttcaggcttattgattgtcaaaacagtacttgatcatatcctacaaaagacagaacgaggagttcgtctccatcctcttgcaaggaccgccaaggtaaaaaatgaggtgaactcctttaaggctgcactcagctccctggccaagcatggagagtatgctcctttcgcccgacttttgaacctttctggagtaaataatcttgagcatggtcttttccctcaactatcggcaattgcactcggagtcgccacagcacacgggagtaccctcgcaggagtaaatgttggagaacagtatcaacaactcagagaggctgccactgaggctgagaagcaactccaacaatatgcagagtctcgcgaacttgaccatcttggacttgatgatcaggaaaagaaaattcttatgaacttccatcagaaaaagaacgaaatcagcttccagcaaacaaacgctatggtaactctaagaaaagagcgcctggccaagctgacagaagctatcactgctgcgtcactgcccaaaacaagtggacattacgatgatgatgacgacattccctttccaggacccatcaatgatgacgacaatcctggccatcaagatgatgatccgactgactcacaggatacgaccattcccgatgtggtggttgatcccgatgatggaagctacggcgaataccagagttactcggaaaacggcatgaatgcaccagatgacttggtcctattcgatctagacgaggacgacgaggacactaagccagtgcctaatagatcgaccaagggtggacaacagaagaacagtcaaaagggccagcatatagagggcagacagacacaatccaggccaattcaaaatgtcccaggccctcacagaacaatccaccacgccagtgcgccactcacggacaatgacagaagaaatgaaccctccggctcaaccagccctcgcatgctgacaccaattaacgaagaggcagacccactggacgatgccgacgacgagacgtctagccttccgcccttggagtcagatgatgaagagcaggacagggacggaacttccaaccgcacacccactgtcgccccaccggctcccgtatacagagatcactctgaaaagaaagaactcccgcaagacgagcaacaagatcaggaccacactcaagaggccaggaaccaggacagtgacaacacccagtcagaacactcttttgaggagatgtatcgccacattctaagatcacaggggccatttgatgctgttttgtattatcatatgatgaaggatgagcctgtagttttcagtaccagtgatggcaaagagtacacgtatccagactcccttgaagaggaatatccaccatggctcactgaaaaagaggctatgaatgaagagaatagatttgttacattggatggtcaacaattttattggccggtgatgaatcacaagaataaattcatggcaatcctgcaacatcatcagtga

SEQ ID NO: 4 - protein, 739 aa, Ebola virus
MDSRPQKVWMAPSLTESDMDYHKILTAGLSVQQGIVRQRVIPVYQVNNLEEICQLIIQAFEAGVDFQESADSFLLMLCLHHAYQGDYKLFLESGAVKYLEGHGFRFEVKKRDGVKRLEELLPAVSSGKNIKRTLAAMPEEETTEANAGQFLSFASLFLPKLVVGEKACLEKVQRQIQVHAEQGLIQYPTAWQSVGHMMVIFRLMRTNFLIKFLLIHQGMHMVAGHDANDAVISNSVAQARFSGLLIVKTVLDHILQKTERGVRLHPLARTAKVKNEVNSFKAALSSLAKHGEYAPFARLLNLSGVNNLEHGLFPQLSAIALGVATAHGSTLAGVNVGEQYQQLREAATEAEKQLQQYAESRELDHLGLDDQEKKILMNFHQKKNEISFQQTNAMVTLRKERLAKLTEAITAASLPKTSGHYDDDDDIPFPGPINDDDNPGHQDDDPTDSQDTTIPDVVVDPDDGSYGEYQSYSENGMNAPDDLVLFDLDEDDEDTKPVPNRSTKGGQQKNSQKGQHIEGRQTQSRPIQNVPGPHRTIHHASAPLTDNDRRNEPSGSTSPRMLTPINEEADPLDDADDETSSLPPLESDDEEQDRDGTSNRTPTVAPPAPVYRDHSEKKELPQDEQQDQDHTQEARNQDSDNTQSEHSFEEMYRHILRSQGPFDAVLYYHMMKDEPVVFSTSDGKEYTYPDSLEEEYPPWLTEKEAMNEENRFVTLDGQQFYWPVMNHKNKFMAILQHHQ

SEQ ID NO: 5 - DNA, 465 bp, Ebola virus
aacagagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctgacaacccttgccacaatctccacgagtcctcaaccccccacaaccaaaccaggtccggacaacagcacccacaatacacccgtgtataaacttgacatctctgaggcaactcaagttgaacaacatcaccgcagaacagacaacgacagcacagcctccgacactccccccgccacgaccgcagccggacccctaaaagcagagaacaccaacacgagcaagggtaccgacctcctggaccccgccaccacaacaagtccccaaaaccacagcgagaccgctggcaacaacaacactcattagtag

SEQ ID NO: 6 - protein, 153 aa, Ebola virus
NRAKNISGQSPARTSSDPGTNTTTEDHKIMASENSSAMVQVHSQGREAAVSHLTTLATISTSPQPPTTKPGPDNSTHNTPVYKLDISEATQVEQHHRRTDNDSTASDTPPATTAAGPLKAENTNTSKGTDLLDPATTTSPQNHSETAGNNNTH

SEQ ID NO: 7 - DNA, 981 bp, Ebola virus
atgagaagggtcactgtgccgactgcaccacctgcatatgctgacattggctatcctatgagcatgcttccaatcaagtcaagcagggctgtaagtggaattcaacagaaacaagaggtccttcctggaatggatacaccatcgaactctatgagacctgttgctgatgataacattgatcacacaagtcataccccaaacggagtggcctcagcattcatcttggaggcaactgtcaatgtgatctcggggcccaaagtcctcatgaaacaaatccctatttggttgccactcggaattgctgaccaaaaaacatacagctttgactcaacaacagcagcaattatgctcgcatcctacacgatcactcattttggaaaggccaacaaccccctcgtcagagtgaatcgacttggtcaaggaataccggatcacccactcagattgctcaggatggggaaccaggctttccttcaagagtttgtgctaccaccagttcaactgccgcaatatttcacttttgatctgactgcactcaaattagtgacacagcctctccctgctgcaacatggacagatgagactccgagcaacctttcaggagcactccgtccagggctctcatttcacccgaaactgagacccgttctacttccaggcaagacgggaaagaaagggcatgtttctgatctgaccgccccagacaaaatccagacaattgtgaacctgatgcaagatttcaagattgtgccaatcgacccagccaagagcatcattgggatcgaggttccagaattgctggtccacaagctcaccgggaagaaaatgagtcagaagaacggacagcctataattcctgtcttactcccaaaatacattgggctagatccaatctcgcccggagacctaactatggtcataacaccagattatgatgattgtcattcacccgccagttgctcttatctcagtgaaaagtga

SEQ ID NO: 8 - protein, 326 aa, Ebola virus
MRRVTVPTAPPAYADIGYPMSMLPIKSSRAVSGIQQKQEVLPGMDTPSNSMRPVADDNIDHTSHTPNGVASAFILEATVNVISGPKVLMKQIPIWLPLGIADQKTYSFDSTTAAIMLASYTITHFGKANNPLVRVNRLGQGIPDHPLRLLRMGNQAFLQEFVLPPVQLPQYFTFDLTALKLVTQPLPAATWTDETPSNLSGALRPGLSFHPKLRPVLLPGKTGKKGHVSDLTAPDKIQTIVNLMQDFKIVPIDPAKSIIGIEVPELLVHKLTGKKMSQKNGQPIIPVLLPKYIGLDPISPGDLTMVITPDYDDCHSPASCSYLSEK

SEQ ID NO: 9 - DNA, 2217 bp, Ebola virus
atggataaacgggtgagaggttcatgggccctgggaggacaatctgaggttgatcttgactaccacaagatattaacagccgggctttcagtccaacaggggattgtgcgacagagagtcatcccggtatatgtcgtgaatgatcttgagggtatttgtcaacatatcattcaggcttttgaagcaggtgtagatttccaggataatgctgatagcttccttttacttttatgtttacatcatgcctaccaaggagatcataggctcttcctcaaaagtgatgcagttcaatatttagagggccatggcttcaggtttgaggtccgagaaaaggagaatgtgcaccgtctggatgaattgttgcccaatgttaccggtggaaaaaatctcaggagaacattggctgctatgcccgaagaggagacaacggaagctaatgctggtcagtttctatcctttgccagtttgtttctacccaaacttgtcgttggggagaaagcgtgcctggaaaaagtacaaaggcaaattcaggtccatgcagaacaagggctcattcaatatccaacttcctggcaatcagttggacacatgatggtgatcttccgtttgatgaggacaaactttttaatcaagtttctactaatacatcaagggatgcacatggttgcaggtcatgatgcgaatgacacagtaatatctaattctgttgcccaggcaaggttctctggtcttctgattgtaaagactgttctggatcacatcctacaaaaaacagatctcggagtacgacttcatccactggccaggacagcaaaagtgaagaatgaggtcagttcattcaaggcggctcttggttcacttgccaagcatggagaatatgctccgtttgcacgtctccttaatctttctggagtcaacaacttggaacatgggctttatccacaactttcagccatcgctttgggtgttgcaactgcccacgggagtacgcttgctggtgtgaatgtaggggagcaatatcagcaactgcgtgaggctgctactgaggctgaaaagcaactccaacaatatgctgaaacacgtgagttggataaccttgggcttgatgaacaggagaagaagattctcatgagcttccaccagaagaagaatgagatcagcttccagcagactaatgcaatggtaaccttaaggaaagaacggctggctaaattgaccgaagccatcacgactgcatcgaagatcaaggttggagaccgttatcctgatgacaatgatattccatttcccgggccgatctatgatgacactcaccccaatccctctgatgacaatcctgatgattcacgtgatacaactattccaggtggtgttgttgacccgtatgatgatgagagtaataattatcctgactacgaggattcggctgaaggcaccacaggagatcttgatctcttcaatttggacgacgacgatgatgacagccgaccaggaccaccagacagggggcagaacaaggagagggcggcccggacatatggcctccaagatccgaccttggacggagcgaaaaaggtgccggagttgaccccaggttcccatcaaccaggcaacctccacatcaccaagtcgggttcaaacaccaaccaaccacaaggcaatatgtcatctactctccatagtatgacccctatacaggaagaatcagagcccgatgatcaaaaagataatgatgacgagagtctcacatcccttgactctgaaggtgacgaagatggtgagagcatctctgaggagaacaccccaactgtagctccaccagcaccagtctacaaagacactggagtagacactaatcagcagaatggaccaagcagtactgtagatagtcaaggttctgaaagtgaagctctcccaatcaactctaaaaagagttccgcactagaagaaacatattatcatctcctaaaaacacagggtccatttgaggcaatcaattattatcacctaatgagtgatgaacccattgcttttagcactgaaagtggcaaggaatatatctttccagactcccttgaagaagcctacccgccgtggttgagtgagaaggaggccttagagaaggaaaatcgttatctggtcattgatggccagcaattcctctggccggtaatgagcctacgggacaagttccttgctgttcttcaacatgactga

SEQ ID NO: 10 - protein, 738 aa, Ebola virus
MDKRVRGSWALGGQSEVDLDYHKILTAGLSVQQGIVRQRVIPVYVVNDLEGICQHIIQAFEAGVDFQDNADSFLLLLCLHHAYQGDHRLFLKSDAVQYLEGHGFRFEVREKENVHRLDELLPNVTGGKNLRRTLAAMPEEETTEANAGQFLSFASLFLPKLVVGEKACLEKVQRQIQVHAEQGLIQYPTSWQSVGHMMVIFRLMRTNFLIKFLLIHQGMHMVAGHDANDTVISNSVAQARFSGLLIVKTVLDHILQKTDLGVRLHPLARTAKVKNEVSSFKAALGSLAKHGEYAPFARLLNLSGVNNLEHGLYPQLSAIALGVATAHGSTLAGVNVGEQYQQLREAATEAEKQLQQYAETRELDNLGLDEQEKKILMSFHQKKNEISFQQTNAMVTLRKERLAKLTEAITTASKIKVGDRYPDDNDIPFPGPIYDDTHPNPSDDNPDDSRDTTIPGGVVDPYDDESNNYPDYEDSAEGTTGDLDLFNLDDDDDDSRPGPPDRGQNKERAARTYGLQDPTLDGAKKVPELTPGSHQPGNLHITKSGSNTNQPQGNMSSTLHSMTPIQEESEPDDQKDNDDESLTSLDSEGDEDGESISEENTPTVAPPAPVYKDTGVDTNQQNGPSSTVDSQGSESEALPINSKKSSALEETYYHLLKTQGPFEAINYYHLMSDEPIAFSTESGKEYIFPDSLEEAYPPWLSEKEALEKENRYLVIDGQQFLWPVMSLRDKFLAVLQHD

SEQ ID NO: 11 - DNA, 465 bp, Ebola virus
ctcaacgagacagaagacgatgatgcgacatcgtcgagaactacaaagggaagaatctccgaccgggccaccaggaagtattcggacctggttccaaaggattcccctgggatggtttcattgcacgtaccagaaggggaaacaacattgccgtctcagaattcgacagaaggtcgaagagtagatgtgaatactcaggaaactatcacagagacaactgcaacaatcataggcactaacggtaacaacatgcagatctccaccatcgggacaggactgagctccagccaaatcctgagttcctcaccgaccatggcaccaagccctgagactcagacctccacaacctacacaccaaaactaccagtgatgaccaccgaggaatcaacaacaccaccgagaaactctcctggctcaacaacagaagcacccactctcaccaccccagagaatataacatagtag

SEQ ID NO: 12 - protein, 153 aa, Ebola virus
LNETEDDDATSSRTTKGRISDRATRKYSDLVPKDSPGMVSLHVPEGETTLPSQNSTEGRRVDVNTQETITETTATIIGTNGNNMQISTIGTGLSSSQILSSSPTMAPSPETQTSTTYTPKLPVMTTEESTTPPRNSPGSTTEAPTLTTPENIT

SEQ ID NO: 13 - DNA, 981 bp, Ebola virus
atgaggagggcaattctacctactgcaccgccagaatacatagaggctgtctacccaatgagaacggttagtactagtatcaacagtactgccagtggtccgaactttccagcaccggatgtaatgatgagtgatacaccctccaactcactccgaccaattgctgatgataacatcgatcatccaagtcatacaccaaccagtgtttcatcagcctttatactcgaggcaatggtgaatgtgatatcggggccgaaggtactaatgaagcaaattcctatatggctccccttgggtgttgctgatcaaaaaacatatagttttgactcaactacagctgcaattatgctcgcatcgtacaccatcactcactttggcaaaacctccaatccgcttgtgagaatcaatcgacttggtcctgggatccccgatcacccgttgcggcttctaagaataggaaatcaagccttcttgcaagagtttgtgctgcctccagttcaattgccgcagtatttcacttttgacctgacggctctaaagctgatcactcaacctctcccggcagcaacctggacggatgatactccgaccggtcctacaggaatacttcgtcctggaatttcctttcatcccaaactgagacctatcctattgccagggaagaccgggaaaagaggatccagctccgatcttacttctcctgataaaatacaagcaataatgaactttctccaagacctcaaactcgtgccgattgatccagccaagaacattatgggtattgaagtgccggaactcttggtccacagactaactggaaagaaaatcacaacaaaaaatggtcaaccaataattcctattcttctaccaaagtatattggcatggatcccatttctcagggagacctcacaatggtcatcactcaagactgtgacacttgccattctcctgctagtcttcctccagtcagcgagaaatga

SEQ ID NO: 14 - protein, 326 aa, Ebola virus
MRRAILPTAPPEYIEAVYPMRTVSTSINSTASGPNFPAPDVMMSDTPSNSLRPIADDNIDHPSHTPTSVSSAFILEAMVNVISGPKVLMKQIPIWLPLGVADQKTYSFDSTTAAIMLASYTITHFGKTSNPLVRINRLGPGIPDHPLRLLRIGNQAFLQEFVLPPVQLPQYFTFDLTALKLITQPLPAATWTDDTPTGPTGILRPGISFHPKLRPILLPGKTGKRGSSSDLTSPDKIQAIMNFLQDLKLVPIDPAKNIMGIEVPELLVHRLTGKKITTKNGQPIIPILLPKYIGMDPISQGDLTMVITQDCDTCHSPASLPPVSEK

SEQ ID NO: 15 - DNA, 2220 bp, Ebola virus
atggatcctcgtccaatcagaacctggatgatgcataacacatctgaagttgaagcagactaccataagattctaactgccggattgtccgtccagcaaggcattgtgagacaaagaatcattcctgtttaccaaatctcaaacctggaggaagtatgtcaactcatcatacaggcattcgaggctggcgtcgacttccaggatagtgcagatagctttttgttaatgctatgtctgcatcatgcctatcaaggggattataaacaatttttggaaagtaatgcggtaaaataccttgaaggtcatggattccgttttgagatgaagaaaaaggaaggtgtcaagcgcctggaggaactactccctgctgcctcgagtggaaagaacatcaagagaacattggctgcaatgcccgaggaggaaacaacagaagcaaatgctggacaatttctttcatttgctagtctgtttctcccaaaattggttgtcggagaaaaggcctgtctggagaaggttcaacgacaaatccaagtgcacgcagaacaaggtctgattcaatacccgacatcttggcaatcggtgggacatatgatggtcatcttcagactaatgcgaaccaacttcctgattaagttcctcctaatacatcaaggaatgcatatggttgcagggcatgatgctaatgatgccgtcattgccaactctgtagctcaagctcgtttctccggattgttgatagtcaaaacagtgcttgatcatatcctccaaaaaacagagcacggagttcgcctgcatcccttggcgcgaacagccaaagtcaaaaatgaggtgagctcttttaaggccgctttagcctcactagcacaacatggagaatatgccccgtttgctcgtctgctgaatctatctggggttaataatcttgagcatgggcttttccctcaactttctgcaattgctttgggagtagcaactgcacatgggagcactctggctggagtcaatgtaggagagcaataccaacaactgcgagaagcagccactgaggccgaaaagcagttgcagaaatatgctgaatctcgtgaacttgatcacctaggtcttgatgatcaggaaaagaaaatcctaaaagacttccatcagaaaaagaatgagatcagcttccagcagacgacagccatggtcacactgcggaaagagagattggccaaattgaccgaagctattacttccacctctatcctcaaaacaggaaggcggtatgatgatgacaatgatataccctttccagggccaatcaatgataacgagaactctggtcagaacgatgacgatccaacagactcccaggataccacaatcccggatgtaataatcgatccaaacgatggtgggtataataattacagcgattatgcaaatgatgctgcaagtgctcctgatgacctagttctttttgaccttgaggacgaggatgatgctgataacccggctcaaaacacgccagaaaaaaatgatagaccagcaacaacaaagctgagaaatggacaggaccaggatggaaaccaaggcgaaactgcatccccacgggtagcccccaaccaatacagagacaagccaatgccacaagtacaggacagatccgaaaatcatgaccaaacccttcaaacacagtccagggttttgactcctatcagcgaggaagcagaccccagcgaccacaacgatggtgacaatgaaagcattcctcccctggaatcagacgacgagggtagcactgatactactgcagcagaaacaaagcctgccactgcacctcccgctcccgtctaccgaagtatctccgtagatgattctgtcccctcagagaacattcccgcacagtccaatcaaacgaacaatgaggacaatgtcaggaacaatgctcagtcggagcaatccattgcagaaatgtatcaacatatcttgaaaacacaaggaccttttgatgccatcctttactaccatatgatgaaagaagagcccatcattttcagcactagtgatgggaaggagtatacatatccagactctcttgaagatgagtatccaccctggctcagcgagaaggaagccatgaacgaagacaatagattcataaccatggatggtcagcagttttactggcctgtgatgaatcatagaaataaattcatggcaatcctccagcatcacaggtga

SEQ ID NO: 16 - protein, 739 aa, Ebola virus
MDPRPIRTWMMHNTSEVEADYHKILTAGLSVQQGIVRQRIIPVYQISNLEEVCQLIIQAFEAGVDFQDSADSFLLMLCLHHAYQGDYKQFLESNAVKYLEGHGFRFEMKKKEGVKRLEELLPAASSGKNIKRTLAAMPEEETTEANAGQFLSFASLFLPKLVVGEKACLEKVQRQIQVHAEQGLIQYPTSWQSVGHMMVIFRLMRTNFLIKFLLIHQGMHMVAGHDANDAVIANSVAQARFSGLLIVKTVLDHILQKTEHGVRLHPLARTAKVKNEVSSFKAALASLAQHGEYAPFARLLNLSGVNNLEHGLFPQLSAIALGVATAHGSTLAGVNVGEQYQQLREAATEAEKQLQKYAESRELDHLGLDDQEKKILKDFHQKKNEISFQQTTAMVTLRKERLAKLTEAITSTSILKTGRRYDDDNDIPFPGPINDNENSGQNDDDPTDSQDTTIPDVIIDPNDGGYNNYSDYANDAASAPDDLVLFDLEDEDDADNPAQNTPEKNDRPATTKLRNGQDQDGNQGETASPRVAPNQYRDKPMPQVQDRSENHDQTLQTQSRVLTPISEEADPSDHNDGDNESIPPLESDDEGSTDTTAAETKPATAPPAPVYRSISVDDSVPSENIPAQSNQTNNEDNVRNNAQSEQSIAEMYQHILKTQGPFDAILYYHMMKEEPIIFSTSDGKEYTYPDSLEDEYPPWLSEKEAMNEDNRFITMDGQQFYWPVMNHRNKFMAILQHHR

SEQ ID NO: 17 - DNA, 465 bp, Ebola virus
agagcccaggatccaggtagcaaccagaagacgaaggtcactcccaccagcttcgccaacaaccaaacctccaagaaccacgaagacttggttccagaggatcccgcttcagtggttcaagtgcgagacctccagagggaaaacacagtgccgaccccacccccagacacagtccccacaactctgatccccgacacaatggaggaacaaaccaccagccactacgaaccaccaaacatttccagaaaccatcaagagaggaacaacaccgcacaccccgaaactctcgccaacaatcccccagacaacacaaccccgtcgacaccacctcaagacggtgagcggacaagttcccacacaacaccctccccccgcccagtcccaaccagcacaatccatcccaccacgcgagagactcacattcccaccacaatgacaacaagccatgacaccgacagctagtag

SEQ ID NO: 18 - protein, 153 aa, Ebola virus
RAQDPGSNQKTKVTPTSFANNQTSKNHEDLVPEDPASVVQVRDLQRENTVPTPPPDTVPTTLIPDTMEEQTTSHYEPPNISRNHQERNNTAHPETLANNPPDNTTPSTPPQDGERTSSHTTPSPRPVPTSTIHPTTRETHIPTTMTTSHDTDS

SEQ ID NO: 19 - DNA, 981 bp, Ebola virus
atgaggagaatcatcctacccacggcaccacctgaatacatggaggctgtttacccaatgagaacaatgaattctggtgcagacaacactgccagtggccctaattacacaacaactggtgtgatgacaaatgatactccctctaattcactccgaccagttgcagatgataatattgatcatccgagccacacgcctaacagtgttgcctctgcatttatattggaagctatggtgaatgtaatatctggcccgaaagtgctgatgaagcaaatcccaatctggcttcctctgggtgtctctgaccagaagacatatagctttgattcaaccactgctgccattatgctagcatcatataccatcactcattttggcaaaacctcaaatccccttgtgagaatcaaccgacttggtcctggcatacctgatcacccactacgactcctaagaataggaaatcaagccttcctacaagagtttgtgctacctcctgtacaactgccacaatacttcacttttgatctgacagcgctgaagctgatcacccagccactcccagcggcaacctggacagatgaaactccagctgtgtcaactggcacgctccgcccagggatctcattccatcccaaattaaggcctatcctgctaccaggaagagctggaaagaagggctccaactccgatctaacatctcctgacaaaatccaggctataatgaatttcctacaagacctcaaaattgtaccaatcgatccaaccaagaatatcatgggtattgaagtgccagaactcctggttcacaggctgactgggaagaagacaactaccaagaatggtcaaccaatcattccaattctgctaccaaagtacattggtcttgatcctctatctcaaggtgatctcacaatggtgatcactcaggactgtgattcctgccactccccggccagtcttcccccagtcaatgaaaaatga

SEQ ID NO: 20 - protein, 326 aa, Ebola virus
MRRIILPTAPPEYMEAVYPMRTMNSGADNTASGPNYTTTGVMTNDTPSNSLRPVADDNIDHPSHTPNSVASAFILEAMVNVISGPKVLMKQIPIWLPLGVSDQKTYSFDSTTAAIMLASYTITHFGKTSNPLVRINRLGPGIPDHPLRLLRIGNQAFLQEFVLPPVQLPQYFTFDLTALKLITQPLPAATWTDETPAVSTGTLRPGISFHPKLRPILLPGRAGKKGSNSDLTSPDKIQAIMNFLQDLKIVPIDPTKNIMGIEVPELLVHRLTGKKTTTKNGQPIIPILLPKYIGLDPLSQGDLTMVITQDCDSCHSPASLPPVNEK

SEQ ID NO: 21 - DNA, 2220 bp, Ebola virus
atggagagtcgggcccacaaagcatggatgacgcacaccgcatcaggtttcgaaacagattaccataagattttaacagcaggattgtcagtccaacaaggcattgtgagacaacgggtcattcaagtccaccaggttacaaacctagaagaaatatgccaattgatcattcaagcctttgaagctggtgttgattttcaagagagtgcagacagtttcttgctgatgctatgtttacatcatgcttatcagggtgactacaagcaattcttggaaagcaatgcagtcaagtaccttgagggtcatggctttcgctttgaggtcaggaaaaaggaaggagtcaagcgactcgaagaattgcttcctgctgcatccagtggcaagagcatcaggagaacactggctgcaatgcctgaagaggagacaacagaagcaaatgccggacagttcctctcttttgctagcttatttcttcctaagctagttgtcggagaaaaagcctgtctagaaaaggtgcagcggcaaattcaagttcattctgagcagggattgatccaataccccacagcctggcagtcagttggacacatgatggtcattttcagactgatgagaacaaattttctaattaagttcctccttatacatcaagggatgcatatggtagcaggacacgatgctaacgatgctgtcatcgcaaactctgtagctcaagcacgtttttcaggattattgatcgttaaaacagtgctagatcacatccttcagaaaacagagcacggagtgcgtcttcatcctttggcaagaactgctaaggtcaagaacgaagtaaattcctttaaggctgcccttagctcgctagcacaacatggagagtatgctccttttgctcgcttgctgaatctttctggagtcaacaatctcgagcacggactgtttcctcagctttctgcaattgccctaggtgtcgcaacggcacacggcagtaccctggcaggagtaaatgtgggggaacagtatcagcaactacgagaagcagccactgaggcagaaaaacaattgcagaaatacgctgaatctcgcgagcttgaccatctaggtctcgatgatcaagagaagaagatcttgaaagacttccatcagaagaaaaatgaaatcagcttccagcagacaacagccatggtcacactacggaaggaaaggctagccaagctcactgaggcaatcacctccacatcccttctcaagacaggaaaacagtatgatgatgacaacgatatcccctttcctgggcccatcaatgataacgaaaactcagaacagcaagacgatgatccaacagattctcaggacactaccatccctgatatcattgttgacccggatgatggcagatacaacaattatggagactatcctagtgagacggcgaatgcccctgaagaccttgttctttttgaccttgaagatggtgacgaggatgatcaccgaccgtcaagttcatcagagaacaacaacaaacacagtcttacaggaactgacagtaacaaaacaagtaactggaatcgaaacccgactaatatgccaaagaaagactccacacaaaacaatgacaatcctgcacagcgggctcaagaatacgccagggataacatccaggatacaccaacaccccatcgagctctaactcccatcagcgaagaaaccggctccaatggtcacaatgaagatgacattgatagcatccctcctttggaatcagacgaagaaaacaacactgagacaaccattaccaccacaaaaaataccactgctccaccagcacctgtttatcggagtaattcagaaaaggagcccctcccgcaagaaaaatcccagaagcaaccaaaccaagtgagtggtagtgagaataccgacaataaacctcactcagagcaatcagtggaagaaatgtatcgacacatcctccaaacacaaggaccatttgatgccatcctatactattacatgatgacggaggagccgattgtctttagcactagtgatgggaaagaatacgtataccctgattctcttgaaggggagcatccaccgtggctcagtgaaaaagaggccttgaatgaggacaataggtttatcacaatggatgatcaacaattctactggcctgtaatgaatcacaggaacaaattcatggctatccttcagcaccacaagtaa

SEQ ID NO: 22 - protein, 739 aa, Ebola virus
MESRAHKAWMTHTASGFETDYHKILTAGLSVQQGIVRQRVIQVHQVTNLEEICQLIIQAFEAGVDFQESADSFLLMLCLHHAYQGDYKQFLESNAVKYLEGHGFRFEVRKKEGVKRLEELLPAASSGKSIRRTLAAMPEEETTEANAGQFLSFASLFLPKLVVGEKACLEKVQRQIQVHSEQGLIQYPTAWQSVGHMMVIFRLMRTNFLIKFLLIHQGMHMVAGHDANDAVIANSVAQARFSGLLIVKTVLDHILQKTEHGVRLHPLARTAKVKNEVNSFKAALSSLAQHGEYAPFARLLNLSGVNNLEHGLFPQLSAIALGVATAHGSTLAGVNVGEQYQQLREAATEAEKQLQKYAESRELDHLGLDDQEKKILKDFHQKKNEISFQQTTAMVTLRKERLAKLTEAITSTSLLKTGKQYDDDNDIPFPGPINDNENSEQQDDDPTDSQDTTIPDIIVDPDDGRYNNYGDYPSETANAPEDLVLFDLEDGDEDDHRPSSSSENNNKHSLTGTDSNKTSNWNRNPTNMPKKDSTQNNDNPAQRAQEYARDNIQDTPTPHRALTPISEETGSNGHNEDDIDSIPPLESDEENNTETTITTTKNTTAPPAPVYRSNSEKEPLPQEKSQKQPNQVSGSENTDNKPHSEQSVEEMYRHILQTQGPFDAILYYYMMTEEPIVFSTSDGKEYVYPDSLEGEHPPWLSEKEALNEDNRFITMDDQQFYWPVMNHRNKFMAILQHHK

SEQ ID NO: 23 - DNA, 465 bp, Ebola virus
gaaacccagaaccaggtccttgacacgacagcgacggtctctcctcccatctccgcccacaaccacgcagccgaagaccacaaagaattggtttcagaggattccactccagtggttcagatgcaaaacatcaagggaaaggacacaatgccaaccacagtgacgggtgtaccaacaaccacaccctctccatttccaatcaatgctcgcaacactgatcataccaaatcatttatcggcctggaggggccccaagaagaccacagcaccacacagcctgccaagaccaccagccaaccaaccaacagcacagaatcgacgacactaaacccaacatcagagccctccagtagaggcacgggaccatccagccccacggtccccaacaccacagaaagccacgccgaacttggcaagacaaccccaaccacactcccagaacagcacactgccgccagttagtag

SEQ ID NO: 24 - protein, 153 aa, Ebola virus
ETQNQVLDTTATVSPPISAHNHAAEDHKELVSEDSTPVVQMQNIKGKDTMPTTVTGVPTTTPSPFPINARNTDHTKSFIGLEGPQEDHSTTQPAKTTSQPTNSTESTTLNPTSEPSSRGTGPSSPTVPNTTESHAELGKTTPTTLPEQHTAAS

SEQ ID NO: 25 - DNA, 996 bp, Ebola virus
atgagacgcggagtgttaccaacggctcctccagcatataatgatattgcataccctatgagcatactcccaacccgaccaagtgtcatagtcaatgagaccaaatcagatgtactggcagtgccaggggcagatgttccatcaaactccatgagaccagtggctgatgataacattgatcactcaagccatactccaagcggagtagcttctgcctttatattggaagctacagtgaatgtaatttcgggaacaaaagtcctgatgaagcaaatacctatttggcttccactgggtgtagctgatcagaagatatacagctttgattcaacaacagccgcaattatgttggcttcctacacagtgacacacttcgggaagatatctaacccgctggtacgtgtcaacaggctaggcccaggaatacccgatcatccgctacgactcctaaggttgggcaatcaggcattccttcaagagtttgttcttccaccagtccagcttccccagtatttcacatttgatctaacagctctaaagctcatcactcaaccattgccagctgcaacctggacagacgaaactccagcaggagcagtcaatgctcttcgtcctgggctctcactccatcccaagcttcgtccaattctcctgccggggaagacaggaaagaaaggacatgcttcagacttaacatcacctgacaagattcaaacaatcatgaatgcaataccggacctcaaaattgtcccgattgatccaaccaagaacatagttggaattgaggttccagaattactagttcaaaggctgaccggcaaaaaaccacaacccaaaaatggccaaccaattattccagttcttcttccgaaatatgttggacttgatcctatatcgccaggggacttaactatggttatcacccaggattgtgattcatgccactctccagccagccatccgtatcacatggacaagcaggatagttaccaataa

SEQ ID NO: 26 - protein, 331 aa, Ebola virus
MRRGVLPTAPPAYNDIAYPMSILPTRPSVIVNETKSDVLAVPGADVPSNSMRPVADDNIDHSSHTPSGVASAFILEATVNVISGTKVLMKQIPIWLPLGVADQKIYSFDSTTAAIMLASYTVTHFGKISNPLVRVNRLGPGIPDHPLRLLRLGNQAFLQEFVLPPVQLPQYFTFDLTALKLITQPLPAATWTDETPAGAVNALRPGLSLHPKLRPILLPGKTGKKGHASDLTSPDKIQTIMNAIPDLKIVPIDPTKNIVGIEVPELLVQRLTGKKPQPKNGQPIIPVLLPKYVGLDPISPGDLTMVITQDCDSCHSPASHPYHMDKQDSYQ

SEQ ID NO: 27 - DNA, 2220 bp, Ebola virus
atggatcgtgggaccagaagaatctgggtgtcgcaaaatcaaggtgatactgatttagattatcataaaattttgacagctggccttactgttcaacagggaattgtcaggcagaaaataatttctgtatatcttgttgataacttggaggctatgtgtcaattggtaatacaagcctttgaggccggaattgatttccaagaaaatgccgacagcttccttctgatgctttgcctacatcatgcttaccaaggtgactataaattgttcttggagagcaatgctgtacagtatttggaaggtcatggattcaaatttgagctccggaagaaggacggtgtcaatcggctcgaggaattgcttcctgctgcaacgagtggaaaaaacatcaggcgtacgttggccgcactgcctgaagaggagactacagaagcaaatgcagggcaatttctctcatttgcgagtttgtttcttcccaaactggttgtgggagagaaggcttgcttggaaaaagtccagcgacaaattcaggttcatgcagaacagggtttaattcaatatcccactgcatggcaatcagttggacacatgatggtaatcttcagattgatgaggactaatttcttgattaaatatttactgatccaccagggtatgcatatggtagctggccacgatgccaatgatgctgtcattgctaattcagttgctcaggctcgcttttcaggactcctaattgtcaaaaccgttcttgatcatattctgcaaaaaaccgaccaaggagtaagacttcaccctttggcccgaacagccaaagtgcgtaatgaggttaatgcatttaaggccgccctaagctcacttgctaagcatggggaatatgccccttttgctcgccttctcaatctctcgggagttaacaacctagaacatggtctctacccacagttatcagcaattgctcttggagttgccacagcacatggtagcacccttgcaggagttaatgttggtgagcagtatcagcagcttagagaggctgccactgaagctgagaagcaactccaacaatatgctgagtccagagaactcgacagcctaggcctggacgatcaggaaagaagaatactaatgaacttccatcagaagaaaaacgaaattagtttccagcagaccaatgcaatggtaacccttaggaaagagcgactggctaaattaacagaagctataacgctggcctcaagacctaacctcgggtctagacaagacgacggcaatgaaataccgttccctgggcctataagcaacaacccagaccaagatcatctggaggatgatcctagagactccagagacaccatcattcctaatggtgcaattgaccccgaggatggtgattttgaaaattacaatggctatcatgatgatgaagttgggacggcaggtgacttggtcctgttcgatcttgacgatcatgaggatgacaataaagcttttgagccacaggacagctcgccacaatcccaaagggaaatagagagagaaagattaattcatccacccccaggcaacaacaaggacgacaatcgagcctcagacaacaatcaacaatcagcagattctgaggaacaaggaggtcaatacaactggcaccgaggcccagaacgtacgaccgccaatcgaagactctcaccagtgcacgaagaggacacccttatggatcaaggcgatgatgatccctcaagcttacctccgctggaatctgatgatgacgatgcatcaagtagccaacaagatcccgattatacagctgttgcccctcctgctcctgtataccgcagtgcagaagcccacgagcctccccacaaatcctcgaacgagccagctgaaacatcacaattgaatgaagaccctgatatcggtcaatcaaagtctatgcaaaaattagaagagacatatcaccatctgctgagaactcaaggtccatttgaagccatcaattattatcacatgatgaaggatgagccggtaatatttagcactgatgatgggaaggaatacacctacccggattcacttgaggaagcctatcctccatggctcaccgagaaagaacgactggacaaagagaatcgctacatttacataaataatcaacagttcttctggcctgtcatgagtcccagagacaaatttcttgcaatcttgcagcaccatcagtaa

SEQ ID NO: 28 - protein, 739 aa, Ebola virus
MDRGTRRIWVSQNQGDTDLDYHKILTAGLTVQQGIVRQKIISVYLVDNLEAMCQLVIQAFEAGIDFQENADSFLLMLCLHHAYQGDYKLFLESNAVQYLEGHGFKFELRKKDGVNRLEELLPAATSGKNIRRTLAALPEEETTEANAGQFLSFASLFLPKLVVGEKACLEKVQRQIQVHAEQGLIQYPTAWQSVGHMMVIFRLMRTNFLIKYLLIHQGMHMVAGHDANDAVIANSVAQARFSGLLIVKTVLDHILQKTDQGVRLHPLARTAKVRNEVNAFKAALSSLAKHGEYAPFARLLNLSGVNNLEHGLYPQLSAIALGVATAHGSTLAGVNVGEQYQQLREAATEAEKQLQQYAESRELDSLGLDDQERRILMNFHQKKNEISFQQTNAMVTLRKERLAKLTEAITLASRPNLGSRQDDGNEIPFPGPISNNPDQDHLEDDPRDSRDTIIPNGAIDPEDGDFENYNGYHDDEVGTAGDLVLFDLDDHEDDNKAFEPQDSSPQSQREIERERLIHPPPGNNKDDNRASDNNQQSADSEEQGGQYNWHRGPERTTANRRLSPVHEEDTLMDQGDDDPSSLPPLESDDDDASSSQQDPDYTAVAPPAPVYRSAEAHEPPHKSSNEPAETSQLNEDPDIGQSKSMQKLEETYHHLLRTQGPFEAINYYHMMKDEPVIFSTDDGKEYTYPDSLEEAYPPWLTEKERLDKENRYIYINNQQFFWPVMSPRDKFLAILQHHQ

SEQ ID NO: 29 - DNA, 465 bp, Ebola virus
acccacaccaacaactcctcagatcagagcccggcgggaactgtccaaggaaaaattagctaccacccacccgccaacaactccgagctggttccaacggattcccctccagtagtttcagtgctcactgcaggacggacagaggaaatgtcgacccaaggtctaaccaacggagagacaatcacaggtttcaccgcgaacccaatgacaaccaccattgccccaagtccaaccatgacaagcgaggttgataacaatgtaccaagtgaacaaccgaacaacacagcatccattgaagactcccccccatcggcaagcaacgagacaatttaccactccgagatggatccgatccaaggctcgaacaactccgcccagagcccacagaccaagaccacgccagcacccacaacatccccgatgacccaggacccgcaagagacggccaacagcagcaaatagtag

SEQ ID NO: 30 - protein, 153 aa, Ebola virus
THTNNSSDQSPAGTVQGKISYHPPANNSELVPTDSPPVVSVLTAGRTEEMSTQGLTNGETITGFTANPMTTTIAPSPTMTSEVDNNVPSEQPNNTASIEDSPPSASNETIYHSEMDPIQGSNNSAQSPQTKTTPAPTTSPMTQDPQETANSSK

SEQ ID NO: 31 - DNA, 912 bp, Marburg virus
atggccagttccagcaattacaacacatacatgcaatacttgaacccccctccttatgctgatcacggtgcaaaccagttgatcccggcggatcagctatcaaatcagcagggtataactccaaattacgtgggtgatttaaacctagatgatcagttcaaagggaatgtctgccatgctttcactttagaggcaataattgacatatctgcatataacgagcgaacagtcaaaggcgttccggcatggctgcctcttgggattatgagcaattttgaatatcctttagctcatactgtggccgcgttgctcacaggcagctatacaatcacccaatttactcacaacgggcaaaaattcgtccgtgttaatcgacttggtacaggaatcccagcacacccactcagaatgttgcgtgaaggaaatcaagcttttattcagaatatggtgatccccaggaatttttcaactaatcaattcacctacaatctcactaatttagtattgagtgtgcaaaaacttcctgatgatgcctggcgcccatccaaggacaaattaattgggaacactatgcatcccgcagtctccatccacccgaatctgccgcctattgttctaccaacagtcaagaagcaggcttatcgtcagcacaaaaatcccaacaatggaccattgctggccatatctggcatcctccatcaactgagggtcgaaaaagtcccagagaagacgagcctgtttaggatctcgcttcctgccgacatgttctcagtaaaagagggtatgatgaagaaaaggggagaaaattcccccgtggtttattttcaagcacctgagaacttccctttgaatggcttcaataacagacaagttgtgctagcgtatgcgaatccaacgctcagtgccgtttga

SEQ ID NO: 32 - protein, 303 aa, Marburg virus
MASSSNYNTYMQYLNPPPYADHGANQLIPADQLSNQQGITPNYVGDLNLDDQFKGNVCHAFTLEAIIDISAYNERTVKGVPAWLPLGIMSNFEYPLAHTVAALLTGSYTITQFTHNGQKFVRVNRLGTGIPAHPLRMLREGNQAFIQNMVIPRNFSTNQFTYNLTNLVLSVQKLPDDAWRPSKDKLIGNTMHPAVSIHPNLPPIVLPTVKKQAYRQHKNPNNGPLLAISGILHQLRVEKVPEKTSLFRISLPADMFSVKEGMMKKRGENSPVVYFQAPENFPLNGFNNRQVVLAYANPTLSAV

SEQ ID NO: 33 - DNA, 2088 bp, Marburg virus
atggatttacacagtttgttggagttgggtacaaaacccactgcccctcatgttcgtaataagaaagtgatattatttgacacaaatcatcaggttagtatctgtaatcagataatagatgcaataaactcagggattgatcttggagatctcctagaagggggtttgctgacgttgtgtgttgagcattactataattctgataaggataaattcaacacaagtcctatcgcgaagtacttacgtgatgcgggctatgaatttgatgtcatcaagaatgcagatgcaacccgctttctggatgtgattcctaatgaacctcattacagccctttaattctagcccttaagacattggaaagtactgaatctcagagggggagaattgggctctttttatcattttgcagtcttttcctcccaaaacttgtcgtcggagaccgagctagtatcgaaaaggctttaagacaagtaacagtgcatcaagaacaggggatcgtcacataccctaatcattggcttaccacaggccacatgaaagtaattttcgggattttgaggtccagcttcattttaaagtttgtgttgattcatcaaggagtaaatttggtgacaggtcatgatgcctatgacagtatcattagtaattcagtaggtcaaactagattctcaggacttcttatcgtgaaaacagttctcgagttcatcttgcaaaaaactgattcaggggtgacactacatcctttggtgcggacctccaaagtaaaaaatgaagttgctagtttcaagcaggcgttgagcaacctagcccgacatggggaatacgcaccatttgcacgggttctgaatttatcagggattaacaacctcgaacatggactctatcctcagctttcagcaattgcgctgggtgtggcaacagcacacggcagtacattggctggtgtcaatgttggcgaacaatatcaacaactacgagaggcggcacatgatgcggaagtaaaactacaaaggcgacatgaacatcaggaaattcaagctattgccgaggatgacgaggaaaggaagatattagaacaattccaccttcagaaaactgaaatcacacacagtcagacactagccgtcctcagccagaaacgagaaaaattagctcgtctcgctgcagaaattgaaaacaatattgtggaagatcagggatttaagcaatcacagaatcgggtgtcacagtcgtttttgaatgaccctacacctgtggaagtaacggttcaagccaggcccatgaatcgaccaactgctctgcctcccccagttgacgacaagattgagcatgaatctacagaagatagctcttcttcaagtagctttgttgacttgaatgatccatttgcactgctgaatgaggacgaggatactcttgatgacagtgtcatgatcccgggcacaacatcgagagaatttcaagggattcctgaaccgccaagacaatcccaagacctcaataacagccaaggaaagcaggaagatgaatccacaaatccgattaagaaacagtttctgagatatcaagaattgcctcctgttcaagaggatgatgaatcggaatacacaactgactctcaagaaagcatcgaccaaccaggatccgacaatgaacaaggagttgatcttccacctcctccgttgtacgctcaggaaaaaagacaggacccaatacagcacccagcagcaaaccctcaggatcccttcggcagtattggtgatgtaaatggtgatatcttagaacctataagatcaccttcttcaccatctgctcctcaggaagacacaaggatgagggaagcctatgaattgtcgcctgatttcacaaatgatgaggataatcagcagaattggccacaaagagtggtgacaaagaagggtagaactttcctttatcctaatgatcttctgcaaacaaatcctccagagtcacttataacagccctcgttgaggaataccaaaatcctgtctcagctaaggagcttcaagcagattggcccgacatgtcatttgatgaaaggagacatgttgcgatgaacttgtag

SEQ ID NO: 34 - protein, 695 aa, Marburg virus
MDLHSLLELGTKPTAPHVRNKKVILFDTNHQVSICNQIIDAINSGIDLGDLLEGGLLTLCVEHYYNSDKDKFNTSPIAKYLRDAGYEFDVIKNADATRFLDVIPNEPHYSPLILALKTLESTESQRGRIGLFLSFCSLFLPKLVVGDRASIEKALRQVTVHQEQGIVTYPNHWLTTGHMKVIFGILRSSFILKFVLIHQGVNLVTGHDAYDSIISNSVGQTRFSGLLIVKTVLEFILQKTDSGVTLHPLVRTSKVKNEVASFKQALSNLARHGEYAPFARVLNLSGINNLEHGLYPQLSAIALGVATAHGSTLAGVNVGEQYQQLREAAHDAEVKLQRRHEHQEIQAIAEDDEERKILEQFHLQKTEITHSQTLAVLSQKREKLARLAAEIENNIVEDQGFKQSQNRVSQSFLNDPTPVEVTVQARPMNRPTALPPPVDDKIEHESTEDSSSSSSFVDLNDPFALLNEDEDTLDDSVMIPGTTSREFQGIPEPPRQSQDLNNSQGKQEDESTNPIKKQFLRYQELPPVQEDDESEYTTDSQESIDQPGSDNEQGVDLPPPPLYAQEKRQDPIQHPAANPQDPFGSIGDVNGDILEPIRSPSSPSAPQEDTRMREAYELSPDFTNDEDNQQNWPQRVVTKKGRTFLYPNDLLQTNPPESLITALVEEYQNPVSAKELQADWPDMSFDERRHVAMNL

SEQ ID NO: 35 - DNA, 657 bp, Marburg virus
gggctttcatcaacaatgccacccactccctcaccacaaccaagcacgccacagcaaggaggaaacaacacaaaccattcccaagatgctgtgactgaacttgacaaaaataacacaactgcacaaccgtccatgccccctcataacactaccacaatctctactaacaacacctccaaacacaacttcagcactctctctgcaccattacaaaacaccaccaatgacaacacacagagcacaatcactgaaaatgagcaaaccagtgccccctcgataacaaccctgcctccaacgggaaatcccaccacagcaaagagcaccagcagcaaaaaaggccccgccacaacggcaccaaacacgacaaatgagcatttcaccagtcctccccccacccccagctcgactgcacaacatcttgtatatttcagaagaaagcgaagtatcctctggagggaaggcgacatgttcccttttctggatgggttaataaatgctccaattgattttgacccagttccaaatacaaaaacaatctttgatgaatcctctagttctggtgcctcggctgaggaagatcaacatgcctcccccaatattagtttaactttatcttattttcctaatataaatgagaacactgcctactcttagtag

SEQ ID NO: 36 - protein, 217 aa, Marburg virus
GLSSTMPPTPSPQPSTPQQGGNNTNHSQDAVTELDKNNTTAQPSMPPHNTTTISTNNTSKHNFSTLSAPLQNTTNDNTQSTITENEQTSAPSITTLPPTGNPTTAKSTSSKKGPATTAPNTTNEHFTSPPPTPSSTAQHLVYFRRKRSILWREGDMFPFLDGLINAPIDFDPVPNTKTIFDESSSSGASAEEDQHASPNISLTLSYFPNINENTAYS

SEQ ID NO: 37 - protein, 454 aa, Zika virus
IRCIGVSNRDFVEGMSGGTWVDIVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFK

SEQ ID NO: 38 - DNA, 25 bp, Artificial Sequence
  Zika virus strain SPH2015 E gene forward primer
caccatccggtgcataggtgtttcg

SEQ ID NO: 39 - DNA, 27 bp, Artificial Sequence
  Zika virus strain SPH2015 E gene reverse primer
tcatttaaaggcggctccgaagatttg

SEQ ID NO: 40 - protein, 353 aa, Zika virus
VGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSHFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGS

SEQ ID NO: 41 - DNA, 22 bp, Artificial Sequence
  Zika virus strain SPH2015 NS1 gene forward primer
caccgtgggctgttcagtcgac

SEQ ID NO: 42 - DNA, 30 bp, Artificial Sequence
  Zika virus strain SPH2015 NS1 gene reverse primer
ttaagaacccgctgtaaccattgaacgcac

SEQ ID NO: 43 - protein, 454 aa, Zika virus
IRCIGVSNRDFVEGMSGGTWVDVVLEHGGCVTVMAQDKPAVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFK

SEQ ID NO: 44 - DNA, 22 bp, Artificial Sequence
  Zika virus strain Yap E gene forward primer
caccattcgttgcatcggcgtg

SEQ ID NO: 45 - DNA, 27 bp, Artificial Sequence
  Zika virus strain Yap  E gene reverse primer
ttatttaaaggctgcgccaaagatctg

SEQ ID NO: 46 - protein, 103 aa, Zika virus
KGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRS

SEQ ID NO: 47 - DNA, 22 bp, Artificial Sequence
  Zika virus strain Yap EDIII gene forward primer
caccaaaggcgtaagctactca

SEQ ID NO: 48 - DNA, 21 bp, Artificial Sequence
  Zika virus strain Yap EDIII gene reverse primer
ttagctgcgatgccaatgatg

SEQ ID NO: 49 - protein, 165 aa, Zika virus
TRRGSAYYMYLDRSDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYS

SEQ ID NO: 50 - DNA, 24 bp, Artificial Sequence
  Zika virus strain Yap pM gene forward primer
caccacgcggcgcggttcagcata

SEQ ID NO: 51 - DNA, 27 bp, Artificial Sequence
  Zika virus strain Yap pM gene reverse primer
ttaactatacgccggagcgatgagcag

SEQ ID NO: 52 - protein, 353 aa, Zika virus
  MISC_FEATURE (50)..(50): Xaa may be any amino acid
VGCSVDFSKKETRCGTGVFVYNDVDAWRDRYKYHPDSPRRLAAAVKQAWXDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAAKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGS

SEQ ID NO: 53 - DNA, 22 bp, Artificial Sequence
  Zika virus strain Yap NS1 gene forward primer
caccgtggggtgttcagttgat

SEQ ID NO: 54 - DNA, 21 bp, Artificial Sequence
  Zika virus strain Yap NS1 gene reverse primer
ttaagaacccgccgtgaccat

SEQ ID NO: 55 - protein, 454 aa, Zika virus
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFTCSKKMTGKSIQPENLEYRIMLSVHGSQHSGMIVNDTGHETDENRAKVEVTPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLFSGHLKCRLKMDKLRLKGVSYSLCTAAFTFTKVPAETLHGTVTVEVQYAGTDGPCKVPAQMAVDMQTLTPVGRLITANPVITESTENSKMMLELDPPFGDSYIVIGVGDKKITHHWHRSGSTIGKAFEATVRGAKRMAVLGDTAWDFGSVGGVFNSLGKGIHQIFGAAFK

SEQ ID NO: 56 - DNA, 25 bp, Artificial Sequence
  Zika virus strain ArD_41519 E gene forward primer
caccattcggtgcataggggtcagc

SEQ ID NO: 57 - DNA, 30 bp, Artificial Sequence
  Zika virus strain ArD_41519 E gene reverse primer
tcatttaaacgcagctccgaaaatctgatg

SEQ ID NO: 58 - protein, 454 aa, Zika virus
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFTCSKKMTGKSIQPENLEYRIMLSVHGSQHSGMIVNDIGHETDENRAKVEVTPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLFSGHLKCRLKMDKLRLKGVSYSLCTAAFTFTKVPAETLHGTVTVEVQYAGTDGPCKVPAQMAVDMQTLTPVGRLITANPVITESTENSKMMLELDPPFGDSYIVIGVGDKKITHHWHRSGSTIGKAFEATVRGAKRMAVLGDTAWDFGSVGGVFNSLGKGVHQIFGAAFK

SEQ ID NO: 59 - DNA, 24 bp, Artificial Sequence
  Zika virus strain ARB7701 E gene forward primer
caccattcgttgcattggtgttag

SEQ ID NO: 60 - DNA, 33 bp, Artificial Sequence
  Zika virus strain ARB7701 E gene reverse primer
ttatttgaaggctgcaccaaaaatctgatgcac

SEQ ID NO: 61 - protein, 450 aa, Zika virus
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFTCSKKMTGKSIQPENLEYRIMLSVHGSQHSGMIGYETDEDRAKVEVTPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLFSGHLKCRLKMDKLRLKGVSYSLCTAAFTFTKVPAETLHGTVTVEVQYAGTDGPCKIPVQMAVDMQTLTPVGRLITANPVITESTENSKMMLELDPPFGDSYIVIGVGDKKITHHWHRSGSTIGKAFEATVRGAKRMAVLGDTAWDFGSVGGVFNSLGKGIHQIFGAAFK

SEQ ID NO: 62 - DNA, 22 bp, Artificial Sequence
  Zika virus strain MR-766 E gene forward primer
caccaaaggcgttagctatagt

SEQ ID NO: 63 - DNA, 27 bp, Artificial Sequence
  Zika virus strain MR-766 E gene reverse primer
ttatttaaatgcggcgccgaaaatctg

SEQ ID NO: 64 - protein, 103 aa, Zika virus
KGVSYSLCTAAFTFTKVPAETLHGTVTVEVQYAGTDGPCKIPVQMAVDMQTLTPVGRLITANPVITESTENSKMMLELDPPFGDSYIVIGVGDKKITHHWHRS

SEQ ID NO: 65 - DNA, 22 bp, Artificial Sequence
  Zika virus strain MR-766 E-DIII gene forward primer
caccaagggcgtgtcatattcc

SEQ ID NO: 66 - DNA, 24 bp, Artificial Sequence
  Zika virus strain MR-766 E-DIII gene reverse primer
ttatgaacgatgccaatggtgcgt

SEQ ID NO: 67 - protein, 165 aa, Zika virus
TRRGSAYYMYLDRSDAGKAISFATTLGVNKCHVQIMDLGHMCDATMSYECPMLDEGVEPDDVDCWCNTTSTWVVYGTCHHKKGEARRSRRAVTLPSHSTRKLQTRSQTWLESREYTKHLIKVENWIFRNPGFALVAVAIAWLLGSSTSQKVIYLVMILLIAPAYS

SEQ ID NO: 68 - DNA, 19 bp, Artificial Sequence
  Zika virus strain MR-766 pM gene forward primer
caccactcgccgcgggtca

SEQ ID NO: 69 - DNA, 24 bp, Artificial Sequence
  Zika virus strain MR-766 pM gene reverse primer
ttaggaatacgccggtgcgatcag

SEQ ID NO: 70 - protein, 353 aa, Zika virus
VGCSVDFSKKETRCGTGVFIYNDVEAWRDRYKYHPDSPRRLAAAVKQAWEEGICGISSVSRMENIMWKSVEGELNAILEENGVQLTVVVGSVKNPMWRGPQRLPVPVNELPHGWKAWGKSYFVRAAKTNNSFVVDGDTLKECPLEHRAWNSFLVEDHGFGVFHTSVWLKVREDYSLECDPAVIGTAVKGREAAHSDLGYWIESEKNDTWRLKRAHLIEMKTCEWPKSHTLWTDGVEESDLIIPKSLAGPLSHHNTREGYRTQVKGPWHSEELEIRFEECPGTKVYVEETCGTRGPSLRSTTASGRVIEEWCCRECTMPPLSFRAKDGCWYGMEIRPRKEPESNLVRSMVTAGS

SEQ ID NO: 71 - DNA, 19 bp, Artificial Sequence
  Zika virus strain MR-766 NS1 gene forward primer
caccgtcggctgtagcgtc

SEQ ID NO: 72 - DNA, 24 bp, Artificial Sequence
  Zika virus strain MR-766 NS1 gene reverse primer
ttaagagccagccgtaaccatact

SEQ ID NO: 73 - protein, 442 aa, Dengue virus type 1
  MISC_FEATURE (272)..(272): Xaa may be any amino acid
MRCVGIGNRDFVEGLSGATWVDVVLEHGSCVTTMAKDKPTLDIELLKTEVTNPAVLRKLCIEAKISNTTTDSRCPTQGEATLVEEQDANFVCRRTFVDRGWGNGCGLFGKGSLITCAKFKCVTKLEGKIVQYENLKYSVIVTVHTGDQHQVGNETTEHGTTATITPQAPTSEIQLTDYGALTLDCSPRTGLDFNEMVLLTMKEKSWLVHKQWFLDLPLPWTSGASTPQETWNRQDLLVTFKTAHAKKQEVVVLGSQEGAMHTALTGATEIQXSGTTTIFAGHLKCRLKMDKLTLKGMSYVMCTGSFKLEKEVAETQHGTVLVQVKYEGTDAPCKIPFSTQDEKGVTQNGRLITANPIVTDKEKPVNIEAEPPFGESYIVVGAGEKALKLSWFKKGSSIGKMLEATARGARRMAILGDTAWDFGSIGGVFTSVGKLVHQIFGT

SEQ ID NO: 74 - DNA, 22 bp, Artificial Sequence
  Dengue virus 1 E gene forward primer
caccatgcggtgcgtgggaata

SEQ ID NO: 75 - DNA, 24 bp, Artificial Sequence
  Dengue virus 1 E gene reverse primer
ttaagttccaaagatctggtgtac

SEQ ID NO: 76 - protein, 99 aa, Dengue virus type 1
YVMCTGSFKLEKEVAETQHGTVLVQVKYEGTDAPCKIPFSTQDEKGVTQNGRLITANPIVTDKEKPVNIEAEPPFGESYIVVGAGEKALKLSWFKKGSS

SEQ ID NO: 77 - DNA, 28 bp, Artificial Sequence
  Dengue virus 1 E-DIII gene forward primer
cacctatgtgatgtgcacaggctcattc

SEQ ID NO: 78 - DNA, 27 bp, Artificial Sequence
  Dengue virus 1 E-DIII gene reverse primer
ttagctgcttcctttcttgaaccagct

SEQ ID NO: 79 - protein, 74 aa, Dengue virus type 1
SVALAPHVGLGLETRAETWMSSEGAWKQIQKVETWALRHPGFTVTALFLAHAIGTSITQKGIIFILLMLVTPSM

SEQ ID NO: 80 - DNA, 21 bp, Artificial Sequence
  Dengue virus 1 M gene forward primer
tccgtcgcactggcaccacac

SEQ ID NO: 81 - DNA, 21 bp, Artificial Sequence
  Dengue virus 1 M gene reverse primer
ggccatggatggagttaccag

SEQ ID NO: 82 - protein, 351 aa, Dengue virus type 1
DSGCVINWKGRELKCGSGIFVTNEVHTWTEQYKFQADSPKRLSAAIGKAWEEGVCGIRSATRLENIMWKQISNELNHILLENDMKFTVVVGDVSGILAQGKKMIGPQPMEHKYSWKSWGKAKIIGADVQNTTFIIDGPNTPECPDDQRAWNIWEVEDYGFGIFTTNIWLKLRDSYTQVCDPRLMSAAIKDSKAVHADMGYWIESEKNETWKLARASFIEVKTCVWPKSHTLWSNGVLESEMIIPKIYGGPISQHNYRPGYFTQTAGPWHLGKLELDFDLCEGTTVVVDEHCGNRGPSLRTTTVTGKIIHEWCCRSCTLPPLRFKGEDGCWYGMEIRPVKDKEENLVKSLVS

SEQ ID NO: 83 - DNA, 21 bp, Artificial Sequence
  Dengue virus 1 NS1 gene forward primer
gactcgggatgtgtaatcaac

SEQ ID NO: 84 - DNA, 21 bp, Artificial Sequence
  Dengue virus 1 NS1 gene reverse primer
tgcagagaccattgacttaac

SEQ ID NO: 85 - protein, 618 aa, Dengue virus type 1
  MISC_FEATURE (480)..(480): Xaa may be any amino acid
  MISC_FEATURE (515)..(515): Xaa may be any amino acid
SGVLWDTPSPPEVERAVLDDGIYRILQRGLLGRSQVGVGVFQEGVFHTMWHVTRGAVLMYQGKRLEPSWASVKKDLISYGGGWRFQGSWNTGEEVQVIAVEPGKNPKNVQTAPGTFKTPEGEVGAIALDFKPGTSGSPIVNREGKIVGLYGNGVVTTSGTYVSAIAQAKASQEGPLPEIEDEVFRKRNLTIMDLHPGSGKTRRYLPAIVREAIKRKLRTLILAPTRVVASEMAEALKGMPIRYQTTAVKSEHTGKEIVDLMCHATFTMRLLSPVRVPNYNMIIMDEAHFTDPASIAARGYISTRVGMGEAAAIFMTATPPGSVEAFPQSNAVIQDEERDIPERSWNSGYDWITDFPGKTVWFVPSIKSGNDIANCLRKNGKRVIQLSRKTFDTEYQKTKNNDWDYVVTTDISEMGANFRADRVIDPRRCLKPVILKDGPERVILAGPMPVTVASAAQRRGRIGRNQNKEGDQYIYMGQPXNNDEDHAHWTEAKMLLDNINTPEGIIPALFEPERXKSAAIDGEYRLRGEARKTFVELMRRGDLPVWLSYKVASEGFQYSDRKWCFDGERNNQVLEENMDVEIWTKEGERKKLRPRWLDARTYSDPQALREFKEFAAGR

SEQ ID NO: 86 - DNA, 24 bp, Artificial Sequence
  Dengue virus 1 NS3 gene forward primer
tcaggagtgctatgggacacaccc

SEQ ID NO: 87 - DNA, 21 bp, Artificial Sequence
  Dengue virus 1 NS3 gene reverse primer
tcttcttcctgctgcgaactc

SEQ ID NO: 88 - protein, 442 aa, Dengue virus type 2
MRCIGISNRDFVEGVSGGSWVDIVLEHGSCVTTMAKNKPTLDFELIKTEAKQPATLRKYCIEAKLTNTTTDSRCPTQGEPSLNEEQDKRFVCKHSMVDRGWGNGCGLFGKGGIVTCAMFTCKKNMKGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMIETTMRGAKRMAILGDTAWDFGSLGGVFTSIGKALHQVFGA

SEQ ID NO: 89 - DNA, 25 bp, Artificial Sequence
  Dengue virus 2 E gene forward primer
caccatgcgttgcataggaatatca

SEQ ID NO: 90 - DNA, 24 bp, Artificial Sequence
  Dengue virus 2 E gene reverse primer
ttatgctccgaaaacttggtggag

SEQ ID NO: 91 - protein, 101 aa, Dengue virus type 2
KGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKG

SEQ ID NO: 92 - DNA, 28 bp, Artificial Sequence
  Dengue virus 2 E-DIII gene forward primer
caccaaaggaatgtcatactctatgtgc

SEQ ID NO: 93 - DNA, 27 bp, Artificial Sequence
  Dengue virus 2 E-DIII gene reverse primer
ttatcctttcttaaaccagttgagctt

SEQ ID NO: 94 - protein, 74 aa, Dengue virus type 2
SVALVPHVGMGLETRTETWMSSEGAWKHAQRIETWILRHPGFTIMAAILAYTIGTTHFQRALIFILLTAVAPSM

SEQ ID NO: 95 - DNA, 24 bp, Artificial Sequence
  Dengue virus 2 M gene forward primer
tcagtggcactcgttccacatgtg

SEQ ID NO: 96 - DNA, 21 bp, Artificial Sequence
  Dengue virus 2 M gene reverse primer
tgtcattgaaggagtgacagc

SEQ ID NO: 97 - protein, 352 aa, Dengue virus type 2
DSGCVVSWKNKELKCGSGIFITDNVHTWTEQYKFQPESPSKLASAIQKAHEEGICGIRSVTRLENLMWKQITPELNHILSENEVKLTIMTGDIKGIMQAGKRSLQPQPTELKYSWKTWGKAKMLSTESHNQTFLIDGPETAECPNTNRAWNSLEVEDYGFGVFTTNIWLKLREKQDVFCDSKLMSAAIKDNRAVHADMGYWIESALNDTWKIEKASFIEVKSCHWPKSHTLWSNGVLESEMIIPKNFAGPVSQHNYRPGYHTQTAGPWHLGKLEMDFDFCEGTTVVVTEDCGNRGPSLRTTTASGKLITEWCCRSCTLPPLRYRGEDGCWYGMEIRPLKEKEENLVNSLVTA

SEQ ID NO: 98 - DNA, 21 bp, Artificial Sequence
  Dengue virus 2 NS1 gene forward primer
gatagtggttgcgttgtgagc

SEQ ID NO: 99 - DNA, 21 bp, Artificial Sequence
  Dengue virus 2 NS1 gene reverse primer
agctgtgaccaaggagttgac

SEQ ID NO: 100 - protein, 617 aa, Dengue virus type 2
AGVLWDVPSPPPVGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRKLTIMDLHPGAGKTKRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAGIFMTATPPGSRDPFPQSNAPIMDEEREIPERSWSSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGIKNNQILEENVEVEIWTKEGERKKLKPRWLDARIYSDPLTLKEFKEFAAGR

SEQ ID NO: 101 - DNA, 21 bp, Artificial Sequence
  Dengue virus 2 NS3 gene forward primer
gccggagtattgtgggatgtt

SEQ ID NO: 102 - DNA, 21 bp, Artificial Sequence
  Dengue virus 2 NS3 gene reverse primer
ctttcttccggctgcaaattc

SEQ ID NO: 103 - protein, 442 aa, Dengue virus type 3
MRCVGVGNRDFVEGLSGATWVDVVLEHGGCVTTMAKNKPTLDIELQKTEATQLATLRKLCIEGKITNITTDSRCPTQGEAILPEEQDQNYVCKHTYVDRGWGNGCGLFGKGSLVTCAKFQCLESIEGKVVQHENLKYTVIITVHTGDQHQVGNETQGVTAEITSQASTAEAILPEYGTLGLECSPRTGLDFNEMILLTMKNKAWMVHRQWFFDLPLPWTSGATTKTPTWNRKELLVTFKNAHAKKQEVVVLGSQEGAMHTALTGATEIQTSGGTSIFAGHLKCRLKMDKLKLKGMSYAMCLNTFVLKKEVSETQHGTILIKVEYKGEDAPCKIPFSTEDGQGKAHNGRLITANPVVTKKEEPVNIEAEPPFGESNIVIGIGDKALKINWYRKGSSIGKMFEATARGARRMAILGDTAWDFGSVGGVLNSLGKMVHQIFGSAY

SEQ ID NO: 104 - DNA, 25 bp, Artificial Sequence
  Dengue virus 3 E gene forward primer
caccatgagatgtgtgggagtagga

SEQ ID NO: 105 - DNA, 25 bp, Artificial Sequence
  Dengue virus 3 E gene reverse primer
ttagtaagcactcccaaatatttgg

SEQ ID NO: 106 - protein, 99 aa, Dengue virus type 3
GMSYAMCLNTFVLKKEVSETQHGTILIKVEYKGEDAPCKIPFSTEDGQGKAHNGRLITANPVVTKKEEPVNIEAEPPFGESNIVIGIGDKALKINWYRK

SEQ ID NO: 107 - DNA, 25 bp, Artificial Sequence
  Dengue virus 3 E-DIII gene forward primer
caccgggatgagctatgcaatgtgc

SEQ ID NO: 108 - DNA, 27 bp, Artificial Sequence
  Dengue virus 3 E-DIII gene reverse primer
ttacttcttgtaccagttgattttcag

SEQ ID NO: 109 - protein, 74 aa, Dengue virus type 3
SVALAPHVGMGLDTRTQTWMSAEGAWRQVEKVETWALRHPGFTILALFLAHYIGTSLTQKVVIFILLMLVTPSM

SEQ ID NO: 110 - DNA, 21 bp, Artificial Sequence
  Dengue virus 3 M gene forward primer
tcagtggcgttagctccccat

SEQ ID NO: 111 - DNA, 24 bp, Artificial Sequence
  Dengue virus 3 M gene reverse primer
tgtcatggatggggtgaccagcat

SEQ ID NO: 112 - protein, 352 aa, Dengue virus type 3
DMGCVINWKGKELKCGSGIFVTNEVHTWTEQYKFQADSPKRVATAIAGAWENGVCGIRSTTRMENLLWKQIANELNYILWENDIKLTVVVGDITGVLEQGKRTLTPQPMELKYSWKTWGLAKIVTAETQNSSFIIDGPSTPECPSASRAWNVWEVEDYGFGVFTTNIWLKLREVYTQLCDHRLMSAAVKDERAVHADMGYWIESQKNGSWKLEKASLIEVKTCTWPKSHTLWSNGVLESDMIIPKSLAGPISQHNHRPGYHTQTAGPWHLGKLELDFNYCEGTTVVISENCGTRGPSLRTTTVSGKLIHEWCCRSCTLPPLRYMGEDGCWYGMEIRPINEKEENMVKSLASA

SEQ ID NO: 113 - DNA, 21 bp, Artificial Sequence
  Dengue virus 3 NS1 gene forward primer
gacatggggtgtgtcataaac

SEQ ID NO: 114 - DNA, 18 bp, Artificial Sequence
  Dengue virus 3 NS1 gene reverse primer
cgctgagactaaagactt

SEQ ID NO: 115 - protein, 618 aa, Dengue virus type 3
SGVLWDVPSPPETQKAELEEGVYRIKQQGIFGKTQVGVGVQKEGVFHTMWHVTRGAVLTHNGKRLEPNWASVKKDLISYGGGWRLSAQWQKGEEVQVIAVEPGKNPKNFQTMPGIFQTTTGEIGAIALDFKPGTSGSPIINREGKVVGLYGNGVVTKNGGYVSGIAQTNAEPDGPTPELEEEMFKKRNLTIMDLHPGSGKTRKYLPAIVREAIKRRLRTLILAPTRVVAAEMEEAMKGLPIRYQTTATKSEHTGREIVDLMCHATFTMRLLSPVRVPNYNLIIMDEAHFTDPASIAARGYISTRVGMGEAAAIFMTATPPGTADAFPQSNAPIQDEERDIPERSWNSGNEWITDFVGKTVWFVPSIKAGNVIANCLRKNGKKVIQLSRKTFDTEYQKTKLNDWDFVVTTDISEMGANFIADRVIDPRRCLKPVILTDGPERVILAGPMPVTVASAAQRRGRVGRNPQKENDQYIFMGQPLNKDEDHAHWTEAKMLLDNINTPEGIIPALFEPEREKSAAIDGEYRLKGESRKTFVELMRRGDLPVWLAHKVASEGIKYTDRKWCFDGERNNQILEENMDVEIWTKEGEKKKLRPRWLDARTYSDPLALKEFKDFAAGR

SEQ ID NO: 116 - DNA, 21 bp, Artificial Sequence
  Dengue virus 3 NS3 gene forward primer
tccggcgttttatgggacgta

SEQ ID NO: 117 - DNA, 18 bp, Artificial Sequence
  Dengue virus 3 NS3 gene reverse primer
ctttctgccagccgcaaa

SEQ ID NO: 118 - protein, 442 aa, Dengue virus type 4
MRCVGVGNRDFVEGVSGGAWVDLVLEHGGCVTTMAQGKPTLDFELIKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDIPNHGVTATITPRSPSVEVKLPDYGELTLDCEPRSGIDFNEMILMKMKKKTWLVHKQWFLDLPLPWAAGADTSEVHWNYKERMVTFKVPHAKRQDVTVLGSQEGAMHSALTGATEVDSGDGNHMFAGHLKCKVRMEKLRIKGMSYTMCSGKFSIDKEMAETQHGTTVVKVKYEGAGAPCKVPIEIRDVNKEKVVGRIISSTPFAEYTNSVTNIELEPPFGDSYIVIGVGDSALTLHWFRKGSSIGKMLESTYRGAKRMAILGETAWDFGSVGGLLTSLGKAVHQVFGS

SEQ ID NO: 119 - DNA, 22 bp, Artificial Sequence
  Dengue virus 4 E gene forward primer
caccatgcgatgcgtgggagtg

SEQ ID NO: 120 - DNA, 24 bp, Artificial Sequence
  Dengue virus 4 E gene reverse primer
ttaactaccaaaaacctggtgtac

SEQ ID NO: 121 - protein, 103 aa, Dengue virus type 4
KGMSYTMCSGKFSIDKEMAETQHGTTVVKVKYEGAGAPCKVPIEIRDVNKEKVVGRIISSTPFAEYTNSVTNIELEPPFGDSYIVIGVGDSALTLHWFRKGSS

SEQ ID NO: 122 - DNA, 28 bp, Artificial Sequence
  Dengue virus 4 E-DIII gene forward primer
caccaagggaatgtcatacacgatgtgc

SEQ ID NO: 123 - DNA, 27 bp, Artificial Sequence
  Dengue fever 4 E-DIII gene reverse primer
ttaggaactccctttcctgaaccaatg

SEQ ID NO: 124 - protein, 74 aa, Dengue virus type 4
SVALTPHSGMGLETRAETWMSSEGAWKHAQRVESWILRNPGFALLAGFMAYMIGQTGIQRTVFFVLMMLVAPSY

SEQ ID NO: 125 - DNA, 24 bp, Artificial Sequence
  Dengue virus 4 M gene forward primer
tcagtagctttaacaccacattca

SEQ ID NO: 126 - DNA, 21 bp, Artificial Sequence
  Dengue virus 4 M gene reverse primer
tccgtaggatggggcgaccag

SEQ ID NO: 127 - protein, 351 aa, Dengue virus type 4
DTGCAVSWSGKELKCGSGIFVIDNVHTWTEQYKFQPESPARLASAILNAHEDGVCGIRSTTRLENIMWKQITNELNYVLWEGGHDLTVVAGDVKGVLSKGKRALAPPVNDLKYSWKTWGKAKIFTPEAKNSTFLIDGPDTSECPNERRAWNFLEVEDYGFGMFTTNIWMKFREGSSEVCDHRLMSAAIKDQKAVHADMGYWIESSKNQTWQIEKASLIEVKTCLWPKTHTLWSNGVLESQMLIPKAYAGPFSQHNYRQGYATQTVGPWHLGKLEIDFGECPGTTVTIQEDCDHRGPSLRTTTASGKLVTQWCCRSCTMPPLRFLGEDGCWYGMEIRPLSEKEENMVKSQVS

SEQ ID NO: 128 - DNA, 18 bp, Artificial Sequence
  Dengue virus 4 NS1 forward primer
gacatgggttgtgtggcg

SEQ ID NO: 129 - DNA, 18 bp, Artificial Sequence
  Dengue virus 4 NS1 gene reverse primer
ggccgtcacctgtgattt

SEQ ID NO: 130 - protein, 618 aa, Dengue virus type 4
RSGALWDVPSPAAAQKATLTEGVYRIMQRGLFGKTQVGVGIHMEGVFHTMWHVTRGSVICHETGRLEPSWADVRNDMISYGGGWRLGDKWDKEEDVQVLAIEPGKNPKHVQTKPGLFKTLTGEIGAVTLDFKPGTSGSPIINRKGKVIGLYGNGVVTKSGDYVSAITQAERTGEPDYEVDEDIFRKKRLTIMDLHPGAGKTKRILPSIVREALKRRLRTLILAPTRVVAAEMEEALRGLPIRYQTPAVKSEHTGREIVDLMCHATFTTRLLSSTRVPNYNLIVMDEAHFTDPSSVAARGYISTRVEMGEAAAIFMTATPPGATDPFPQSNSPIEDIEREIPERSWNTGFDWITDYQGKTVWFVPSIKAGNDIANCLRKSGKKVIQLSRKTFDTEYPKTKLTDWDFVVTTDISEMGANFRAGRVIDPRRCLKPVISTDGPERVILAGPIPVTPASAAQRRGRIGRNPAQEDDQYVFSGDPLKNDEDHAHWTEAKMLLDNIYTPEGIIPTLFGPEREKNQAIDGEFRLRGEQRKTFVELMRRGDLPVWLSYKVASAGISYKDREWCFTGERNNQILEENMEVEIWTREGEKKKLRPKWLDARVYADPMALKDFKEFASGR

SEQ ID NO: 131 - DNA, 18 bp, Artificial Sequence
  Dengue virus 4 NS3 gene forward primer
tcaggagccctgtgggac

SEQ ID NO: 132 - DNA, 21 bp, Artificial Sequence
  Dengue virus 4 NS3 gene reverse primer
cttccttccactggcaaactc

SEQ ID NO: 133 - protein, 451 aa, West Nile virus
FNCLGMSNRDFLEGVSGATWVDLVLEGDSCVTIMSKDKPTIDVKMMNMEAANLAEVRSYCYLATVSDLSTKAACPTMGEAHNDKRADPAFVCRQGVVDRGWGNGCGLFGKGSIDTCAKFACSTKAIGRTILKENIKYEVAIFVHGPTTVESHGNYSTQVGATQAGRLSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGSSIGKAFTTTLKGAQRLAALGDTAWDFGSVGGVFTSVGKAVHQVFGGAFR

SEQ ID NO: 134 - DNA, 28 bp, Artificial Sequence
  West nile virus E gene forward primer
caccttcaactgccttggaatgagcaac

SEQ ID NO: 135 - DNA, 27 bp, Artificial Sequence
  West nile virus E gene reverse primer
ttagcggaatgctcctccgaacacttg

SEQ ID NO: 136 - protein, 120 aa, West Nile virus
QLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGSSIGKAFTTTLKGA

SEQ ID NO: 137 - DNA, 28 bp, Artificial Sequence
  West nile virus E-DIII gene forward primer
cacccagttgaagggaacaacctatggc

SEQ ID NO: 138 - DNA, 24 bp, Artificial Sequence
  West nile virus E-DIII gene reverse primer
ttacgctcctttgagggtggttgt

SEQ ID NO: 139 - protein, 167 aa, West Nile virus
VTLSNFQGKVMMTVNATDVTDVITIPTAAGKNLCIVRAMDVGYMCDDTITYECPVLSAGNDPEDIDCWCTKSAVYVRYGRCTKTRHSRRSRRSLTVQTHGESTLANKKGAWMDSTKATRYLVKTESWILRNPGYALVAAVIGWMLGSNTMQRVVFVVLLLLVAPAYS

SEQ ID NO: 140 - DNA, 28 bp, Artificial Sequence
  West nile virus pM gene forward primer
caccgttaccctctctaacttccaaggg

SEQ ID NO: 141 - DNA, 24 bp, Artificial Sequence
  West nile virus pM gene reverse primer
ttagctgtaagctggggccaccaa

SEQ ID NO: 142 - protein, 352 aa, West Nile virus
DTGCAIDISRQELRCGSGVFIHNDVEAWMDRYKYYPETPQGLAKIIQKAHKEGVCGLRSVSRLEHQMWEAVKDELNTLLKENGVDLSVVVEKQEGMYKSAPKRLTATTEKLEIGWKAWGKSILFAPELANNTFVVDGPETKECPTQNRAWNSLEVEDFGFGLTSTRMFLKVRESNTTECDSKIIGTAVKNNLAIHSDLSYWIESRLNDTWKLERAVLGEVKSCTWPETHTLWGDGILESDLIIPVTLAGPRSNHNRRPGYKTQNQGPWDEGRVEIDFDYCPGTTVTLSESCGHRGPATRTTTESGKLITDWCCRSCTLPPLRYQTDSGCWYGMEIRPQRHDEKTLVQSQVNA

SEQ ID NO: 143 - DNA, 19 bp, Artificial Sequence
  West nile virus NS1 gene forward primer
caccgacactgggtgtgcc

SEQ ID NO: 144 - DNA, 21 bp, Artificial Sequence
  West nile virus NS1 gene reverse primer
agcattcacttgtgactgcac

SEQ ID NO: 145 - protein, 443 aa, Yellow fever virus
AHCIGITDRDFIEGVHGGTWVSATLEQDKCVTVMAPDKPSLDISLETVAIDRPAEVRKVCYNAVLTHVKINDKCPSTGEAHLAEENEGDNACKRTYSDRGWGNGCGLFGKGSIVACAKFTCAKSMSLFEVDQTKIQYVIRAQLHVGAKQENWTTDIKTLKFDALSGSQEVEFIGYGKATLECQVQTAVDFGNSYIAEMETESWIVDRQWAQDLTLPWQSGSGGVWREMHHLVEFEPPHAATIRVLALGNQEGSLKTALTGAMRVTKDTNDNNLYKLHGGHVSCRVKLSALTLKGTSYKICTDKMFFVKNPTDTGHGTVVMQVKVSKGAPCRIPVIVADDLTAAINKGILVTVNPIASTNDDEVLIEVNPPFGDSYIIVGRGDSRLTYQWHKEGSSIGKLFTQTMKGVERLAVMGDTAWDFSSAGGFFTSVGKGIHTVFGSAFQ

SEQ ID NO: 146 - DNA, 22 bp, Artificial Sequence
  Yellow fever virus E gene forward primer
caccgctcactgcattggaatt

SEQ ID NO: 147 - DNA, 24 bp, Artificial Sequence
  Yellow fever virus E gene reverse primer
ttactgaaaggcagagccaaacac

SEQ ID NO: 148 - protein, 111 aa, Yellow fever virus
SALTLKGTSYKICTDKMFFVKNPTDTGHGTVVMQVKVSKGAPCRIPVIVADDLTAAINKGILVTVNPIASTNDDEVLIEVNPPFGDSYIIVGRGDSRLTYQWHKEGSSIGK

SEQ ID NO: 149 - DNA, 25 bp, Artificial Sequence
  Yellow fever virus E-DIII gene forward primer
cacctcagctttgacactcaagggg

SEQ ID NO: 150 - DNA, 24 bp, Artificial Sequence
  Yellow fever virus E-DIII gene reverse primer
ttactttcctattgagcttccctc

SEQ ID NO: 151 - protein, 164 aa, Yellow fever virus
VTLVRKNRWLLLNVTSEDLGKTFSVGTGNCTTNILEAKYWCPDSMEYNCPNLSPREEPDDIDCWCYGVENVRVAYGKCDSAGRSRRSRRAIDLPTHENHGLKTRQEKWMTGRMGERQLQKIERWFVRNPFFAVTALTIAYLVGSNMTQRVVIALLVLAVGPAYS

SEQ ID NO: 152 - DNA, 25 bp, Artificial Sequence
  Yellow fever virus pM gene forward primer
caccgtgaccttggtgcggaaaaac

SEQ ID NO: 153 - DNA, 21 bp, Artificial Sequence
  Yellow fever virus pM gene reverse primer
tgagtaggccggaccaacagc

SEQ ID NO: 154 - protein, 352 aa, Yellow fever virus
DQGCAINFGKRELKCGDGIFIFRDSDDWLNKYSYYPEDPVKLASIVKASFEEGKCGLNSVDSLEHEMWRSRADEINAIFEENEVDISVVVQDPKNVYQRGTHPFSRIRDGLQYGWKTWGKNLVFSPGRKNGSFIIDGKSRKECPFSNRVWNSFQIEEFGTGVFTTRVYMDAVFEYTIDCDGSILGAAVNGKKSAHGSPTFWMGSHEVNGTWMIHTLEALDYKECEWPLTHTIGTSVEESEMFMPRSIGGPVSSHNHIPGYKVQTNGPWMQVPLEVKREACPGTSVIIDGNCDGRGKSTRSTTDSGKVIPEWCCRSCTMPPVSFHGSDGCWYPMEIRPRKTHESHLVRSWVTA

SEQ ID NO: 155 - DNA, 25 bp, Artificial Sequence
  Yellow fever virus NS1 gene forward primer
caccgatcaaggatgcgccatcaac

SEQ ID NO: 156 - DNA, 21 bp, Artificial Sequence
  Yellow fever virus NS1 gene reverse primer
agctgtaacccaggagcgcac

SEQ ID NO: 157 - protein, 623 aa, Yellow fever virus
SGDVLWDIPTPKIIEECEHLEDGIYGIFQSTFLGASQRGVGVAQGGVFHTMWHVTRGAFLVRNGKKLIPSWASVKEDLVAYGGSWKLEGRWDGEEEVQLIAAVPGKNVVNVQTKPSLFKVRNGGEIGAVALDYPSGTSGSPIVNRNGEVIGLYGNGILVGDNSFVSAISQTEVKEEGKEELQEIPTMLKKGMTTVLDFHPGAGKTRRFLPQILAECARRRLRTLVLAPTRVVLSEMKEAFHGLDVKFHTQAFSAHGSGREVIDAMCHATLTYRMLEPTRVVNWEVIIMDEAHFLDPASIAARGWAAHRARANESATILMTATPPGTSDEFPHSNGEIEDVQTDIPSEPWNTGHDWILADKRPTAWFLPSIRAANVMAASLRKAGKSVVVLNRKTFEREYPTIKQKKPDFILATDIAEMGANLCVERVLDCRTAFKPVLVDEGRKVAIKGPLRISASSAAQRRGRIGRNPNRDGDSYYYSEPTSENNAHHVCWLEASMLLDNMEVRGGMVAPLYGVEGTKTPVSPGEMRLRDDQRKVFRELVRNCDLPVWLSWQVAKAGLKTNDRKWCFEGPEEHEILNDSGETVKCRAPGGAKKPLRPRWCDERVSSDQSALSEFIKFAEGRR

SEQ ID NO: 158 - DNA, 21 bp, Artificial Sequence
  Yellow fever virus NS3 gene forward primer
agtggggatgtcttgtgggat

SEQ ID NO: 159 - DNA, 24 bp, Artificial Sequence
  Yellow fever virus NS3 gene reverse primer
cctcctaccttcagcaaacttaat

SEQ ID NO: 160 - protein, 446 aa, Japanese encephalitis virus
GMGNRDFIEGASGATWVDLVLEGDSCLTIMANDKPTLDVRMINIEASQLAEVRSYCYHASVTDISTVARCPTTGEAHNEKRADSSYVCKQGFTDRGWGNGCGFFGKGSIDTCAKFSCTSKAIGRTIQPENIKYKVGIFVHGTTTSENHGNYSAQVGASQAAKFTVTPNAPSVALKLGDYGEVTLDCEPRSGLNTEAFYVMTVGSKSFLVHREWFHDLALPWTSPSSTAWRNRELLMEFEGAHATKQSVVALGSQEGGLHHALAGAIVVEYSSSVMLTSGHLKCRLKMDKLALKGTTYGMCTEKFSFAKNPVDTGHGTVVIELSYSGSDGPCKIPIVSVASLNDMTPVGRLVTVNPFVATSSANSKVLVEMEPPFGDSYIVVGRGDKQINHHWHKAGSTLGKAFSTTLKGAQRLAALGDTAWDFGSIGGVFNSIGRAVHQVFGDAFR

SEQ ID NO: 161 - DNA, 25 bp, Artificial Sequence
  Japanese encephalitis virus E gene forward primer
cacctttaattgtctgggaatgggc

SEQ ID NO: 162 - DNA, 27 bp, Artificial Sequence
  Japanese encephalitis virus E gene reverse primer
ttatctgaaggcatcaccaaacacttg

SEQ ID NO: 163 - protein, 111 aa, Japanese encephalitis virus
DKLALKGTTYGMCTEKFSFAKNPVDTGHGTVVIELSYSGSDGPCKIPIVSVASLNDMTPVGRLVTVNPFVATSSANSKVLVEMEPPFGDSYIVVGRGDKQINHHWHKAGST

SEQ ID NO: 164 - DNA, 25 bp, Artificial Sequence
  Japanese encephalitis virus E-DIII gene forward primer
caccgacaaactggctctgaaaggc

SEQ ID NO: 165 - DNA, 27 bp, Artificial Sequence
  Japanese encephalitis virus E-DIII gene reverse primer
ttacgtgcttccagctttgtgccaatg

SEQ ID NO: 166 - protein, 167 aa, Japanese encephalitis virus
MKLSNFQGKLLMTINNTDIADVIVIPTSKGENRCWVRAIDVGYMCEDTITYECPKLTMGNDPEDVDCWCDNQEVYVQYGRCTRTRHSKRSRRSVSVQTHGESSLVNKKEAWLDSTKATRYLMKTENWIIRNPGYAFLAAVLGWMLGSNNGQRVVFTILLLLVAPAYS

SEQ ID NO: 167 - DNA, 25 bp, Artificial Sequence
  Japanese encephalitis virus pM gene forward virus
caccatgaagttgtcgaatttccag

SEQ ID NO: 168 - DNA, 25 bp, Artificial Sequence
  Japanese encephalitis virus pM gene reverse primer
ttaactgtaagccggagcgaccaac

SEQ ID NO: 169 - protein, 352 aa, Japanese encephalitis virus
DTGCAIDITRKEMRCGSGIFVHNDVEAWVDRYKYLPETPRSLAKIVHKAHKEGVCGVRSVTRLEHQMWEAVRDELNVLLKENAVDLSVVVNKPVGRYRSAPKRLSMTQEKFEMGWKAWGKSILFAPELANSTFVVDGPETKECPDEHRAWNSMQIEDFGFGITSTRVWLKIREESTDECDGAIIGTAVKGHVAVHSDLSYWIESRYNDTWKLERAVFGEVKSCTWPETHTLWGDDVEESELIIPHTIAGPKSKHNRREGYKTQNQGPWDENGIVLDFDYCPGTKVTITEDCSKRGPSVRTTTDSGKLITDWCCRSCSLPPLRFRTENGCWYGMEIRPVMHDETTLVRSQVHA

SEQ ID NO: 170 - DNA, 25 bp, Artificial Sequence
  Japanese encephalitis virus NS1 gene forward primer
caccgacactggatgtgccattgac

SEQ ID NO: 171 - DNA, 24 bp, Artificial Sequence
  Japanese encephalitis virus NS1 gene reverse primer
agcatgaacctgtgatctgacgag

SEQ ID NO: 172 - protein, 451 aa, St. Louis encephalitis virus
FNCLGTSNRDFVEGASGATWIDLVLEGGSCVTVMAPEKPTLDFKVMKMEATELATVREYCYEATLDTLSTVARCPTTGEAHNTKRSDPTFVCKRDVVDRGWGNGCGLFGKGSIDTCAKFTCKNKATGKTFLRENIKYEVAIFVHGSTDSTSHGNYFEQIGKNQAARFTISPQAPSFTASMGEYGTVTIDCEARSGINTEDYYVFTVKEKSWLVNRDWFHDLNLPWTSPATTDWRNRETLVEFEEPHATKQTVVALGSQEGALHTALAGAIPATVSSSTLTLQSGHLKCRAKLDKVKIKGTTYGMCDSAFTFSKNPTDTGHGTVIVELQYTGSNGPCRVPISVTANLMDLTPVGRLVTVNPFISTGGANNKVMIEVEPPFGDSYIVVGRGTTQINYHWHKEGSSIGKALATTWKGAQRLAVLGDTAWDFGSIGGVFNSIGKAVHQVFGGAFR

SEQ ID NO: 173 - DNA, 25 bp, Artificial Sequence
  Saint Louis encephalitis virus E gene forward primer
caccttcaactgtttgggaacatca

SEQ ID NO: 174 - DNA, 24 bp, Artificial Sequence
  Saint Louis encephalitis virus E gene reverse primer
ttacctaaatgctcctccaaaaac

SEQ ID NO: 175 - protein, 106 aa, St. Louis encephalitis virus
GTTYGMCDSAFTFSKNPTDTGHGTVIVELQYTGSNGPCRVPISVTANLMDLTPVGRLVTVNPFISTGGANNKVMIEVEPPFGDSYIVVGRGTTQINYHWHKEGSSI

SEQ ID NO: 176 - DNA, 28 bp, Artificial Sequence
  Saint Louis encephalitis virus E-DIII gene forward primer
caccggaacgacatatggtatgtgtgac

SEQ ID NO: 177 - DNA, 24 bp, Artificial Sequence
  Saint Louis encephalitis virus E-DIII gene reverse primer
ttaaatgctgcttccctctttgtg

SEQ ID NO: 178 - protein, 167 aa, St. Louis encephalitis virus
LQLSTYQGKVLMSINKTDAQSAINIPSANGANTCIVRALDVGVMCKDDITYLCPVLSAGNDPEDIDCWCDVKEVWVHYGRCTRMGHSRRSRRSISVQHHGDSTLATKNTPWLDTVKTTKYLTKVENWVLRNPGYALVALAIGWMLGSNNTQRVVFVIMLMLIAPAYS

SEQ ID NO: 179 - DNA, 28 bp, Artificial Sequence
  Saint Louis encephalitis virus pM gene forward primer
caccttgcagttatcaacctatcagggg

SEQ ID NO: 180 - DNA, 24 bp, Artificial Sequence
  Saint Louis encephalitis virus pM gene reverse primer
ttagctgtatgccggagcaatcag

SEQ ID NO: 181 - protein, 356 aa, St. Louis encephalitis virus
  MISC_FEATURE (355)..(355): Xaa may be any amino acid
DSGCAIDLQRRELKCGGGIFVYNDVEKWKSDYKYFPLTPTGLARVIQEAHANGICGIRSTSRLEHLMWENIQRELNAIFEDNEIDLSVVVQEDPKYYKRAPRRLKKLEDELDYGWKKWGKTLFVEPILGNNTFVVDGPETKECPTANRAWNSFKVEDFGFGMVFTRLWLTIREENTTECDSAIIGTAIKGDRAVHSDLSYWIESKKNETWQLERAVMGEVKSCTWPETHTLWGDGVVESEMIIPVTLGGPKSHHNKRNGYHTQTKGPWSEGEITLDFDYCPGTTVTVTEHCGNRGASLRTTTASGKLVTDWCCRSCSLPPLRYTTKDGCWYGMEIRPVKEEEAKLVKSRVTAGVXG

SEQ ID NO: 182 - DNA, 22 bp, Artificial Sequence
  Saint Louis encephalitis virus NS1 gene forward primer
caccgattcgggatgtgcaatt

SEQ ID NO: 183 - DNA, 21 bp, Artificial Sequence
  Saint Louis encephalitis virus NS1 gene reverse primer
agctgtcactcgagatttcac

SEQ ID NO: 184 - protein, 451 aa, Murray Valley encephalitis virus
FNCLGMSSRDFIEGASGATWVDLVLEGDSCITIMAADKPTLDIRMMNIEATNLALVRNYCYAATVSDVSTVSNCPTTGESHNTKRADHNYLCKRGVTDRGWGNGCGLFGKGSIDTCAKFTCSNSAAGRLILPEDIKYEVGVFVHGSTDSTSHGNYSTQIGANQAVRFTISPNAPAITAKMGDYGEVTVECEPRSGLNTEAYYVMTIGTKHFLVHREWFNDLLLPWTSPASTEWRNREILVEFEEPHATKQSVVALGSQEGALHQALAGAIPVEFSSSTLKLTSGHLKCRVKMEKLKLKGTTYGMCTEKFTFSKNPADTGHGTVVLELQYTGSDGPCKIPISSVASLNDMTPVGRMVTANPYVASSTANAKVLVEIEPPFGDSYIVVGRGDKQINHHWHKEGSSIGKAFSTTLKGAQRLAALGDTAWDFGSVGGVFNSIGKAVHQVFGGAFR

SEQ ID NO: 185 - DNA, 22 bp, Artificial Sequence
  Murray Valley encephalitis virus E gene forward primer
cacctttaattgcctgggtatg

SEQ ID NO: 186 - DNA, 27 bp, Artificial Sequence
  Murray Valley encephalitis virus E gene reverse primer
ttaacgaaatgcaccaccaaaaacctg

SEQ ID NO: 187 - protein, 109 aa, Murray Valley encephalitis virus
KGTTYGMCTEKFTFSKNPADTGHGTVVLELQYTGSDGPCKIPISSVASLNDMTPVGRMVTANPYVASSTANAKVLVEIEPPFGDSYIVVGRGDKQINHHWHKEGSSIGK

SEQ ID NO: 188 - DNA, 25 bp, Artificial Sequence
  Murray Valley encephalitis virus E-DIII gene forward primer
caccaaaggaaccacttatgggatg

SEQ ID NO: 189 - DNA, 24 bp, Artificial Sequence
  Murray Valley encephalitis virus E-DIII gene reverse primer
ttatttgccaattgaactaccctc

SEQ ID NO: 190 - protein, 75 aa, Murray Valley encephalitis virus
SITVQTHGESTLVNKKDAWLDSTKATRYLTKTENWIIRNPGYALVAVVLGWMLGSNTGQKVIFTVLLLLVAPAYS

SEQ ID NO: 191 - DNA, 28 bp, Artificial Sequence
  Murray Valley encephalitis virus M gene forward primer
caccttaaagctttccaccttccagggc

SEQ ID NO: 192 - DNA, 24 bp, Artificial Sequence
  Murray Valley encephalitis virus M gene reverse primer
ctcgttgctcctgcctacagttaa

SEQ ID NO: 193 - protein, 352 aa, Murray Valley encephalitis virus
DTGCAIDITRRELKCGSGIFIHNDVEAWIDRYKYLPETPKQLAKVVENAHKSGICGIRSVNRFEHQMWESVRDELNALLKENAIDLSVVVEKQKGMYRAAPNRLRLTVEELDIGWKAWGKSLLFAAELANSTFVVDGPETAECPNSKRAWNSFEIEDFGFGITSTRGWLKLREENTSECDSTIIGTAVKGNHAVHSDLSYWIESGLNGTWKLERAIFGEVKSCTWPETHTLWGDAVEETELIIPVTLAGPRSKHNRREGYKVQVQGPWDEEDIKLDFDYCPGTTVTVSEHCGKRGPSVRTTTDSGKLVTDWCCRSCTLPPLRFTTASGCWYGMEIRPMKHDESTLVKSRVQA

SEQ ID NO: 194 - DNA, 18 bp, Artificial Sequence
  Murray Valley encephalitis virus NS1 gene forward primer
caccgataccggttgtgc

SEQ ID NO: 195 - DNA, 24 bp, Artificial Sequence
  Murray Valley encephalitis virus NS1 gene reverse primer
ttatgcctgaacacggcttttaac

SEQ ID NO: 196 - protein, 451 aa, Rocio virus
INCLGVTNRDFVEGMSGGTWVDIVLEGDGCVTIMAKDKPTLDIRLLKMEAKDLATVRSYCYHATVTSVSSEARCPTMGEAHNPKALDSSYLCKSTYVDRGWGNGCGLFGKGSLQTCVKFGCTQKAMGMTIQRENLDYELAIYVHGPTSVAAHGNYTTQLGAKHAAKFSITPSSPSFTANLGEYGEATVDCEPRAALDIDNYYVMSMNNKHWLVNRDWFHDLDLPWTGPATDVWKNRESLVEFEEAHATRQTVVALAAQEGALHTALAGAIPVTVAGTTLTLTSGHLKCRMKLDKLKIKGSTYLMCKDKFAFAKNPVDTGHGTIVTEVQYAGSDGPCRIPITMTENLHDLTPIGRLVTVNPFVPSSETAQKILIELEPPFGTSFILVGTGPNQVKYQWHKSGSVIGSAFKTTIKGAQRMAVLGETAWDFGSVGGVFNSIGKGIHGLFGGAFR

SEQ ID NO: 197 - DNA, 25 bp, Artificial Sequence
  Rocio virus E gene forward primer
caccatcaactgcttgggtgtgacc

SEQ ID NO: 198 - DNA, 24 bp, Artificial Sequence
  Rocio virus E gene reverse primer
ttatctaaacgctcctccgaagag

SEQ ID NO: 199 - protein, 109 aa, Rocio virus
KGSTYLMCKDKFAFAKNPVDTGHGTIVTEVQYAGSDGPCRIPITMTENLHDLTPIGRLVTVNPFVPSSETAQKILIELEPPFGTSFILVGTGPNQVKYQWHKSGSVIGS

SEQ ID NO: 200 - DNA, 28 bp, Artificial Sequence
  Rocio virus E-DIII gene forward primer
caccaaagggtcaacatacctgatgtgc

SEQ ID NO: 201 - DNA, 24 bp, Artificial Sequence
  Rocio virus E-DIII gene reverse primer
ttagctcccgatcacactaccaga

SEQ ID NO: 202 - protein, 167 aa, Rocio virus
LRLGTYQGKVLMSINKTDVAEIIPIPTTKGDNLCTVRAMDVGYMCQKDITYECPRLEPGMDPEDIDCWCDREAIYVHYGLCTKNHRERRGRRSVNIPSHGESQLENRGTPWLDTAKTTKYLTKVENWMIRNPGYAIVAVAAAWMLGSNTSQKVIFTIMLLLIAPAYS

SEQ ID NO: 203 - DNA, 25 bp, Artificial Sequence
  Rocio virus pM gene forward primer
caccctgcgcctggggacatatcaa

SEQ ID NO: 204 - DNA, 24 bp, Artificial Sequence
  Rocio virus pM gene reverse primer
ttagctgtaggctggggctatcag

SEQ ID NO: 205 - protein, 355 aa, Rocio virus
TGCAIDITRRELKCGSGIFIHNDVETWRDNYKYHPSTPKNFAKIIHKAYKEGICGVRSASRLEHEMWKHIAPELNAILEDNEVDLSVVVEEHKGIYKKAPLRLENTSDEMHFGWKNWGKSFLFKTQMANSTFVVDGPETKECPTERRAWNSLEIEDFGVGIMSTKVFLKVNGDKTEVCDSMVMGTAIKGNRAVHSDLGYWIESGKNTSWRLERAVLGEVRSCTWPESHTLWNEGVEDSDLIIPPTLGGPRTHHNKREGYKTQLKGPWNEEGPIIIEFGECPGTKVTQEESCRNRAASARTTTASGKVIRDWCCKNCTMPPLRFTTKNGCWYGMEIRPKHESEETLIKSKVTAGTG

SEQ ID NO: 206 - DNA, 22 bp, Artificial Sequence
  Rocio virus NS1 gene forward primer
caccacaggatgcgcgattgac

SEQ ID NO: 207 - DNA, 21 bp, Artificial Sequence
  Rocio virus NS1 gene reverse primer
tccggtcccagctgtcacttt

SEQ ID NO: 208 - protein, 619 aa, Rocio virus
GGVLWDLPAPKQMGRSDMKPGVYRVMTSGVLGSYQSGVGVMYDGVFHTMWHVTQGAALRNGEGRLNPTWGSVRDDLITYGGKWKLSATWDGTEEVQLIAAEPGKPVKNFQTRPGVFKTPAGEVGAITLDFPKGTSGSPIVNKAGAVIGLYGNGLVLSSGAYVSAISQGERQEEEAPEAFTPEMLRKRQLTILDLHPGAGKTRRVIPQIVREAVKQRLRTVILAPTRVVAAEIAEALRGLPVRFQTSAVKAEHSGTEIVDVMCHATLTQRLMTPMRVPNYNVFVMDEAHFTDPASIAARGYISTKVESGEAAAIFMTATPPGTIDPFPDSNSPIIDQEAEIPDRAWNSGFEWITDYTGKTVWFVPSVRSGNEIAMCLTKAGKKVIQLNRKSYETEYQKCKGNDWDYVVTTDISEMGANFGAHRVIDSRKCVKPVIINDGEGRVQLNGPLPITASSAAQRRGRVGRDPTQSGDEYYYGGPITNDDTGHAHWIEAKMLLDNIQLQNGLVAQLYKPERDKVFATDGEYRLRGEQKKHFVELMRTGELPVWLSYKVAEAGINYTDRRWCFDGPHNNTILEDNTEVEIWTRQGERKVLRPRWSDARVYSDNQALRAFKEFAAGKR

SEQ ID NO: 209 - DNA, 19 bp, Artificial Sequence
  Rocio virus NS3 gene forward primer
caccggtggtgttctgtgg

SEQ ID NO: 210 - DNA, 21 bp, Artificial Sequence
  Rocio virus NS3 gene reverse primer
gcgcttccccgccgcaaattc

SEQ ID NO: 211 - protein, 447 aa, Powassan virus
TRCTHLENRDFVTGTQGTTRVSLVLELGGCVTITAEGKPSIDVWLEDIFQESPAETREYCLHAKLTNTKVEARCPTTGPATLPEEHQANMVCKRDQSDRGWGNHCGFFGKGSIVACAKFECEEAKKAVGHVYDSTKITYVVKVEPHTGDYLAANETNSNRKSAQFTVASEKVILRLGDYGDVSLTCKVASGIDVAQTVVMSLDSSKDHLPSAWQVHRDWFEDLALPWKHKDNQDWNSVEKLVEFGPPHAVKMDVFNLGDQTAVLLKSLAGVPLASVEGQKYHLKSGHVTCDVGLEKLKLKGTTYSMCDKAKFKWKRVPVDSGHDTVVMEVSYTGSDKPCRIPVRAVAHGVPAVNVAMLITPNPTIETNGGGFIEMQLPPGDNIIYVGDLSQQWFQKGSTIGRMFEKTRRGLERLSVVGEHAWDFGSVGGVLSSVGKAIHTVLGGAFN

SEQ ID NO: 212 - DNA, 21 bp, Artificial Sequence
  Powassan virus E gene forward primer
caccactcgctgtacacacct

SEQ ID NO: 213 - DNA, 21 bp, Artificial Sequence
  Powassan virus E gene reverse primer
ttagttaaatgcgccgccgag

SEQ ID NO: 214 - protein, 96 aa, Powassan virus
KGTTYSMCDKAKFKWKRVPVDSGHDTVVMEVSYTGSDKPCRIPVRAVAHGVPAVNVAMLITPNPTIETNGGGFIEMQLPPGDNIIYVGDLSQQWFQ

SEQ ID NO: 215 - DNA, 25 bp, Artificial Sequence
  Powassan virus E-DIII gene forward primer
caccaaagggacgacgtacagtatg

SEQ ID NO: 216 - DNA, 24 bp, Artificial Sequence
  Powassan virus E-DIII gene reverse primer
ttattgaaaccactgctgtgacag

SEQ ID NO: 217 - protein, 168 aa, Powassan virus
MAMATTIHRDREGYMVMRASGRDAASQVRVQNGTCVILATDMGEWCEDSITYSCVTIDQEEEPVDVDCFCRGVDRVKLEYGRCGRQAGSRGKRSVVIPTHAQKDMVGRGHAWLKGDNIRDHVTRVEGWMWKNKLLTAAIVALAWLMVDSWMARVTVILLALSLGPVYA

SEQ ID NO: 218 - DNA, 21 bp, Artificial Sequence
  Powassan virus pM gene forward primer
caccatggcgatggctaccac

SEQ ID NO: 219 - DNA, 21 bp, Artificial Sequence
  Powassan virus pM gene reverse primer
tcacgcgtacacaggacccag

SEQ ID NO: 220 - protein, 353 aa, Powassan virus
DYGCAIDPERMEIRCGEGLVVWKEVSEWYDGYAYHPESPDTLAQALREAFERGVCGVVPQNRLEMAMWRSTAPELNLVLSEGEANLTIVVDKTDPADYRGGTPMVLKKTGKESKVSWKSWGKSILWSVPDSPRRMMMGVDGVGECPLYRRATGVFTVAEFGVGLRTKVFLDLRGEASKECDTGVMGAAVKNGKAIHTDQSMWMSSFRNDTGTYIHELILTDLRNCTWPASHTIDNDGVLDSHLFLPVTLAGPRSKYNRIPGYSEQVRGPWDQTPLRVVRDHCPGTSVRIDSHCDKRGASVRSTTESGKIIPEWCCRACELPPVTFRSGTDCWYAMEIRPVHSQGGLVRSMVVA

SEQ ID NO: 221 - DNA, 21 bp, Artificial Sequence
  Powassan virus NS1 gene forward primer
caccgattacggctgcgcaat

SEQ ID NO: 222 - DNA, 21 bp, Artificial Sequence
  Powassan virus NS1 gene reverse primer
ttacgcaacgaccatagaacg

SEQ ID NO: 223 - protein, 446 aa, Tick-borne encephalitis virus
SRCTHLENRDFVTGTQGTTRVTLVLELGGCVTITAEGKPSMDVWLDSIYQENPAKTREYCLHAKLSDTKVAARCPTMGPATLAEEHQSGTVCKRDQSDRGWGNHCGLFGKGSIVTCVKASCEAKKKATGHVYDANKIVYTVKVEPHTGDYVAVNETHSGRKTASFTVSSEKTILTMGDYGDVSLLCRVASGVDLAQTVILELDKTSEHLPTAWQVHRDWFNDLALPWKHEGAQNWNNAERLVEFGAPHAVKMDVYNLGDQTGVLLKSLAGVPVAHIDGTKYHLKSGHVTCEVGLEKLKMKGLTYTMCDKTKFTWKRIPTDSGHDTVVMEVAFSGTKPCRIPVRAVAHGSPDVNVAMLITPNPTIENNGGGFIEMQLPPGDNIIYVGELSHQWFQKGSSIGRVFQKTRKGIERLTVIGEHAWDFGSTGGFLTSVGKALHTVLGGAFN

SEQ ID NO: 224 - DNA, 22 bp, Artificial Sequence
  Tick-borne encephalitis virus strain SOFJIN-HO E gene forward primer
cacctcacgttgcacacatctg

SEQ ID NO: 225 - DNA, 24 bp, Artificial Sequence
  Tick-borne encephalitis virus strain SOFJIN-HO E gene reverse primer
ttaattaaacgcaccaccgagtac

SEQ ID NO: 226 - protein, 96 aa, Tick-borne encephalitis virus
KGLTYTMCDKTKFTWKRIPTDSGHDTVVMEVAFSGTKPCRIPVRAVAHGSPDVNVAMLITPNPTIENNGGGFIEMQLPPGDNIIYVGELSHQWFQK

SEQ ID NO: 227 - DNA, 22 bp, Artificial Sequence
  Tick-borne encephalitis virus strain SOFJIN-HO E-DIII gene forward primer
caccaagggcctgacgtatacg

SEQ ID NO: 228 - DNA, 24 bp, Artificial Sequence
  Tick-borne encephalitis virus strain SOFJIN-HO E-DIII gene reverse primer
ttatttctgaaaccactggtggct

SEQ ID NO: 229 - protein, 168 aa, Tick-borne encephalitis virus
VTLAATVRKERDGTTVIRAEGKDAATQVRVENGTCVILATDMGSWCDDSLTYECVTIDQGEEPVDVDCFCRNVDGVYLEYGRCGKQEGSRTRRSVLIPSHAQGDLTGRGHKWLEGDSLRTHLTRVEGWVWKNKMLTLAVIAVVWLTVESVVTRVAVVVVLLCLAPVYA

SEQ ID NO: 230 - DNA, 22 bp, Artificial Sequence
  Tick-borne encephalitis virus strain SOFJIN-HO pM gene forward primer
caccatgaccttggcagcgacg

SEQ ID NO: 231 - DNA, 24 bp, Artificial Sequence
  Tick-borne encephalitis virus strain SOFJIN-HO pM gene reverse primer
tcaagcgtataccggagccaggca

SEQ ID NO: 232 - protein, 352 aa, Tick-borne encephalitis virus
DVGGAVDTERMELRCGEGLVVWREVSEWYDNYAYYPETPGALASAIKETFEEGTCGIVPQNRLEMAMWRSSATELNLALAEGDANLTVVVDKLDPTDYRGGIPGLLKKGKDIKVSWKSWGHSMIWSVPEAPRRFMVGTEGSSECPLERRKTGVFTVAEFGVGLRTKVFLDFRQESTHECDTGVMGAAVKNGMAVHTDQSLWMKSVRNDTGTYIVELLVTDLRNCSWPASHTIDNAEVVDSELFLPASLAGPRSWYNRIPGYSEQVKGPWKYSPIRVTREECPGTRVTINADCDKRGASVRSTTESGKVIPEWCCRTCTLPPVTFRTGTDCWYAMEIRPVHDQGGLIRSMVVA

SEQ ID NO: 233 - DNA, 19 bp, Artificial Sequence
  Tick-borne encephalitis virus strain SOFJIN-HO NS1 gene forward primer
caccgatgttggttgtgcg

SEQ ID NO: 234 - DNA, 21 bp, Artificial Sequence
  Tick-borne encephalitis virus strain SOFJIN-HO NS1 gene reverse primer
ttacgcgaccaccattgagcg

SEQ ID NO: 235 - protein, 446 aa, Tick-borne encephalitis virus
SRCTHLENRDFVTGTQGTTRVTLVLELGGCVTITAEGKPSMDVWLDAIYQENPAKTREYCLHAKLSDTKVAARCPTMGPATLAEEHQGGTVCKRDQSDRGWGNHCGLFGKGSIVACVKAACEAKKKATGHVYDANKIVYTVKVEPHTGDYVAANETHSGRKTASFTISSEKTILTMGEYGDVSLLCRVASGVDLAQTVILELDKTVEHLPTAWQVHRDWFNDLALPWKHEGAQNWNNAERLVEFGAPHAVKMDVYNLGDQTGVLLKALAGVPVAHIEGTKYHLKSGHVTCEVGLEKLKMKGLTYTMCDKTKFTWKRAPTDSGHDTVVMEVTFSGTKPCRIPVRAVAHGSPDVNVAMLITPNPTIENNGGGFIEMQLPPGDNIIYVGELSHQWFQKGSSIGRVFQKTKKGIERLTVIGEHAWDFGSAGGFLSSIGKAVHTVLGGAFN

SEQ ID NO: 236 - DNA, 22 bp, Artificial Sequence
  Tick-borne encephalitis virus strain NEUDOERFL E gene forward primer
caccagccgttgtactcatctg

SEQ ID NO: 237 - DNA, 24 bp, Artificial Sequence
  Tick-borne encephalitis virus strain NEUDOERFL E gene reverse primer
ttaattgaaagctccgccaagcac

SEQ ID NO: 238 - protein, 96 aa, Tick-borne encephalitis virus
KGLTYTMCDKTKFTWKRAPTDSGHDTVVMEVTFSGTKPCRIPVRAVAHGSPDVNVAMLITPNPTIENNGGGFIEMQLPPGDNIIYVGELSHQWFQK

SEQ ID NO: 239 - DNA, 28 bp, Artificial Sequence
  Tick-borne encephalitis virus strain NEUDOERFL E-DIII gene forward primer
caccaaaggcttaacctacacaatgtgt

SEQ ID NO: 240 - DNA, 27 bp, Artificial Sequence
  Tick-borne encephalitits virus strain NEUDOERFL E-DIII gene reverse primer
ttatttctgaaaccactgatgagacag

SEQ ID NO: 241 - protein, 168 aa, Tick-borne encephalitis virus
MTLAATVRKERDGSTVIRAEGKDAATQVRVENGTCVILATDMGSWCDDSLSYECVTIDQGEEPVDVDCFCRNVDGVYLEYGRCGKQEGSRTRRSVLIPSHAQGELTGRGHKWLEGDSLRTHLTRVEGWVWKNKLLALAMVTVVWLTLESVVTRVAVLVVLLCLAPVYA

SEQ ID NO: 242 - DNA, 19 bp, Artificial Sequence
  Tick-borne encephalitis virus strain NEUDOERFL pM gene forward primer
caccgtgactctggcagcg

SEQ ID NO: 243 - DNA, 18 bp, Artificial Sequence
  Tick-borne encephalitis virus strain NEUDOERFL pM reverse primer
tcatgcgtagaccggcgc

SEQ ID NO: 244 - protein, 352 aa, Tick-borne encephalitis virus
DVGCAVDTERMELRCGEGLVVWREVSEWYDNYAYYPETPGALASAIKETFEEGSCGVVPQNRLEMAMWRSSVTELNLALAEGEANLTVVVDKFDPTDYRGGVPGLLKKGKDIKVSWKSWGHSMIWSIPEAPRRFMVGTEGQSECPLERRKTGVFTVAEFGVGLRTKVFLDFRQEPTHECDTGVMGAAVKNGMAIHTDQSLWMRSMKNDTGTYIVELLVTDLRNCSWPASHTIDNADVVDSELFLPASLAGPRSWYNRIPGYSEQVKGPWKYTPIRVIREECPGTTVTINAKCDKRGASVRSTTESGKVIPEWCCRACTMPPVTFRTGTDCWYAMEIRPVHDQGGLVRSMVVA

SEQ ID NO: 245 - DNA, 22 bp, Artificial Sequence
  Tick-borne encephalitis virus strain NEUDOERFL NS1 gene forward primer
caccgacgtgggtggtgccgtt

SEQ ID NO: 246 - DNA, 24 bp, Artificial Sequence
  Tick-borne encephalitis virus strain NEUDOERFL NS1 gene reverse primer
ttaagccaccaccatgctccggat

SEQ ID NO: 247 - DNA, 720 bp, Artificial Sequence
  SARS coronavirus spike protein coding sequence
ggtatctatcaaacttccaactttcgtgtggtcccatcgggcgatgtcgttcgctttccgaacatcactaatctgtgcccgttcggcgaagtgttcaacgcaacaaagtttccatccgtgtacgcctgggaacgcaagaagatctcgaactgcgtagccgactattccgttttatataattctaccttcttcagcacgttcaaatgttacggtgtgtccgcgacgaaactcaacgacctgtgttttagcaacgtttacgcggactcgttcgttgtgaaaggtgacgacgtgcgccagattgctccgggtcagaccggagtgatcgcagactacaactacaagctgccggacgactttatgggctgcgttctggcatggaacacccgtaatatcgacgcgacctctaccggcaactacaactacaagtaccgttacttacgccacggtaaactgcgccctttcgaacgcgatattagcaacgtcccgtttagcccggacggtaagccgtgtactccaccggcattgaactgctactggcctcttaacgactacggcttctatacgacaacgggaatcggttatcagccgtatcgtgtcgtcgttctgtcattcgagctgctgaacgcgccagcgacagtatgcggcccgaagctgagtaccgatctgatcaagaatcaatgcgtgaacttcaacttcaacggcttgaccgggaccgggtga

SEQ ID NO: 248 - protein, 239 aa, Artificial Sequence
  SARS coronavirus spike protein
GIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTG

SEQ ID NO: 249 - DNA, 1131 bp, Artificial Sequence
  Human coronavirus NL63 nucleocapsid protein coding sequence
gcctcggttaactgggccgacgatcgtgccgcgcgtaagaagttcccgcccccgagtttctatatgccgctgctggtctcaagcgacaaagcgccgtaccgtgttatcccgcgcaacttggtgccgatcggcaaaggcaacaaggacgaacaaatcgggtactggaacgtgcaggaacgctggcgcatgcgtcgtggtcagcgcgtggaccttccgccaaaggtacacttctactatcttgggaccggtccccacaaagatctgaagtttcgccagcgcagcgacggcgtagtgtgggtcgcgaaagagggcgcgaagacggtgaacacaagcctgggcaaccgtaagcgcaaccaaaagccgctggagccgaaatttagcatcgcccttccaccggaactgagcgtggtcgaattcgaagaccgtagtaacaattccagtcgcgcatctagccgctccagcacgcgcaataatagccgtgatagcagccgctctaccagtcgccagcaaagccgtacgcgcagcgacagcaatcaatccagctccgacttagtggccgccgtaacactggccctcaagaatctgggcttcgacaatcaatcaaagagcccatcctcgtcgggcacgtctacaccgaagaagccgaacaaaccgctgtcccagccgcgcgccgacaaaccgtcacaactgaagaagccccgctggaaacgcgtgccgacgcgtgaagagaacgtcatccaatgtttcggcccgcgcgacttcaaccataacatgggtgactcggacctggtgcaaaacggcgtggacgcgaagggattcccgcaattagcagagctgatcccgaaccaagccgccctgttcttcgactccgaagtatcaaccgacgaggttggcgacaacgtgcaaatcacttatacatataagatgctcgttgccaaagacaacaagaatctcccgaaatttatcgaacaaatcagcgcgttcaccaagccgagcagtattaaagagatgcaaagccagtcgagccacgtggcacaaaatactgttttaaacgcgagcatcccggagtcgaagccgctcgcggacgacgacagtgcaatcattgagatcgtgaatgaagtgttacactga

SEQ ID NO: 250 - protein, 376 aa, Artificial Sequence
  Human coronavirus NL63 nucleocapsid protein
ASVNWADDRAARKKFPPPSFYMPLLVSSDKAPYRVIPRNLVPIGKGNKDEQIGYWNVQERWRMRRGQRVDLPPKVHFYYLGTGPHKDLKFRQRSDGVVWVAKEGAKTVNTSLGNRKRNQKPLEPKFSIALPPELSVVEFEDRSNNSSRASSRSSTRNNSRDSSRSTSRQQSRTRSDSNQSSSDLVAAVTLALKNLGFDNQSKSPSSSGTSTPKKPNKPLSQPRADKPSQLKKPRWKRVPTREENVIQCFGPRDFNHNMGDSDLVQNGVDAKGFPQLAELIPNQAALFFDSEVSTDEVGDNVQITYTYKMLVAKDNKNLPKFIEQISAFTKPSSIKEMQSQSSHVAQNTVLNASIPESKPLADDDSAIIEIVNEVLH

SEQ ID NO: 251 - DNA, 1257 bp, Artificial Sequence
  SARS coronavirus 2 nucleocapsid protein coding sequence
tcggacaacggccctcagaaccaacgcaacgcccctcgtatcaccttcggcggtccgagcgactctacgggttctaatcaaaacggcgaacgtagtggcgcccgttcgaagcagcgccgcccgcaggggcttcctaacaacacggcatcatggtttactgcactgacccagcacggtaaagaggatttgaagtttccgcgcggccagggtgtgccgatcaatacgaactcaagcccggacgatcagatcggttattatcgtcgtgcaactcgtcgcatccgcggcggcgatggcaagatgaaggacctgtcgccgcgctggtacttttattatctgggcaccggaccagaggccgggctgccgtacggagcgaataaggatggaattatttgggtagcgacagaaggcgcactgaacacccctaaggaccatatcggtacgcgtaacccggcgaataacgccgccattgtcctgcagttaccccagggcacgacccttccgaagggattttatgccgagggctcgcgtggtggtagccaggctagtagccgttcaagttcccgctctcgtaattcttcgcgcaacagtacccccggatcttcacgtggcaccagcccggcccgcatggccggtaacggtggcgacgccgccttagccctgttgttattagatcgtcttaatcaattagaatcaaagatgagcggcaaggggcagcagcagcagggacagaccgtgaccaagaagtcagccgcggaagcctcgaagaaaccgcgtcagaaacgcaccgcgactaaggcgtataacgtgacccaggcatttgggcgccgtgggccggagcagacgcagggcaacttcggcgatcaagagttgattcgtcagggcacggactataagcactggccacagatcgctcagttcgcgccaagtgcctcggccttctttggcatgagtcgtatcggtatggaagttacgccatctggcacttggttaacatatacgggagcgattaagcttgacgataaggacccgaactttaaggaccaggtgatcttactcaacaaacacatcgatgcgtataagacctttccgcctaccgaaccgaagaaagataagaagaagaaagcggacgagacccaggctctcccacaacgtcaaaagaagcaacagaccgtcacgctgctgcccgcagctgacctggacgactttagcaagcagctgcagcagtctatgtcctctgccgattcgacccaagcttga

SEQ ID NO: 252 - protein, 418 aa, Artificial Sequence
  SARS coronavirus 2 nucleocapsid protein
SDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 253 - DNA, 1167 bp, Artificial Sequence
  Human coronavirus 229E nucleocapsid protein coding sequence
gcgacagttaaatgggcggatgcatcagaaccgcaacgtgggcgccaaggccgtattccgtacagcttgtactcgccgttactggtagacagcgagcagccatggaaagtcattccccgcaacctggtgcctattaataagaaggataagaacaaactgatcgggtactggaacgtgcagaagcgctttcgtacgcgcaaaggtaagcgcgtcgacttaagcccgaaattacacttctactacttgggtaccggccctcacaaagacgcgaagttccgtgaacgcgtggaaggggtggtgtgggtcgcggtcgacggtgcgaagaccgagcccactggctatggagtacgccgtaagaacagcgagccggaaattccgcattttaaccagaaacttccgaacggcgtaaccgtcgtagaggagccagattcgcgcgcgccgtcacgtagtcaatcacgtagccaatcgcgtggccgtggcgagagcaagccgcaatcgcgcaacccgagcagcgatcgtaatcacaatagccaagacgatattatgaaagcggtagccgcagcgctgaagtcgcttggcttcgataaaccacaagagaaggacaagaaaagtgcaaagacaggaaccccgaaaccgagccgcaaccaaagcccggcgtcatcgcagaccagtgctaaatcactggcgcgcagtcaatctagcgagaccaaggagcagaaacacgagatgcagaaaccgcgttggaagcgccaaccgaacgacgacgtaacctcaaacgtgacccagtgcttcgggcctcgtgatctggatcataatttcggcagcgccggcgtagtagcgaacggggtgaaggcaaagggttacccgcagttcgccgaattagttcctagtaccgccgccatgttattcgacagccatatcgtcagtaaggaaagcggtaataccgtcgttctgacctttacaacccgcgtaacggtacctaaggatcacccgcatctgggaaaattcctggaagaattgaacgcctttacccgtgagatgcagcagcacccgctgttgaatccgtcggccctggagtttaatccgagtcagacaagcccggcgacggcagagccggtacgcgacgaggtcagcatcgagaccgatattatcgacgaggtgaattga

SEQ ID NO: 254 - protein, 388 aa, Artificial Sequence
  Human coronavirus 229E nucleoprotein
ATVKWADASEPQRGRQGRIPYSLYSPLLVDSEQPWKVIPRNLVPINKKDKNKLIGYWNVQKRFRTRKGKRVDLSPKLHFYYLGTGPHKDAKFRERVEGVVWVAVDGAKTEPTGYGVRRKNSEPEIPHFNQKLPNGVTVVEEPDSRAPSRSQSRSQSRGRGESKPQSRNPSSDRNHNSQDDIMKAVAAALKSLGFDKPQEKDKKSAKTGTPKPSRNQSPASSQTSAKSLARSQSSETKEQKHEMQKPRWKRQPNDDVTSNVTQCFGPRDLDHNFGSAGVVANGVKAKGYPQFAELVPSTAAMLFDSHIVSKESGNTVVLTFTTRVTVPKDHPHLGKFLEELNAFTREMQQHPLLNPSALEFNPSQTSPATAEPVRDEVSIETDIIDEVN

SEQ ID NO: 255 - DNA, 723 bp, Artificial Sequence
  SARS coronavirus 2 spike protein coding sequence
ggcatttaccagacctcaaatttccgtgttcagccgactgagagtatcgtgcgcttcccgaacatcacgaatttatgtccgttcggcgaggtgttcaatgcgacacgcttcgcttcggtgtacgcctggaatcgcaaacgcatttctaattgcgtggcggactactcggtgctgtacaacagtgctagcttctcaacgttcaaatgctacggtgtatcgccgaccaagctgaacgacctgtgtttcaccaacgtttacgccgactcgttcgtgatccgcggcgacgaggttcgccagattgccccgggacagacaggcaaaatcgcggactacaactacaagctgccggacgacttcacgggatgtgtcatcgcatggaacagcaataacttagacagcaaagtgggcggcaactacaactatctctaccgtctgttccgcaaatccaacctgaagccgttcgaacgtgacatctcgaccgagatttaccaagcgggctcgactccgtgcaacggcgtagagggattcaactgctatttccccttacagagttacgggtttcagcctaccaacggcgtcgggtatcagccgtatcgcgtggtggtcttgagcttcgagctcctgcacgcgcctgcgacagtctgcggccccaagaaatcaaccaacctggtgaagaataagtgcgtgaactttaattttaacggccttaccggtactggctga

SEQ ID NO: 256 - protein, 240 aa, Artificial Sequence
  SARS coronavirus 2 spike protein
GIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTG

SEQ ID NO: 257 - DNA, 1323 bp, Artificial Sequence
  Human coronavirus HKU1 nucleocapsid protein coding sequence
agttacacaccgggtcaccacgcaggcagtcgttcctcaagcggtaaccgctccggcattttaaagaagaccagttgggtagatcagagcgaacgttcacaccagacatacaaccgcggtcgcaagcctcagccgaagtttacggttagcacgcagccgcagggcaatccgattccgcactacagctggtttagcggcatcactcagttccagaagggccgtgatttcaagttccctgacggacagggtgtgccgatcgcctatggtattccgccgtcagaggcgaagggttactggtacaagcataatcgtcgctccttcaagaccgcggacgggcagcagaaacaacttctcccgcgctggtacttttattacctgggaacgggcccgtacgcatcttctagttacggcgacgcgcatgagggcattttctgggtagcgagccatcaggcggatacatcgatcccgtcggacgtgtctgcccgcgacccgaccatccaagaggcgattcccacccgtttcagcccgggaaccatccttccgcagggttactacgtcgaaggttcaggtcgtagcgcgtcgaacagtcgccccggctcccgcagccaaagtcgcggtcctaacaaccgctctctgtctcgcagcaactccaatttccgccacagcgacagcattgttaagcccgacatggccgacgaaatcgcatctctcgttctggcgaaattgggcaaggacagcaagccgcaacaggtaacgaaacagaacgccaaagagatccgtcacaagatcctgatgaagccccgtcagaaacgcaccccgaacaagttctgcaacgtgcagcaatgcttcgggaagcgcggccctttacagaatttcggcaactcggagatgctgaaactgggaacgaacgacccgcagttcccgatcctggccgagctggcgccaactcccggcgcgttcttcttcggaagcaagcttgaattattcaagcgcgattcggacgcagactcgccgagcaaggatacattcgagctgcgctacagcggaagcatccgcttcgacagcacacttccgggcttcgaaactatcatgaaggtactcaaggaaaacctggacgcatacgtcaacagcaaccaaaataccgtcagcggctcattatccccgaagccgcaacgcaagcgcggcgtcaagcagagtcccgagtctttcgattcattgaacctgtcggccgacacacaacatatctcgaacgacttcaccccggaagaccacagcttactggccaccctggacgacccgtacgtggaagatagcgtagcatga

SEQ ID NO: 258 - protein, 440 aa, Artificial Sequence
  Human coronavirus HKU1 nucleocapsid protein
SYTPGHHAGSRSSSGNRSGILKKTSWVDQSERSHQTYNRGRKPQPKFTVSTQPQGNPIPHYSWFSGITQFQKGRDFKFPDGQGVPIAYGIPPSEAKGYWYKHNRRSFKTADGQQKQLLPRWYFYYLGTGPYASSSYGDAHEGIFWVASHQADTSIPSDVSARDPTIQEAIPTRFSPGTILPQGYYVEGSGRSASNSRPGSRSQSRGPNNRSLSRSNSNFRHSDSIVKPDMADEIASLVLAKLGKDSKPQQVTKQNAKEIRHKILMKPRQKRTPNKFCNVQQCFGKRGPLQNFGNSEMLKLGTNDPQFPILAELAPTPGAFFFGSKLELFKRDSDADSPSKDTFELRYSGSIRFDSTLPGFETIMKVLKENLDAYVNSNQNTVSGSLSPKPQRKRGVKQSPESFDSLNLSADTQHISNDFTPEDHSLLATLDDPYVEDSVA

SEQ ID NO: 259 - DNA, 1344 bp, Artificial Sequence
  Human coronavirus OC43 nucleocapsid protein coding sequence
agcttcacccccggcaaacagtctagctcccgtgcatctagcggcaaccgcagtgggaacggtattctgaaatgggcggaccaaagtgatcaattccgtaacgtgcagacgcgtgggcgccgtgcgcagccaaaacagaccagcacctcacaacagccgagcggcggcaacgtagtgccgtattatagctggttttcaggcatcacccagttccagaaaggcaaagaattcgaattcgccgaggggcagggcgttccaatcgccccaggggtgccggccaccgaggccaaaggttattggtatcgtcataaccgtcgcagtttcaagacggcggacggtaatcaacgtcagcttttaccgcgctggtacttctattaccttggcactggtcctcacgccaaggatcaatatggtacggacatcgatggcgttttctgggtagcatcgaatcaagccgacgttaacacgccagccgatatcgtggaccgtgatccgtcctcggacgaagctatcccaacccgcttcccgccgggaaccgtgctgccgcaaggctattatatcgagggtagcggtcgctcggccccaaactcgcgttcaacaagtcgtacttctagtcgcgcgagctcagcgggctcacgctctcgcgcgaactccggtaatcgtgcgccgacttcaggcgtgactccggatatggccgatcagatcgcgtcattggtgctcgcgaagctgggtaaagacgcgaccaaaccgcaacaggttacaaaacacacggcgaaggaagtacgtcaaaagatcctgaacaaaccgcgtcaaaagcgttctccgaacaagcagtgtacggtgcaacaatgcttcggcaaacgcggaccgaaccaaaacttcggcggcggtgagatgctcaagctgggtacatccgatccgcaatttccgatcttagccgagctggccccgactgcgggcgccttcttcttcggctcgcgtttggaactcgctaaggttcaaaacctgagcggaaacccggatgaacctcaaaaggacgtatacgagttacgttacaatggtgccatccgcttcgattcgaccctgagtggcttcgaaactattatgaaagttctgaacgaaaacctgaacgcgtaccagcagcaggacggaatgatgaacatgtcaccgaagccgcaacgccaacgcggactgaacaacggccagggtgagaacgacaacatttcggtggccgccccgaagtcgcgcgtccaacagaacaaatcgcgcgaactgacggcggaagatatttcactgctgaagaaaatggacgaaccgtacacggaagatacatccgagatttga

SEQ ID NO: 260 - protein, 447 aa, Artificial Sequence
  Human coronavirus OC43 nucleocapsid protein
SFTPGKQSSSRASSGNRSGNGILKWADQSDQFRNVQTRGRRAQPKQTSTSQQPSGGNVVPYYSWFSGITQFQKGKEFEFAEGQGVPIAPGVPATEAKGYWYRHNRRSFKTADGNQRQLLPRWYFYYLGTGPHAKDQYGTDIDGVFWVASNQADVNTPADIVDRDPSSDEAIPTRFPPGTVLPQGYYIEGSGRSAPNSRSTSRTSSRASSAGSRSRANSGNRAPTSGVTPDMADQIASLVLAKLGKDATKPQQVTKHTAKEVRQKILNKPRQKRSPNKQCTVQQCFGKRGPNQNFGGGEMLKLGTSDPQFPILAELAPTAGAFFFGSRLELAKVQNLSGNPDEPQKDVYELRYNGAIRFDSTLSGFETIMKVLNENLNAYQQQDGMMNMSPKPQRQRGLNNGQGENDNISVAAPKSRVQQNKSRELTAEDISLLKKMDEPYTEDTSEI

SEQ ID NO: 261 - DNA, 1266 bp, Artificial Sequence
  SARS coronavirus nucleocapsid protein coding sequence
tcggacaacggcccgcagtcgaatcagcgtagcgcaccgcgtatcacgttcggcggtccgaccgacagcaccgataacaatcaaaacggtggccgtaacggtgcccgcccgaagcaacgtcgccctcagggactcccgaacaacaccgccagctggtttacggccttgacccaacacggtaaagaagagttgcgttttccccgcggacaaggagtccccattaatacgaactctggccccgacgatcagatcggatattatcgtcgtgcgactcgccgcgtgcgcggcggagatggtaagatgaaggaactgtccccgcgctggtacttttactatttaggtaccggtccggaagccagcttaccgtatggtgccaataaagaggggattgtgtgggtggcgacggaaggcgctctgaacacccctaaggatcatatcggaactcgtaacccgaacaataacgccgcaacagtattgcagctgccacagggcacgacgctgcccaagggattttatgcggaaggctcacgtggtggatctcaggctagtagtcgttcttcgagccgctctcgtggcaacagccgtaactcgacccctggtagttcacgtggtaacagcccagcccgtatggcgtctggcggtggcgagaccgcacttgcccttctgttgctggatcgcctgaatcaattagaaagtaaggtgagtggcaagggtcagcagcagcagggacagacagtgacgaagaagtcagccgcagaagcctcaaagaaaccgcgtcagaagcgcaccgcaaccaagcaatataatgtaacgcaggcgttcggtcgccgcggcccggagcagacgcagggtaactttggagatcaggatttgattcgccaggggacggactataagcactggccccagatcgcccagttcgcaccctctgcaagcgcgttcttcggtatgtcccgtatcggaatggaagttactccgagcggtacgtggcttacataccacggcgcgatcaagctggacgataaggacccccagtttaaggataatgtaattttactcaataaacatatcgatgcgtataagacctttccgccgaccgaaccgaagaaagataagaagaagaaaaccgacgaggcgcaaccgcttccacaacgccagaagaaacaaccgaccgttacactgctgccggccgcggatatggacgactttagccgtcagttacagaacagcatgagcggcgcaagtgccgactccacgcaagcgtga

SEQ ID NO: 262 - protein, 421 aa, Artificial Sequence
  SARS coronavirus nucleocapsid protein
SDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 263 - DNA, 558 bp, Artificial Sequence
  Human coronavirus 229E spike protein coding sequence
ggcttttatagcacctctccgatccagagcgtagaactgccggtttccatcgtatcattaccggtataccacaagcacaccttcatcgttctttatgtagattttaagccgcaaagcggtggtggtaaatgtttcaattgctacccggcgggcgtcaacatcaccttggcaaacttcaacgagacgaagggtccgctttgcgtggatacttcccattttaccactaagtatgtagcagtatacgcgaacgtcggccgttggtctgccagcatcaataccgggaactgtccgttctcattcggtaaggtaaacaatttcgtcaagttcggatcagtgtgcttctctttgaaagacatcccaggcggctgtgcgatgccgattgtagcgaactgggcgtacagcaaatattacaccatcgggagtctgtacgtctcgtggtctgacggagacggcatcaccggtgtaccccagccggtcgaaggcgtgtcttctttcatgaacgtaaccttagataagtgcaccaagtacaacatctacgacgttagcggcgtcgggtga

SEQ ID NO: 264 - protein, 185 aa, Artificial Sequence
  Human coronavirus 229E spike protein
GFYSTSPIQSVELPVSIVSLPVYHKHTFIVLYVDFKPQSGGGKCFNCYPAGVNITLANFNETKGPLCVDTSHFTTKYVAVYANVGRWSASINTGNCPFSFGKVNNFVKFGSVCFSLKDIPGGCAMPIVANWAYSKYYTIGSLYVSWSDGDGITGVPQPVEGVSSFMNVTLDKCTKYNIYDVSGVG

SEQ ID NO: 265 - DNA, 552 bp, Artificial Sequence
  Human coronavirus NL63 spike protein coding sequence
gggttctactcggctaacttcctggacgacaacgtactgccggaaacctacgtcgcgctgccgatctactaccagcacaccgatattaacttcacggcgacggcctcgttcggtggaagctgctacgtgtgcaagccccgtcaagtaaacattagtctgaacggcaataccagcgtttgcgtccgcaccagtcacttctcgatccgctacatctacaatcgtgtgaaatcgggcagcccgggcgatagttcgtggcatatctacctgaaaagcgggacctgccccttcagcttcagtaaactcaacaacttccagaaattcaaaacaatctgcttttcgactgtagaggttccggggtcctgcaacttcccgctggaagcgacttggcactatacgagctacaccatcgtgggcgcgctgtacgtcacctggagcgagggcaactcaatcacgggcgtgccctacccggtgtcggggatccgcgaattctcgaacctggtgctgaacaactgcacaaagtacaacatctacgactacgtaggcaccggctga

SEQ ID NO: 266 - protein, 183 aa, Artificial Sequence
  Human coronavirus NL63 spike protein
GFYSANFLDDNVLPETYVALPIYYQHTDINFTATASFGGSCYVCKPRQVNISLNGNTSVCVRTSHFSIRYIYNRVKSGSPGDSSWHIYLKSGTCPFSFSKLNNFQKFKTICFSTVEVPGSCNFPLEATWHYTSYTIVGALYVTWSEGNSITGVPYPVSGIREFSNLVLNNCTKYNIYDYVGTG

SEQ ID NO: 267 - DNA, 993 bp, Artificial Sequence
  Human coronavirus HKU1 spike protein coding sequence
ggcgtgtacgatctgagtggattcacggtgaaaccagtggctacggtgtaccgccgcatcccgaacctgccggactgcgatatcgataattggctgaacaacgtcagcgtgccctcgccgttgaactgggagcgccgtatcttcagcaactgcaattttaacctctctacgctgctgcgcctggtgcacgtagactcgttctcatgcaacaacttggacaagagcaagatcttcggatcgtgtttcaactcaatcaccgtagataaattcgcaatcccgaaccgtcgccgtgacgacttgcagctggggagctcgggattcctgcagagtagcaactataagattgacatcagtagttcttcctgccagttatactactcactcccgttggtcaacgtcacgatcaacaatttcaacccgagcagctggaaccgtcgttacggattcggctcgttcaacgtatcctcgtacgatgtggtatacagcgaccactgcttctcagtaaatagtgatttctgtccgtgcgccgacccaagcgtggtaaactcgtgcgtcaagtccaaacccttaagcgctatctgcccggcgggcaccaagtaccgccactgtgatctggacaccaccctctacgtgaacaattggtgccgctgcagctgcttaccggatccaatctcaacctactccccaaatacctgcccccagaagaaagtagtcgtcgggatcggcgagcactgccctggactgggcatcaacgaagagaagtgcggcacccagcttaaccacagcagttgcagctgctcgcccgacgcattccttggatggagtttcgactcttgcatctctaacaaccgctgtaacatcttcagcaacttcatcttcaacgggatcaacagcggtacgacgtgcagcaacgacctgctgtacagcaataccgaggtctcaacgggcgtctgcgtgaactacgacttatacgggattaccggtcagggctga

SEQ ID NO: 268 - protein, 330 aa, Artificial Sequence
  Human coronavirus HKU1 spike protein
GVYDLSGFTVKPVATVYRRIPNLPDCDIDNWLNNVSVPSPLNWERRIFSNCNFNLSTLLRLVHVDSFSCNNLDKSKIFGSCFNSITVDKFAIPNRRRDDLQLGSSGFLQSSNYKIDISSSSCQLYYSLPLVNVTINNFNPSSWNRRYGFGSFNVSSYDVVYSDHCFSVNSDFCPCADPSVVNSCVKSKPLSAICPAGTKYRHCDLDTTLYVNNWCRCSCLPDPISTYSPNTCPQKKVVVGIGEHCPGLGINEEKCGTQLNHSSCSCSPDAFLGWSFDSCISNNRCNIFSNFIFNGINSGTTCSNDLLYSNTEVSTGVCVNYDLYGITGQG

SEQ ID NO: 269 - DNA, 978 bp, Artificial Sequence
  Human coronavirus OC43 spike protein coding sequence
ggagtgtacgagcttaacgggtataccgtacaacctatcgccgacgtgtatcgtcgcaagccgaacttaccgaactgcaacatcgaggcctggctgaacgacaaaagcgttccgagcccgctcaactgggagcgcaaaacgttcagtaactgcaacttcaacatgagttcgctcatgagcttcatccaagcggattcgttcacctgcaacaacatcgacgcagccaaaatttacggcatgtgcttctcgtcaattacgattgacaaattcgccatcccaaacggtcgcaaagtggatctgcagctgggcaacctcggatacctgcaaagcttcaattaccgtatcgacaccaccgccacctcttgccaactgtactacaacctgccggcggccaacgtaagcgtgtcacgcttcaacccgtcaacctggaacaaacgcttcggcttcattgagaacagtgtgttcaaaccgcagccggccggcgtgttcacgaaccacgacgtggtctacgcgcagcactgcttcaaggccccgaagaacttttgcccttgtaagctgaacggcagcctgtgtgtgggcagcgggccgggaaagaacaacggcatcggaacctgcccggccggcaccaattacctgacctgccataatctctgcaacccggacccgatcacgttcgcgggaccatacaaatgtccgcagacaaagagccttgtcggcattggcgaacattgcagcgggctcgcggtgaaatcggactactgcggtggaaacccgtgcacctgtcagccgcaggcgttcctcggatggtcagccgattcctgccttcagggcgataaatgcaacatcttcgcgaacctgatccttcacgacgtcaacagcggcctgacctgctcgaccgacctgcagaaggcaaatacggatatcatcctcggcgtgtgcgtaaactacgatttatacggcatcagcgggcagggatga

SEQ ID NO: 270 - protein, 325 aa, Artificial Sequence
  Human coronavirus OC43 spike protein
GVYELNGYTVQPIADVYRRKPNLPNCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNKRFGFIENSVFKPQPAGVFTNHDVVYAQHCFKAPKNFCPCKLNGSLCVGSGPGKNNGIGTCPAGTNYLTCHNLCNPDPITFAGPYKCPQTKSLVGIGEHCSGLAVKSDYCGGNPCTCQPQAFLGWSADSCLQGDKCNIFANLILHDVNSGLTCSTDLQKANTDIILGVCVNYDLYGISGQG

SEQ ID NO: 271 - DNA, 774 bp, Artificial Sequence
  MERS coronavirus spike protein coding sequence
ggggtgtattcagtgagcagctttgaagcgaagccgtcgggtagtgtcgttgagcaagcagagggcgtagagtgcgacttcagtcccttattatccggtaccccaccacaagtgtacaactttaaacgccttgtcttcacaaactgtaactacaacctgacaaagctgctgtccctgttcagtgtcaacgacttcacgtgctcccagatctcaccggcggccatcgccagtaattgctactcgtccttgatcctggactatttcagctatccgttatcgatgaagagtgacctgtcggtatccagcgcaggcccgatttcacaattcaactacaagcaaagcttcagcaacccgacctgcctgatcctggccacggtcccccacaatttaaccaccatcacaaaaccgctgaaatatagttacatcaataaatgttcgcgcctgctcagcgacgaccgcaccgaggtgccccaattggttaatgccaaccagtattcgccgtgcgtttcgatcgtgccgagcacagtatgggaagatggcgactactaccgtaagcagctgagtccgctggaaggcggcggttggttggtggcctcaggatcgacggtggcaatgaccgaacagctccaaatgggtttcgggatcaccgtgcagtacggcactgatactaactctgtatgtccgaaactggagttcgccaacgataccaagatcgcgtcgcagctgggtaactgtgtagagtacagtttatacggcgtgagcggtcgcgggtga

SEQ ID NO: 272 - protein, 257 aa, Artificial Sequence
  MERS coronavirus spike protein
GVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRG

SEQ ID NO: 273 - DNA, 1239 bp, Artificial Sequence
  MERS coronavirus nucleocapsid protein coding sequence
gcctcgcctgcggccccgcgtgccgtttcatttgccgacaataacgacattaccaacacgaatcttagtcgtggacgtggccgcaaccccaagccgcgcgccgcgccgaacaataccgtgagctggtatacaggcctcactcagcatggcaaggttccgttaactttcccgccgggtcaaggcgtgccgctgaacgcgaactctactccagcccagaacgcgggttactggcgtcgccaagatcgcaagatcaacactggaaacgggatcaaacagcttgcgccgcgctggtatttctattatacgggtaccgggcctgaggcggccctgccgtttcgtgccgtgaaagacgggattgtatgggtgcacgaggatggtgcgacagacgcgccctctacgttcggcacccgtaatccgaataacgactctgccatcgtaacgcagtttgcaccaggcaccaaattaccgaagaattttcatatcgaaggtacgggcggaaacagccagtcgtcgagccgcgctagctcagtgagtcgtaattcgagccgttccagcagccagggctcccgctcaggcaattcaactcgtgggaccagccctggccccagcggtattggcgccgttggcggcgatctgttgtatctggacctgctcaatcgtctgcaggcgctggaaagcggtaaggtgaaacagagccaaccgaaggtgattaccaagaaggacgccgccgcggccaagaacaaaatgcgtcataaacgtacctcaacgaagtcctttaatatggtccaggccttcggcttacgtgggccaggggatttgcaaggcaatttcggagacctgcagctgaacaagcttggtaccgaagatccgcgctggccgcagatcgcggaactggcgccgaccgctagcgccttcatgggcatgagccagttcaagttaacgcaccaaaataacgacgaccacggtaatccagtctattttctgcgctattcaggggcgatcaagctggatccgaagaacccgaattataacaaatggctggaactgttggaacagaacatcgacgcgtataagacatttccgaagaaagagaagaagcagaaagccccgaaagaggagtcgaccgatcagatgtccgagccaccgaaagaacaacgcgttcagggctccattacccaacgtacccgtacacgcccttccgtgcaaccgggccctatgatcgacgtcaataccgactga

SEQ ID NO: 274 - protein, 412 aa, Artificial Sequence
  MERS coronavirus nucleocapsid protein
ASPAAPRAVSFADNNDITNTNLSRGRGRNPKPRAAPNNTVSWYTGLTQHGKVPLTFPPGQGVPLNANSTPAQNAGYWRRQDRKINTGNGIKQLAPRWYFYYTGTGPEAALPFRAVKDGIVWVHEDGATDAPSTFGTRNPNNDSAIVTQFAPGTKLPKNFHIEGTGGNSQSSSRASSVSRNSSRSSSQGSRSGNSTRGTSPGPSGIGAVGGDLLYLDLLNRLQALESGKVKQSQPKVITKKDAAAAKNKMRHKRTSTKSFNMVQAFGLRGPGDLQGNFGDLQLNKLGTEDPRWPQIAELAPTASAFMGMSQFKLTHQNNDDHGNPVYFLRYSGAIKLDPKNPNYNKWLELLEQNIDAYKTFPKKEKKQKAPKEESTDQMSEPPKEQRVQGSITQRTRTRPSVQPGPMIDVNTD

SEQ ID NO: 275 - DNA, 1584 bp, SARS-CoV-2
  UKS, B1.1.1.7 S1
tttgtgtttctggtactgttgccgttggtcagtagccagtgcgtgaatttaactacccgtacccagctgccgccggcttataccaatagtttcacccgtggcgtgtactacccggataaagtgtttcgttccagtgttcttcatagcacccaggacttatttctgccatttttctcgaatgtgacgtggttccatgccattagtggtaccaacggtaccaaacgctttgataacccggtgctgccattcaacgatggtgtctacttcgccagcacggaaaaaagcaacattatccgcggttggatctttggtaccacgttagactctaaaacgcaatcattgttaatcgtaaataatgctacaaatgtggttatcaaagtttgcgagtttcaattctgtaacgatccttttctgggtgtataccataaaaacaataaatcgtggatggaatcagaattccgcgtgtattcgagcgcgaacaactgcacctttgaatacgtctcacaaccgtttctgatggatctggagggcaagcagggaaattttaaaaacctgcgtgaatttgtctttaaaaacatcgatggatacttcaaaatttacagcaaacataccccgattaacctggtacgggacctcccgcagggcttttcagccctggaaccgttagtcgatctgcctattgggatcaatattacccgttttcaaacattgctcgcgctgcatcgttcctacttgaccccaggcgatagctcttcgggctggacggcgggcgcggcggcctactatgtaggctatctccagcctcgtacctttttgttaaaatataatgaaaacggtacgatcaccgacgccgtggactgtgccctggatccattgagcgaaaccaaatgtacgttaaaatccttcacagtcgaaaaaggtatttatcaaaccagcaattttcgcgtacaaccgacggaaagcattgtacgctttccgaatattaccaacctgtgcccgtttggtgaagtatttaacgccacacgtttcgcatcggtgtatgcatggaaccgcaaacggatttccaattgtgtcgcagattatagcgttttatacaacagcgcgagttttagcacctttaaatgttacggtgtatctcctacaaaactgaatgatttatgttttaccaacgtgtacgcagattcatttgttattcgtggtgacgaggtacgccagattgcccccgggcaaacgggcaaaattgctgactacaactacaagctgccggatgattttaccggctgcgttatcgcctggaacagtaacaacttggactctaaagtgggagggaactacaactatttgtaccgcttatttcgtaaatccaaccttaaacctttcgaacgtgatatctctaccgaaatctatcaagccggctccaccccgtgcaacggtgtggaaggtttcaactgctactttcctctgcaatcttacggttttcagcctacatatggagtgggataccagccgtaccgtgtggtggtgctgtcttttgagctgctgcacgcccctgccactgtgtgcggtccgaaaaaaagcacctaa

SEQ ID NO: 276 - protein, 527 aa, SARS-CoV-2
  UKS, B.1.1.7 S1
FVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKST

SEQ ID NO: 277 - DNA, 1593 bp, SARS-CoV-2
  SAS, B.1.351/501Y.V2 S1
ttcgtcttcctggtcctgctgccgctcgtatcaagtcagtgcgttaactttaccactcgcacccagttaccgccggcttatactaactcttttacccgtggcgtttattacccggataaagtatttcgctctagcgtcttacatagtacacaggatttgttccttccgttcttcagtaatgtcacttggttccatgcaattcacgtttccgggaccaacgggaccaaacgctttgcgaaccccgtcctcccgtttaatgatggtgtctatttcgcctcgacggagaaatcaaacattatccgcggatggatctttggtaccacgctggatagcaagacccagtcgttgctgatcgtgaataacgctaccaacgtggtgattaaagtctgcgaattccaattttgcaatgatccgtttctgggcgtgtactaccataaaaataataaatcgtggatggaaagtgaattccgcgtatatagcagcgcgaataactgtaccttcgagtatgtaagccagccgttcttaatggatctggaaggcaaacagggtaatttcaaaaacctccgcgaattcgtgtttaaaaacattgacggttatttcaaaatctatagtaaacacaccccgattaacttggtgcggggtctgccgcaaggctttagcgccttagagccgctggttgatctgccgattggcatcaacattacgcgcttccagacgctcctggcgctgcacatctcttatctgacccctggagacagttcttccggttggacggccggcgccgctgcgtattatgtcggctatctgcagccccgcactttccttttaaagtacaacgaaaacggtacaattaccgatgctgtagattgcgcgctggatccgttatcggaaacgaaatgtacactgaaatctttcacggttgaaaagggcatctatcaaacgagtaacttccgcgtgcaaccgactgaaagtatcgtacgctttccgaatattacgaatctgtgccccttcggcgaagttttcaatgccacccggtttgcttccgtatatgcgtggaatcgtaagcgcattagtaattgcgttgcggactatagcgtgttgtataatagtgcaagtttcagtacgttcaagtgttatggcgtctcgcctaccaaactgaacgatctgtgctttaccaatgtatacgccgatagctttgtgatccgcggggacgaagttcgccagatcgccccgggccaaactggcaacatcgccgattataattataagctgcccgatgactttaccggctgtgtaattgcttggaactccaacaacctggattctaaagttggcggcaactacaactatctgtatcgtctgtttcgtaaatctaatctgaagccgtttgaacgcgacatttctacagagatctaccaagccggctctaccccgtgtaatggcgtgaaaggctttaactgctatttccctttacaaagctatggatttcagccaacctatggtgtaggctatcaaccataccgtgtggtagtgttgagttttgaactgctgcacgctcctgcaacggtttgcggacccaaaaaaagcacgtaa

SEQ ID NO: 278 - protein, 530 aa, SARS-CoV-2
  SAS, B.1.351/501Y.V2 S1
FVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLLALHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKST

SEQ ID NO: 279 - DNA, 1593 bp, SARS-CoV-2
  BRS, P.1 S1
ttcgttttcctggttctgctgcctttggtatctagtcaatgtgtaaactttaccaaccgtacacaactgccatcggcatatactaattcctttacgcgcggagtatattatccagacaaagtgtttcgttcttcggtgctgcattcaactcaagatttattcctgccattcttctcaaacgtgacttggttccatgctatccatgtgagcgggacgaacggaacaaagcgtttcgacaatccggtcttacccttcaacgacggcgtgtactttgcatcgaccgaaaagagcaacattattcgcggctggatcttcgggaccacactcgactcgaaaactcaatcacttttgattgtcaataatgcaacaaatgtcgtgatcaaagtttgtgaatttcagttctgtaactacccctttctgggagtttactaccacaagaacaacaagtcttggatggagagcgaattccgcgtctacagctcagcgaataactgcaccttcgagtatgtctctcagccgtttctgatggaccttgagggaaagcaaggtaactttaagaaccttagcgagtttgtgttcaagaatatcgacggctacttcaagatctattcgaagcacactccgattaacctcgttcgcgacttaccacaaggcttttccgcgcttgagccattagttgatttaccaattgggatcaacatcacgcgctttcagacgttactggcgctccatcgtagctatctcacgccaggtgatagctcgtctggttggactgcgggtgccgctgcctactatgttggataccttcaacctcgtactttcttgctcaagtataacgagaacgggaccatcacggacgccgtggactgcgcccttgacccgctgtcggagacaaagtgtactctgaagtctttcaccgtcgaaaagggcatctatcaaactagcaattttcgtgtacaacctacagaatctatcgttcgcttcccaaatattactaacttatgtcctttcggagaagtattcaatgctactcgcttcgcaagcgtttatgcttggaatcgtaagcgtatttcaaactgtgtagcggattactctgtgttgtacaactcggctagtttcagcaccttcaaatgctatggcgttagtccgacgaaattaaatgatttgtgttttaccaacgtgtacgccgacagcttcgtgattcgcggagacgaggtgcgccaaatcgcgcctggtcagacgggcactattgccgattataattataagcttccggatgattttacaggctgcgtaattgcgtggaatagtaataatctggactccaaggtcggcggtaattacaactatctctatcgtctctttcgtaagagtaaccttaagcccttcgagcgcgacatttcaactgaaatctatcaagccggtagcacgccgtgtaatggcgtaaagggcttcaattgttactttccgctgcagtcatatggctttcaaccgacgtacggggttggctatcaaccgtaccgtgtagtcgtgctctcgtttgaactccttcacgcgcctgctaccgtctgtggtccgaagaagtcaacatga

SEQ ID NO: 280 - protein, 530 aa, SARS-CoV-2
  BRS, P.1 S1
FVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKST

SEQ ID NO: 281 - DNA, 666 bp, SARS-CoV-2
  BRbd, P.1 S-RBD
ggcatttaccaaacaagcaacttccgcgtccagcctacggagtcgatcgtgcgcttccccaacattaccaatttgtgtcctttcggtgaagtattcaatgccacccgcttcgcgagcgtatacgcgtggaatcggaagcgcatcagcaattgtgtggcggattattctgtcctttataatagcgccagtttcagtacattcaaatgctacggtgttagcccgacgaaactgaatgatctgtgctttacgaacgtttacgctgactcattcgtgatccggggtgacgaggttcgtcagattgcccctggccagactggtactatcgctgactacaattacaaattgccggacgattttacgggttgtgtaatcgcctggaattcgaacaacctggattcgaaagtcggcggcaactacaactacctgtaccggctcttccgcaaaagcaatctcaaaccgtttgaacgtgacattagtacggaaatctatcaagcgggttctactccttgcaacggcgtgaaaggctttaattgttatttcccattgcagtcatacgggttccaaccgacctatggcgttggataccagccataccgtgttgtcgtactgagttttgagctgctccacgcccctgcgactgtctgcgggccgaaaaaaagcacgtga

SEQ ID NO: 282 - protein, 221 aa, SARS-CoV-2
  BRbd, P.1 S-RBD
GIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKST

SEQ ID NO: 283 - DNA, 1590 bp, SARS-CoV-2
  WRSS, Wuhan Reference S1
tttgtttttctggttctgctgccattggtaagttctcaatgcgtcaacctcacgacgcgtacccagttaccaccggcatacacgaacagcttcacacgcggcgtctattatccggataaagtattccgtagctccgttctgcattcgacacaagatctcttccttcctttcttctctaacgttacgtggtttcatgcgatccatgtctcaggcacaaacgggacgaaacgctttgataatccggttcttcccttcaacgatggcgtatacttcgcctcgacggaaaaaagcaatatcattcgtggctggatttttggcactaccttggactctaaaacccaaagcctgctgattgttaataatgcgacaaacgtcgtgattaaagtctgtgaattccagttttgtaacgatccattcctgggtgtatactatcacaaaaataataaatcatggatggagtccgagttccgtgtttacagctcggccaacaactgcacttttgagtacgttagccagccgtttctcatggacctggaaggaaaacagggtaattttaaaaacctgcgcgaatttgtgtttaaaaatattgacgggtattttaaaatttactcaaagcacacgccgattaacctggtacgcgatctgccgcagggtttttcggcattagaacctctcgtggatctgccgattggcatcaacattacgcgcttccaaaccctgctggcgctgcaccgtagctacttgactccgggggattctagcagcggctggactgcaggcgcggccgcgtactatgtaggatatcttcaaccgcgcacctttctgctgaaatacaacgaaaacgggaccattacagacgcagtggattgtgcgctggatcctttaagcgagacaaagtgcaccctgaaatctttcaccgtggaaaaaggcatctatcaaacgagtaactttcgggtgcagccgaccgaaagtattgtgcgcttcccgaatatcacgaatctttgcccgtttggcgaagtatttaatgcaacccgctttgcctcggtgtatgcgtggaaccgtaaacgcattagtaattgcgtggcagattacagcgttctgtacaacagcgcaagtttttcaacctttaaatgctacggcgttagcccgactaaactcaacgacctgtgttttacaaatgtgtatgccgatagctttgtgatccggggcgatgaagttcgccagatcgccccggggcagaccggtaaaattgccgataattataaactcccagacgattttacgggctgcgtcattgcatggaactcgaataacctggattccaaagtcggcggcaactacaactacctgtatcgcctgttccgcaaatcgaatctcaaaccattcgaacgcgatatctctacagaaatctaccaagcaggtagcaccccgtgcaacggcgtggaaggttttaactgttatttcccgctgcagagctacggcttccagccaacgaacggcgttggttaccaaccctatcgcgtagtggttctttcgtttgagctgttacacgctccggcgacagtttgcggacctaaaaaatcgacctaa

SEQ ID NO: 284 - protein, 529 aa, SARS-CoV-2
  WRSS, Wuhan Reference S1
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 285 - DNA, 666 bp, SARS-CoV-2
  UKSbd, B.1.1.7 S-RBD
ggcatttatcagacctccaactttcgcgttcagccgaccgagagtattgttcgtttcccaaacatcacaaacttgtgtccgttcggggaagtgttcaacgccacgcgttttgctagtgtttatgcttggaaccgcaaacgtattagtaactgcgtggcggattatagtgttttgtataactccgccagctttagtacgtttaaatgctacggcgtgagtccaacaaaactgaacgatctgtgcttcaccaacgtgtacgcggattcatttgtgatccgcggcgatgaggttcgccagatcgctccgggccagacaggcaagatcgccgattacaattataaattaccagatgatttcaccgggtgtgtcatcgcctggaattccaataatctggacagcaaagtgggcggcaactataattatctgtatcgtctgttccgcaaaagcaatctgaaaccgttcgaacgcgatatttccactgagatttaccaggccggcagtaccccgtgtaatggcgttgaaggtttcaattgctacttccccctgcagtcgtacggctttcagccgacatatggcgtcggctaccagccgtatcgcgtcgttgtgctgagtttcgagttgctgcatgccccggccaccgtgtgcggcccgaaaaaaagcacgtaa

SEQ ID NO: 286 - protein, 221 aa, SARS-CoV-2
  UKSbd, B.1.1.7 S-RBD
GIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKST

SEQ ID NO: 287 - DNA, 666 bp, SARS-CoV-2
  SASbd, B.1.351/501Y.V2 S-RBD
ggtatctaccagacgagcaattttcgcgtacagcccaccgaaagtatcgtccgttttccaaacattaccaatctgtgtccgtttggagaagtgttcaacgccacccgcttcgcgtcagtttatgcctggaaccgtaagcgcatcagtaactgcgtggcggattattctgtactttataatagcgccagcttttcaactttcaaatgttatggcgtttcgcccactaaattaaacgatttgtgcttcacaaatgtgtacgcggattccttcgttatccgtggtgacgaagtccgtcagatcgcgccaggccagactggaaacattgcagattacaattataaactccctgacgatttcaccggctgtgttattgcctggaattcgaataacctggatagcaaagttggcggtaattataattatctgtaccgcctgtttcgcaaatctaacctgaaaccgttcgaacgtgacatctcgaccgaaatctaccaggcgggttccacaccgtgtaatggcgtgaaaggttttaactgctattttccgctgcagagctatggattccagccgacctatggcgtgggttaccagccgtatcgggttgtggtcctgtctttcgagctgctccatgcgcctgcgacagtgtgcggcccgaaaaaaagtacgtaa

SEQ ID NO: 288 - protein, 221 aa, SARS-CoV-2
  SASbd, B.1.351/501Y.V2 S-RBD
GIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKST

SEQ ID NO: 289 - DNA, 1587 bp, SARS-CoV-2
  IDS B.1.617.1 S1
tttgtgtttctggtgctgctgccgctggtgagcagccagtgcgtgaacctgacccgcacccgcacccagctgccgccggcgtataccaacagctttacccgcggcgtgtattatccggataaagtgtttcgcagcagcgtgctgcatagcacccaggatctgtttctgccgttttttagcaacgtgacctggtttcatgcgattcatgtgagcggcaccaacggcaccaaacgctttgataacccggtgctgccgtttaacgatggcgtgtattttgcgagcaccgaaaaaagcaacattattcgcggctggatttttggcaccaccctggatagcaaaacccagagcctgctgattgtgaacaacgcgaccaacgtggtgattaaagtgtgcgaatttcagttttgcaacgatccgtttctggatgtgtattatcataaaaacaacaaaagctggatggaaagcggcgtgtatagcagcgcgaacaactgcacctttgaatatgtgagccagccgtttctgatggatctggaaggcaaacagggcaactttaaaaacctgcgcgaatttgtgtttaaaaacattgatggctattttaaaatttatagcaaacataccccgattaacctggtgcgcgatctgccgcagggctttagcgcgctggaaccgctggtggatctgccgattggcattaacattacccgctttcagaccctgctggcgctgcatcgcagctatctgaccccgggcgatagcagcagcggctggaccgcgggcgcggcggcgtattatgtgggctatctgcagccgcgcacctttctgctgaaatataacgaaaacggcaccattaccgatgcggtggattgcgcgctggatccgctgagcgaaaccaaatgcaccctgaaaagctttaccgtggaaaaaggcatttatcagaccagcaactttcgcgtgcagccgaccgaaagcattgtgcgctttccgaacattaccaacctgtgcccgtttggcgaagtgtttaacgcgacccgctttgcgagcgtgtatgcgtggaaccgcaaacgcattagcaactgcgtggcggattatagcgtgctgtataacagcgcgagctttagcacctttaaatgctatggcgtgagcccgaccaaactgaacgatctgtgctttaccaacgtgtatgcggatagctttgtgattcgcggcgatgaagtgcgccagattgcgccgggccagaccggcaaaattgcggataactataaactgccggatgattttaccggctgcgtgattgcgtggaacagcaacaacctggatagcaaagtgggcggcaactataactatctgtatcgcctgtttcgcaaaagcaacctgaaaccgtttgaacgcgatattagcaccgaaatttatcaggcgggcagcaaaccgtgcaacggcgtggaaggctttaactgctattttccgctgcagagctatggctttcagccgaccaacggcgtgggctatcagccgtatcgcgtggtggtgctgagctttgaactgctgcatgcgccggcgaccgtgtgcggcccgaaaaaaagcacctaa

SEQ ID NO: 290 - protein, 528 aa, SARS-CoV-2
  IDS B.1.617.1 S1
FVFLVLLPLVSSQCVNLTRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADNYKLPDDFTGCVIAWNSNNLDSKVGGNYNYLYRLFRKSNLKPFERDISTEIYQAGSKPCNGVEGFNCYFPLQSYGFQPTNGVGYQPYRVVVLSFELLHAPATVCGPKKST

SEQ ID NO: 291 - DNA, 663 bp, SARS-CoV-2
  IDS B.1.617.1 S-RBD
ggcatttatcagaccagcaactttcgcgtgcagccgaccgaaagcattgtgcgctttccgaacattaccaacctgtgcccgtttggcgaagtgtttaacgcgacccgctttgcgagcgtgtatgcgtggaaccgcaaacgcattagcaactgcgtggcggattatagcgtgctgtataacagcgcgagctttagcacctttaaatgctatggcgtgagcccgaccaaactgaacgatctgtgctttaccaacgtgtatgcggatagctttgtgattcgcggcgatgaagtgcgccagattgcgccgggccagaccggcaaaattgcggattataactataaactgccggatgattttaccggctgcgtgattgcgtggaacagcaacaacctggatagcaaagtgggcggcaactataactatctgtatcgcctgtttcgcaaaagcaacctgaaaccgtttgaacgcgatattagcaccgaaatttatcaggcgggcagcaaaccgtgcaacggcgtggaaggctttaactgctattttccgctgcagagctatggctttcagccgaccaacggcgtgggctatcagccgtatcgcgtggtggtgctgagctttgaactgctgcatgcgccggcgaccgtgtgcggcccgaaaaaaagctaa

SEQ ID NO: 292 - protein, 220 aa, SARS-CoV-2
  IDS B.1.617.1 S-RBD
GIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSKPCNGVEGFNCYFPLQSYGFQPTNGVGYQPYRVVVLSFELLHAPATVCGPKKS

(19) United States
(12) Patent Application Publication
(10) Pub. No.: US 2024/0302369 A1
(43) Pub. Date: Sep. 12, 2024

(54) SYSTEM AND METHODS FOR DETECTION OF PATHOGENIC VIRUSES

(71) Applicant: Viroforge Technologies, LLC, Frederick, MD (US)

(72) Inventors: Robert G. ULRICH, Frederick, MD (US); Mohan NATESAN, Lorton, VA (US)

(21) Appl. No.: 18/006,361

(22) PCT Filed: Jul. 23, 2021
(86) PCT No.: PCT/US2021/043002

§ 371 Date: Jan. 20, 2023

Related U.S. Application Data
(60) Provisional application No. 63/056,043, filed on Jul. 24, 2020.

(51) Int. Cl. G01N 33/569; B01L 3/00; G01N 33/68

(52) U.S. Cl. CPC G01N 33/56983; B01L 3/502761; G01N 33/6854; B01L 2200/16; B01L 2300/046; B01L 2300/0819; B01L 2400/049; G01N 2333/165; G01N 2333/18

(57) ABSTRACT

The present invention provides a system and methods for the detection of peptides from and antibodies against coronaviruses, filoviruses, flaviviruses, or combination thereof, in a sample. A method for detecting the presence of a coronavirus in a sample may comprise: collecting a small sample of a biological fluid from the test subject, adding sample to a viral test cassette, initiating sample processing by the assay cassette to incubate the diluted sample and developing reagents with an antibody microarray contained within the cassette, and results of the test are read by visual examination and process the results to determine if the subject is infected by a coronavirus.

### CROSS-REFERENCE TO RELATED PATENT APPLICATIONS

This international patent application claims priority to U.S. Provisional Patent Application No. 63/056,043, filed Jul. 24, 2020, the disclosure of which is herein incorporated by reference in its entirety.

### REFERENCE TO SEQUENCE LISTING SUBMITTED ELECTRONICALLY

The official copy of the sequence listing is submitted electronically via EFS-Web as an ASCII-formatted sequence listing with a file named “3000071-001977_Sequence_Lising_ST25.txt” created on Jul. 23, 2021, and having a size of 419,119 bytes, and is filed concurrently with the specification. The sequence listing contained in this ASCII-formatted document is part of the specification and is herein incorporated by reference in its entirety.

### BACKGROUND

### 1. Field

The present disclosure relates to a system and methods for the detection of viral infections.

### 2. Description of Related Art

Viral infections are a continued problem for public health. In the 20^{th} and 21^{st} centuries, pandemics have been caused by novel viruses. Additionally, efficient means for the detection of known viral pathogens are still needed to allow better control and treatment of infections.

### Coronavirus

Coronavirus disease (COVID-19) is an infectious disease caused by a newly discovered coronavirus, Severe Acute Respiratory Syndrome CoronaVirus 2 (SARS-CoV-2). The SARS-CoV-2 was discovered in Wuhan Viral Pneumonia cases in late 2019, and was named by the World Health Organization on Jan. 12, 2020. It belongs to the beta genera of the Coronaviridae family identified in 2003, together with SARS coronavirus (SARS CoV), identified in 2003 and MERS coronavirus (MERS CoV) identified in 2012. The SARS-CoV-2 genome shares about 70% sequence identity with the SARS CoV virus and about 40% sequence similarity with the MERS CoV virus. WHO website (2020).

Most people infected with the COVID-19 virus will experience mild to moderate respiratory illness and recover without requiring special treatment. Older patients, e.g., over 60 years of age, and those with underlying medical problems such as cardiovascular disease, diabetes, chronic respiratory disease, and cancer, are more likely to develop serious illness. Centers for Disease Control website (2020). In susceptible populations, severe acute respiratory syndrome coronavirus 2 (SARS-CoV-2) may cause fatal human respiratory disease. Patients with SARS-CoV-2 often display the characteristics of acute lung injury (ALI), including diffuse alveolar damage (DAD), epithelial necrosis, and fibrin and hyaline deposition. Many patients who die of SARS-CoV-2 develop acute respiratory distress syndrome (ARDS), a severe form of acute lung injury. Li & Ma Critical Care (2020) 24: 198Post-COVID conditions are a wide range of new, returning, or ongoing health problems that can occur more than four weeks after symptomatic or asymptomatic infection. Health problems include fatigue, dizziness, loss of smell and taste, difficulty in mental concentration, and heart palpitations. CDC Website 2021. Outbreaks of severe acute respiratory infections of emerging viruses, including Middle Eastern respiratory syndrome CoVs (MERS-CoV) and the novel coronavirus (SARS-CoV-2; the disease referred to as “COVID-19”) show a need in the art for effective detection of these viral infections and an assessment of specific serological immunity to infection and vaccination. The availability of effective COVID-19 vaccines in the U.S. and a limited number of other countries has decreased the infection rate among the vaccinated. However, the unknown duration of vaccine-induced immunity to infection and the risk of re-infection also demonstrate a need in the art for effective detection of these viral infections and an assessment of specific serological immunity

### Ebola Virus Disease (EVD) and Marburg Virus Disease (MVD)

Marburg virus was first recognized in 1967, when outbreaks of hemorrhagic fever occurred simultaneously in laboratories in Marburg and Frankfurt, Germany and in Belgrade, Yugoslavia (now Serbia). Thirty-one people became ill, initially laboratory workers followed by several medical personnel and family members who had cared for them. Seven deaths were reported. The first people infected had been exposed to imported African green monkeys or their tissues while conducting research. One additional case was diagnosed retrospectively. A reservoir host of Marburg virus is the African fruit bat, Rousettus aegyptiacus, which show no obvious signs of illness. Primates including humans can become infected with Marburg virus, and may develop serious disease (MVD) with high mortality. Ebola virus was discovered in 1976 after two consecutive outbreaks of fatal hemorrhagic fever (EVD) occurred in different parts of Central Africa. The first outbreak occurred in the Democratic Republic of Congo (formerly Zaire) in a village near the Ebola River, which gave the virus its name. The second outbreak occurred in what is now South Sudan. These two outbreaks were caused by two genetically distinct viruses: Zaire ebolavirus and Sudan ebolavirus. The virus came from two different sources and spread independently to people in each of the affected areas. Viral and epidemiologic data suggest that Ebola virus existed before these recorded outbreaks occurred. Factors like population growth, encroachment into forested areas, and direct interaction with wildlife (such as bushmeat consumption) may have contributed to the spread of the Ebola virus.

Ebola virus disease (EVD) (the viral hemorrhagic fever caused by infection with an Ebola virus) is a deadly disease with occasional outbreaks that occur primarily on the African continent. EVD most commonly affects people and nonhuman primates (such as monkeys, gorillas, and chimpanzees). It is caused by an infection with a group of viruses within the genus Ebolavirus: Ebola virus (species Zaire ebolavirus); Sudan virus (species Sudan ebolavirus); Taï Forest virus (species Taï Forest ebolavirus, formerly Côte d'Ivoire ebolavirus); Bundibugyo virus (species Bundibugyo ebolavirus); Reston virus (species Reston ebolavirus); and Bombali virus (species Bombali ebolavirus). Only four viruses—Ebola, Sudan, Taï Forest, and Bundibugyo viruses—are known to cause disease in people. Reston virus causes disease in nonhuman primates and pigs. It is unknown if Bombali virus, identified in bats, causes disease in either animals or people.

The Ebola virus spreads to people initially through direct contact with the blood, body fluids and tissues of animals. Ebola virus then spreads to other people through direct contact with body fluids of a person who is infected or has died from EVD. This can occur when a person touches these infected body fluids (or objects that are contaminated with them), and the virus gets in through broken skin or mucous membranes in the eyes, nose, or mouth. People can get the virus through sexual contact with someone who is sick with EVD, and also after recovery from EVD. The virus can persist in certain body fluids after recovery from the illness and this persistent infection is a newly recognized cause of new disease outbreaks. Lancet (2018) 18:1015 Infected animals carrying the virus can transmit it to other animals, like apes, monkeys, duikers, and humans. Ebola survivors may experience side effects after their recovery, such as tiredness, muscle aches, eye and vision problems and stomach pain. CDC Website (2020).

The largest Ebola virus disease (EVD) outbreak in West Africa (2013-2016), resulted in more than 11,200 deaths and 28,000 suspected cases in Guinea, Liberia and Sierra Leone. An outbreak of EVD in the Democratic Republic of Congo (“the Kivu Ebola epidemic”) began on Aug. 1, 2018 resulting in over 2,000 deaths, despite the vaccination of more than 250,000. These Ebola epidemics exposed many weaknesses in existing healthcare monitoring and management systems. For the West African outbreak, regional and international responses were compromised by inaccessible, incomplete, and underutilized health information. Accessible methods to rapidly monitor biological fluids for detection of suspected infections will result in actionable data for professional healthcare workers. These methods must not only provide accurate diagnosis but also overcome the difficulties created by untrained personnel and scattered or corrupted test records. However, there are no truly portable diagnostic devices that can be used by care providers with minimal training, especially those designed for detecting Ebola and Marburg virus emergence within the community. There exists a need in the art for a rapid, efficient means for the detection of Ebola virus infections.

### Yellow Fever

The yellow fever virus is found in tropical and subtropical areas of Africa and South America. The virus is spread to humans by the bite of an infected mosquito. Illness ranges from a fever with aches and pains to severe liver disease with bleeding and yellowing skin (jaundice). CDC Website (2020).

The majority of people infected with yellow fever virus will either not have symptoms, or have mild symptoms and completely recover. For people who develop symptoms, the time from infection until illness is typically 3 to 6 days. Typical yellow fever illness with initial symptoms including: sudden onset of fever, chills, severe headache, back pain, general body aches, nausea, vomiting, fatigue, and weakness. Severe symptoms of yellow fever include: high fever, yellow skin (jaundice), bleeding, shock, and organ failure. Patients who develop severe yellow fever infections suffer about a 30% to 60% fatality rate. CDC Website (2020).

There is no medicine to treat or cure infection from yellow fever. Yellow fever infection is diagnosed based on laboratory testing, a person's symptoms, and travel history. CDC Website (2020). There exists a need in the art for a rapid, efficient means for the detection of Yellow fever infection and immunity.

### BRIEF SUMMARY

In an embodiment, a system for detecting the presence of a coronavirus in a sample comprising: venous, capillary or arterial blood or plasma separated from the same blood sources; urine, feces, saliva; oral, nasal, and respiratory secretions; and cerebrospinal fluid.

In an embodiment, a system for detecting the presence of antibodies specific for a coronavirus in a sample comprising: venous, capillary or arterial blood or plasma separated from the same blood sources; urine, feces, saliva; oral, nasal, and respiratory secretions; and cerebrospinal fluid.

In an embodiment, a method for detecting the presence of a coronavirus in a sample comprises the following steps: collecting a small sample of a biological fluid from the test subject, most commonly a drop of blood from a finger tip, adding sample to a viral test cassette, initiating sample processing by the assay cassette to incubate the diluted sample and developing reagents with an antibody microarray contained within the cassette, and results of the test are read by visual examination or preferably by use of a reader that will capture a digital image of the assay microarray and process the results to determine if the subject is infected by a coronavirus.

In an embodiment, a method for detecting the presence of antibodies specific for a coronavirus in a sample comprises the following steps: collecting a small sample of a biological fluid from the test subject, adding sample to a viral test cassette, initiating sample processing by the assay cassette to incubate the diluted sample and developing reagents with an protein antigen microarray contained within the cassette, and read the results of the test by visual examination or preferably by use of a reader that will capture a digital image of the assay microarray and process the results to determine if the subject is infected or has been infected by a coronavirus in the past.

In one embodiment, a system for detecting the presence of a virus in a sample may comprise a platform comprising a buffer chamber in fluid communication with a sample receiver, the sample receiver comprising a sample chamber and a membrane and is in fluid communication with a secondary agent depot, the secondary agent depot comprising a secondary agent and is in fluid communication with a reaction chamber, the reaction chamber comprising an array comprising at least one viral peptide and an optical window and is in fluid communication with a waste chamber.

In an embodiment, the virus may be a coronavirus, preferably SARS-CoV-1, MERS-CoV, SARS-CoV-2 (COVID-19), HCoV-OC43, HCoV-HKU1, HCoV-NL63, HCoV-229E or a combination thereof. The viral peptide may be a coronavirus peptide hemmaglutinin esterase (He), membrane protein (M), envelope small membrane protein (E), nucleocapsid (N), spike (S), or a combination thereof. The viral peptide may be a SARS-CoV peptide, MERS-CoV peptide, SARS-CoV-2 (COVID-19) peptide, HCoV-OC43 peptide, HCoV-HKU1 peptide, HCoV-NL63 peptide, HCoV-229E peptide, an antigenic fragment thereof, or a combination thereof. The viral peptide may be a SARS-CoV-2 (COVID-19) peptide, an antigenic fragment thereof, or a combination thereof. The S protein may be SSARS-2, SSARS, SMERS, SOC43, SHKU1, SNL63, S229E, antigenic fragments thereof, or a combination thereof. The N protein may be NSARS-2, NSARS, NMERS, NOC43, NHKU1, NNL63, N229E, antigenic fragments thereof, or a combination thereof. The coronavirus viral peptide may comprise a sequence selected from the group consisting of the sequences listed in Tables 2, 3, 4, or any combination thereof. The coronavirus viral peptide may comprise a sequence selected from the group consisting of an amino acid sequence with at least about 90% sequence homology to the amino acid sequences of 248, 250, 252, 254, 256, 258, 260, 262, 264, 266, 268, 270, 272, 274, antigenic fragments, epitopes contained therein, or combinations thereof. The coronavirus viral peptide may comprise a sequence selected from the group consisting of an amino acid sequence of 248, 250, 252, 254, 256, 258, 260, 262, 264, 266, 268, 270, 272, 274, antigenic fragments, epitopes contained therein, or combinations thereof.

In an embodiment, the virus may be a filovirus, optionally a Zaire ebolavirus (Ebola Virus), Sudan ebolavirus (Sudan virus), Taï Forest ebolavirus (Côte d'Ivoire ebolavirus) (Taï Forest virus), Bundibugyo ebolavirus (Bundibugyo virus), Reston ebolavirus (Reston virus), Bombali ebolavirus (Bombali virus), Marburg marburgvirus (Marburg virus), or a combination thereof. The viral peptide may be an Ebola virus peptide or an antigenic fragment thereof. The filovirus peptide may be a Zaire ebolavirus (Ebola Virus) peptide, Sudan ebolavirus (Sudan virus) peptide, Taï Forest ebolavirus (Côte d'Ivoire ebolavirus) (Taï Forest virus) peptide, Bundibugyo ebolavirus (Bundibugyo virus) peptide, Reston ebolavirus (Reston virus) peptide, Bombali ebolavirus (Bombali virus) peptide, Marburg marburgvirus (Marburg virus) an antigenic fragment thereof, or a combination thereof. The Filoviral peptide may be glycoprotein (GP), nucleocapsid protein (NP), minor nucleoprotein (VP30), polymerase complex protein (VP35), matrix (VP40), VP24, an antigenic fragment thereof, or a combination thereof. The Filoviral peptide may be a glycoprotein, nucleocapsid protein (NP), an antigenic fragment thereof, or a combination thereof. The Filoviral peptide may comprise a sequence selected from the group consisting of the sequences listed in Table 2, Table 5, or any combination thereof. The Filoviral peptide may comprise a sequence selected from the group consisting of the amino acid sequences of SEQ ID NOs: 2, 4, 6, 8, 10, 12, 16, 18, 20, 22, 24, 26, 28, 30, 32, 34, 36, an antigenic fragment thereof, an epitope therein, or a combination thereof. The Filoviral peptide may comprise a NP sequence selected from the group consisting of an amino acid sequence with at least about 90% sequence homology to the amino acid sequences of SEQ ID NOs: 4, 10, 16, 22, 28, 34, an antigenic fragment thereof, an epitope therein, or a combination thereof. The Filoviral peptide may comprise a VP sequence selected from the group consisting of the amino acid sequences of SEQ ID NOs: 4, 10, 16, 22, 28, 34, an antigenic fragment thereof, an epitope therein, or a combination thereof.

In an embodiment, the virus may be a Yellow Fever virus (flavivirus). The viral peptide may be a Yellow Fever virus peptide. The viral peptide may be a structural protein from the Yellow Fever virus, optionally Capsid, pM, E, or a combination thereof. The viral peptide may be a non-structural protein from the Yellow Fever virus, optionally NS1, NS2a, NS2b, NS3, NS4a, NS4b, NS5, or a combination thereof. The flaviviral peptide may comprise a sequence selected from the group consisting of the sequences listed in Tables 2, 7, or any combination thereof. The flaviviral peptide may comprise an amino acid sequence with at least 90% sequence homology to the amino acid sequence of SEQ ID NO: 37, 40, 43, 46, 49, 52, 55, 58, 61, 64, 67, 70, 73, 76, 79, 82, 88, 91, 94, 97, 100, 103, 106, 109, 112, 115, 118, 121, 124, 127, 130, 133, 136, 139, 142, 145, 148, 151, 154, 157, 160, 163, 166, 169, 172, 175, 178, 181, 184, 187, 190, 193, 196, 199, 202, 205, 208, 211, 214, 217, 220, 223, 226, 229, 232, 235, 238, 241, 244, antigenic fragments thereof, eptiopes contained therein, or a combination thereof. The flaviviral peptide may comprise an amino acid sequence of SEQ ID NO: 37, 40, 43, 46, 49, 52, 55, 58, 61, 64, 67, 70, 73, 76, 79, 82, 88, 91, 94, 97, 100, 103, 106, 109, 112, 115, 118, 121, 124, 127, 130, 133, 136, 139, 142, 145, 148, 151, 154, 157, 160, 163, 166, 169, 172, 175, 178, 181, 184, 187, 190, 193, 196, 199, 202, 205, 208, 211, 214, 217, 220, 223, 226, 229, 232, 235, 238, 241, 244, antigenic fragments thereof, eptiopes contained therein, or a combination thereof.

In an embodiment, the reaction chamber may further comprise a viral peptide selected from the group consisting of influenza A virus peptides, influenza B virus peptides, influenza C peptides, enterovirus peptides, respiratory syncytial virus (RSV) peptides, parainfluenza peptides, adenovirus peptides, or a combination thereof. The influenza A virus may be H1N1, H1N2, H3N2, H5N1. H1N2, H7N9, or a combination thereof.

In an embodiment, a system for detecting the presence of a coronavirus in a sample may comprise a platform comprising a buffer chamber in fluid communication with a sample receiver, the sample receiver comprising a sample chamber and a membrane and is in fluid communication with a secondary agent depot, the secondary agent depot comprising a secondary agent and is in fluid communication with a reaction chamber, the reaction chamber comprising an array comprising at least one anti-viral antibody or antigen binding fragment thereof and an optical window and is in fluid communication with a waste chamber.

In an embodiment, the anti-viral antibody or antigen-binding fragment thereof may specifically bind a coronavirus peptide hemmaglutinin esterase (He), membrane protein (M), envelope small membrane protein (E), nucleocapsid (N), spike (S), or a combination thereof. The anti-viral antibody or antigen-binding fragment thereof may specifically bind a coronavirus selected from the group consisting of SARS-CoV, MERS-CoV, SARS-CoV-2 (COVID-19), or a combination thereof. The anti-viral antibody or antigen-binding fragment thereof may specifically bind a SARS-CoV-2 (COVID-19). The anti-viral antibody or antigen-binding fragment thereof may specifically bind a S protein, preferably SSARS-2, SSARS, SMERS, SOC43, SHKU1, SNL63, S229E, antigenic fragments thereof, or a combination thereof. The anti-viral antibody or antigen-binding fragment thereof may specifically bind a N protein, preferably NSARS-2, NSARS, NMERS, NOC43, NHKU1, NNL63, N229E, antigenic fragments thereof, or a combination thereof.

In an embodiment, the anti-viral antibody or antigen-binding fragment thereof may specifically bind an Ebola virus peptide. The filovirus may be Zaire ebolavirus (Ebola Virus), Sudan ebolavirus (Sudan virus), Taî Forest ebolavirus (Côte d'Ivoire ebolavirus) (Taï Forest virus), Bundibugyo ebolavirus (Bundibugyo virus), Reston ebolavirus (Reston virus), Bombali ebolavirus (Bombali virus), Marburg marburgvirus (Marburg virus) or a combination thereof. The filovirus may be Ebola virus, Sudan virus, Taï Forest virus, Bundibugyo virus, Reston virus, Marburg virus or a combination thereof. The anti-viral antibody or antigen-binding fragment thereof may specifically bind a Filoviral peptide selected from the group consisting of glycoprotein (GP), nucleocapsid protein (NP), minor nucleoprotein (VP30), polymerase complex protein (VP35), matrix (VP40), VP24, an antigenic fragment thereof, or a combination thereof. The anti-viral antibody or antigen-binding fragment thereof may specifically bind a Filoviralpeptide selected from the group consisting of glycoprotein, nucleocapsid protein (NP), an antigen fragment thereof, or a combination thereof.

In an embodiment, the anti-viral antibody or antigen-binding fragment thereof may specifically bind a Yellow Fever virus peptide. The anti-viral antibody or antigen-binding fragment thereof may specifically bind a structural protein from the Yellow Fever virus. The structural protein may be Capsid, pM, E, an antigenic fragment thereof, or a combination thereof. The anti-viral antibody or antigen-binding fragment thereof may specifically bind a non-structural protein from the Yellow Fever virus. The non-structural protein may be NS1, NS2a, NS2b, NS3, NS4a, NS4b, NS5, an antigenic fragment thereof, or a combination thereof. The anti-viral antibody or antigen-binding fragment thereof may comprise an anti-viral antibody or antigen-binding fragment thereof listed in Table 1.

In an embodiment, the reaction chamber may further comprise an anti-viral peptide antibody or binding fragment thereof that selectively bind a viral peptide selected from the group consisting of influenza A virus peptides, influenza B virus peptides, enterovirus peptides, respiratory syncytial virus (RSV) peptides, parainfluenza peptides, adenovirus peptides, or a combination thereof. The influenza A virus may be H1N1, H1N2, H3N2, H5N1, H1N2, H7N9, or a combination thereof.

In an embodiment, the viral peptides may be recombinant. The antigenic fragment may be about 5-25 amino acids in length. The epitope may be about 5-12 amino acids in length.

In an embodiment, the sample may be a biological material that contains antibodies. The sample may be blood, serum, plasma, saliva, mucus, tears, breast milk, colostrum, vaginal secretions, or a combination thereof.

In an embodiment, the secondary antibody may be an anti-IgG antibody. The secondary antibody may be labeled. The label may be a fluorescent label, luminescent label, bioluminescent label, radioactive label, chemiluminescent label, colorimetric label, fluorogenic label, enzymatic label, or a combination thereof.

In an embodiment, the reaction chamber may have a volume of between about 1-500 μL, preferably between about 10 μL and 70 μL. The reaction chamber may have a volume of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 220, 225, 230, 240, 250, 260, 270, 275, 280, 290, 300, 310, 320, 325, 330, 340, 350, 360, 370, 475, 380, 390, 400, 410, 420, 425, 430, 440, 450, 460, 470, 475, 480, 490, or 500 μL. The reaction chamber may have a volume between about 10-50 μL, 10-100 μL, 25-50 μL, 50-150 μL, 100-500 μL, 250-500 μL, 100-250 μL, or 50-100 μL.

In an embodiment, the buffer chamber may have a volume of between about 1-500 μL, preferably between about 10 μL and 70 μL. The buffer chamber may have a volume of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 220, 225, 230, 240, 250, 260, 270, 275, 280, 290, 300, 310, 320, 325, 330, 340, 350, 360, 370, 475, 380, 390, 400, 410, 420, 425, 430, 440, 450, 460, 470, 475, 480, 490, or 500 μL. The buffer chamber may have a volume between about 10-50 μL, 10-100 μL, 25-50 μL, 50-150 μL, 100-500 μL, 250-500 μL, 100-250 μL, or 50-100 μL.

In an embodiment, the waste chamber may have a volume of between about 1-500 μL, preferably between about 10 μL and 70 μL. The waste chamber may have a volume of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 220, 225, 230, 240, 250, 260, 270, 275, 280, 290, 300, 310, 320, 325, 330, 340, 350, 360, 370, 475, 380, 390, 400, 410, 420, 425, 430, 440, 450, 460, 470, 475, 480, 490, or 500 μL. The waste chamber may have a volume between about 10-50 μL, 10-100 μL, 25-50 μL, 50-150 μL, 100-500 μL, 250-500 μL, 100-250 μL, or 50-100 μL.

In an embodiment, the method for detecting the presence of a virus in a sample may comprise using the systems described herein. The method may comprise obtaining a sample from a patient, adding the sample to the system, reacting the sample, and obtaining the result.

In an embodiment, a positive result may be indicative of the presence of a virus in the sample or antibodies specific for a virus. The virus may be a coronavirus. The virus may be a SARS-CoV, MERS-CoV, SARS-CoV-2 (COVID-19), or a combination thereof. The virus may be a Filovirus. The Filovirus may be a Zaire ebolavirus (Ebola Virus), Sudan ebolavirus (Sudan virus), Taï Forest ebolavirus (Côte d'Ivoire ebolavirus)(Taï Forest virus), Bundibugyo ebolavirus (Bundibugyo virus), Reston ebolavirus (Reston virus), Bombali ebolavirus (Bombali virus), Marburg marburgvirus (Marburg virus), or a combination thereof. The virus may be a flavivirus. The virus may be a Yellow Fever virus, Zika virus, dengue virus, or a combination thereof.

### BRIEF DESCRIPTION OF THE DRAWINGS

FIGS. 1A-1C. The system comprises a platform comprising a buffer chamber, sample receiver, microfluidic network, reaction chamber comprising an array, and waste chamber. Blood (or other bodily fluid) is added to a sample receiver where a plasma membrane (1) separates the fluid component from cellular and particulate components of the blood (or bodily fluid). Capillary action pulls the plasma through the membrane to separate the blood cells and particulate matter from the plasma. A blister pack (not shown) is pierced by depressing a button (2) to release buffer. Turning vacuum cap (3) to the first stop pulls buffer through to dilute plasma and rapidly resuspend lyophilized reagents (4). The vacuum cap (3) houses a plunger (3a) that is sealed by an o-ring (3b) to prevent fluid leakage, and a compression spring (3c) that controls resistance of cap movements. The mixture of diluted plasma and reagents flows through a capillary channel to the array chamber (5) for a timed incubation. Turning the vacuum cap to the second stop (6) pulls buffer through to wash the microarray surface (5) and remove unbound analytes. The second buffer cycle also pulls the diluted plasma through a capillary channel to a waste chamber (7). FIG. 1A shows the top view of the device and the location of the plasma separation membrane filled with blood. FIG. 1B shows the bottom view and the separated plasma in the collection chamber. FIG. 1C shows the array chamber filled with buffer mixed with the plasma.

FIG. 2. An exemplary placement of printed microarray of antigens or antibodies for a specific assay. The microarray is positioned on a plastic chip of cyclic olefin copolymer (COC) or other material to provide optimal viewing through the optical window. The measurements are provided in millimeters (mm). The dimension are for illustrative purposes only and are not limiting.

FIGS. 3A-3B. An exemplary integrated reagent storage and plasma separation on an assay cassette. A main chip (9) configured to comprise a buffer chamber, blood chamber comprising a chamber, membrane, and compression means (e.g., compression ring), secondary agent chamber (e.g., lyophilized secondary reagents may be included in bead form, e.g., lyophilized encapsulated anti-human IgG-Alexa Fluor 488), array, and waste repository all in fluidic communication (e.g., microfluidic) with each other in that order. The secondary reagent may be provided by means of a lyophilized bead or reagent pellet (10) about 1-5 mm in diameter. A pressure sensitive adhesive (PSA) cover (8) may be placed over the secondary agent chamber, array, waste repository, or any combination thereof. The bottom of the main chip may be sealed with a PSA. An optical window may be affixed to the PSA on the bottom of the chip. Solvent, laser, or other sealing methods may be used instead of PSA to assemble cassette components. A preferred optical window may be fabricated with a COC that provides high transparency, low birefringence, high Abbe number, and high heat resistance. A lyophilized bead containing the secondary reagent may be stored within the assay cassette. The manufactured lypholized beads comprise 10% trehalose (by weight) and secondary antibodies that are conjugated to a fluorescent dye and specific for human IgA, IgM or IgG antibodies. The lyophilized beads are spherical, have a preferred diameter of 3 mm, and dissolve in <15 seconds of contact with buffer stored within the cassette without the requirement for additional mixing. Negative fluidic pressure is preferred above positive pressure. Pushing buffer through microfluidic channels introduces bubbles through the plasma filter, whereas negative fluidic pressure reduces introduction of air bubbles that can interfere with visualization of the assay results through the optical window. The use of negative pressure by a mechanical actuator eliminates the need for any electrical means for moving the reagents, sample, and buffer through the cassette microfluidic network. Twisting the vacuum actuator (3), which is optionally encased in a twist cap (11), to the first stop pulls buffer through to dilute plasma and rapidly resuspend the lyophilized reagent pellet (10). Optionally, a cap (12) may cover the blister pack of liquid buffer.

FIGS. 4A-4B. Operation of assay steps with cassette: A drop of blood from a finger prick with a lancet, or about 50-100 μL, is added to a receptacle where capillary action draws the sample across a membrane, separating the whole blood components from the plasma. No electrical means are required to move the blood across the membrane. Second, pressure applied to the buffer cap pierces a blister capsule (a first buffer chamber) and a twist cap is turned to the first stop, releasing buffer into the microfluidic channel, mixing with the plasma and hydrating the lyophilized reagents in a sufficient volume for the assay reaction to occur in the microarray. Third, a second introduction of buffer by moving the twist cap to a second stop washes the non-bound reagents and samples out of the microarray, leaving only samples, primary and second reagents bound to the microarray probes. A barcode (2 or 3D) (not shown) placed on the cassette at a position that is not over the microarray window can encode reference information for test identity, production lots, subject identification, time and place of assay performance, and other information as needed. Design modifications for scaled production by injection-molding (FIG. 4B) include adding wings on the twist cap and mold-release guide holes in the cassette body.

FIGS. 5A-5B. Specificity of the polyclonal antibody response to yellow fever virus 17D. FIG. 5A. Microarrays of YFV and DENV1-4 antigens (envelope (E), non-structural protein 1 (NS1), precursor membrane (pM), YFV virus (strain 17D) were probed with mouse polyclonal antibodies against YFV or FIG. 5B. sera from YFV-vaccinated non-human primates (NHP, collected 30 days post-immunization). Antibody binding to select microarray antigens is shown as the mean fluorescence intensity (MFI) of signals f SEM of replicate protein spots (A, n=6) or sera samples (B, n=3). In Antibody recognition of control probes (left axis; species-specific IgG, black; all others, gray) is compared to viral antigens (right axis; DENV, unfilled; YFV, patterned).

FIG. 6. Correlation of antibody responses to envelope (E), non-structural protein 1 (NS1), pre-membrane (pM) antigen and whole virus. Pearson's correlation analysis was performed using results of serum IgG binding to E from yellow fever virus and whole virus as measured by microarray assays of all vaccinated (n=67) and non-vaccinated (n=11) subjects. Values shown are log 2 transformed mean fluorescence intensity of antibody binding (IgG) signal to the dilution of 17D whole virus (x axis) and YFV recombinant protein (y-axis) with the highest AUC value as noted in Table 8. The dotted lines indicate the 95% confidence bands of the best fit line. Pearson's rank r value and p value are displayed on the scatter plot. E vs whole virus, r=0.70; NS1 vs whole virus, r=0.66; pM vs whole virus, r=0.55; p<0.0001.

FIG. 7. Kinetics of humoral immune response to YFV vaccination. Human IgG binding to (A) YFV proteins (envelope: E (open circles on solid line), non-structural protein 1: NS1 (open squares on solid line), precursor membrane protein: pM (open triangles on solid line) and (B) whole virus strain 17D (solid triangle on dashed line). 30-90d, n=19; 91-270d, n=17, 271-364d, n=10; 1-6 yr, n=7; 7-16 yr, n=8: non-vaccinated individuals, n=11. Serum antibody binding to printed antigens was detected with Alexa647 conjugated anti-human IgG (7 specific) secondary antibody. The relative amount of antibody bound was measured as mean fluorescence intensity (MFI). The values shown are the signal ratio of the immunized antibody response after dividing results for non-vaccinated MFI by antigen. Error bars indicate+/−standard error of the mean.

FIG. 8. Levels of virus-specific antibodies are elevated in subjects boosted with 17D vaccine. Antibody responses to yellow fever virus proteins (envelope: E, non-structural protein 1: NS1, and precursor membrane protein: pM) and vaccine strain whole virus in primary 17D vaccines (white bars, 1-6 yr post-primary vaccination (n=7); light gray bars, 7-16 yr post-primary vaccination (n=8)) compared to boosted (gray patterned bars, 1-6 yr post-boost or 7-16 yr post-primary before receiving boost (n=10)). Signal ratios of the immunized antibody response divided by non-vaccinated (n=11) by antigen. The dashed line indicates the cut-off value. Bars on the whisker plots indicate the minimum and maximum signals, while individual signals are shown as circles and the horizontal lines indicating cohort mean. Statistically significant differences between mean antibody binding to yellow fever virus antigens of primary and boosted vaccinations were calculated by two-way analysis of variance (ANOVA) analyses, with multiple comparison's corrected with Tukey's (****, p<0.0001, ns=not significant).

FIG. 9. Levels of virus-neutralizing antibodies are elevated in subjects boosted with the 17D vaccine. A flow cytometry infectivity assay was used to calculate the neutralization titers of YFV vaccinated subjects following two independent experiments. The serum dilution that neutralized 50% of YFV (strain 17D) was calculated for each primary (1-6 yr, n=6 and 7-16 yr, n=6) and secondary vaccinated individual (n=6), by nonlinear, dose-response regression analysis (F parameter constrained to 50). Bars indicate the mean 50% neutralization titer for each cohort (primary, non-filled bars; boosted, gray pattern bars) with error bars indicating standard error of the mean. Individual data points are also shown for primary and secondary vaccinated subjects (open circles) and boosted. Statistically significant differences between mean 50% neutralizing titers to yellow fever virus in 1-6 yr, 7-16 yr-primary and boosted vaccinations were calculated by Kruskal-Wallis non-parametric test (*, p<0.05, ns=not significant).

FIG. 10. Antibodies from 17D vaccination present minimal level of antibody cross reactivity towards structural proteins of other viruses within Flavivaridae.

FIGS. 11A-11C. Filovirus microarray chip detects EBOV-specific antibodies in serum from survivors of Ebola Virus disease. FIG. 11A depicts 2×2 microarray layout within a 4×4 mm space. The dimensions being provided for illustrative purposes only and are not intended to be limiting. FIG. 11B depicts Filovirus microarray probed with serum from Ebola Virus Disease detects antibodies to EBOV GP (spot #2, circled in the figure) and EBOV NP (spot #8, circled in the figure). FIG. 11C depicts the microarray key. The GP antigens confer greater specificity to the filovirus assay whereas antibodies to NP antigens are more abundant and provide greater sensitivity for detection of disease. Serum obtained from survivors of Ebola virus disease contains antibodies that bind specifically to EBOV GP and more highly to EBOV NP than to NPs from other filoviruses.

FIGS. 12A-12B. Filovirus microarray chip distinguishes Ebola Virus disease from EBOV vaccination. FIG. 12A depicts Vaccine formulated with EBOV GP does not contain EBOV NP and produces antibodies only to GP. FIG. 12B depicts Ebola virus disease produces antibodies to both EBOV GP and NP antigens (disease dark boxes; controls light boxes). Ebola vaccination can be distinguished from Ebola virus disease within the same assay by using the filovirus microarray chip printed with NP and GP antigens from 6 filoviruses. In this example, blood serum antibodies were examined in subjects 30 days after receiving a live virus-vectored vaccine that displayed the Ebola (EBOV) GP antigen. Antibodies from EBOV vaccination bind only to EBOV GP but not NP antigens (cut-off values, dashed line). Antibody bound is expressed as relative fluorescent units (mean±SEM) from 30 human subjects. Antibodies from survivors of Ebola virus disease bind to both GP and NP from EBOV. Dark bars denote Ebola virus disease survivors and light bars non-infected controls from the same location (mean±SD, black bar).

FIG. 13. Infections by coronaviruses other than SARS-CoV-2 are common within the population and theses can be detected by microarray chip. FIG. 13 depicts coronavirus antibodies from randomly selected individuals.

Human infections from non-COVID-19 coronaviruses are common and can be detected with microarray chips printed with NP and S antigens. The antigens selected for inclusion in assays to detect infections from distinct coronaviruses were designed to minimize antibody cross-reactivity.

FIG. 14 depicts that the SARS-CoV-2 and SARS-CoV-1 Spike antigens are cross-reactive with antibodies from COVID-19 or animal antisera as detected by microarray chip. While antigens from the closely related coronaviruses SARS-CoV and SARS-CoV-2 exhibit antibody cross-reactivity antigens with rabbit anti-CoV-2 antigen antibodies and sera from COVID-19 patients (relative fluorescent units, mean f SD) by microarray chip, antibody cross-reactivity is minimal or not detectable against the other human coronaviruses. Antibody binding was visualized with anti-IgFc Dylight488ut and the cut-off value is shown as dashed line.

FIG. 15 depicts that the coronavirus microarray chips described herein can be used to measure virus-neutralizing antibodies by inhibition of ACE2 binding, using serum from COVID-19 or SARS-CoV-2 vaccination. In addition to the determination of total HCoV-specific antibody, a unique assay will simultaneously assess total antibody and neutralizing antibody by a surrogate assay-on-a-chip. The amino-terminal domain (Si) of the viral S protein controls cell receptor interactions and the membrane-proximal S2 domain is responsible for cell fusion. Angiotensin converting enzyme 2 (ACE2), which is expressed in both the upper and lower human respiratory tracts, is utilized as a host cell receptor by SARS-CoV-2, SARS-CoV, and HCoV-NL63, while CD26 (DPP4) is the primary receptor for MERS-CoV. Amino acid residues Lys31 and Lys353 in human ACE2 are critical for binding to virus, and naturally selected mutations in viral Si (e.g. K479N, S487T and N501Y) enhance the affinity of Si for human ACE2. Interactions between ACE2 and the receptor binding domain (RBD) from the S protein can be measured in vitro, and these antibody-inhabitable interactions form the basis of neutralizing antibody assay. Microarray chips printed with RBD proteins can be used to detect inhibition of ACE2 binding by anti-coronavirus antibodies, or ACE2 printed microarray chips can detect inhibition of RBD binding by anti-coronavirus antibodies. While there is no direct evidence that virus neutralization correlates with protection from infection in human COVID-19 patients, neutralizing antibodies have received approval from the Food and Drug Administration (FDA) for treating COVID-19 patients. New strains of CoV-SARS-2 are appearing in greater numbers due to selection of sequence shifts from the original L strain that appeared in Wuhan in December 2019, and the fastest spreading strains share the N501Y mutation of the S protein that increases affinity for the ACE2 cellular receptor. The phylogenetic lineage B.1.1.7 (Alpha) was first detection in the UK and exhibits deletion of S residues 69-70 (H,V) and the mutation N501Y. The B.1.351 (also known as 501Y.V2 or Beta) originated in South Africa and presents the mutations D614G, D80A, D215G, E484K, N501Y, A701V, L18F, R246I and K417N. The P.1 lineage (Gamma), first identified in Brazil, contains the mutations L18F, T20N, P26S, D138Y, R190S, K417T, E484K, N501Y, H655Y, and T1027IS, and a Delta lineage, originating in India, is rapidly becoming the most dominant infecting strain. Microarray chips printed with RBD or Spike proteins from SARS-CoV-2 variants can be used to assay binding of antibodies from COVID-19 and vaccinations. In this example, serum from patients who have recovered from COVID-19 caused by the L strain effectively block binding of the L strain RBD to ACE2 (COVID-19 median inhibition of 94.61%, control=7.879%; p<0.0001), whereas these same antibodies inhibit ACE2 binding to the RBD from B.1.351 to a much lessor extent (COVID-19 median inhibition of 32.54%, control=5.873%; p=0.0886).

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

### Definitions

Unless otherwise indicated, all terms used herein have the same meaning as they would to one skilled in the art.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

“About,” as used herein, refers broadly to up to a 5% variance in a given numeric value.

1 “Antibodies” as used herein refer broadly to antibodies or immunoglobulins of any isotype, fragments of antibodies, which retain specific binding to antigen, including, but not limited to, Fab, Fab′, Fab′-SH, (Fab′)z Fv, scFv, divalent scFv, and Fd fragments, chimeric antibodies, humanized antibodies, single-chain antibodies, and fusion proteins including an antigen-specific targeting region of an antibody and a non-antibody protein. Antibodies are organized into five classes-IgG, IgE, IgA, IgD, and IgM.

“Antigen” or “Antigenic,” as used herein, refers broadly to a peptide or a portion of a peptide capable of being bound by an antibody which is additionally capable of inducing an animal to produce an antibody capable of binding to an epitope of that antigen. An antigen may have one epitope, or have more than one epitope. The specific reaction referred to herein indicates that the antigen will react, in a highly selective manner, with its corresponding antibody and not with the multitude of other antibodies which may be evoked by other antigens. Antigens may be pathogen specific (e.g., expressed by COVID-19.)

“Array,” as used herein, refers broadly to a population of targets, such as viral peptides or anti-viral antibodies, that can be attached to a surface in a spatially distinguishable manner. An individual feature of an array can include a single copy of a target, such as a viral peptide or anti-viral peptide, or a population of targets, such as viral peptides or anti-viral antibodies, at an individual feature of the array. The population of viral peptides or anti-viral antibodies at each feature typically is homogenous, having a single species of the particular target. However, in some embodiments a heterogeneous population of viral peptides or anti-viral antibodies can be present at a feature. Thus, a feature need not include only a single viral peptide or anti-viral antibody species and can instead contain a plurality of different viral peptide or anti-viral antibody species.

“Epitope,” as used herein, refers broadly to the part of an antigen to which an antibody attaches itself. Generally, epitopes are between about 5-12 amino acids and may be contiguous (e.g., a string of amino acid residues) or non-contiguous or conformational (e.g., two different stretches of amino acids residues folded together as to be in close enough proximity to form an antibody binding site).

In the present invention, the term “homologous” refers to the degree of identity (see percent identity above) between sequences of two amino acid sequences, i.e. peptide or polypeptide sequences. The aforementioned “homology” is determined by comparing two sequences aligned under optimal conditions over the sequences to be compared. Such a sequence homology can be calculated by creating an alignment using, for example, the ClustalW algorithm. Commonly available sequence analysis software, more specifically, Vector NTI, GENETYX or other tools are provided by public databases.

The terms “sequence homology” or “sequence identity” are used interchangeably herein. For the purpose of this invention, it is defined here that in order to determine the percentage of sequence homology or sequence identity of two amino acid sequences or of two nucleotide sequences, the sequences are aligned for optimal comparison purposes. In order to optimize the alignment between the two sequences, gaps may be introduced in any of the two sequences that are compared. Such alignment can be carried out over the full-length of the sequences being compared. Alternatively, the alignment may be carried out over a shorter length, for example over about 5, about 10, about 20, about 50, about 100 or more nucleotides or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison. In D. Sankoff and J. B. Kruskal, (ed.), Time warps, string edits and macromolecules: the theory and practice of sequence comparison, Addison Wesley). The percent sequence identity between two amino acid sequences or between two nucleotide sequences may be determined using the Needleman and Wunsch algorithm for the alignment of two sequences. (Needleman, S. B. and Wunsch, C. D. (1970) J. Mal. Biol. 48, 443-453). Both amino acid sequences and nucleotide sequences can be aligned by the algorithm. The Needleman-Wunsch algorithm has been implemented in the computer program NEEDLE. For the purpose of this invention, the NEEDLE program from the EMBOSS package was used (version 2.8.0 or higher, EMBOSS: The European Molecular Biology Open Software Suite (2000) Rice, Longden, and Bleasby, Trends in Genetics 16, (6) 276-277, emboss.bioinformatics.nl/). For amino acid sequences, EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment. The identity defined as herein can be obtained from NEEDLE by using the NOBRIEF option and is labeled in the output of the program as “longest-identity”. The nucleotide and amino acid sequences of the present invention can further be used as a “query sequence” to perform a search against sequence databases to, for example, identify other family members or related sequences. Such searches can be performed using the NBLAST and XBLAST programs (version 2.0) of Altschul, et al. (1990) J. Mal. Biol. 215:403-10. BLAST nucleotide searches can be performed with the NBLAST program, score=100, word length=12 to obtain nucleotide sequences homologous to polynucleotides of the invention. BLAST protein searches can be performed with the XBLAST program, score=50, word length=3 to obtain amino acid sequences homologous to polypeptides of the invention. To obtain gapped alignments for comparison purposes, Gapped BLAST can be utilized as described in Altschul et al. (1997) Nucleic Acids Res. 25(17): 3389-3402. When utilizing BLAST and Gapped BLAST programs, the default parameters of the respective programs (e.g., XBLAST and NBLAST) can be used.

“Label,” as used herein, refers broadly to any atom or molecule that can be used to provide a detectable (preferably quantifiable) signal. Labels can be attached to a molecule of interest such as a secondary reagent. Labels may provide signals detectable by such non-limited techniques as fluorescence, radioactivity, colorimetry, gravimetry, X-ray diffraction or absorption, magnetism, enzymatic activity, and combinations thereof.

“Seropositive,” as used herein, refers broadly to a condition where a patient has a positive serum reaction to a test, especially in a test for the presence of an antibody specific for a pathogen. Seropositivity is an indicator of exposure to the pathogen. For example, a seropositive patient may show a positive serum reaction for the presence of anti-coronavirus antibodies.

“Substantially free,” as used herein, refers broadly to the presence of a specific component in an amount less than 1%, preferably less than 0.1% or 0.01%. More preferably, the term “substantially free” refers broadly to the presence of a specific component in an amount less than 0.001%. The amount may be expressed as w/w or w/v depending on the composition.

“Solid support,” “support,” and “substrate,” as used herein, refers broadly to any material that provides a solid or semi-solid structure with which another material can be attached including but not limited to smooth supports (e.g., metal, glass, plastic, silicon, and ceramic surfaces) as well as textured and porous materials. Substrate materials include, but are not limited to acrylics, carbon (e.g., graphite, carbon-fiber, nanotubes), ceramics, controlled-pore glass, cross-linked polysaccharides (e.g., agarose or SEPHAROSE®), gels, glass (e.g., modified or functionalized glass), graphite, inorganic glasses, inorganic polymers, metal oxides (e.g., SiO₂, TiO₂, stainless steel), nanomaterials (e.g., highly oriented pyrolitic graphite (HOPG) nanosheets), organic polymers, plastics, polacryloylmorpholide, poly(4-methylbutene), poly(ethylene terephthalate), poly(vinyl butyrate), polybutylene, polydimethylsiloxane (PDMS), polyethylene, polyformaldehyde, polymethacrylate, polypropylene, polystyrene, polyurethanes, polyvinylidene difluoride (PVDF), resins, silica, silicon (e.g., surface-oxidized silicon), or a combination thereof.

“Surface,” as used herein, refers broadly to a part of a support structure (e.g., substrate) that is accessible to contact with reagents, beads or analytes. The surface can be substantially flat or planar. Alternatively, the surface can be rounded or contoured. Exemplary contours that can be included on a surface are wells, depressions, pillars, ridges, channels or the like. The terms “surface” and “substrate” are used interchangeably herein.

“Treatment,” as used herein, refers broadly to alleviating signs and/or symptoms of a disease or injury condition. Treatment may encompass prophylactic measures, where the therapeutic composition is administered prior to the development of signs and/or symptoms or exposure to the disease or injury condition to lessen the development of signs and/or symptoms of a disease or injury condition.

### Cassette

There is an elevated concern for disease transmission to other parts of the world by infected travelers, prompting intense screenings at international airports, and travel bans for individuals from the affected countries. Polymerase chain reaction (PCR) and antigen detection tests for virus detection are performed and interpreted only in the clinic, and thus rely on movement of patients or specimens to operational laboratories. While PCR methodologies demonstrate sensitivity and specificity, data can only be obtained during the first few days of viremia, using dedicated equipment and highly trained personnel. A major drawback is that it is a laboratory tool, requiring venipuncture and sample transport to the laboratory creating long delays. A further drawback is that PCR tools are relatively expensive and are not widely implemented in the highest disease burden countries. Similarly, antigen detection tests are only useful during the first few days of infection, and few assays have been commercialized. There are no serological assays that are available in common clinical practice for detection of antibody responses to viral infections, and most experimental methods in development are capable of only measuring antibodies against a few viruses that cause human disease. An additional concern for the methods described above is requirement for milliliter volumes of blood, obtained by venipuncture.

Commonly practiced diagnostic methods for infectious diseases fall into three broad categories: (1) detection of viral nucleic acids, (2) detection of antigens that are expressed by the pathogen, or (3) analysis of antibodies that are produced by the host in response to infection. Antigen, antibody, and viral nucleic acids tests are useful only during certain periods during the infection. While nucleic acid amplification tests (NAAT) for pathogens demonstrate sensitivity and specificity, data can generally be obtained if the pathogen is present in biological fluids, for example only within the first days of active viremia, and results require dedicated equipment that is operated by highly-trained personnel. Similarly, antigen detection tests are only useful during the first few days of acute viral infections. A further complication is that most diagnostic tests are performed and interpreted only in specialized laboratories, and thus rely on movement of patients or specimens to operational facilities. The actual rate of infection, which includes unreported asymptomatic and mild cases, is also difficult to predict without reliable evidence. Microbead-based immunoassays, for example those that use the Luminex® instrument are useful for large scale seroprevalence studies that can only be performed by skilled users in a laboratory setting. However, there are no multiplexed antibody immunoassays that are highly portable and that can also be adapted to perform biomarker assays.

The inventors created rapid, high-throughput methods for the multiplexed analysis of serological immune responses to coronaviruses, Ebola, and Yellow Fever infections. The antibody detection system and methods described herein can monitor and report data from infections caused by any given virus at the point of use, and will use only a drop of blood. The device may also be able to acquire antibody data from both symptomatic and non-symptomatic cases, and thus enable an additional application and unmet need for disease surveillance. The diagnostic system and methods described herein address important gaps in studying the seroprevalence and surveillance of viral infections. In particular, the system and methods can detect the presence of anti-viral antibodies in general populations in endemic areas and identify any possible outbreaks. The inclusion of multiple antigens from closely or distantly-related viruses that are known or are perceived to stimulate cross-reactive antibodies enables the most accurate determination of the specific cause of infections by the multiplexed antibody detection system. This multiplexed information is important for determining the virus responsible for a current or previous infection, and may, for the case of COVID-19, provide guidance for the return to non-quarantined activities by individuals who present specific anti-SARS-2 antibodies, or for the case of yellow fever and filovirus diseases, the need for the individual to be vaccinated for protection from infections.

The system comprises a disposable microfluidic assay that detects disease-specific antibodies or biomarkers (e.g., viral peptides) within 15 minutes and a separate reader to analyze the results. Plasma separation from blood, reconstitution of test reagents, and all other assay steps are performed by the cassette. The assay cassette is sealed and single-use to greatly reduce risk from biohazards. The device can acquire serological results from both symptomatic and non-symptomatic cases, and thus enables an additional application and unmet need for disease surveillance.

In an embodiment, a length of the cassette may be in a range from about 40 mm to about 90 mm; in a range from about 45 mm to about 85 mm; in a range from about 50 mm to about 80 mm; in a range from about 52 mm to about 78 mm; in a range from about 54 to about 76 mm; in a range from about 56 to about 74 mm; in a range from about 58 mm to about 72 mm; in a range from about 60 mm to about 70 mm; in a range from about 61 mm to about 69 mm; in a range from about 62 mm to about 68 mm; in a range from about 63 mm to about 67 mm; or in a range from about 64 mm to about 66 mm. In another embodiment, the length of cassette may be in a range from 40 mm to 90 mm; in a range from 45 mm to 85 mm; in a range from 50 mm to 80 mm; in a range from 52 mm to 78 mm; in a range from 54 to 76 mm; in a range from 56 to 74 mm; in a range from 58 mm to 72 mm; in a range from 60 mm to 70 mm; in a range from 61 mm to 69 mm; in a range from 62 mm to 68 mm; in a range from 63 mm to 67 mm; or in a range from 64 mm to 66 mm. In an embodiment, the length of the cassette may be about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 mm. In an embodiment, the length of the cassette is about 64 mm, or is in a range from about 60 mm to about 70 mm.

In an embodiment, a width of the cassette may be in a range from about 10 mm to about 40 mm; in a range from about 15 mm to about 35 mm; in a range from about 16 mm to about 34 mm; in a range from about 17 mm to about 33 mm; in a range from about 18 to about 32 mm; in a range from about 19 to about 31 mm; in a range from about 20 mm to about 30 mm; in a range from about 21 mm to about 29 mm; in a range from about 22 mm to about 28 mm; in a range from about 23 mm to about 27 mm; or in a range from about 24 mm to about 26 mm. In another embodiment, the width of cassette may be in a range from 10 mm to 40 mm; in a range from 15 mm to 35 mm; in a range from 16 mm to 34 mm; in a range from 17 mm to 33 mm; in a range from 18 to 32 mm; in a range from 19 to 31 mm; in a range from 20 mm to 30 mm; in a range from 21 mm to 29 mm; in a range from 22 mm to 28 mm; in a range from 23 mm to 27 mm; or in a range from 24 mm to 26 mm. In an embodiment, the width of the cassette may be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mm. In an embodiment, the width of the cassette is about 25 mm, or is in a range from about 22 mm to about 28 mm.

In an embodiment of the cassette, a height of the cassette may be in a range from about 0.1 mm to about 10 mm; in a range from about 0.5 mm to about 8 mm; in a range from about 1 mm to about 7 mm; in a range from about 1.5 mm to about 6.5 mm; or in a range from about 2 to about 6 mm. In another embodiment, the height of cassette may be in a range from 0.1 mm to 10 mm; in a range from 0.5 mm to 8 mm; in a range from 1 mm to 7 mm; in a range from 1.5 mm to 6.5 mm; or in a range from 2 to 6 mm. In an embodiment, the height of the cassette may be about 0.1, 0.2, 0.3, 0.4, 0.5, 0.6, 0.7, 0.8, 0.9, 1, 2, 3, 4, 5, 6, 7, 8, 9, or 10 mm. In an embodiment of the cassette, the printed microarray of antigens or antibodies for an assay may be in a range from about 20 mm to about 27 mm from a rear edge of the cassette (where a rear edge and a front edge refer to lengthwise ends of the cassette). In an embodiment, the distance between the printed microarray of antigens or antibodies for an assay and the rear edge of the cassette may be in a range from about 20.5 mm to about 26.5 mm; in a range from about 21 mm to about 26 mm; in a range from about 21.5 mm to about 25.5 mm; in a range from about 22 mm to about 25 mm; in a range from about 22.5 mm to about 24.5 mm; or in a range from about 23 mm to about 24 mm. In an embodiment, the distance between the printed microarray of antigens or antibodies for an assay and the rear edge of the cassette may be in a range from 20.5 mm to 26.5 mm; in a range from 21 mm to 26 mm; in a range from 21.5 mm to 25.5 mm; in a range from 22 mm to 25 mm; in a range from 22.5 mm to 24.5 mm; or in a range from 23 mm to 24 mm. In an embodiment, the distance between the printed microarray of antigens or antibodies for an assay and the rear edge of the cassette may be about 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, or 27.0 mm. In an embodiment, the distance between the printed microarray of antigens or antibodies for an assay and the rear edge of the cassette may be about 23.70 mm, or in a range of from about 22 mm to about 25 mm.

In an embodiment of the cassette, the printed microarray of antigens or antibodies for an assay may be in a range from about 28 mm to about 35 mm from the front edge of the cassette. In an embodiment, the distance between the printed microarray of antigens or antibodies for an assay and the front edge of the cassette may be in a range from about 28.5 mm to about 34.5 mm; in a range from about 29 mm to about 34 mm; in a range from about 29.5 mm to about 33.5 mm; in a range from about 30 mm to about 33 mm; in a range from about 30.5 mm to about 32.5 mm; or in a range from about 31 mm to about 32 mm. In an embodiment, the distance between the printed microarray of antigens or antibodies for an assay and the front edge of the cassette may be in a range from 28.5 mm to 34.5 mm; in a range from 29 mm to 34 mm; in a range from 29.5 mm to 33.5 mm; in a range from 30 mm to 33 mm; in a range from 30.5 mm to 32.5 mm; or in a range from 31 mm to 32 mm. In an embodiment, the distance between the printed microarray of antigens or antibodies for an assay and the rear edge of the cassette may be about 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, or 35.0 mm. In an embodiment, the distance between the printed microarray of antigens or antibodies for an assay and the front edge of the cassette may be about 31.70 mm, or in a range of from about 30 mm to about 3 mm.

In an embodiment of the cassette, the printed microarray of antigens or antibodies for an assay may be about equidistant from the widthwise edges of the cassette. In an embodiment, the printed microarray may be in a range from about 7 mm to about 13 mm from the widthwise edges of the cassette. In an embodiment, the distance between the printed microarray and the widthwise edges of the cassette may be in a range from about 7.5 mm to about 12.5 mm; in a range from about 8.0 mm to about 12.0 mm; in a range from about 8.5 mm to about 11.5 mm; in a range from about 9.0 mm to about 11 mm; in a range from about 10.0 mm to about 11.0 mm; in a range from about 10.1 mm to about 10.9 mm; in a range from about 10.2 mm to about 10.8 mm; in a range from about 10.3 mm to about 10.7 mm; in a range from about 10.4 mm to about 10.6 mm. In an embodiment, the distance between the printed microarray and the widthwise edges of the cassette may be in a range from 7.5 mm to 12.5 mm; in a range from 8.0 mm to 12.0 mm; in a range from 8.5 mm to 11.5 mm; in a range from 9.0 mm to 11 mm; in a range from 10.0 mm to 11.0 mm; in a range from 10.1 mm to 10.9 mm; in a range from 10.2 mm to 10.8 mm; in a range from 10.3 mm to 10.7 mm; in a range from 10.4 mm to 10.6 mm. In an embodiment, the distance between the printed microarray and the widthwise edges of the cassette may be about 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or 12.0 mm. In an embodiment, the distance between the printed microarray and the widthwise edges of the cassette may be about 10.50 mm, or in a range from about 10.0 mm to 11.0 mm.

In an embodiment of the cassette, a length of the printed microarray may be in a range from about 4 mm to about 15 mm; in a range from about 5 mm to about 14 mm; in a range from about 6 mm to about 13 mm; in a range from about 6.5 mm to about 12 mm; in a range from about 7 mm to about 11 mm; in a range from about 7.5 mm to about 10 mm; in a range from about 8 mm to about 9 mm; in a range from about 8.2 mm to about 8.8 mm; or in a range from about 8.3 mm to about 8.7 mm. In an embodiment, the length of the printed microarray may be in a range from 4 mm to 15 mm; in a range from 5 mm to 14 mm; in a range from 6 mm to 13 mm; in a range from 6.5 mm to 12 mm; in a range from 7 mm to 11 mm; in a range from 7.5 mm to 10 mm; in a range from 8 mm to 9 mm; in a range from 8.2 mm to 8.8 mm; or in a range from 8.3 mm to 8.7 mm. In an embodiment, the length of the printed microarray may be about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, or 13.0 mm. In an embodiment, the length of the printed microarray may be about 8.61 mm, or in a range from about 8 mm to about 9 mm.

In an embodiment of the cassette, a width of the printed microarray may be in a range from about 2 mm to about 6 mm; in a range from about 2.2 mm to about 5.8 mm; in a range from about 2.4 mm to about 5.6 mm; in a range from about 2.6 mm to about 5.4 mm; in a range from about 2.8 mm to about 5.2 mm; in a range from about 3.0 mm to about 5.0 mm; in a range from about 3.2 mm to about 4.8 mm; in a range from about 3.4 mm to about 4.6 mm; in a range from about 3.6 mm to about 4.4 mm; or in a range from about 3.8 mm to about 4.2 mm. In an embodiment, the width of the printed microarray may be in a range from 2 mm to 6 mm; in a range from 2.2 mm to 5.8 mm; in a range from 2.4 mm to 5.6 mm; in a range from 2.6 mm to 5.4 mm; in a range from 2.8 mm to 5.2 mm; in a range from 3.0 mm to 5.0 mm; in a range from 3.2 mm to 4.8 mm; in a range from 3.4 mm to 4.6 mm; in a range from 3.6 mm to 4.4 mm; or in a range from 3.8 mm to 4.2 mm. In an embodiment, the width of the printed microarray may be about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 mm. In an embodiment, the width of the printed microarray may be about 4.00 mm, or in a range from about 3.0 mm to about 5.0 mm.

In an embodiment of the cassette, the sample receiver (1) may be between about 0.5 mm and about 10 mm from the buffer-release button (2). In an embodiment, the distance between the sample receiver (1) and the buffer-release button (2) may be about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 mm.

In an embodiment of the cassette, the sample receiver (1) may be between about 0.5 mm and about 10 mm from the lyophilized reagents (4). In an embodiment, the distance between the sample receiver (1) and the lyophilized reagents (4) may be about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 mm.

In an embodiment of the cassette, the buffer-release button (2) may be between about 0.5 mm and about 10 mm from the vacuum cap (3). In an embodiment, the distance between the buffer-release button (2) and the vacuum cap (3) may be about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 mm.

In an embodiment of the cassette, the lyophilized reagents (4) may be between about 0.5 mm and about 10 mm from the vacuum cap (3). In an embodiment, the distance between the lyophilized reagents (4) and the vacuum cap (3) may be about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 mm.

In an embodiment of the cassette, the lyophilized reagents (4) may be between about 0.5 mm and about 10 mm from the microarray surface (5). In an embodiment, the distance between the lyophilized reagents (4) and the microarray surface (5) may be about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 mm.

In an embodiment of the cassette, a diameter of each of the sample receiver (1), the buffer-release button and chamber (2), and vacuum actuator (3) may be, independently of one another, in a range from about 3 mm to about 13 mm; in a range of from about 4.5 mm to about 11.5 mm; in a range from about 5 mm to about 10 mm; in a range from about 5.5 mm to about 9.5 mm; in a range from about 6 mm to about 9 mm; or in a range from about 6.5 mm to about 8.5 mm. In an embodiment, the diameter of each of the sample receiver (1), the buffer-release button and chamber (2), and vacuum actuator (3) may be, independently of one another, in a range from 4 mm to 12 mm; in a range of from 4.5 mm to 11.5 mm; in a range from 5 mm to 10 mm; in a range from 5.5 mm to 9.5 mm; in a range from 6 mm to 9 mm; or in a range from 6.5 mm to 8.5 mm. In an embodiment, the diameter of each of the sample receiver (1), the buffer-release button and chamber (2), and vacuum actuator (3) may, independently of one another, be about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.1, 12.2, 12.3, 12.4, 12.5, 12.6, 12.7, 12.8, 12.9, or 13.0 mm.

Plasma separation from blood, storage and reconstitution of storage test reagents, reagent mixing, incubation of plasma with antigen microarrays, and waste collection are performed by the cassette. Each assay function is separated by microfluidic channels that exhibit reduced protein-binding qualities. A sample of biological fluid, which could be saliva or a drop of blood collected from the subject's fingertip, is inserted into the cassette and the assay is initiated by turning a small actuator button. Test results are captured from the assay cassette by a reader that is capable of detecting the assay signals (e.g., fluorescence), and the data can be visually interpreted, or more accurately by the use of automated data processing algorithms that installed in the reader or accessible through another peripheral device. Several concepts for assay cassettes were evaluated to reach a final design that would provide reduced assembly steps, user manipulations, technical development risks, and manufacturing costs. The inventors varied numerous parameters and tried each of numerous possible choices until they arrived at a successful result, where the prior art gave either no indication of which parameters were critical or no direction as to which of many possible choices is likely to be successful. The final design is an integrated, sealed disposable cassette that enables onboard storage of secondary reagent and a reactive chamber that allows interaction of antibody from plasma with immobilized antigens.

Using a microarray printer, for example non-contact printers manufactured by Scienion (Berlin, Germany) or Arrayjet (Glasgow, Scotland), assay proteins are patterned in a 1 cm² or other sized microarray on a transparent (e.g., cyclic olefin copolymer (COC)) assay window that is bonded to the device chassis. Glass slides, nitrocellulose coated surfaces, activated polymeric surfaces and other materials can be used as substrates to print the microarrays. The microarray surface is chemically activated prior to printing to immobilize proteins and can accommodate greater than 50 individual test antigens or probes. Proteins are spotted as microarrays of assay probes that are immobilized to COC or other surfaces comprising the detection window of the cassette. Other surfaces for immobilizing the microarrays include but are not limited to many polymeric plastics or nitrocellulose coated glass surfaces. COCs are the preferred class for polymeric plastic surfaces due to high transparency, low birefringence, high Abbe number and favorable heat resistance. Other classes of plastics that can also be used for microarray surfaces include cyclic olefin polymers, poly(methyl methacrylate), polycarbonate, and polystyrene.

### Cassette-Samples and Sample Receiver

The detection systems and methods described herein allow for the monitoring of viral infections or antibody responses at the point of care and for home use, and only require a drop of blood (e.g., about 60 μL). The volume of sample required by the system and methods described herein may be relatively small compared to conventional diagnostic methods. For example, the volume of the sample may be between 1-500 μL, preferably between about 40 μL and 90 μL. The volume of the sample may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 220, 225, 230, 240, 250, 260, 270, 275, 280, 290, 300, 310, 320, 325, 330, 340, 350, 360, 370, 475, 380, 390, 400, 410, 420, 425, 430, 440, 450, 460, 470, 475, 480, 490, or 500 μL. the volume of the sample may be between about 20-70 μL, 10-100 μL, 25-75 μL, 50-150 μL, 100-500 μL, 250-500 μL, 100-250 μL, or 50-100 μL. A preferred volume is about 60 μL and a preferred volume range is about 10-100 μL.

The sample may be serum, plasma, blood, semen, mucus, saliva, vaginal secretions, colostrum, urine, cerebrospinal fluid, ascities, breast milk, or tears, for example, or any other biological material that contains antibodies or pathogen antigens. The sample may be blood. The sample may be plasma.

The sample may be added to the sample receiver configured to comprise a membrane, a chamber, and a cover, e.g., cap. When the sample is added to the sample receiver, capillary action pulls the sample across the membrane, separating out larger components, to leave a fluid comprising antibodies. For example, if a blood sample is added to the sample receiver, capillary action will draw the blood across the membrane separating out the plasma and leaving the larger blood components, e.g., cells, inside the chamber. The inventors found that no electrical means were required to move the sample into the microfluidic network of the cassette, an advantage over conventional methods and systems. This allows the cassette to be used in a variety of field conditions where electricity may not be readily available. Components of the assay cassette are linked by microfluidic channels. Blood or other biological fluids move across a filtration membrane that removes cellular and particulate components, and capillary action combined with negative fluidic pressure applied by a spring-loaded actuator valve drives fluid movement through each step of the assay. The microfluidic channels may vary from 5-150 μM in height, with circular or rectangular cross-sections, though the channels can be larger or smaller. The channels can be fabricated as closed systems or as open systems that are closed during further assembly of the cassette. A hydrophobic air vent may be used to ensure that liquid introduced into the cassette is able to fill microfluidic channels by displacing air. A microfluidic channel carries non-bound substances in biological fluids from the microarray reaction chamber to a waste storage depot in the base of the twist valve, and the disposable cassette is sealed to prevent leaks of biological samples. A burst blister that is filled with buffer is pierced by pressing a button on the top of the cassette. The multi-layered foil burst blister consists of a lidding foil and forming foil that can hold 10-500 μL of fluid that is stored within the assay cassette. Such blisters can also be constructed from plastics or other non-permeable materials that are inert to assay buffers. The preferred volume range is 100-300 μL while the preferred volume is 250 μL. The buffer flow is regulated by a spring-loaded, twist actuator that causes the buffer to move through microfluidic channels to each assay activity. The buffer moves through a channel connecting the plasma that was separated from blood to a depot that holds lyophilized reagents. The diluted biological sample reconstitutes and mixes with the reagents and moves through a channel to a reaction chamber that contains the test microarray. A forked channel can also be used to deliver buffer diluted biological sample to two separate reagents depots that are connected by channels to two separate test microarray reaction chambers.

The membrane may be any hydrophilic membrane with a mean pore size between about 0.1 μm to about 100 μm. For example, the membrane may have a mean flow pore size from about 0.1 μm to about 10 μm. The membrane may be polysulfone, polyethersulfone, polyarylsulfone, polyvinylpyrrolidone. The membrane may be asymmetrical. For example, the membrane may be a plasma separation membrane, e.g., PALL Vivid® plasma separation membrane. See also U.S. Pat. Nos. 5,846,422; 5,906,742; 5,979,670; 6,045,899; 6,110,369; 6,277,281; 6,440,306; 6,565,782; 6,939,468; 7,125,493.

The system and methods described herein may be operated at a variety of temperatures. For example, the system and methods described herein may be operated at any temperature between about 0° C. and 50° C. For example, the system and methods described herein may be operated at a temperature at about 0° C., 1C, 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C. The system and methods described herein may be operated at a temperature between about 0° C. and 35° C., 10° C. and 30° C., 20° C. and 30° C., 20° C. and 45° C., or 20° C. and 40° C.

### Cassette-Buffer and Buffer Chambers

After the sample is added to the cassette, a first buffer chamber may be emptied into the microfluidic network. The buffer may serve at least three purposes, diluting the sample, hydrating the lyphollized reagents, and providing sufficient volume for the reaction. The buffer acts to move the sample into the array where the reaction occurs, surprisingly the dilution by the buffer also acts to reduce background signals (“noise”). The buffer also hydrates the lyophilized primary and secondary reagents. Finally, the buffer provides sufficient volume for the selective binding reactions to occur on the array. After a sufficient time for the reaction, a second buffer chamber may be emptied into the microfluidic network by means of a mechanical action. This second introduction of buffer acts as a wash, removing any unbound secondary agent and targets from the array.

For the system and methods described herein, a sufficient reaction time may be between about 1 minute and 60 minutes. The sufficient reaction time may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes. For example, a sufficient reaction time may be between about 1-5 minutes, 1-10 minutes, 25-50 minutes, 50-60 minutes, 10-50 minutes, 2.5-5 minutes, 1-2.5 minutes, 1-25 minutes, 5-25 minutes, 5-20 minutes, 10-20 minutes, 5-10 minutes, 15-20 minutes, 10-15 minutes, or 1-30 minutes.

The buffer may be any an isotonic solution, e.g., normal saline solution, buffered saline solution, lactated Ringer's solution, 5% dextrose in water (DSW), Ringer's solution, or 0.9% saline solution. The buffer may be a mineral buffer, balanced saline solution (BSS), TRIS buffer solution (TBS), phosphate buffered saline (PBS), organic buffers, borate buffer solution, carbonate buffer solution, carbonate buffered solution, citrate buffer solution, glycine buffer solution, TRIS buffered saline, Dulbecco's Phosphate saline buffer (DPBS), Dulbecco's Eagle Media (DMEM), Hank's Balanced Salts and Saline Solution (HBSS), Tyrode's Balanced Salts and Saline Solutions (TBSS), Minimum Essential Media, Eagle Basal Medium (EBM), Earle's Balanced Salts and Solutions (EBSS), Puk's Saline, Krebs-Ringer Bicarbonate Buffer, Krebs-Henseleit Buffer, Gey's Balanced Salt Solution (GBSS), Good's Buffers, ACES Buffer, BES Buffer, Bicine Buffer, Bis-Tris Buffer, CAPS Buffer, CAPSO Buffer, CHES Buffer, Glycyl-Glycyl Buffer, MES Buffer, HEPES Buffer, MOPS Buffer, Imidazole Buffer, Succinic Acid Buffer, or a combination thereof. For example, two or more different buffers may be used for the reaction and wash steps, or the same buffer may be used. Preferred buffers are 20 mM HEPES, 140 mM NaCl, 1% bovine serum albumin (BSA) and 0.01% Tween 20. Alternatively, 10 mM phosphate buffered saline with 140 mM NaCl, 1% BSA and 0.01% Tween 20 can be used. The pH of the buffer is preferably about pH 7.4. For example, the pH of the buffer may be close to mammalian plasma pH, e.g., between about pH 7.2 and pH 7.4. The pH of the buffer may be at about pH 7.0, pH 7.1, pH 7.2, pH 7.3, pH 7.4, pH 7.5, pH 7.6, pH 7.7, pH 7.8, or pH 7.9.

The volume of buffer required by the system and methods described herein may be relatively small compared to conventional diagnostic methods. For example, the volume of the buffer may be between 1-500 μL, preferably between about 10 μL and 70 μL. The volume of the buffer may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 220, 225, 230, 240, 250, 260, 270, 275, 280, 290, 300, 310, 320, 325, 330, 340, 350, 360, 370, 475, 380, 390, 400, 410, 420, 425, 430, 440, 450, 460, 470, 475, 480, 490, or 500 μL. The volume of the buffer may be between about 10-50 μL, 10-100 μL, 25-50 μL, 50-150 μL, 100-500 μL, 250-500 μL, 100-250 μL, or 50-100 μL. Preferred volumes are about 100-500 μL, more preferably about 100-200 μL.

The buffer may be stored in two or more separate chambers, or in a single chamber configured to deliver at least two separate infusions of buffer into the microfluidic network. In one embodiment, the buffer is stored in two separate chambers, and in another embodiment the buffer is stored in one chamber and the volume released is controlled by a fluidic actuator valve. The first chamber is released into the microfluidic network by means of pressing a cap to pierce a blister capsule. Any suitable chamber may be used that does not require electrical means to release the buffer or move it through the microfluidic network of channels in the cassette. An advantage of the present invention is that no electricity is required to move or deliver the reagents or samples to the microfluidic network or drive the reaction. Another advantage is that only microliter sample volumes of biological fluids are needed for a complete analysis, reducing the amount of test sample required. Only 1 μL from an average drop of blood with a volume of 50 μL may be needed for a typical assay. The larger volumes of biological fluids required by ELISA and other commonly practiced methods are more difficult to obtain from patients and increase the risk of transmitting infectious diseases. A further advantage is that only minute amounts of reagents, antigens or antibodies are needed to process the small volumes of biological fluids, thereby reducing cost and consumption of assay components.

### Cassette-Array

The system and methods described herein may utilize a peptide array or an antibody array. In a peptide array, peptides (e.g., antigens, epitopes) from a viral protein may be immobilized on the array. In an antibody array, an antibody specific for a virus is immobilized on an array. There are several methods by which proteins are spotted onto substrates. The choice of technology used impacts spotting consistency, speed, spot diameter, and ease of use. In turn, the final spot quality required for printed proteins will depend on the method of detection. Control of the laboratory environment to maintain constant temperature, humidity, and clean-room conditions provides the best printing consistency. The peptides or antibodies can be spotted in many patterns and the spot density can be increased by decreasing the spot diameters and pitch, which is the distance between individual spots. The amount of fluid delivered to the array surface determines the final density of peptide or antibody per spot. An array pattern of six columns and six rows of 150-300 μM spots, evenly spaced, is preferred for use with the assay cassette, while other spot arrangements, sizes, and numbers can also be used. Printing with solid pins relies on capillary forces to release spots on contact with the surface, resulting in 60-600 μM diameter spots depending on buffer composition and pin diameter. Dip-pen lithography (DPN) uses atomic force microscopy microcantilevers to deposit spots in the range of 1-60 μM diameter, and inkjet printers use piezoelectric elements to transfer the protein solution in the form of droplets to the target surface, resulting in spot diameters of 10-350 μM. Both pin and inkjet spotting methods deposit a very small amount of protein, requiring highly concentrated samples. Continuous flow microspotting uses microfluidic channel networks to continuously circulate protein samples over spots that are arranged by a fixed print mask to achieve uniform and maximum protein adsorption and is a technique that is useful for dilute and crude protein samples.

### Peptide Array

In a peptide array, a viral peptide comprising at least an epitope is immobilized on a solid support. The viral peptide may be a viral protein, a peptide thereof, an antigen thereof, an epitope thereof, or a combination thereof. The viral peptide may be a fusion protein between a carrier and a complete viral protein, a peptide thereof, an antigen thereof, an epitope thereof, or a combination thereof. Further the viral peptide may comprise a viral protein, a peptide thereof, an antigen thereof, an epitope thereof, or a combination thereof linked by means of a linker to the solid support.

The viral peptide may be an epitope comprising about 5-12 amino acids. For example, the viral peptide epitope may be about 5, 6, 7, 8, 9, 10, 11, or 12 amino acids in length. The viral peptide epitope may be between about 5-10 residues in length or 8-12 residues in length.

A single viral epitope may be immobilized on a solid support or a mixture of viral peptide epitopes may be immobilized on a solid support. For example, the peptide array may comprise between about 1-100 epitopes, although >100 epitopes may also be included. The peptide array may comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 viral peptide epitopes. The peptide array may comprise about 1-5, 10-100, 5-50, 5-15, 10-50, 25-50, 10-25, or 5-10 viral peptide epitopes.

The viral peptide may be a viral peptide comprising about 1-100 amino acids in length, although >100 amino acids may also be used. For example, the viral peptide may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length. The viral peptide may be about 1-5, 10-100, 5-50, 5-15, 10-50, 25-50, 10-25, or 5-10 amino acids in length. Antigenic fragments may be of a similar length.

The viral peptide may comprise a sequence listed in Table 2. The viral peptide for coronavirus cassettes may comprise a sequence listed in Table 3, Table 4, or a combination thereof. The viral peptide for Ebola virus cassettes may comprise a sequence listed in Table 5. The viral peptide for Yellow fever virus cassettes may comprise a sequence listed in Table 6.

The viral peptide may comprise an amino acid sequence having at least about 90% sequence homology to the amino acid sequences listed in Table 2. The viral peptide for coronavirus cassettes may comprise an amino acid sequence having at least about 90% sequence homology to the amino acid sequences listed in Table 3, Table 4, or a combination thereof. The viral peptide for Ebola virus cassettes may comprise an amino acid sequence having at least about 90% sequence homology to the amino acid sequences listed in Table 5. The viral peptide for Yellow fever virus cassettes may comprise an amino acid sequence having at least about 90% sequence homology to the amino acid sequences listed in Table 6. The amino acid sequence may have at least about 90%, 91%, 92%, 93%, 94%, 95%, 96%, 97%, 98%, 99%, or 100% sequence homology to the amino acid sequences listed in Tables 2, 3, 4, 5, 6, or a combination thereof. Further, the viral peptide may comprise an antigenic fragment of a sequence listed in Tables 2, 3, 4, 5, 6, or a combination thereof. The viral peptide may comprise an epitope contained within a sequence listed in Tables 2, 3, 4, 5, 6, or a combination thereof. The antigenic fragment may be about 5-25 amino acids in length. The epitope may be about 5-12 amino acids in length.

The coronavirus peptide may be selected from the group consisting of amino acid sequences having at least 90% homology to the amino acid sequences of SEQ ID NOs: 248, 250, 252, 254, 256, 258, 260, 262, 264, 266, 268, 270, 272, 274, or a combination thereof. The amino acid sequence may have at least about 90%, 91%, 92%, 93%, 94%, 95%, 96%, 97%, 98%, 99%, or 100% sequence homology to the amino acid sequences of SEQ ID NOs: 248, 250, 252, 254, 256, 258, 260, 262, 264, 266, 268, 270, 272, 274, or a combination thereof. Further, the viral peptide may comprise an antigenic fragment of the amino acid sequences of SEQ ID NOs: 248, 250, 252, 254, 256, 258, 260, 262, 264, 266, 268, 270, 272, 274, or a combination thereof. The viral peptide may comprise an epitope contained within a sequence the amino acid sequences of SEQ ID NOs: 248, 250, 252, 254, 256, 258, 260, 262, 264, 266, 268, 270, 272, 274, or a combination thereof. The antigenic fragment may be about 5-25 amino acids in length. The epitope may be about 5-12 amino acids in length.

The yellow fever viral peptide may be selected from the group consisting of amino acid sequences having at least 90% homology to the amino acid sequences of SEQ ID NOs: 37, 40, 43, 46, 49, 52, 55, 58, 61, 64, 67, 70, 73, 76, 79, 82, 88, 91, 94, 97, 100, 103, 106, 109, 112, 115, 118, 121, 124, 127, 130, 133, 136, 139, 142, 145, 148, 151, 154, 157, 160, 163, 166, 169, 172, 175, 178, 181, 184, 187, 190, 193, 196, 199, 202, 205, 208, 211, 214, 217, 220, 223, 226, 229, 232, 235, 238, 241, 244, or a combination thereof. The amino acid sequence may have at least about 90%, 91%, 92%, 93%, 94%, 95%, 96%, 97%, 98%, 99%, or 100% sequence homology to the amino acid sequences of SEQ ID NOs: 37, 40, 43, 46, 49, 52, 55, 58, 61, 64, 67, 70, 73, 76, 79, 82, 88, 91, 94, 97, 100, 103, 106, 109, 112, 115, 118, 121, 124, 127, 130, 133, 136, 139, 142, 145, 148, 151, 154, 157, 160, 163, 166, 169, 172, 175, 178, 181, 184, 187, 190, 193, 196, 199, 202, 205, 208, 211, 214, 217, 220, 223, 226, 229, 232, 235, 238, 241, 244, or a combination thereof. Further, the viral peptide may comprise an antigenic fragment of the amino acid sequences of SEQ ID NOs: 37, 40, 43, 46, 49, 52, 55, 58, 61, 64, 67, 70, 73, 76, 79, 82, 88, 91, 94, 97, 100, 103, 106, 109, 112, 115, 118, 121, 124, 127, 130, 133, 136, 139, 142, 145, 148, 151, 154, 157, 160, 163, 166, 169, 172, 175, 178, 181, 184, 187, 190, 193, 196, 199, 202, 205, 208, 211, 214, 217, 220, 223, 226, 229, 232, 235, 238, 241, 244, or a combination thereof. The viral peptide may comprise an epitope contained within a sequence the amino acid sequences of SEQ ID NOs: 37, 40, 43, 46, 49, 52, 55, 58, 61, 64, 67, 70, 73, 76, 79, 82, 88, 91, 94, 97, 100, 103, 106, 109, 112, 115, 118, 121, 124, 127, 130, 133, 136, 139, 142, 145, 148, 151, 154, 157, 160, 163, 166, 169, 172, 175, 178, 181, 184, 187, 190, 193, 196, 199, 202, 205, 208, 211, 214, 217, 220, 223, 226, 229, 232, 235, 238, 241, 244, or a combination thereof. The antigenic fragment may be about 5-25 amino acids in length. The epitope may be about 5-12 amino acids in length.

The Ebola peptide may be selected from the group consisting of amino acid sequences having at least 90% homology to the amino acid sequences of SEQ ID NOs: 2, 4, 6, 8, 10, 12, 14, 16, 18, 20, 22, 24, 26, 28, 30, 32, 34, 36, or a combination thereof. The amino acid sequence may have at least about 90%, 91%, 92%, 93%, 94%, 95%, 96%, 97%, 98%, 99%, or 100% sequence homology to the amino acid sequences of SEQ ID NOs: 2, 4, 6, 8, 10, 12, 14, 16, 18, 20, 22, 24, 26, 28, 30, 32, 34, 36, or a combination thereof. Further, the viral peptide may comprise an antigenic fragment of the amino acid sequences of SEQ ID NOs: 2, 4, 6, 8, 10, 12, 14, 16, 18, 20, 22, 24, 26, 28, 30, 32, 34, 36, or a combination thereof. The viral peptide may comprise an epitope contained within a sequence the amino acid sequences of SEQ ID NOs: 2, 4, 6, 8, 10, 12, 14, 16, 18, 20, 22, 24, 26, 28, 30, 32, 34, 36, or a combination thereof. The antigenic fragment may be about 5-25 amino acids in length. The epitope may be about 5-12 amino acids in length.

The viral peptides, including antigenic fragments and epitopes, may be synthesized or isolated from a virus. The viral peptides, including antigenic fragments and epitopes, may be from the same virus, same virus family, or a mixture of different viruses. Preferred lengths are 50-100 amino acid residues, although peptides <50 and >100 may also be used. Peptides from separate proteins or from different viruses may be fused together by chemical synthesis or by recombinant DNA technology. New tests can be rapidly prepared from information provided by genomic sequences of viruses that can be used to chemically synthesize genes that encode viral peptides, including antigenic fragments and epitopes, for use as templates to produce peptide or polypeptide products. Nucleic acid sequences from a known or newly discovered virus can be analyzed by genomic analysis algorithms to identify protein translation units that encode viral peptides, including antigenic fragments and epitopes, which can be included to prepare new tests. Preparations of purified or semi-purified whole virus can also be linked to the solid support for inclusion in serological tests. Viruses are preferably or optionally inactivated by gamma radiation prior to printing.

Densities of peptides or virus on the array can be adjusted by printing from 10 picoliters to 1 microliter of a peptide solution that can be from 10 μg/mL to 1000 μg/mL of peptide in buffer or 10⁴-10⁹ plaque forming units/mL of virus in buffer. 50-150 picoliters is the preferred range of volumes for printing. Protein unfolding agents can be added to the peptide to increase solubility for printing and to allow greater access of printed peptides to antibodies. Unfolding agents can include 0.1-1% sodium dodecyl sulfate, 0.1-2 M urea or other agents. Glycerol can be added to the peptide solution before printing to stabilize the printed peptide. Preferred concentrations of glycerol are 5-30% by volume of peptide solution. The buffer may be any an isotonic solution, e.g., normal saline solution, buffered saline solution, lactated Ringer's solution, 5% dextrose in water (D5W), Ringer's solution, or 0.9% saline solution. The buffer may be a mineral buffer, balanced saline solution (BSS), TRIS buffer solution (TBS), phosphate buffered saline (PBS), organic buffers, borate buffer solution, carbonate buffer solution, carbonate buffered solution, citrate buffer solution, glycine buffer solution, TRIS buffered saline, Dulbecco's Phosphate saline buffer (DPBS), Dulbecco's Eagle Media (DMEM), Hank's Balanced Salts and Saline Solution (HBSS), Tyrode's Balanced Salts and Saline Solutions (TBSS), Minimum Essential Media, Eagle Basal Medium (EBM), Earle's Balanced Salts and Solutions (EBSS), Puk's Saline, Krebs-Ringer Bicarbonate Buffer, Krebs-Henseleit Buffer, Gey's Balanced Salt Solution (GBSS), Good's Buffers, ACES Buffer, BES Buffer, Bicine Buffer, Bis-Tris Buffer, CAPS Buffer, CAPSO Buffer, CHES Buffer, Glycyl-Glycyl Buffer, MES Buffer, HEPES Buffer, MOPS Buffer, Imidazole Buffer, Succinic Acid Buffer, or a combination thereof. For example, two different buffers may be used for the reaction and wash steps, or the same buffer may be used. Preferred buffers are 20 mM HEPES, 140 mM NaCl. Alternatively, 10 mM phosphate buffered saline with 140 mM NaCl can be used. The pH of the buffer is preferably about pH 7.4. For example, the pH of the buffer may close to mammalian plasma pH, e.g., between about pH 7.2 and pH 7.4. The pH of the buffer may be at about pH 7.0, pH 7.1, pH 7.2, pH 7.3, pH 7.4, pH 7.5, pH 7.6, pH 7.7, pH 7.8, or pH 7.9.

### Antibody Array

In anti-viral antibody array, an anti-viral antibody, Fab fragment, binding fragment thereof, or a combination thereof are linked by a linking means to a solid support. The anti-viral antibody or binding fragment thereof may be arranged in a pattern. Different anti-viral antibodies including test and control anti-viral antibodies may be included in the same array. The molecular form of the antibody used is dependent on performance within the assay. The binary antigen-combining sites of IgG antibodies or antibody fragments can bind more antigen than a single-chain or monovalent antibody, resulting in a more stable antibody-antigen complex and a higher assay signal, which is preferred for some antibodies linked to the solid support. Increased assay specificity and decreased background noise may be obtained in some cases by using monovalent antibody fragments instead of bivalent antibodies. In general, a bivalent antibody or antibody fragment is preferred for inclusion in an antibody array, and monovalent or reduced sized antibody fragments can be preferred for optimal antibody binding to the cognate antigen.

A single anti-viral antibody or binding fragment thereof may be immobilized on a solid support or a mixture of anti-viral antibodies or binding fragments thereof may be immobilized on a solid support. For example, the anti-viral antibody array may comprise between about 1-100 anti-viral antibodies or binding fragments thereof. The anti-viral antibody array may comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 anti-viral antibodies or binding fragments thereof. The anti-viral antibody array may comprise about 1-5, 10-100, 5-50, 5-15, 10-50, 25-50, 10-25, or 5-10 anti-viral antibodies or binding fragments thereof.

The anti-viral antibodies or binding fragments thereof may selectively bind the same virus, same virus family, or a mixture of different viruses, e.g., a mixture of different anti-viral antibodies or binding fragments thereof. A capture antibody that is immobilized on the microarray surface will capture a specific virus or antigenic fragment from the virus. A second antibody, described as a detection antibody, which will bind to the same virus is stored within the assay cassette and is labeled with a fluorescent or colorimetric dye that will enable visualization of virus or viral antigen that is bound to the microarrayed antibody. A monoclonal antibody is preferred for attachment to the microarray surface and either a monoclonal antibody that binds to another epitope or a polyclonal antibody are preferred for detection antibodies. A polyclonal antibody can also be used as a capture antibody if it was produced by administering a single peptide epitope to a rabbit or other animal to generate an antibody response to the peptide. Table 1 lists antibodies that can be used in various combinations for capture or detection of filoviruses (e.g., Ebola, Sudan Taï Forest, and Bundibugyo viruses), coronaviruses (e.g., MERS CoV, SARS-CoV, SARS-CoV-2) and flaviviruses (e.g., yellow fever virus, dengue, Zika virus).

### Solid Support

The viral protein, in embodiments that use viral peptide arrays, or antibody or antigen-binding fragment, in embodiments that utilize an antibody array, may be attached to a solid support.

The substrate for the array used in the systems and methods described herein can be any material that provides a solid or semi-solid structure with which another material can be attached including but not limited to smooth supports (e.g., metal, glass, plastic, silicon, and ceramic surfaces) as well as textured and porous materials. Substrate materials include, but are not limited to acrylics, carbon (e.g., graphite, carbon-fiber, nanotubes), ceramics, controlled-pore glass, cross-linked polysaccharides (e.g., agarose or SEPHAROSE®), gels, glass (e.g., modified or functionalized glass), graphite, inorganic glasses, inorganic polymers, metal oxides (e.g., SiO₂, TiO₂, stainless steel), nanomaterials (e.g., highly oriented pyrolitic graphite (HOPG) nanosheets), organic polymers, plastics, polacryloylmorpholide, poly(4-methylbutene), poly(ethylene terephthalate), poly(vinyl butyrate), polybutylene, polydimethylsiloxane (PDMS), polyethylene, polyformaldehyde, polymethacrylate, polypropylene, polystyrene, polyurethanes, polyvinylidene difluoride (PVDF), resins, silica, silicon (e.g., surface-oxidized silicon).

Substrates need not be flat and can include any type of shape including spherical shapes (e.g., beads) or cylindrical shapes (e.g., fibers). Materials attached to solid supports may be attached to any portion of the solid support (e.g., may be attached to an interior portion of a porous solid support material).

Substrates may be patterned, where a pattern (e.g., stripes, swirls, lines, triangles, rectangles, circles, arcs, checks, plaids, diagonals, arrows, squares, or cross-hatches) is etched, printed, treated, sketched, cut, carved, engraved, imprinted, fixed, stamped, coated, embossed, embedded, or layered onto a substrate. The pattern can comprise one or more cleavage regions or modified regions on the substrate.

The viral peptide or anti-viral antibody can be attached to a substrate when it is associated with the solid substrate through a stable chemical or physical interaction. The attachment can be through a covalent bond. However, attachments need not be covalent or permanent. Materials may be attached to a substrate through a “spacer molecule” or “linker group.” Such spacer molecules are molecules that have a first portion that attaches to the viral peptide or anti-viral antibody and a second portion that attaches to the substrate. Thus, when attached to the substrate, the spacer molecule separates the substrate and the viral peptide or anti-viral antibody, but is attached to both. Methods of attaching biological material (e.g., nucleic acid, affinity ligand receptor, enzyme, chemical hydroxyl radical generator) to a substrate are known in the art, and include but are not limited to chemical coupling.

The surface of a substrate can be substantially flat or planar. Alternatively, the surface can be rounded or contoured. Exemplary contours that can be included on a surface are wells, depressions, pillars, ridges, channels, or a combination thereof. The polymeric surfaces are inert and do not possess any functional groups for attaching proteins or other biomolecules. They are also hydrophobic and need to be rendered hydrophilic. COC surfaces can be modified by oxygen plasma or under UV light, and the surfaces are reacted with aminosilane to allow the introduction of reactive groups to attach proteins. In one method, the COC slides are washed in water and ethanol, and dried under nitrogen. The slide surfaces are further cleaned and activated with UV irradiation and ozone for 5 minutes to oxidize the COC surfaces. The activated slides are immersed in a solution containing 3.0% aminopropyl triethoxysilane (APTES) in methanol: de-ionised water (95:5) for 1 h (22° C.). The slides are rinsed with methanol to remove unreacted reagents and cured at RT for 1 hour. The APTES-modified COC slides are immersed in a 25 mM 1, 4-Phenylene diisothiocyanate (PDITC) cross-linker in DMF:pyridine (9:1, v/v) solution for 2 h. The slides are then rinsed with DMF and MeOH and dried under a stream of nitrogen. The surfaces can also be fabricated to form nanostructures based on surface organization of 3D polymer brushes. Polymer backbones are tethered to a solid surface by one chain end. Polyethylene glycol, dextrans and other polymers terminated on another end with reactive functional groups (epoxy, NHS and others) are grafted to the COC surfaces. These 3D structures have the advantage of having a well-defined nanostructure with abundant functional groups to immobilize proteins, and may improve the sensitivity of the assay with some antigens. The preferred configuration for the array substrate is covalent attachment of peptides, proteins or antibodies by NHS or epoxy groups to COC surfaces. The peptides, proteins or antibodies are spotted in array patterns that consist of columns and rows. An array pattern of six columns and six rows of 150-300 μM spots, evenly spaced, is preferred for use with the assay cassette, while other spot arrangements, sizes, and numbers can also be used. The printed array is fixed in place within the assay cassette and is connected by microfluidic channel to the reagent chamber, and another microfluidic channel moves the diluted test sample from the array reaction chamber to a sealed waste depot. The use of microchannels to connect fluid inflow and outflow from the array chamber is preferred to allow semi-automated performance of the test assay with the cassette. The array is maintained in a dry state until the assay is initiated by fluid movement through the cassette. Keeping the array in a dry state increases product shelf life and reduces or eliminates the need for cold-chain storage. Fluid movement from the array chamber to the waste depot allows the unimpeded and metered flow of test sample, buffer, and assay reagents across the array. The microfluidic channel connecting the array chamber with the upstream buffer chamber allows additional metered movement of buffer across the array to wash away non-bound analytes of antibodies or antigens.

### Cassette-Secondary Reagents

The system and methods described herein may utilize a peptide array or an antibody array. In a peptide array, peptides (e.g., antigens, epitopes) from a viral protein may be immobilized on the array. To visualize the results from a peptide array, a secondary reagent comprising an anti-human antibody may be used to bind to the anti-viral antibodies captured from the sample. In an antibody array, an antibody specific for a virus is immobilized on an array. Viral peptides (e.g., whole viruses, viral peptides, viral antigens, viral epitopes) are captured by the immobilized anti-viral antibodies. The secondary reagents comprise a primary antibody, secondary antibody, or combination thereof. The primary antibody binds the captured virus peptides and the (labeled) secondary antibody binds to the primary antibody. The secondary agent may be stored in a secondary agent depot between the sample receiver and the reaction chamber.

Suitable labels for the secondary antibodies include, but are not limited to, fluorescent labels, luminescent labels, colorimetric labels, and combinations thereof. Fluorescent labels are molecules that absorb light of a specific wavelength and emit light of a different, typically longer, wavelength in a process known as fluorescence. Depending on the label used, the emitted light can be within the visible wavelengths from 380 to 700 nanometers, although labels outside of the visible range can be used. The labels may include the fluorescent dye Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, Alexa Fluor 680, Alexa Fluor 700, Alexa Fluor 750, Alexa Fluor 594, Coumarin, Cy3, Cy5, Fluorescein (FITC), Oregon Green, Pacific Blue, Pacific Green, Pacific Orange, PE-Cyanine7, PerCP-Cyanine5.5, Tetramethylrhodamine (TRITC), Texas Red, eFluor 450, eFluor 506, eFluor 660, PE-eFluor 610, PerCP-eFluor 710, APC-eFluor 780, Super Bright 436, Super Bright 600, Super Bright 645, Super Bright 702, Super Bright 780, Qdot 525, Qdot 565, Qdot 605, Qdot 655, Qdot 705, Qdot 800. Alexa Fluor dyes are the preferred fluorescent tag.

Chromogenic assay readouts can also be used, which are mediated by secondary antibodies that are conjugated to peroxidase, alkaline phosphatase or other enzymes and developed by the addition of chromogenic substrates.

The secondary agents may be provided in the form of a bead, preferably a lyophilized bead in about 1-5 μm in diameter. For example, the lyophilized bead may be 1, 2, 3, 4, 5, 6, 7, 8, 9, or 10 μm in diameter. The lyophilized bead may be about 3 μm in diameter.

The bead may include excipient stabilizers. Suitable excipient stabilizers include but are not limited to trehalose, sucrose, cyclodextrin, maltose, cellulose, and lactose. The excipient stabilizers may be used in amount about 1-75% by weight. For example, the excipient stabilizers may be about 5%, 10%, 15%, 20%, 25%, 30%, 35%, 40%, 45%, 50%, 55%, 60%, 65%, 70%, or 75% by weight. Trehalose 10% is a preferred excipient stabilizer.

A concentration of between about 1 μg/ml and 20 μg/ml of the secondary reagent may be encapsulated on the bead. For example, about 1, 2, 3, 4, 5, 6, 7, 8, 9, 10, 11, 12, 13, 14, 15, 16, 17, 18, 19, or 20 μg/ml of the secondary reagent may be encapsulated on the bead. In some embodiments, between about 1 μg/ml or 4 μg/ml of the secondary reagent may be encapsulated on the bead, e.g., 2 μg/ml or 3 μg/ml of the secondary reagent. A secondary reagent for an antibody assay can be an antibody that binds to human IgG or IgG subtypes, IgM, IgA, or IgE, and the secondary reagent is conjugated to a colorimetric, fluorescent or enzymatic tag that will localize antibody binding events on the assay microarray. The secondary reagent antibody can be a monoclonal or polyclonal antibody that originated in mice, rabbits, goats, camelids, rats, or other animal species, including cells line derived from these species or antibodies created by recombinant DNA methods. A secondary reagent for a viral antigen capture assay can be a detection antibody that is conjugated to a colorimetric, fluorescent or enzymatic tag that will localize antibody binding events on the assay microarray. The secondary reagent antibody can be a monoclonal or polyclonal antibody that originated in mice, rabbits, goats, camelids, rats, or other animal species, including cells line derived from these species or antibodies created by recombinant DNA methods. In the system and methods described herein, no active mixing is required to solubilize the secondary agents. The addition of the buffer resuspends the secondary agent within about 1-120 seconds, e.g., about 60 seconds. Further, negative fluid pressure produces very little air bubbles that could interfere with the imaging results from the reaction chamber.

### Cassette-Optical Window

The cassette may comprise an optical window that allows for detection of any signals generated by the labelled secondary agents. The cassette may comprise a single optical window or multiple windows. The optical windows may be fabricated using a material that provides high transparency, low birefringence, high Abbe number, high heat resistance, or any combination thereof. For example, suitable materials for an optical window include but are not limited to a cyclic olefin copolymer (COC). Cyclic-olefin-copolymer (COC) is preferred due to its low material costs, compatibility with mass-replication methods, chemical resistance to a wide range of solvents and chemicals, and good transparency in the ultraviolet and visible-light regions. The composition of an optical window can include, but are not limited to acrylics, carbon (e.g., graphite, carbon-fiber, nanotubes), ceramics, glass (e.g., silicon based), inorganic glasses, inorganic polymers, nanomaterials (e.g., highly oriented pyrolitic graphite (HOPG) nanosheets), organic polymers, plastics, polacryloylmorpholide, poly(4-methylbutene), poly(ethylene terephthalate), poly(vinyl butyrate), polybutylene, polydimethylsiloxane (PDMS), polyethylene, polyformaldehyde, polymethacrylate, polypropylene, polystyrene, polyurethanes, polyvinylidene difluoride (PVDF), resins, silica, silicon. COC and silicon glass are preferred materials.

### Cassette—Waste Chamber

The cassette may comprise a waste chamber which serves to collect and store any unused buffer, reagents, sample materials, and combinations thereof. In contrast to conventional systems and methods, the system and methods described herein produce relatively little waste. Further the waste may be non-toxic and safe for disposal in general refuse, e.g., may not require specialized disposal. The minute quantities of biological fluids used in the assay that are not inactivated can be sealed in the cassette for safe disposal. Due to the nature of the microfluidic network, no valve or closing means is required to prevent the waste from flowing back into the reaction chamber comprising the array. The waste chamber may be an isolated component of the cassette or can be incorporated into the vacuum twist button.

The volume of the waste chamber may be between 1-500 μL, preferably between about 10 μL and 70 μL. The volume of the waste chamber may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 220, 225, 230, 240, 250, 260, 270, 275, 280, 290, 300, 310, 320, 325, 330, 340, 350, 360, 370, 475, 380, 390, 400, 410, 420, 425, 430, 440, 450, 460, 470, 475, 480, 490, or 500 μL. the volume of the waste chamber may be between about 10-50 μL, 10-100 μL, 25-50 μL, 50-150 μL, 100-500 μL, 250-500 μL, 100-250 μL, or 50-100 μL. Preferred volumes are 200, 210, 220, 225, 230, 240, 250, 260, 270, 275, 280, 290, 300, 310, 320, 325, 330, 340, 350, 360, 370, 475, 380, 390, and 400 μL. The preferred range is 200-400 μL.

### Cassette

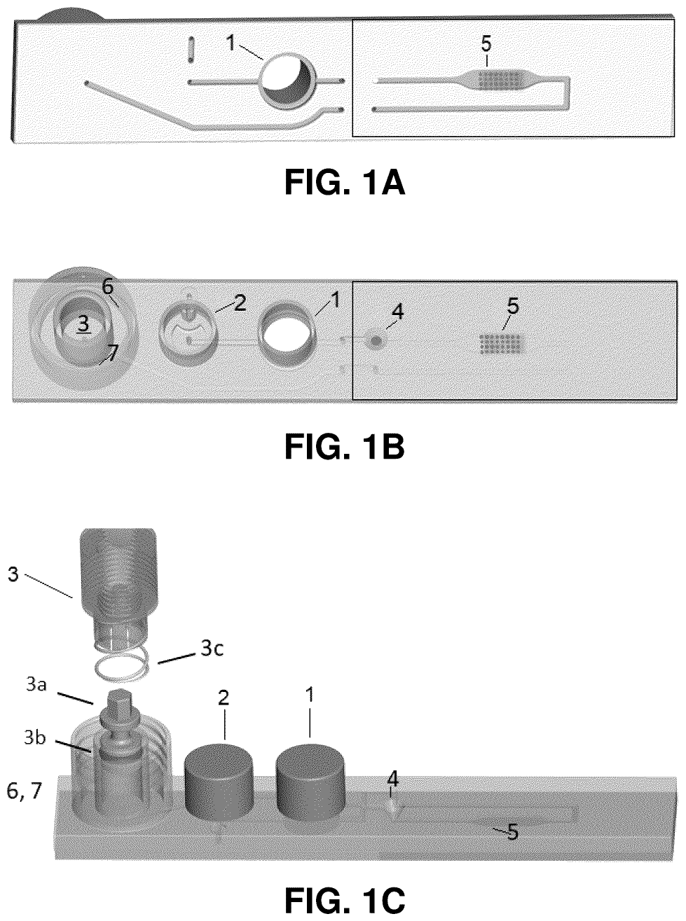

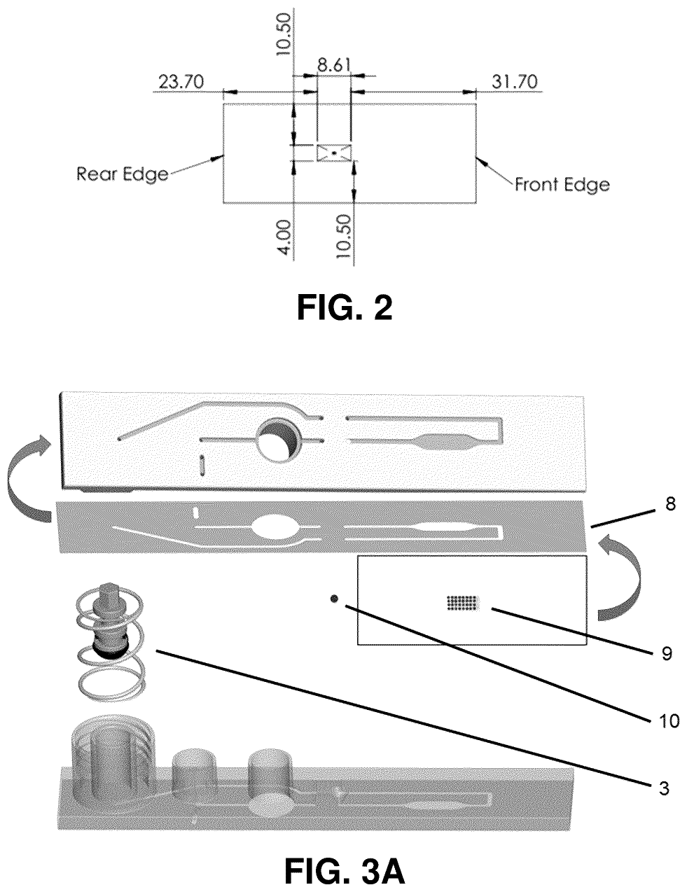

In reference to FIGS. 1A-C, a cassette comprising a sample receiver 1, a buffer chamber 2 comprising a first and second aliquot of buffer, a second chamber 6 comprising a vacuum liquid actuator and waste chamber 7, a reaction chamber comprising an array 5 and primary reagents 4 arranged in fluidic communication on a main chip. The main chip may further comprise an optical window for visualizing the reaction results. The sample receiver 1 comprises a chamber and a plasma separation membrane in fluid communication with a microfluidic channel. The sample receiver 1 is designed to deliver sample to the intake port on the cassette and negative fluidic pressure from 6 assists the movement of the sample to the reaction chamber via microfluidic channels. A cover may be placed over the sample receiver 1 to prevent extraneous material from entering the cassette. An actuator 3 built into the cassette draws the sample through a plasma separation membrane, leaving larger components, e.g., blood cells on the filter, and causes the filtered sample to flow through a microfluidic channel and re-suspend lyophylized primary and secondary reagents.

Pressure is applied to the buffer chamber cover 2 to release buffer into the microfluidic channel in the main chip. The buffer movement by negative fluidic pressure from the vacuum twist button draws the plasma from the sample receiver 1 into the reaction chamber. The buffer acts to dilute the plasma, hydrate primary and secondary reagents, and provide sufficient reaction volume in the reaction chamber. In the reaction chamber, virus peptides or anti-viral antibodies selectively bind to primary reagent bound to the array. The secondary reagent, e.g., labelled anti-Ig antibodies, selectively bind to the anti-viral antibodies, which either originate from the sample or the array. After sufficient reaction time, e.g., 1-60 minutes, the vacuum twist button 6 is turned to the next stop to release a second aliquot of buffer into the microfluidic channels. This second aliquot of buffer washes the reaction chamber, pushing the unreacted sample and secondary reagents through microfluidic channel into a waste chamber 7. The results of the reaction may be visualized by means of an optical window that allows visual access to the reaction chamber.

### Coronaviruses

Early WHO guidelines recommended that suspected cases of COVID-19 should be screened for the virus with nucleic acid amplification tests (NAAT), such as RT-PCR. The interim WHO guidance further affirmed that a single negative test result by NAAT does not exclude infection with SARS-CoV-2, and that patients with a typical clinical presentation or clear epidemic indications should receive clinical treatment and case management, even if negative by PCR at one or two time points. Though respiratory fluids have the greatest yield, the virus can also be detected in stool, blood, and possibly other specimens. Recent unpublished NAAT testing from Iceland suggests that at least half of all positive COVID-19 cases are asymptomatic. Virus-specific antibodies increase during active infection, and are detectable for many years after recovery. For SARS-CoV-2, IgM and IgA antibodies are detectable at 5 days by ELISA, while IgG is detected 8-14 days after symptom onset. Thevarajan, et al. Nature Medicine (2020); Guo, et al. Clin Infect Dis (2020). Paired serum (acute and convalescent) specimens can be useful to define cases, especially those that are negative by NAAT, and specimens collected from mild or asymptomatic cases will be important to document the total number of infections. Thus, antibody detection provides the basis for a diagnostic test as well as a long-term record of encounters with SARS-CoV-2, while serological surveys can aid investigation of an ongoing outbreak and retrospective assessment of the attack rate or extent of an outbreak. An epidemiological tool for continuously assessing infections within the population will be an important asset because it is not known if COVID-19 will disappear after this first wave of cases or return in seasonal cycles like influenza.

This disclosure relates to systems and methods comprising the inclusion of more than one protein (multiplex), or a fragment thereof, which find use in the detection of a coronavirus infection including SARS-CoV-2, otherwise known as COVID-19, and/or the presence of antibodies specific for a coronavirus in a biological sample. This disclosure further relates to a multiplex COVID-19 test system that uses a microfluidic assay cassette that can be used for measuring infection rates and assessing serological immunity. The described test system will detect infections caused by SARS-CoV-2 and human coronaviruses (HCoV) at distributed point-of-care sites or for in-home use by untrained users. The test system may incorporate recombinant spike and nucleoprotein antigens from SARS-CoV-2 and the six other known human coronaviruses into a microarray of test antigens that is inserted into a disposable microfluidic cassette that also houses the assay components and obtains results from a single drop of blood (e.g., less than 60 μL) or other biological fluids. The assay can include proteins from other newly discovered coronaviruses as well as antigens from seasonal influenza and additional pathogens. The non-coronavirus proteins provide results that may suggest infections by other pathogens and these also may serve as control proteins that ensure optimal assay performance.

The assay cassette is low cost (e.g., under $20 USD each), sealed, and single use to reduce the risk from possible biohazards. The serological assay may detect antibodies (IgG, IgA, or IgM) from a SARS-CoV-2 infection and may also report infections from six other HCoV. Antibody detection provides the basis for a diagnostic test as well as a long-term record of encounters with SARS-CoV-2, while test results may further provide important information on previous HCoV exposures and the possible status of protective immunity to COVID-19. Results from the multiplexed HCoV assay can be captured by a device that includes a camera, and the results can be interpreted visually, or more ideally, by the use of image analysis software. The multiplexed HCoV test system, which includes the assay cassette, can be used with a smartphone reader and cloud-based archive to provide rapid, reliable, and real-time digital data to guide containment of the COVID-19 pandemic.

The subject matter is a sensitive, specific, and rapid diagnostic test for 2019-nCoV for use in serological surveillance studies. The assay and assay cassette will provide an important tool for measuring the expansion of infections and to follow trends in infection rates and immunity after the epidemic peaks. The ability to make healthcare decisions based on rapid, reliable, and real-time digital data is a high priority. Recombinant spike and nucleoprotein antigens from SARS CoV-2 and the six other known human coronaviruses are incorporated into a disposable microfluidic cassette that houses the assay and obtains results from a single drop of blood. Generic methods for development and production of recombinant proteins for the serological assay were previously optimized by our research team. Commercially obtained hemagglutinin antigens from seasonal influenza A/B viruses are included as positive controls. Approximately 80% of the US population presents anti-influenza antibodies that are detected in the multiplexed assay. Inclusion of all CoV antigens will facilitate the collection of data for other HCoV infections. Because positive results may be due to past or present infection with non-SARS-CoV-2 coronavirus strains, a scoring algorithm, e.g., Fisher's scoring, can be used to increase assay specificity in case of possible interference from antibody cross-reactivities among strains.

The cassette performs all test functions, and the results may be processed by a portable reader that is the size of a smartphone. The serological assay can detect antibodies (IgG, IgA, and IgM) that result from an infection from SARS CoV-2 and can also report infections from six other HCoV. Data and geospatial-temporal information from the collection site can be uploaded to cloud-based archives for further analysis and actionable results. Methods that are based on the detection of viral RNA can provide results only during the first few days of active viremia. Unlike conventional viral RNA assays, the antibody detection system and method described herein can measure past, present, and asymptomatic infections with SARS-CoV-2 and other HCoV strains, thus capturing the greatest number of COVID-19 cases. Performance testing may confirm high specificity and sensitivity to detect asymptomatic and recovered infections by evaluation of blood and other antibody-containing biological fluids. Performance validation may use confirmed positive and negative control sera, and can include antibody class specificity (IgA, IgM, IgG), cross-reactivities and HCoV specificity.

Precise and timely knowledge of distributed infection rates resulting from disease outbreaks is essential for determining the overall risk and to assess the effectiveness of infection control measures. The described system will support seroprevalence studies of SARS-CoV-2 by the use of a rapid test system that can perform multiplexed analyses across all known human coronaviruses from remote point-of-care sites or at home. The proposed system may be used to acquire digital data from presumptive infections that can be uploaded to HIPAA-compliant cloud archiving to track the spread of infection and immunity through the population. The availability of these infection test results from distributed collection sites that can be uploaded to a centralized database will enhance infection control measures and the medical mission of first responders.

The multiplexed microfluidic assay is a novel contribution to COVID-19 and general coronavirus infection diagnostics. Lateral flow assays (LFA), ELISA and other typical antibody assays generally focus on detection of disease caused by a single infectious agent. There are no multiplexed antibody immunoassays that are highly portable and include COVID-19. The system and methods described herein for detecting antibodies allows for the monitoring of infections caused by the seven species of HCoV at the point of use or at home, and will require only a drop of blood. The disposable microfluidic assay can detect pathogen-specific antibodies within 15 minutes. An extension of the multiplexed HCoV assay and cassette can include a smartphone sized reader that analyzes, displays, and transmits the results through a wireless connection to a central database. Plasma separation from blood, reconstitution of test reagents, and all other assay steps are performed by the cassette. The assay cassette is sealed and single-use to greatly reduce risk from biohazards. The device can acquire serological results from both symptomatic and non-symptomatic cases, and thus enables an additional application and unmet need for disease surveillance.

### Coronavirus Cassette

Coronaviruses are RNA viruses that are spherical, have protrusions, and are crown-like. They are collectively referred to as coronaviruses. The virus has a diameter of 75 to 160 nanometers, and the virus genome is a continuous linear single-stranded RNA, and the molecular weight is usually (5.5 to 6.1)×10⁶. The coronavirus genome encodes a spike protein (S), an envelope protein, a membrane protein, and a nucleoprotein in that order.

Recombinant antigens from SARS-CoV-2 and the six other known human coronaviruses may be incorporated into a single disposable assay cassette. For example, recombinant antigens from MERS CoV, SARS-CoV-2 (COVID-19), and/or SARS-CoV, may be incorporate dinto a single disposable assay cassette. All assay components may be integrated into a plastic cassette that is approximately the size of a microscope slide. The HCoV nucleocapsid (N) and spike (S) proteins may be produced as recombinant products for incorporation into the assay by using proprietary gene synthesis methods. Reference controls for detecting antibodies to influenza or other viruses that cause respiratory-tract infections may also be included. A scoring algorithm may be used to compensate for any possible cross-reactivities among test HCoV protein probes.

In practice, a drop of blood (e.g., about 60 μL) collected from the finger-tip of a test subject may be inserted into the cassette and a button is pressed to begin the analysis. Plasma is separated from blood cells, mixed with stored reagents and delivered to a detection window containing the immobilized HCoV and control antigen probes for quantifying specific antibodies. The cassette is inserted into the reader and results are available in about 15 minutes. Test results and geospatial-temporal information from the collection site can be uploaded to a cloud-based database for further analysis and public health recommendations.

The microarray enclosed within the microfluidic assay cassette contains recombinant NP and S antigens from each of the seven known coronaviruses that infect humans, including SARS-CoV-2. Serum antibodies or antibodies from other biological fluids that bind to the test antigens are detected by fluorescence of tagged antibodies that recognize human or other test antibodies and that are stored in the assay cassette. Binding of secondary antibodies to human IgA, IgG and IgM, or antibodies of animal species that are also printed on the microarray will present a signal that confirms that the detection components of the assay have performed properly for each evaluation. A major limitation of antibody assays that focus on a single virus or pathogen is that the results are unable to distinguish the difference between cross reactivity and evidence of current or previous infections, while the described invention addresses this problem by including the antigens from all seven coronaviruses.

It is also possible that there will be some individuals who will present no antibodies to any of the test antigens, most likely because they have never had a coronavirus infection. To reach this conclusion, it is necessary to confirm that all components of the assay have performed properly. To control for seronegative individuals (e.g., individuals with no antibodies against CoV), a control may be included that tests for antibodies from an infection or vaccination, such as influenza, that most people will have had at some time in their life. High antibody-binding signals resulting from influenza antigens and low signals from HCoV antigens may indicate a recent influenza infection.

The sequences for the coronavirus polypeptides are shown in Tables 2, 3, and 4.

The coronavirus peptides can be synthesized in bacterial or eukaryotic host cells, or can be produced by in vitro translation and transcription reactions. The bacterial protein expression constructs are synthesized to contain codons that are preferred by E. coli. Proteins expressed in mammalian cells can use native viral codons, and proteins produced in yeast or plant hosts can use codons optimized for the eukaryotic host. Mammalian cell expression in HEK-293 cells is preferred to produce spike polypeptide products that include post-translational glycosylation or other modifications that are usually present with coronaviruses that infect humans. In one embodiment, the synthetic coronavirus gene is inserted into the plasmid PET-28a(+) that encodes a kanamycin resistance element. The PET-28a(+) plasmid, which contains a ribosome binding site and ATG start codon, is designed for protein expression from translation signals carried by the cloned DNA. The plasmid includes N-terminal His•Tag® sequences, an internal T7•Tag® sequence, thrombin cleavage site and lac repressor/lac operator to inhibit transcription in E. coli. Expression of the viral polypeptide can be induced by adding lactose or isopropyl-β-D-thiogalactopyranoside (IPTG). For bacteria protein expression, all insert DNAs (the full length N protein and the RBD of the spike proteins) were cloned into the PET-28a(+) vector using the Nde I site. The length of the n-terminal His-tag and the thrombin cleavage site is 20 amino acid long. In another embodiment, the plasmid pTwist CMV BetaGlobin WPRE Neo, which encodes an element for ampicillin resistance, can be used for production of viral proteins by mammalian host cells. The pTwist CMV driven expression vector is used for transient expression in mammalian cells and is designed to deliver exceptionally high levels of transgene expression. This vector can be used in suspension-adapted and adherent cells for transient protein expression and can also be used as a G-418-selectable expression plasmid for stable cell line engineering. In addition to the CMV promoter, this vector contains a beta globin intron and a WPRE that enhance transgene expression. The length of the c-terminal His-tag 6 amino acid long.

Antigenic regions of coronavirus peptides that are preferred sites for antibody binding include the RBD of the S protein and full-length sequences of S proteins, especially those that are folded into a native quaternary structure, and full-length N sequence. Smaller peptides that are derived from the full-length sequences of S and N proteins can also be used that include the least amount of amino acid residues in common with other flaviviruses, but a sufficient number to facilitate specific molecular recognition by anti-coronavirus antibodies. About 10-15 amino acid residues are the minimum number needed for specific molecular recognition by anti-coronavirus antibodies. Longer peptide sequences will allow a greater amount of test antibodies to interact with antigens immobilized on the array, and will produce the greatest signal for the assay readout. The inclusion of sequences within the peptide that are more common to other coronavirus species will lower the specific assay signal for each coronavirus, while amino acid substitutions within these conserved sequences can be used to reduce the amount of non-specific antibody binding. The full-length or shorter peptides can be fused together end-to-end by recombinant DNA technology to produce a single peptide for each species or strain of coronavirus or control virus.

An array pattern of six columns and six rows of 150-300 μM spots, evenly spaced, is preferred for use with the assay cassette, while other spot arrangements, sizes, and numbers can also be used. The 14 coronavirus RBD and N peptides can be printed in duplicate on the array and the remaining spots are used for printing duplicate HA antigens from three seasonal influenza viruses (A, B, C, or A, B, B or A, A, B strains), a positive control consisting of an antibody or other capture reagent that binds to human antibodies, and negative control, buffer only, spots. The non-coronavirus positions on the array can alternatively be unused or used only to spot buffer or other positive and negative control peptides. A secondary reagent for an antibody assay can be an antibody that binds to human IgG or IgG subtypes, IgM, IgA, or IgE, and the secondary reagent is conjugated to a colorimetric, fluorescent or enzymatic tag that will localize antibody binding events on the assay microarray. The secondary reagent antibody can be a monoclonal or polyclonal antibody that originated in mice, rabbits, goats, camelids, rats, or other animal species, including cells line derived from these species or antibodies created by recombinant DNA methods. Antibody arrays may include, but not exclusively, the capture antibodies listed in Table 1, using duplicate capture antibodies for individual coronaviruses and negative control antibodies that do not recognize coronaviruses, negative control spots without antibody, and positive control spots that comprise coronavirus proteins. A secondary reagent for a viral antigen capture assay can be a detection antibody that is conjugated to a colorimetric, fluorescent or enzymatic tag that will localize antibody binding events on the assay microarray. The secondary reagent antibody can be a monoclonal or polyclonal antibody that originated in mice, rabbits, goats, camelids, rats, or other animal species, including cells line derived from these species or antibodies created by recombinant DNA methods. The secondary reagent can also be angiotensin converting enzyme 2 (ACE2), which is the primary cellular receptor for SARS-CoV-2 that is required for infection. Using ACE2 conjugated to a colorimetric, fluorescent or enzymatic tag, reduction in receptor binding to microarrays of RBD can be used to detect neutralizing antibodies that block virus infections. The ACE2 can also be spotted in microarrays to detect RBD binding and inhibition of binding by virus-neutralizing antibodies.

A coronavirus cassette may also comprise nucleic acids, for example DNA and/or RNA, for methods of detecting coronavirus nucleic acids, e.g., DNA or RNA. For example, the coronavirus nucleic acid sequences listed in Table 2, e.g., the nucleic acid sequence of SEQ ID NOs: 247, 249, 251, 253, 255, 257, 259, 261, 263, 265, 267, 269, 271, and/or 273, may be used in a coronavirus cassette described herein.

### Filoviruses Cassettes

Filoviruses generally refer to viruses of the viral family called Filoviridae and infection can cause severe hemorrhagic fever in humans and nonhuman primates. Three members of this virus family have been identified: Marburgvirus, Ebolavirus, and the distantly-related Cuevavirus. The six recognized species of Ebolavirus are Zaire ebolavirus, Sudan ebolavirus, Taï Forest ebolavirus (formerly Côte d'Ivoire ebolavirus), Bundibugyo ebolavirus, Reston ebolavirus, and Bombali ebolavirus. Only Ebola, Sudan, Taï Forest, and Bundibugyo viruses are known to cause human disease. Reston ebolavirus can be fatal in monkeys and it has been recently recovered from infected swine in South-east Asia, while Bombali ebolavirus and Cuevavirus species have only been isolated from bats. Structurally, filovirus virions (complete viral particles) may appear in several shapes, a biological feature called pleomorphism. These shapes include long, sometimes branched filaments, as well as shorter filaments shaped like a “6”, a “U”, or a circle. Viral filaments may measure up to 14,000 nanometers in length, have a uniform diameter of 80 nanometers, and are enveloped in a lipid membrane. Each virion contains one molecule of single-stranded, negative-sense RNA. New viral particles are created by budding from the surface of their hosts' cells; however, filovirus replication strategies are not completely understood.

The genus Ebolavirus (or “Ebola” or “Ebola virus”) is a virological taxon included in the family Filoviridae, order Mononegavirales. The members of this genus are generally referred to as ebolaviruses, and five species were named for the region where each was originally identified: Bundibugyo ebolavirus, Reston ebolavirus, Sudan ebolavirus, Tai Forest ebolavirus (originally Cote d'Ivoire ebolavirus), and Zaire ebolavirus.

The genus Marburgvirus (“Marburg” or “Marburg virus”) refers to the species, Marburg marburgvirus, which includes two main members, Marburg virus (MARV) and Ravn virus (RAVV). Both viruses cause Marburg virus disease, a form of hemorrhagic fever, in humans and nonhuman primates.

Cuevavirus is a genus in the family Filoviridae that has one identified species, Lloviu cuevavirus (LLOV or “cueva virus”) that is found only in bats. Studies indicate that LLOV is a distant relative of the more widely known Ebola and Marburg viruses.

For the described invention, the filovirus diagnostic test is designed to assess infections by measuring antibody binding to a microarray of antigens from six species of Ebola and Marburg viruses that are incorporated into a disposable assay cassette. For example, the antigens may be from Ebola, Sudan Taï Forest, and/or Bundibugyo viruses. The test specimen can be a drop of blood, serum, plasma, oral secretions and other biological material that contains antibodies. The system and methods described herein allow for the assessment of current or previous infections by antibody binding to a multiplexed antigen panel by using a drop of blood (or oral secretions) in a low-cost, field deployable system. The disposable cartridge is assembled with injection molded plastics and uses feature sizes that are within well-established manufacturing tolerances to ensure manufacturing scalability. Each cartridge is designed to be used for a single sample to monitor biological fluids for detection of suspected infections.

In a general aspect, the disclosure provides a detection agent comprising one or more amino acid sequences of a filovirus protein, or a fragment thereof, and a substrate wherein the one or more amino acid sequences of the filovirus protein is attached to substrate. In certain embodiments, the one more amino acid sequences of a filovirus protein may comprise a sequence of a protein from a filovirus selected from Marburg marburgvirus, Sudan ebolavirus, Zaire ebolavirus, Reston ebolavirus, Bundibugyo ebolavirus, and Tai Forest ebolavirus.

The filovirus peptide may comprise a filovirus protein, or fragment thereof, may comprise a sequence from a nucleoprotein (NP), virion protein 40 (VP40), glycoprotein (GP), virion protein (VP35), virion protein (VP30), virion protein (VP24), RNA-dependent RNA polymerase (L), or a fragment thereof, or any combination thereof. Any amino acid sequence that provides for binding and recognition of a filovirus specific antibody may be used in the systems or methods described herein. The amino acid sequence may exhibit little to no cross-reactivity to filovirus specific antibodies that are directed to a particular type of filovirus or a particular filovirus protein. The one or more amino acid sequences of a filovirus protein comprises GP, or fragment thereof. The GP or fragment thereof may comprise a mucin-like domain fragment of GP (GP-mucin) or a GP ectodomain. While the detection agent comprises at least one amino acid sequence of a filovirus protein, it may also comprise a plurality of such amino acid sequences. In some embodiments the detection agent comprises from two or more amino acid sequences to twenty or more amino acid sequences (e.g., 2, 3, 4, 5, 6, 7, 8, 9, 10, 11, 12, 13, 14, 15, 16, 17, 18, 19, 20, or more amino acid sequences) that may be selected from the same or different filovirus and/or the same or different filovirus protein. As a further example, in some embodiments, such as those illustrated in the non-limiting Examples, the detection agent may include at least three different amino acid sequences of at least three different filovirus proteins, or fragments thereof (e.g., NP, VP40, and GP, or fragments thereof).

The filovirus viral peptide amino acid sequences may comprise a sequence that is not identical to the protein sequence from which it is derived. Some minor changes in the primary amino acid sequence and/or post-translational modification and processing of the sequence may be allowable as long as the sequence modification does not interfere with the ability of the filovirus-specific antibody to bind. In some embodiments, the detection agent can comprise one or more amino acid sequences having at least 90% sequence identity (e.g., 90%, 91%, 92%, 93%, 94%, 95%, 96%, 97%, 98%, or 99%) to the filovirus protein from which it is derived. In some embodiments, the amino acid sequences may comprise an NP sequence having at least 90% sequence identity to the sequence selected from the group consisting of SEQ ID NO:4 (Zaire NP); SEQ ID NO: 10 (Sudan NP); SEQ ID NO: 16 (Bundibugyo NP); SEQ ID NO: 22 (Tai Forest NP); SEQ ID NO: 28 (Reston NP); and SEQ ID NO: 34 (Marburg NP); a VP40 sequence having at least 90% sequence identity to the sequence selected from the group consisting of SEQ ID NO: 2 (Zaire VP40); SEQ ID NO: 8 (Sudan VP40); SEQ ID NO: 14 (Bundibugyo VP40); SEQ ID NO: 20 (Tai Forest VP40); SEQ ID NO: 26 (Reston VP40); and SEQ ID NO: 32 (Marburg VP40); and/or a GP-mucin domain having at least 90% sequence identity to the sequence selected from the group consisting SEQ ID NO: 6 (Zaire GP-mucin); SEQ ID NO: 12 (Sudan GP-mucin); SEQ ID NO: 18 (Bundibugyo GP-mucin); SEQ ID NO: 24 (Tai Forest GP-mucin); SEQ ID NO: 30 (Reston GP-mucin); and SEQ ID NO: 36 (Marburg GP-mucin). In further embodiments, the detection agent may comprise an NP sequence selected from the group consisting of SEQ ID NO: 4 (Zaire NP); SEQ ID NO: 10 (Sudan NP); SEQ ID NO: 16 (Bundibugyo NP); SEQ ID NO: 22 (Tao Forest NP); SEQ ID NO: 28 (Reston NP); and SEQ ID NO: 34(Marburg NP); a VP40 selected from the list consisting of SEQ ID NO: 2 (Zaire VP40); SEQ ID NO: 8 (Sudan VP40); SEQ ID NO: 14 (Bundibugyo VP40); SEQ ID 20 (Tai Forest VP40); SEQ ID NO: 26 (Reston VP40); SEQ ID NO: 32 (Marburg VP40); and/or a GP-mucin domain selected from the group consisting of SEQ ID NO: 6 (Zaire GP-mucin); SEQ ID NO: 12 (Sudan GP-mucin); SEQ ID NO: 18 (Bundibugyo GP-mucin); SEQ ID NO: 24 (Tai Forest GP-mucin); SEQ ID NO: 30 (Reston GP-mucin); and SEQ ID NO: 36 (Marburg GP-mucin). See U.S. Patent Application Publication No. 2017/0089920.

Table 2 lists the filoviral peptides sequences that are preferred for antibody assays and Table 1 lists the preferred antibodies for antigen capture assays. The GP and NP peptides are preferred, and a configuration of six columns by six rows is preferred for arrays to include duplicate antigens from the six filoviruses that infect humans. The remaining spots can be used for negative or positive controls, which can include HA proteins from seasonal influenza viruses, a capture reagent that will bind human antibodies, buffer only, or other control proteins and substances. Antigenic regions of filovirus peptides that are preferred sites for antibody binding include the mucin domain of GP and full-length sequences of NP. Smaller peptides that are derived from the full-length sequences of GP and NP from one filovirus species can also be used that include the least amount of amino acid residues in common with other filovirus species or strains, but a sufficient number to facilitate specific molecular recognition by anti-filovirus antibodies. About 10-15 amino acid residues are the minimum number needed for specific molecular recognition by anti-filovirus antibodies. Longer peptide sequences will allow a greater amount of test antibodies to interact with antigens immobilized on the array, and will produce the greatest signal for the assay readout. The inclusion of sequences within the peptide that are more common to other filovirus species will lower the specific assay signal for each species or strain of filovirus, while amino acid substitutions within these conserved sequences can be used to reduce the amount of non-specific antibody binding. The full-length or shorter peptides can be fused together end-to-end by recombinant DNA technology to produce a single peptide for each species or strain of filovirus or control virus.

A filovirus cassette may also comprise nucleic acids, for example DNA and/or RNA, for methods of detecting filovirus nucleic acids, e.g., DNA or RNA. For example, nucleic acid sequences encoding for the polypeptide sequences listed in Table 2, e.g., the nucleic acid sequence of SEQ ID NOs: 247, 249, 251, 253, 255, 257, 259, 261, 263, 265, 267, 269, 271, and/or 273, may be used in a filovirus cassettes and/or methods described herein.

### Flavivirus Cassettes

This disclosure relates to a multiplex test system for evaluating flavivirus, including yellow fever, virus infection or vaccination for disease surveillance and diagnosis. The test can be used in a microfluidic assay cassette system for measuring infection rates and assessing serological immunity. Outbreaks of yellow fever continue to occur in Africa and South America despite the development of a highly-effective vaccine over eighty years ago. Due in part to the limitations of predictive assays that are currently in practice, the duration of protection from disease and the need for re-vaccination of individuals at risk of repeated exposure to the yellow fever virus is unclear. The inventors examined primary and secondary vaccination responses to dominant protein antigens from the yellow fever virus and nearest neighbors from the Flaviviridae family of RNA viruses. Using multiplexed microarrays that required only microliter serum test volumes, the inventors evaluated the changes that occurred in levels of Yellow Fever virus (YFV)-specific antibodies that are detectable shortly after vaccination and >15 years later. Levels of antibody responses detected were highest with envelope (E) antigens, intermediate with pM, and NS1 antibody levels were the lowest. Comparisons with YFV printed on the same microarray demonstrate the utility of using principal flavivirus antigens as an alternative to whole virus preparations for evaluating vaccine responses. There was negligible interference in assay results from antibody cross-reactivities with recombinant antigens from dengue, Zika, West Nile and other flaviviruses that are pathogenic to human, and good concordance with antibody neutralization tests. The inventors observed that the frequency of seropositive subjects detected with recombinant protein peaked during the first year from vaccination, declined 1-6 years after primary vaccination, and rebounded to maximum values for boosted subjects. These results demonstrate the feasibility of assessing multiple trends in antibody responses to yellow fever vaccination within the same assay and provide a foundation for establishing new high-throughput serological assays.

Yellow fever is an acute viral hemorrhagic disease that is transmitted by mosquitoes that carry the yellow fever virus (YFV), as first confirmed by U.S. Army researchers in 1900. Reed, Walter Recent Researches concerning the Etiology, Propagation, and Prevention of Yellow Fever, by the United States Army Commission. J Hyg (Lond), 1902. 2(2): 101-19. YFV and other vector-borne viruses within Flaviviridae family are small (˜9.5-13 kb) positive-stranded and enveloped RNA viruses that enter host cells by receptor-mediated endocytosis. Pierson & Kielian Curr Opin Virol (2013) 3(1): 3-12; Simmonds et al. J Gen Virol. (2017) 98(1): 2-3; Smit et al. Viruses (2011) 3(2): 160-71.

Despite the development of a highly effective vaccine, yellow fever continues to be a major public health problem for many countries in Africa and South America, and outbreaks could easily turn into public health emergencies of international concern. Theiler & Smith J Exp Med (1937) 65(6): 787-800; Eliminate Yellow fever Epidemics (EYE): a global strategy, 2017-2026. Wkly Epidemiol Rec, 2017. 92(16): 193-204.

An urban outbreak began in Angola in 2016 and spread to the Democratic Republic of Congo (DRC), resulting in 965 cases with one third that were fatalities, while an ongoing yellow fever outbreak centered in Brazil has led to over 1,000 confirmed human cases, including over 400 deaths. [PAHO] Pan American Health Organization/[WHO] World Health Organization, Epidemiological Update:Yellow Fever. 2018.

These recent disease outbreaks enhance concerns about the expanding risk areas of nonvaccinated populations, shrinking stockpiles of the yellow fever 17D/17DD vaccines, and the unconfirmed longevity of immunity acquired from vaccination. Agampodi & Wickramage Biomed Res Int, (2013) 2013: 905043; Barrett Vaccine (2017) 35(44): 5951-5955; Cui et al. Int J Infect Dis (2017) 60: 93-95; Wasserman et al. Int J Infect Dis. (2016) 48: 98-103.

Maintaining vaccine stockpiles is hampered by the length of time (up to 6 months) it takes to produce the vaccine in pathogen-free embryonated chicken eggs, the small number of pre-qualified manufacturers of yellow fever vaccine, and the short (2-3 year) storage limitation. Barrett Vaccine (2017) 35(44): 5951-5955; Monath et al. Lancet (2016) 387(10028): 1599-600; [UNICEF] United Nations International Children's Emergency Fund, Yellow fever vaccine: current supply outlook. 2016.

Due to the more than 30 million doses of vaccine that were distributed during the outbreaks, emergency stockpiles were exhausted and a global vaccine shortage ensued. [WHO] World Health Organization. WHO dispatched 3.5 million doses of yellow fever vaccine for outbreak response in Brazil. 2017 [WHO Website 2018]; Gershman et al. MMWR Morb Mortal Wkly Rep (2017) 66(17): 457-459.

Further, a recent report (PMID: 31545372) estimated that up to 472.9 million people in high risk areas of Africa and South/Central America may still require vaccination in order to achieve the WHO-recommended threshold population coverage of 80%. Ndeffo-Mbah & Pandey J Infect Dis. (2019)

Antibody tests are important for evaluating immunological responses to 17D/17DD vaccination, though the actual mechanism of immune protection is not firmly established. Seroconversion is evaluated by virus plaque reduction neutralization tests (PRNT) and is defined as either a fourfold increase in neutralizing antibody or the induction of measurable neutralizing antibody in a previously seronegative individual. Recommendations to assure the quality, safety and efficacy of live attenuated yellow fever vaccines. Geneva: World Health Organization, (WHO Website 2010). Serological assays are also used to identify suspected cases of yellow fever, while the brief window of viremia impedes the reliability of methods that only employ viral RNA detection. Examples of serological diagnostic tests used by the U.S. Centers for Disease Control include IgM-capture and IgG ELISA, as well as Microsphere-based Immunoassays (MIA). CDC Website “Yellow Fever” (2020). Based on a compilation of data from PRNT studies, seropositivity for persons vaccinated ≥10 years previously was >90% and declined to >80% for subjects vaccinated ≥20 years previously. Staples et al. MMWR Morb Mortal Wkly Rep (2015) 64(23): 647-50. However, yellow fever challenge studies may not be allowed by governing authorities and data obtained by PRNT alone are insufficient to prove that standard one-shot vaccination leads to life-time protection from yellow fever. There are conflicting conclusions with regard to the need for re-vaccination of individuals at risk of repeated exposure to YVF, and variable results from diagnostic and surveillance assays contribute to the unclear status of the science. A study by Campi-Azevedo and coworkers among Brazilian subjects noted that discrepancies between published reports regarding yellow fever revaccination may also be attributable to differences in study populations. Campi-Azevedo et al. Emerg Infect Dis (2019) 25(8): 1511-1521.

While there is insufficient evidence to conclude that vaccination provides life-long protection, only 12 confirmed cases of yellow fever have been identified among vaccinated individuals since yellow fever vaccination began in the 1930s. Vaccines and vaccination against yellow fever. WHO position paper—June 2013. Wkly Epidemiol Rec, 2013. 88(27): 269-83. In contrast, a recent report indicated that by 5-10 years after primary vaccination 14%-30% of subjects did not retain sufficient levels of neutralizing antibodies or CD8+ T-cell memory, strengthening the case for booster vaccination. Campi-Azevedo et al. Emerg Infect Dis (2019) 25(8): 1511-1521. Another study, described the rapid waning of immunity during the early years after vaccination of 9-month-old infants, as measured by PRNTs, and suggested a reconsideration of the single-dose recommendation for this target population in endemic countries. Domingo, et al. Lancet Infect Dis, 2019. In another development, the vaccine shortage prompted the World Health Organization (WHO) to recommend the administration of a reduced amount (20%) of the standard dose until stocks are replenished. World Health Organization. Lower doses of yellow fever vaccine could be used in emergencies. (WHO Website, 2016); Casey et al. N Engl J Med. (2019) 381: 444-454. There are not sufficient data to ensure that this strategy of vaccine dose dilution will provide adequate lifelong protection. For further consideration, immune responses to previous flavivirus infections are also known to influence immunoassay results, disease susceptibility, and severity of new flavivirus infections. Keasey, et al. Clin Vaccine Immunol. (2017) 24(4); Anderson, et al. PLoS Negl Trop Dis (2011) 5(10): e1311; Glover, & White J Theor Biol (2020) 484: 110014. Consequently, subjects undergoing vaccination may harbor cross-reactive antibodies from previous exposures to dengue, Zika, and other flaviviruses that are often found in yellow fever endemic areas. Keasey, et al. Clin Vaccine Immunol. (2017) 24(4). These cross-reactive antibodies may produce confounding effects that impact evaluations of vaccination responses, the assessment of sustainable immunity, and vaccine efficacy.

There have been limited advancements in laboratory methods of diagnosis and disease surveillance since the initiation of vaccination for prevention of yellow fever over 80 years. Theiler & Smith J Exp Med (1937) 65(6): 787-800; Barrett Vaccine (2017) 35(44): 5951-5955. In contrast, molecular-level details of the YFV replication cycle are known and provide guidance for accelerating the introduction of new laboratory methods. The 11 kb RNA genome of flaviviruses is translated into a single polyprotein that is cleaved into three structural proteins (capsid (C), envelope (E), and precursor membrane/membrane (pM/M)) and seven non-structural proteins (NS1, NS2A, NS2B, NS3, NS4A, NS4B, and NS5). Heinz & Stiasny J Clin Virol. (2012) 55(4): 289-95; Hollidge et al. J Neuroimmune Pharmacol (2010) 5(3): 428-42; Lindenbach, et al., Flaviviridae: the viruses and their replication. 2007, Philadelphia, USA: Lippincott William & Wilkins. Structural proteins that are incorporated into the virus particle have primary roles in virus assembly, mediating cell attachment, and fusion with the host membrane. Lindenbach, et al., Flaviviridae: the viruses and their replication. 2007, Philadelphia, USA: Lippincott William & Wilkins; Lindenbach & Rice Adv Virus Res (2003) 59: 23-61.

Non-structural proteins are mainly involved in processing of the polyprotein, RNA replication, and evasion of host immune responses. Lindenbach, et al., Flaviviridae: the viruses and their replication. 2007, Philadelphia, USA: Lippincott William & Wilkins; Avirutnan et al. J Exp Med (2010) 207(4): 793-806; Avirutnan et al. PLoS Pathog (2007) 3(11): e183; Chuang et al. J Biomed Sci. (2013) 20: 42; Patkar & Kuhn J Virol (2008)82(7): 3342-52; Perera & Kuhn, Curr Opin Microbiol. (2008) 11(4): 369-77.

To more closely evaluate serological correlates of immunity, the inventors examined vaccination responses with microarrays of recombinant E, pM/M, and NS1 antigens in comparison with whole YFV, and the potential for interference by antibody cross-reactivities with other flaviviruses. The inventors further evaluated the changes that occur to levels of YFV-specific antibodies that are detectable by early (30 days) through late (>15 years) time intervals from vaccination. These results suggested a correlation between data obtained by PRNT and the microarray assay, while demonstrating the utility of key flavivirus antigens as an alternative to the use of whole virus preparations in high-throughput assays. Table 1 lists antibodies that can used in antigen detection assays, and Table 2 lists sequences that can be used in a yellow fever antibody test. A antigen-capture assay can also be used for diagnosis of infection by using a capture antibody array, an example being the mouse anti-yellow fever virus mouse monoclonal antibody clone 2D12 that recognizes the envelope protein of the wild (Asibi) and vaccine strains of yellow fever virus, and a labeled polyclonal antibody that will specifically detect YFV that is bound by the capture antibody. The inclusion of additional pairs of capture and detection antibodies to other viral species can be used for multiplexed assays. The capsid (C), envelope (E), precursor membrane/membrane (pM/M)) and the non-structural protein 1 (NS1) are the preferred antigens to be used as targets for antigen-capture or as arrayed polypeptides in an antibody test.

Antigenic regions of yellow fever virus peptides that are preferred sites for antibody binding include full-length sequences of the C, E, and NS1 proteins. Smaller peptides that are derived from the full-length sequences can also be used that include the least amount of amino acid residues in common with other flaviviruses but a sufficient number to facilitate specific molecular recognition by anti-YFV antibodies. About 10-15 amino acid residues are the minimum number needed for specific molecular recognition by anti-YFV antibodies. Longer peptide sequences will allow a greater amount of test antibodies to interact with antigens immobilized on the array, and will produce the greatest signal for the assay readout. The inclusion of sequences within the peptide that are more common to other flavivirus species will lower the specific assay signal for YFV, while amino acid substitutions within these conserved sequences can be used to reduce the amount of non-specific antibody binding. The full-length or shorter peptides can be fused together end-to-end by recombinant DNA technology to produce a single peptide comprising all YFV or control virus test antigens.

A flavivirus cassette may also comprise nucleic acids, for example DNA and/or RNA, for methods of detecting flavivirus nucleic acids, e.g., DNA or RNA. For example, the flavivirus nucleic acid sequences that encode the polypeptides listed in Table 2, e.g., the amino acid sequence of SEQ ID NOs: 37, 40, 43, 46, 49, 52, 55, 58, 61, 64, 67, 70, 73, 76, 79, 82, 85, 88, 91, 94, 97, 100, 103, 106, 109, 112, 115, 118, 121, 124, 127, 130, 133, 136, 139, 142, 145, 148, 151, 154, 157, 160, 163, 166, 169, 172, 175, 178, 181, 184, 187, 190, 193, 196, 199, 202, 205, 208, 211, 214, 217, 220, 223, 226, 229, 232, 235, 238, 241, 244, or a combination thereof, may be used in a flavivirus cassette and/or method as described herein. Also, flavivirus nucleic acid sequences listed in Table 2, e.g., 38, 39, 41, 42, 44, 45, 47, 48, 50, 51, 53, 54, 56, 57, 59, 60, 62, 63, 65, 66, 68, 69, 71, 72, 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, 123, 125, 126, 128, 129, 131, 132, 134, 135, 137, 138, 140, 141, 143, 144, 146, 147, 149, 150, 152, 153, 155, 156, 158, 159, 161, 162, 164, 165, 167, 168, 170, 171, 173, 174, 176, 177, 179, 180, 182, 183, 185, 186, 188, 189,191, 192, 194, 195, 197, 198, 200, 201, 203, 204, 206, 207, 209, 210, 212, 213, 215, 216, 218, 219, 221, 222, 224, 225, 227, 228, 230, 231, 233, 234, 236, 237, 239, 240, 242, 245, 246, or a combination thereof may be used in a flavivirus cassette and/or method described herein.

TABLE 1
Listing of Commercially Available Antibodies for Capture and Detection
of Filoviruses, Coronaviruses, or Yellow Fever Virus

A. Yellow Fever Virus
Mouse anti-Yellow Fever Virus mouse monoclonal antibody, clone 3576. BioRad or Abcam.
Mouse anti-Yellow fever virus mouse monoclonal antibody, clone 2D12 recognizes the envelope
protein of the wild (Asibi) and vaccine strains of yellow fever virus. No cross reactivity with other
flaviviruses has been reported. BioRad.
Yellow Fever Virus Antibody (0G5) mouse monoclonal antibody specific for the envelope
protein of the wild (Asibi) and vaccine strains of yellow fever virus. No cross reactivity with other
flaviviruses has been reported. Novus Biologics.
Anti-Flavivirus Group Antigen mouse monoclonal antibody, clone D1-4G2-4-15 detects
Flavivirus Group Antigen across all flaviviruses. Sigma-Aldrich and other suppliers.
Anti-Yellow Fever Virus Envelope protein rabbit polyclonal antibody Cat No. GTX134024
GeneTex. (Garg H et al. Sci Rep 2020; 10 (1): 4017 Virus Like Particles (VLP) as multivalent
vaccine candidate against Chikungunya, Japanese Encephalitis, Yellow Fever and Zika Virus.).
Anti-Envelope Glycoprotein Yellow Fever Virus Rabbit polyclonal antibody. Biorbyt.
Anti-Yellow Fever Virus NS1 monoclonal antibody (CE6). The NativeAntigenCompany.
Anti-Yellow Fever Virus NS1 monoclonal antibody (HH7). The NativeAntigenCompany.
Anti-Yellow Fever Virus NS1 mouse monoclonal antibody (HH7). The NativeAntigenCompany.
B. Filoviruses
1. Anti-Zaire ebolavirus antibodies
Chimeric monoclonal antibody anti-EBOV GP mAb (c6D8) 0201-021. IBT Bioservices.
Chimeric monoclonal antibody anti-EBOV GP mAb (h13F6) 0201-0221. IBT Bioservices.
Human monoclonal antibody anti-EBOV GP mAb (KZ52) 0260-001. IBT Bioservices.
Mouse monoclonal antibody anti-(EBOV GP) mAb (4F3) 0201-020. IBT Bioservices.
Mouse monoclonal antibody anti-EBOV VP40 mAb (3G5) 0201-016. IBT Bioservices.
Mouse monoclonal antibody anti-EBOV VP40 mAb (5B12) Ebola Virus 0201-017. IBT
Bioservices.
Mouse monoclonal antibody anti-EBOV VP40 mAb (AE11) Ebola Virus 0201-018. IBT
Bioservices.
Anti-Ebola GP [h-13F6] Recombinant monoclonal antibody to Zaire ebolavirus GP. Manufactured
using Recombinant Platform with variable regions (i.e. specificity) from the hybridoma h-13F6.
Absolute Antibody company.
Ebola Virus NP (subtype Zaire, strain H. sapiens-wt/GIN/2014/Kissidougou-C15) Mouse
monoclonal Antibody. Invitrogen.
Ebola Virus GP-RBD (subtype Zaire, strain Mayinga 1976) Rabbit Polyclonal Antibody.
Invitrogen.
Ebola Virus GP (subtype Zaire, strain H. sapiens-wt/GIN/2014/Kissidougou-C15) Rabbit
Polyclonal Antibody. Invitrogen.
Ebola Virus GP (subtype Zaire, strain Mayinga 1976) rabbit polyclonal antibody. Invitrogen.
Ebola Virus NP (subtype Zaire, strain H. sapiens-wt/GIN/2014/Kissidougou-C15) rabbit
polyclonal antibody. Invitrogen.
Ebola Virus GP (subtype Zaire, strain H. sapiens-wt/GIN/2014/Kissidougou-C15) Mouse
monoclonal antibody. Invitrogen.
Ebola Virus GP2 (subtype Zaire, strain H. sapiens-wt/GIN/2014/Kissidougou-C15) Mouse
monoclonal antibody. Invitrogen.
Ebola Virus NP (subtype Zaire, strain H. sapiens-wt/GIN/2014/Kissidougou-C15) Mouse
monoclonal antibody. Invitrogen.
Ebola Virus GP-RBD (subtype Zaire, strain Mayinga 1976) Polyclonal rabbit antibody.
Invitrogen.
Ebola Virus GP (subtype Zaire, strain Mayinga 1976) Mouse monoclonal antibody. Invitrogen.
Anti-Ebola GP [h-13F6] Recombinant monoclonal antibody to GP from all Ebola viruses.
Manufactured using Recombinant Platform with variable regions (i.e. specificity) from the
hybridoma h-13F6. Absolute Antibody company.
2. Anti-Sudan ebolavirus antibodies
Ebola Virus NP (subtype Sudan, strain Gulu) Mouse monoclonal antibody. Invitrogen.
Ebola Virus GP1 (mucin domain deleted) (subtype Sudan, strain Gulu) Recombinant rabbit
monoclonal antibody. Invitrogen.
Ebola Virus NP (strain New Guinea 2014) rabbit polyclonal antibody. Invitrogen.
Ebola Virus GP (subtype Sudan, strain Gulu) rabbit polyclonal antibody, Invitrogen.
Ebola Virus GP1 (mucin domain deleted) (subtype Sudan, strain Gulu) Recombinant rabbit
monoclonal antibody. Invitrogen.
Mouse monoclonal antibody anti-SUDV GP mAb (16F6) Sudan Virus 0280-001. IBT
Bioservices.
Mouse monoclonal antibody anti-SUDV GP mAb (2H5) Sudan Virus 0202-029. IT
Bioservices.
Mouse monoclonal antibody anti-SUDV VP40 mAb (1G10) Sudan Virus 0202-018. IBT
Bioservices.
Mouse monoclonal antibody Pan-Filovirus Chimeric anti-GP mAb Ebola, Sudan, Marburg
virus 0200-003. IBT Bioservices.
Mouse monoclonal antibody Pan-Filovirus Mouse anti-GP mAb Ebola, Sudan, Marburg virus
0200-001. IBT Bioservices.
Rabbit polyclonal antibody anti-EBOV GP pAb Ebola Virus 0301-015. IBT Bioservices.
Rabbit polyclonal antibody anti-EBOV GP pAb Biotinylated Ebola Virus 0301-015-bio.
IBT Bioservices.
Rabbit polyclonal antibody anti-EBOV GPddmuc pAb Ebola virus 0301-038. IBT
Bioservices.
3. Anti-Bundibugyo ebolavirus antibodies
Ebola Virus GP1 (subtype Bundibugyo, strain Uganda 2007) rabbit polyclonal antibody.
Invitrogen.
Rabbit polyclonal antibody anti-BDBV GP pAb Bundibugyo Virus 0304-001. IBT Bioservices.
4. Anti-Marburg marburgvirus antibodies
Mouse monoclonal antibody anti-MARV GP mAb (2D8) Marburg Virus 0203-025. IBT
Bioservices.
Mouse monoclonal antibody anti-MARV GP mAb (5C1) Marburg Virus 0203-023. IBT
Bioservices.
Mouse monoclonal antibody anti-MARV VP40 mAb (1D1) Marburg Virus 0203-01. IBT
Bioservices.
Mouse monoclonal antibody anti-MMARV VP40 mAb (6B1) Marburg Virus 0203-016. IBT
Bioservices.
C. Coronaviruses
1. Anti- SARS-CoV-1 antibodies
Anti-SARS-CoV Spike Glycoprotein Antibody [3A2] (A334). Antibodies.com
Anti-SARS-CoV Nucleocapsid Antibody (A57918). Antibodies.com
Anti-SARS-CoV-2 ORF3a Antibody (A121524). Antibodies.com
Mouse Monoclonal (IgG1) antibody to Coronavirus SARS-CoV MERS-CoV + SARS-CoV NP
LS-C829114. LSBiosciences.
Mouse Monoclonal (IgG1) antibody to Coronavirus SARS-CoV MERS-CoV + SARS-CoV NP
LS-C829113. LSBiosciences.
Mouse Monoclonal (IgG1) antibody to Coronavirus SARS-CoV MERS-CoV + SARS-CoV NP
LS-C829116. LSBiosciences.
2. Anti-SARS-CoV-2 antibodies
Capture mouse monoclonal antibody: Cat# 9547 MAb to SARS-CoV-2 NP; detection mouse
monoclonal antibody Cat# 9548 MAb to SARS-CoV-2 NP. This antibody pair does not cross-
react with other coronaviruses except SARS (2003). Meridian Bioscience.
Polyclonal Rabbit antibody anti-SARS-CoV-2 (COVID-19) Spike Protein S1 RBD CODE: 130-
10759-100. RayBiotech.
Polyclonal Rabbit antibody anti- SARS-CoV-2 (COVID-19) Nucleocapsid Protein CODE: 130-
10760-100. RayBiotech.
Polyclonal Rabbit antibody anti- SARS-CoV-2 (COVID-19) Spike Subunit 2 (S2F) CODE:
130-10761-100. RayBiotech.
Mouse anti-SARS-COV-2 N-Protein (5F7-A3) CODE: 130-10779-200. RayBiotech.
Mouse anti-SARS-CoV-2 N-Protein (1G1-G1) CODE: 130-10780-10. RayBiotech.
Mouse anti-SARS-CoV-2 N-Protein (1G1-F2) CODE: 130-10781-500. RayBiotech.
Mouse anti-SARS-CoV-2 N-Protein (5F7-B3) CODE: 130-10782-500. RayBiotech.
Rabbit anti-SARS-CoV-2 S1RBD CODE: 130-10784-1000. RayBiotech.
Rabbit anti-SARS-CoV-2 N-Protein CODE: 130-10785-1000. RayBiotech.
Rabbit anti-SARS-CoV-2 S1F CODE: 130-10786-1000. RayBiotech.
Mouse anti-SARS-CoV-2 N-Protein (1G1-B2-F10) CODE: 130-10789-500. RayBiotech.
Mouse anti-SARS-CoV-2 N-Protein (4F12-G1-F1) CODE: 130-10790-1000. RayBiotech.
Mouse anti-SARS-CoV-2 N-Protein (1B5-H3-C8) CODE: 130-10791-1000. RayBiotech.
Mouse anti-SARS-COV-2 N-Protein (1A4-G1-G12-G12) CODE: 130-10793-500. RayBiotech.
Mouse anti-SARS-COV-2 N-Protein (4G3-H12-A1) CODE: 130-10794-200. RayBiotech.
Mouse monoclonal antibody Anti-SARS-CoV-2 S1 Neutralizing Antibody 40591-MM43. Sino
Biological.
Anti- RBD Neutralizing mouse monoclonal antibody 40592-MM57. Sino Biological.
Anti- RBD Neutralizing monoclonal antibody, Rabbit 40592-R001. Sino Biological.
Anti-spike protein rabbit polyclonal antibody 40150-T62-COV2. Sino Biological.
Anti-spike mouse monoclonal antibody IP40150-MM02. Sino Biological.
Anti-spike protein rabbit polyclonal antibody 40150-RP01. Sino Biological.
Anti-spike protein rabbit polyclonal antibody 40150-T52. Sino Biological.
Anti-spike protein rabbit polyclonal antibody 40150-T62. Sino Biological.
Anti-spike mouse monoclonal antibody 40150-D004. Sino Biological.
Anti-spike mouse monoclonal antibody 40150-D001. Sino Biological.
Anti-spike mouse monoclonal antibody 40150-D003. Sino Biological.
Anti-spike mouse monoclonal antibody 40150-D002. Sino Biological.
Anti-spike mouse monoclonal antibody 40150-D005. Sino Biological.
Anti-spike mouse monoclonal antibody 40150-D006. Sino Biological.
Anti-spike mouse monoclonal antibody 40150-MM08. Sino Biological.
Customer No. 84331
Anti-spike mouse monoclonal antibody 40150-MM10. Sino Biological.
Anti-spike mouse chimeric monoclonal antibody 40150-D001-H. Sino Biological.
Anti- Nucleocapsid rabbit polyclonal antibody 40588-T62. Sino Biological.
Anti- Nucleocapsid rabbit polyclonal antibody 40143-R001. Sino Biological.
Anti-SARS-CoV/SARS-CoV-2 Nucleocapsid mouse monoclonal antibody 40143-MM05. Sino
Biological.
Anti-SARS-CoV/SARS-CoV-2 Nucleocapsid rabbit monoclonal antibody 40143-R019. Sino
Biological.
Anti-SARS-CoV/SARS-CoV-2 Nucleocapsid mouse monoclonal antibody 40143-MM08. Sino
Biological.
Anti- Nucleocapsid rabbit polyclonal antibody 40143-R040. Sino Biological.
Anti- Nucleocapsid rabbit polyclonal antibody 40143-R004. Sino Biological.
Anti- Nucleocapsid rabbit monoclonal antibody 40588-R0004. Sino Biological.
Anti-SARS-CoV-2 Nucleocapsid Protein Antibody [5B7] (A121539). Antibodies.com
Anti-SARS-CoV-2 Nucleocapsid Protein Antibody [2D8] (A121540). Antibodies.com
Anti-SARS-CoV-2 S-ECD Antibody (A121532). Antibodies.com
Anti-SARS-CoV-2 S-RBD Antibody (A121533). Antibodies.com
Anti-SARS-CoV-2 Spike Glycoprotein Antibody [2C1] (A121541). Antibodies.com
Anti-SARS-CoV-2 Spike Glycoprotein Antibody [1A1] (A121542). Antibodies.com
3. Anti-MERS-CoV antibodies
Anti-MERS Nucleocapsid (N) [9H10.2.3] monoclonal antibody. Karafast.
Rabbit Polyclonal antibody to Coronavirus MERS EMC/2012 MERS-CoV Nucleoprotein LS-
C483529. LSBiosciences.
Mouse Monoclonal antibody to Coronavirus MERS EMC/2012 MERS-CoV Nucleoprotein LS-
C483525. LSBiosciences.
Rabbit Polyclonal antibody to Coronavirus MERS EMC/2012 MERS-CoV Nucleoprotein LS-
C488273. LSBiosciences.
Mouse Monoclonal antibody to Coronavirus MERS EMC/2012 MERS-CoV S1 LS-C488278.
LSBiosciences.
Rabbit Polyclonal antibody to Coronavirus MERS MERS-CoV S1 LS-C483533. LSBiosciences.
Rabbit Polyclonal antibody to Coronavirus MERS MERS-CoV S1 S-C483534. LSBiosciences.
Rabbit Polyclonal antibody to Coronavirus MERS MERS-CoV S1 LS-C483532. LSBiosciences.
Mouse Monoclonal (IgG2a) antibody to Coronavirus MERS MERS-CoV S1 S-C829109.
LSBiosciences.
Mouse Monoclonal (IgG2a) antibody to Coronavirus MERS MERS-CoV S1 LS-C829110.
LSBiosciences.
Mouse Monoclonal (IgG1) antibody to Coronavirus MERS MERS-CoV S1 LS-C829115.
LSBiosciences.
Rabbit Polyclonal antibody to Coronavirus MERS MERS-CoV S1 LS-C488279. LSBiosciences.
Mouse Monoclonal [clone 11] (IgG1) antibody to Coronavirus MERS MERS-CoV S2 LS-
C820325. LSBiosciences.
Rabbit Polyclonal antibody to Coronavirus MERS MERS-CoV S2 LS-C483535. LSBiosciences.
Rabbit Polyclonal antibody to Coronavirus MERS MERS-CoV S2 LS-C488280. LSBiosciences.
Goat Polyclonal (IgG) antibody to Coronavirus MERS-CoV S2 LS-C757176. LSBiosciences.
Rabbit Polyclonal (IgG) antibody to Coronavirus MERS MERS-CoV Spike LS-C818405.
LSBiosciences.
Mouse Monoclonal (IgG1) antibody to Coronavirus MERS EMC/2012 MERS-CoV Spike LS-
C488274. LSBiosciences.
Rabbit Polyclonal antibody to Coronavirus MERS EMC/2012 MERS-CoV Spike LS-C488500.
LSBiosciences.
Rabbit Polyclonal (IgG) antibody to Coronavirus MERS MERS-CoV Spike LS-C818407.
LSBiosciences.
Rabbit Polyclonal antibody to Coronavirus MERS MERS-CoV Spike LS-C488276.
LSBiosciences.
Rabbit Polyclonal antibody to Coronavirus MERS EMC/2012 MERS-CoV Spike LS-C488275.
LSBiosciences.
Rabbit Polyclonal antibody to Coronavirus MERS EMC/2012 MERS-CoV Nucleoprotein LS-
C483529. LSBiosciences.
Rabbit Polyclonal antibody to Coronavirus MERS MERS-CoV Nucleoprotein LS-C483528.
LSBiosciences.
Mouse Monoclonal antibody to Coronavirus MERS EMC/2012 MERS-CoV Nucleoprotein LS-
C483525. LSBiosciences.
Rabbit Polyclonal antibody to Coronavirus MERS EMC/2012 MERS-CoV Nucleoprotein LS-
C488273. LSBiosciences.
Anti-MERS-CoV Spike Protein polyclonal antibody CABT-B1950. Creative Diagnostics.
Anti-MERS-CoV Spike Protein (aa 1-1297) polyclonal antibody CABT-B1951. Creative
Diagnostics.
Anti-MERS-CoV Spike Protein S2 polyclonal antibody CABT-B1952. Creative Diagnostics.
Anti-MERS-CoV Spike Protein S1 (Center region) polyclonal antibody CABT-B1953. Creative
Diagnostics.
Anti-MERS-CoV Spike Protein S1 (N-terminal) polyclonal antibody CABT-B1954. Creative
Diagnostics.
Anti-MERS-CoV Spike Protein S1 (C-terminal) polyclonal antibody, CABT-B1955. Creative
Diagnostics.
Anti-MERS-CoV Spike Protein S2 polyclonal antibody, CABT-B1956. Creative Diagnostics.
Anti-MERS-CoV Spike Protein (aa 1-1297) polyclonal antibody CABT-B1957. Creative
Diagnostics.
Anti-MERS-CoV Spike Protein (aa 726-1296) monoclonal antibody, clone 13, CABT-B1958.
Creative Diagnostics.
Anti-MERS-CoV Spike Protein (aa 1-1297) monoclonal antibody, clone 834 CABT-B1959.
Creative Diagnostics.
Anti-MERS-CoV Spike Protein S1 (aa 1-725) polyclonal antibody CABT-B1960. Creative
Diagnostics.
Anti-MERS-CoV Nucleoprotein monoclonal antibody, clone 21 CABT-B1961. Creative
Diagnostics.
Anti-MERS-CoV Nucleoprotein (N-terminal) polyclonal antibody CABT-B1963. Creative
Diagnostics.
Anti-MERS-CoV Nucleoprotein (Center region) polyclonal antibody CABT-B1964. Creative
Diagnostics.
Anti-MERS-CoV Nucleoprotein polyclonal antibody CABT-B1965. Creative Diagnostics.
Anti-MERS-CoV Nucleoprotein (Center region) polyclonal antibody CABT-B1966. Creative
Diagnostics.
Mouse anti-MERS-CoV S1 monoclonal antibody, clone MN2163 CABT-RM313. Creative
Diagnostics.
Mouse anti-MERS-CoV S1 monoclonal antibody, clone MN2164 CABT-RM314. Creative
Diagnostics.
4. Anti-CoV-HKU1 antibodies
Mouse Monoclonal (IgG1) antibody to Coronavirus HKU1 CoV HKU1 Spike Glycoprotein
LS-C487049. LSBiosciences.
Rabbit Monoclonal (IgG) antibody to Coronavirus HKU1 CoV HKU1 Spike Glycoprotein LS-
C487046. LSBiosciences.
Rabbit Polyclonal antibody to Coronavirus HKU1 CoV HKU1 Spike Glycoprotein LS-
C487047. LSBiosciences.
Rabbit Polyclonal antibody to Coronavirus HKU1 CoV HKU1 Spike Glycoprotein LS-
C489932. LSBiosciences.
5. Anti-CoV-OC43 antibodies
Anti-Coronavirus Mouse monoclonal antibody, OC-43 strain, clone 541-8F. Sigma-Aldrich
Mouse Monoclonal (IgG) antibody to Human Coronavirus OC43 Coronavirus OC43 LS-
C79763. LSBiosciences.
Mouse Monoclonal (IgG) antibody to Human Coronavirus OC43 CoV OC43 Nucleoprotein
LS-C79764. LSBiosciences.
Rabbit Polyclonal (IgG) antibody to Human CoV OC43 Spike Glycoprotein (aa15-344) LS-
C371066. LSBiosciences.
Rabbit anti-Human coronavirus OC43 S Polyclonal antibody Uniprot No. P36334. Cusabio.
Mouse Anti-HCoV OC43, 229E Nucleoprotein Monoclonal antibody, clone 6538E CABT-
CS025. Creative Diagnostics.
Anti-HCoV OC43 monoclonal antibody, clone 4616 CABT-B343. Creative Diagnostics.
Anti-HCoV OC43 monoclonal antibody, clone 4615 CABT-B342. Creative Diagnostics.
Mouse anti-HCoV spike glycoprotein monoclonal antibody, clone 18 CABT-RM315. Creative
Diagnostics.
Rabbit anti-HCoV spike glycoprotein monoclonal antibody, clone 139 CABT-RM316. Creative
Diagnostics.
Rabbit anti-HCoV spike glycoprotein polyclonal antibody CABT-RM317. Creative Diagnostics.
6. Anti- HCoV-NL63 antibodies
Mouse IgG1 monoclonal antibody 1B12. (Clin Vaccine Immunol. 2011 January; 18(1): 113-118.
doi: 10.1128/CVI.00355-10, PMCID: PMC301978)
Mouse IgG1 monoclonal antibody 2D4. (Clin Vaccine Immunol. 2011 January; 18(1): 113-118.
doi: 10.1128/CVI.00355-10, PMCID: PMC301978)
Mouse IgG1 monoclonal antibody 2E6. (Clin Vaccine Immunol. 2011 January; 18(1): 113-118.
doi: 10.1128/CVI.00355-10, PMCID: PMC301978)
7. Anti-HCoV-229E antibodies
Mouse IgG2b monoclonal antibody 1E7. (Clin Vaccine Immunol. 2011 January; 18(1): 113-118.
doi: 10.1128/CVI.00355-10, PMCID: PMC301978)

TABLE 2
Sequences of Viral Polypeptides for the Viral Test Cassette
A. Sequences of Filovirus Proteins Used in Viral Test Cassette

| | | Amino | | |
|---|---|---|---|---|
| | | Acid | GenBank | |
| Species¹ | Gene | Residues | Sequence | Nucleotide Substitutions³ |
| Bundibugyo | VP40 | 1-326 (SEQ | FJ217161.1 | None |
| ebolavirus | | ID NO: 14) | | |
| | NP | 1-739 (SEQ | FJ217161.1 | C1735T (silent) |
| | | ID NO: 16) | | |
| | GP-mucin | 313-465 | FJ217161.1 | C6973T (silent), A7363G |
| | | (SEQ ID | | (silent) |
| | | NO: 18) | | |
| Tai Forest | VP40 | 1-326 (SEQ | FJ217162.1 | None |
| ebolavirus | | ID NO: 20) | | |
| | NP | 1-739 (SEQ | FJ217162.1 | None |
| | | ID NO: 22) | | |
| | GP-mucin | 313-465 | FJ217162.1 | None |
| | | (SEQ ID | | |
| | | NO: 24) | | |
| Reston ebolavirus | VP40 | 1-331 (SEQ | AF522874.1 | G4490A (silent), A5466G |
| (Pennsylvania) | | ID NO: 26) | | (Asn to Asp) |
| | NP | 1-739 (SEQ | AF522874.1 | T2188C (silent) |
| | | ID NO: 28) | | |
| | GP-mucin | 314-466 | AY769362. | G7093A (silent) |
| | | (SEQ ID | | |
| | | NO: 30) | | |
| Sudan ebolavirus | VP40 | 1-326 (SEQ | FJ968794.1 | T4465C (silent) |
| (Boniface) | | ID NO: 8) | | |
| | NP | 1-738 (SEQ | AF173836.1 | C2581T (silent) |
| | | ID NO: 10) | | |
| | GP-mucin | 313-465 | FJ968794.1 | A7112G (silent) |
| | | (SEQ ID | | |
| | | NO: 12) | | |
| Zaire ebolavirus | VP40 | 1-326 (SEQ | AF499101.1 | G4496A (silent), A4592G |
| (Mayinga) | | ID NO: 2) | | (silent), T5204C (silent) |
| | NP | 1-739 (SEQ | AF086833.2 | A491G (Ile to Val) |
| | | ID NO: 4) | | |
| | GP-mucin² | 313-465 | JQ352763.1 | None |
| | | (SEQ ID | | |
| | | NO: 6) | | |
| Marburg | VP40 | 1-303 (SEQ | DQ217792.1 | None |
| marburgvirus | | ID NO: 32) | | |
| (Musoke) | | | | |
| | NP | 1-695 (SEQ | DQ217792.1 | None |
| | | ID NO: 34) | | |
| | GP-mucin | 289-505 | DQ217792.1 | A6906T (silent) |
| | | (SEQ ID | | |
| | | NO: 36) | | |

¹Known strain names are in parentheses.
²Zaire ebolavirus GP-mucin sequence is from the Kikwit strain.
³The position of nucleotide substitutions compared to GenBank sequence as reference, with the
corresponding amino acid substitution, if any, noted in parentheses.
B. Sequences of Flavivirus Proteins in Viral Test Cassette and Oligonucleotide Primers Used

| | | GenBank | | | | |
|---|---|---|---|---|---|---|
| | | Polyprotein | Amino | | | Amino acid |
| Flavivirus | | Accession | acid | Forward primer | Reverse primer | substi- |
| (strain) | Gene | Number | residues | (5′-3′) | (5′-3′) | tutions¹ |
| ZIKV | E | ALU33341 | 291-744 | CACCATCCGG | CGGCTCCGAA | |
| (str.SPH2015) | | | (SEQ ID | TGCATAGGTG | GATTTG (SEQ | |
| | | | NO:37) | TTTCG (SEQ ID | ID NO: 39) | |
| | | | | NO: 38) | TTAAGAACCC | |
| | NS1 | ALU33341 | 796-1148 | CACCGTGGGC | GCTGTAACCAT | |
| | | | (SEQ ID | TGTTCAGTCGA | TGAACGCAC | |
| | | | NO: 40) | C (SEQ ID NO: | (SEQ ID NO: 42) | |
| | | | | 41) | TTATTTAAAGG | |
| ZIKV_AS | E | ACD75819 | 291-744 | CACCATTCGTT | CTGCGCCAAA | |
| (str.YAP) | | | (SEQ ID | GCATCGGCGT | GATCTG (SEQ | |
| | | | NO: 43) | G (SEQ ID NO: | ID NO: 45) | |
| | | | | 44) | TTAGCTGCGAT | |
| | E-DIII | ACD75819 | 591-693 | CACCAAAGGC | GCCAATGATG | |
| | | | (SEQ ID | GTAAGCTACTC | (SEQ ID NO: 48) | |
| | | | NO: 46) | A (SEQ ID NO: | | |
| | | | | 47) | TTAACTATACG | |
| | pM | ACD75819 | 126-290 | CACCACGCGG | CCGGAGCGAT | |
| | | | (SEQ ID | CGCGGTTCAG | GAGCAG (SEQ | |
| | | | NO: 49) | CATA (SEQ ID | ID NO: 51) | |
| | | | | NO: 50) | TTAAGAACCC | |
| | NS1 | ACD75819 | 796-1148 | CACCGTGGGG | GCCGTGACCA | |
| | | | SEQ ID | TGTTCAGTTGA | T (SEQ ID NO: | |
| | | | NO: 52) | T (SEQ ID NO: | 54) | |
| | | | | 53) | TCATTTAAACG | |
| ZIKV (str. | E | AEN75266 | 291-744 | CACCATTCGG | CAGCTCCGAA | |
| ArD_41519) | | | (SEQ ID | TGCATAGGGG | AATCTGATG | |
| | | | NO: 55) | TCAGC (SEQ ID | (SEQ ID NO: 57) | |
| | | | | NO: 56) | TTATTTGAAGG | |
| ZIKV (str. | E | AHF49785 | 291-744 | CACCATTCGTT | CTGCACCAAA | |
| ARB7701) | | | (SEQ ID | GCATTGGTGTT | AATCTGATGCA | |
| | | | NO: 58) | AG (SEQ ID NO: | C (SEQ ID NO: | |
| | | | | 59) | 60) | |
| | | | | | TTATTTAAATG | |
| ZIKV_AFR | E | AAV34151 | 291-740 | CACCAAAGGC | CGGCGCCGAA | |
| (str.MR-766) | | | (SEQ ID | GTTAGCTATAG | AATCTG (SEQ | |
| | | | NO: 61) | T (SEQ ID NO: | ID NO: 63) | |
| | | | | 62) | TTATGAACGAT | |
| | E-DIII | AAV34151 | 587-689 | CACCAAGGGC | GCCAATGGTG | |
| | | | (SEQ ID | GTGTCATATTC | CGT (SEQ ID | |
| | | | NO: 64) | C (SEQ ID NO: | NO: 66) | |
| | | | | 65) | TTAGGAATACG | |
| | pM | AAV34151 | 126-290 | CACCACTCGC | CCGGTGCGAT | |
| | | | (SEQ ID | CGCGGGTCA | CAG (SEQ ID | |
| | | | NO: 67) | (SEQ ID NO: 68) | NO: 69) | |
| | NS1 | AAV34151 | 792-1144 | CACCGTCGGC | TTAAGAGCCA | |
| | | | (SEQ ID | TGTAGCGTC | GCCGTAACCA | |
| | | | NO: 70) | (SEQ ID NO: 71) | TACT (SEQ ID | |
| | | | | | NO: 72) | |
| DENV1 | E | AIU47321 | 281-722 | CACCATGCGG | TTAAGTTCCAA | T4421, |
| | | | (SEQ ID | TGCGTGGGAA | AGATCTGGTG | E485G, T505R, |
| | | | NO: 73) | TA (SEQ ID NO: | TAC (SEQ ID | Q515E |
| | | | | 74) | NO: 75) | |
| | E-DIII | AIU47321 | 579-677 | CACCTATGTGA | TTAGCTGCTTC | |
| | | | (SEQ ID | TGTGCACAGG | CTTTCTTGAAC | |
| | | | NO: 76 | CTCATTC (SEQ | CAGCT (SEQ ID | |
| | | | | ID NO: 77) | NO: 78) | |
| | M | AIU47321 | 206-279 | TCCGTCGCAC | GGCCATGGAT | T2501 |
| | | | (SEQ ID | TGGCACCACA | GGAGTTACCA | |
| | | | NO: 79) | C (SEQ ID NO: | G (SEQ ID NO: | |
| | | | | 80) | 81) | |
| | NS1 | AIU47321 | 776-1126 | GACTCGGGAT | TGCAGAGACC | G880R |
| | | | (SEQ ID | GTGTAATCAAC | ATTGACTTAAC | |
| | | | NO: 82) | (SEQ ID NO: 83) | (SEQ ID NO: 84) | |
| | NS3 | AIU47321 | 1476-2093 | TCAGGAGTGC | TCTTCTTCCTG | GSVEAF1796- |
| | | | (SEQ ID | TATGGGACAC | CTGCGAACTC | 1801RIGGGL, |
| | | | NO: 85) | ACCC (SEQ ID | (SEQ ID NO: 87) | Q2081L |
| | | | | NO: 86) | | |
| DENV2 | E | AAC59275 | 281-722 | CACCATGCGT | TTATGCTCCGA | P331S, E407K, |
| | | | (SEQ ID | TGCATAGGAAT | AAACTTGGTG | V4211, V4451 |
| | | | NO: 88) | ATCA (SEQ ID | GAG (SEQ ID | |
| | | | | NO: 89) | NO: 90) | |
| | E-DIII | AAC59275 | 575-675 | CACCAAAGGA | TTATCCTTTCT | |
| | | | (SEQ ID | ATGTCATACTC | TAAACCAGTTG | |
| | | | NO: 91) | TATGTGC (SEQ | AGCTT (SEQ ID | |
| | | | | ID NO: 92) | NO: 93) | |
| | M | AAC59275 | 206-279 | TCAGTGGCAC | TGTCATTGAAG | |
| | | | (SEQ ID | TCGTTCCACAT | GAGTGACAGC | |
| | | | NO: 94) | GTG (SEQ ID | (SEQ ID NO: 96) | |
| | | | | NO: 95) | | |
| | NS1 | AAC59275 | 776-1127 | GATAGTGGTT | AGCTGTGACC | A1127S |
| | | | (SEQ ID | GCGTTGTGAG | AAGGAGTTGA | |
| | | | NO: 97) | C (SEQ ID NO: | C (SEQ ID NO: | |
| | | | | 98) | 99) | |
| | NS3 | AAC59275 | 1476-2092 | GCCGGAGTAT | CTTTCTTCCGG | SIAA1768- |
| | | | (SEQ ID | TGTGGGATGT | CTGCAAATTC | 1771GMGV, |
| | | | NO: 100) | T (SEQ ID NO: | (SEQ ID NO: | G1773R, |
| | | | | 101) | 102) | GE1782- |
| | | | | | | 1783VK |
| DENV3 | E | AAA99437 | 281-722 | CACCATGAGA | TTAGTAAGCAC | K571E |
| | | | (SEQ ID | TGTGTGGGAG | TCCCAAATATT | |
| | | | NO: 103) | TAGGA (SEQ ID | TGG (SEQ ID | |
| | | | | NO: 104) | NO: 105) | |
| | E-DIII | AAA99437 | 574-672 | CACCGGGATG | TTACTTCTTGT | R671K |
| | | | (SEQ ID | AGCTATGCAAT | ACCAGTTGATT | |
| | | | NO: 106) | GTGC (SEQ ID | TTCAG (SEQ ID | |
| | | | | NO: 107) | NO: 108) | |
| | M | AAA99437 | 206-279 | TCAGTGGCGT | TGTCATGGAT | |
| | | | (SEQ ID | TAGCTCCCCAT | GGGGTGACCA | |
| | | | NO: 109) | (SEQ ID NO: | GCAT (SEQ ID | |
| | | | | 110) | NO: 111) | |
| | NS1 | AAA99437 | 774-1125 | GACATGGGGT | CGCTGAGACT | A1125S |
| | | | (SEQ ID | GTGTCATAAAC | AAAGACTT | |
| | | | NO: 112) | (SEQ ID NO: | (SEQ ID NO: | |
| | | | | 113) | 114) | |
| | NS3 | AAA99437 | 1474-2091 | TCCGGCGTTTT | CTTTCTGCCAG | T1593A, |
| | | | (SEQ ID | ATGGGACGTA | CCGCAAA | G1606R, |
| | | | NO: 115) | (SEQ ID NO: | (SEQ ID NO: | M1709L, |
| | | | | 116) | 117) | VP1749- |
| | | | | | | 1750GS, |
| | | | | | | V1845D, |
| | | | | | | 11892K, |
| | | | | | | K1955N |
| DENV4 | E | AAX48017 | 280-721 | CACCATGCGA | TTAACTACCAA | D375A, P436S, |
| | | | (SEQ ID | TGCGTGGGAG | AAACCTGGTG | N437S, F636L, |
| | | | NO: 118) | TG (SEQ ID NO: | TAC (SEQ ID | L682F |
| | | | | 119) | NO: 120) | |
| | E-DIII | AAX48017 | 574-676 | CACCAAGGGA | TTAGGAACTCC | F636L |
| | | | (SEQ ID | ATGTCATACAC | CTTTCCTGAAC | |
| | | | NO: 121) | GATGTGC | CAATG (SEQ ID | |
| | | | | (SEQ ID NO: | NO: 123) | |
| | | | | 122 | | |
| | M | AAX48017 | 205-278 | TCAGTAGCTTT | TCCGTAGGAT | |
| | | | (SEQ ID | AACACCACATT | GGGGCGACCA | |
| | | | NO: 124) | CA (SEQ ID NO: | G (SEQ ID NO: | |
| | | | | 125) | 126) | |
| | NS1 | AAX48017 | 775-1125 | GACATGGGTT | GGCCGTCACC | E825K |
| | | | (SEQ ID | GTGTGGCG | TGTGATTT | |
| | | | NO: 127) | (SEQ ID NO: | (SEQ ID NO: | |
| | | | | 128) | 129) | |
| | NS3 | AAX48017 | 1474-2091 | TCAGGAGCCC | CTTCCTTCCAC | T1747N, |
| | | | (SEQ ID | TGTGGGAC | TGGCAAACTC | F1787L, |
| | | | NO: 130) | (SEQ ID NO: | (SEQ ID NO: | S1908L |
| | | | | 131) | 132) | |
| WNV | E | YP_0015278 | 291-741 | CACCTTCAACT | TTAGCGGAAT | L571F |
| | | 77 | (SEQ ID | GCCTTGGAAT | GCTCCTCCGA | |
| | | | NO: 133) | GAGCAAC | ACACTTG (SEQ | |
| | | | | (SEQ ID NO: | ID NO: 135) | |
| | | | | 134) | | |
| | E-DIII | YP_0015278 | 586-705 | CACCCAGTTG | | |
| | | 77 | (SEQ ID | AAGGGAACAA | TGAGGGTGGT | |
| | | | NO: 136) | CCTATGGC | TGT (SEQ ID | |
| | | | | (SEQ ID NO: | NO: 138) | |
| | | | | 137) | | |
| | pM | YP_0015278 | 124-290 | CACCGTTACC | TTAGCTGTAAG | |
| | | 77 | (SEQ ID | CTCTCTAACTT | CTGGGGCCAC | |
| | | | NO: 139) | CCAAGGG | CAA (SEQ ID | |
| | | | | (SEQ ID NO: | NO: 141) | |
| | | | | 140) | | |
| | NS1 | YP_0015278 | 792-1143 | CACCGACACT | AGCATTCACTT | V1076A |
| | | 77 | (SEQ ID | GGGTGTGCC | GTGACTGCAC | |
| | | | NO: 142) | (SEQ ID NO: | (SEQ ID NO: | |
| | | | | 143) | 144) | |
| YFV | E | AFR54324 | 286-728 | CACCGCTCAC | TTACTGAAAGG | |
| | | | (SEQ ID | TGCATTGGAAT | CAGAGCCAAA | |
| | | | NO: 145) | T (SEQ ID NO: | CAC (SEQ ID | |
| | | | | 146) | NO: 147) | |
| | E-DIII | AFR54324 | 573-683 | CACCTCAGCTT | TTACTTTCCTA | |
| | | | (SEQ ID | TGACACTCAA | TTGAGCTTCCC | |
| | | | NO: 148) | GGGG (SEQ ID | TC (SEQ ID NO: | |
| | | | | NO: 149) | 150) | |
| | pM | AFR54324 | 122-285 | CACCGTGACC | TGAGTAGGCC | |
| | | | (SEQ ID | TTGGTGCGGA | GGACCAACAG | |
| | | | NO: 151) | AAAAC (SEQ ID | C (SEQ ID NO: | |
| | | | | NO: 152) | 153) | |
| | NS1 | AFR54324 | 779-1130 | CACCGATCAA | AGCTGTAACC | |
| | | | (SEQ ID | GGATGCGCCA | CAGGAGCGCA | |
| | | | NO: 154) | TCAAC (SEQ ID | C (SEQ ID NO: | |
| | | | | NO: 155) | 156) | |
| | NS3 | AFR54324 | 1485-2107 | AGTGGGGATG | CCTCCTACCTT | |
| | | | (SEQ ID | TCTTGTGGGAT | CAGCAAACTTA | |
| | | | NO: 157) | (SEQ ID NO: | AT (SEQ ID NO: | |
| | | | | 158) | 159) | |
| JEV | E | AAK11279 | 299-744 | CACCTTTAATT | TTATCTGAAGG | |
| | | | (SEQ ID | GTCTGGGAAT | CATCACCAAAC | |
| | | | NO: 160) | GGGC (SEQ ID | ACTTG (SEQ ID | |
| | | | | NO: 161) | NO: 162) | |
| | E-DIII | AAK11279 | 586-696 | CACCGACAAA | TTACGTGCTTC | |
| | | | (SEQ ID | CTGGCTCTGA | CAGCTTTGTG | |
| | | | NO: 163) | AAGGC (SEQ | CCAATG (SEQ | |
| | | | | ID NO: 164) | ID NO: 165) | |
| | pM | AAK11279 | 128-294 | CACCATGAAG | TTAACTGTAAG | |
| | | | (SEQ ID | TTGTCGAATTT | CCGGAGCGAC | |
| | | | NO: 166) | CCAG (SEQ ID | CAAC (SEQ ID | |
| | | | | NO: 167) | NO: 168) | |
| | NS1 | AAK11279 | 795-1146 | CACCGACACT | AGCATGAACC | N876D |
| | | | (SEQ ID | GGATGTGCCA | TGTGATCTGAC | |
| | | | NO: 169) | TTGAC (SEQ ID | GAG (SEQ ID | |
| | | | | NO: 170) | NO: 171) | |
| SLEV | E | ABN11832 | 289-739 | CACCTTCAACT | TTACCTAAATG | |
| | | | (SEQ ID | GTTTGGGAAC | CTCCTCCAAAA | |
| | | | NO: 172) | ATCA (SEQ ID | AC (SEQ ID NO: | |
| | | | | NO: 173) | 174) | |
| | E-DIII | ABN11832 | 587-692 | CACCGGAACG | TTAAATGCTGC | |
| | | | (SEQ ID | ACATATGGTAT | TTCCCTCTTTG | |
| | | | NO: 175) | GTGTGAC | TG (SEQ ID NO: | |
| | | | | (SEQ ID NO: | 177) | |
| | | | | 176) | | |
| | pM | ABN11832 | 122-288 | CACCTTGCAG | TTAGCTGTATG | |
| | | | (SEQ ID | TTATCAACCTA | CCGGAGCAAT | |
| | | | NO: 178) | TCAGGGG | CAG (SEQ ID | |
| | | | | (SEQ ID NO: | NO: 180) | |
| | | | | 179) | | |
| | NS1 | ABN11832 | 790-1145 | CACCGATTCG | AGCTGTCACT | |
| | | | (SEQ ID | GGATGTGCAA | CGAGATTTCAC | |
| | | | NO: 181) | TT (SEQ ID NO: | (SEQ ID NO: | |
| | | | | 182 | 183) | |
| MVEV | E | NP_051124 | 293-743 | CACCTTTAATT | TTAACGAAATG | |
| | | | (SEQ ID | GCCTGGGTAT | CACCACCAAA | |
| | | | NO: 184) | G (SEQ ID NO: | AACCTG (SEQ | |
| | | | | 185) | ID NO: 186) | |
| | E-DIII | NP_051124 | 590-698 | CACCAAAGGA | TTATTTGCCAA | |
| | | | (SEQ ID | ACCACTTATGG | TTGAACTACCC | |
| | | | NO: 187) | GATG (SEQ ID | TC (SEQ ID NO: | |
| | | | | NO: 188) | 189) | |
| | M | NP_051124 | 218-292 | CACCTTAAAGC | CTCGTTGCTC | |
| | | | (SEQ ID | TTTCCACCTTC | CTGCCTACAG | |
| | | | NO: 190) | CAGGGC (SEQ | TTAA (SEQ ID | |
| | | | | ID NO: 191) | NO: 192) | |
| | NS1 | NP_051124 | 794-1145 | CACCGATACC | TTATGCCTGAA | |
| | | | (SEQ ID | GGTTGTGC | CACGGCTTTTA | |
| | | | NO: 193) | (SEQ ID NO: | AC (SEQ ID NO: | |
| | | | | 194) | 195) | |
| ROCV | E | ATG32103 | 286-736 | CACCATCAACT | TTATCTAAACG | |
| | | | (SEQ ID | GCTTGGGTGT | CTCCTCCGAA | |
| | | | NO: 196) | GACC (SEQ ID | GAG (SEQ ID | |
| | | | | NO: 197) | NO: 198) | |
| | E-DIII | ATG32103 | 583-691 | CACCAAAGGG | TTAGCTCCCG | |
| | | | (SEQ ID | TCAACATACCT | ATCACACTACC | |
| | | | NO: 199) | GATGTGC | AGA (SEQ ID | |
| | | | | (SEQ ID NO: | NO: 201) | |
| | | | | 200) | | |
| | pM | ATG32103 | 119-285 | CACCCTGCGC | TTAGCTGTAG | K165N |
| | | | (SEQ ID | CTGGGGACAT | GCTGGGGCTA | |
| | | | NO: 202) | ATCAA (SEQ ID | TCAG (SEQ ID | |
| | | | | NO: 203) | NO: 204) | |
| | NS1 | ATG32103 | 788-1142 | CACCACAGGA | TCCGGTCCCA | |
| | | | (SEQ ID | TGCGCGATTG | GCTGTCACTTT | |
| | | | | AC (SEQ ID NO: | | |
| | | | NO: 205) | 206) | (SEQ ID NO: | |
| | | | | | 207) | |
| | NS3 | ATG32103 | 1498-2116 | CACCGGTGGT | GCGCTTCCCC | S1665H, |
| | | | (SEQ ID | GTTCTGTGG | GCCGCAAATT | T1722S |
| | | | NO: 208) | (SEQ ID NO: | C (SEQ ID NO: | |
| | | | | 209) | 210) | |
| POWV | E | NP_620099 | 279-725 | CACCACTCGC | TTAGTTAAATG | |
| | | | (SEQ ID | TGTACACACCT | CGCCGCCGAG | |
| | | | NO: 211) | (SEQ ID NO: | (SEQ ID NO: | |
| | | | | 212) | 213 | |
| | E-DIII | NP_620099 | 578-673 | CACCAAAGGG | TTATTGAAACC | |
| | | | (SEQ ID | ACGACGTACA | ACTGCTGTGA | |
| | | | NO: 214) | GTATG (SEQ ID | CAG (SEQ ID | |
| | | | | NO: 215) | NO: 216) | |
| | PM | NP_620099 | 111-278 | CACCATGGCG | TCACGCGTAC | |
| | | | (SEQ ID | ATGGCTACCA | ACAGGACCCA | |
| | | | NO: 217) | C (SEQ ID NO: | G (SEQ ID NO: | |
| | | | | 218) | 219) | |
| | NS1 | NP_620099 | 776-1128 | CACCGATTAC | TTACGCAACG | |
| | | | (SEQ ID | GGCTGCGCAA | ACCATAGAAC | |
| | | | NO: 220) | T (SEQ ID NO: | G (SEQ ID NO: | |
| | | | | 221) | 222) | |
| TBEV_E (str. | E | BAB72162 | 281-726 | CACCTCACGTT | TTAATTAAACG | |
| SOFJIN-HO) | | | (SEQ ID | GCACACATCT | CACCACCGAG | |
| | | | NO: 223) | G (SEQ ID NO: | TAC (SEQ ID | |
| | | | | 224) | NO: 225) | |
| | E-DIII | BAB72162 | 580-675 | CACCAAGGGC | TTATTTCTGAA | |
| | | | (SEQ ID | CTGACGTATAC | ACCACTGGTG | |
| | | | NO: 226) | G (SEQ ID NO: | GCT (SEQ ID | |
| | | | | 227) | NO: 228) | |
| | pM | BAB72162 | 113-280 | CACCATGACC | TCAAGCGTATA | |
| | | | (SEQ ID | TTGGCAGCGA | CCGGAGCCAG | |
| | | | NO: 229) | CG (SEQ ID NO: | GCA (SEQ ID | |
| | | | | 230) | NO: 231) | |
| | NS1 | BAB72162 | 777-1128 | CACCGATGTT | TTACGCGACC | |
| | | | (SEQ ID | GGTTGTGCG | ACCATTGAGC | |
| | | | NO: 232) | (SEQ ID NO: | G (SEQ ID NO: | |
| | | | | 233) | 234) | |
| TBEV_EUR | E | NP_043135 | 281-726 | CACCAGCCGT | TTATTGAAAG | |
| (str. NEUDOE | | | (SEQ ID | TGTACTCATCT | CTCCGCCAAG | |
| RFL) | | | NO: 235) | G (SEQ ID NO: | CAC (SEQ ID | |
| | | | | 236) | NO: 237) | |
| | E-DIII | NP_043135 | 580-675 | CACCAAAGGC | TTATTTCTGAA | |
| | | | (SEQ ID | TTAACCTACAC | ACCACTGATG | |
| | | | NO: 238) | AATGTGT (SEQ | AGACAG (SEQ | |
| | | | | ID NO: 239) | ID NO: 240) | |
| | pM | NP_043135 | 113-280 | CACCGTGACT | TCATGCGTAG | |
| | | | (SEQ ID | CTGGCAGCG | ACCGGCGC | |
| | | | NO: 241) | (SEQ ID NO: | (SEQ ID NO: | |
| | | | | 242 | 243) | |
| | NS1 | NP_043135 | 777-1128 | CACCGACGTG | TTAAGCCACCA | |
| | | | (SEQ ID | GGTGGTGCCG | CCATGCTCCG | |
| | | | NO: 244) | TT (SEQ ID NO: | GAT (SEQ ID | |
| | | | | 245) | NO: 246) | |

¹The position of nucleotide substitutions compared to GenBank sequence as reference, with the
corresponding amino acid change, if any, noted in parentheses.

| | Insert | |
|---|---|---|
| Name | Length | Insert Sequence |
| SARSS | 720 | Nucleic acid (SEQ ID NO: 247) |
| | | Protein (SEQ ID NO: 248) |
| NL63N | 1131 | Nucleic acid (SEQ ID NO: 249) |
| | | Protein (SEQ ID NO: 250) |
| SARS2N | 1257 | Nucleic acid (SEQ ID NO: 251) |
| | | Protein (SEQ ID NO: 252) |
| 229EN | 1167 | Nucleic acid (SEQ ID NO: 253) |
| | | Protein (SEQ ID NO: 254) |
| SARS2S | 723 | Nucleic acid (SEQ ID NO: 255) |
| | | Protein (SEQ ID NO: 256) |
| HKUIN | 1323 | Nucleic acid (SEQ ID NO: 257) |
| | | Protein (SEQ ID NO: 258) |
| OC43N | 1344 | Nucleic acid (SEQ ID NO: 259) |
| | | Protein (SEQ ID NO: 260) |
| SARSN | 1266 | Nucleic acid (SEQ ID NO: 261) |
| | | Protein (SEQ ID NO: 262) |
| 229ES | 558 | Nucleic acid (SEQ ID NO: 263) |
| | | Protein (SEQ ID NO: 264) |
| NL63S | 552 | Nucleic acid (SEQ ID NO: 265) |
| | | Protein (SEQ ID NO: 266) |
| HKUIS | 993 | Nucleic acid (SEQ ID NO: 267) |
| | | Protein (SEQ ID NO: 268) |
| OC43S | 978 | Nucleic acid (SEQ ID NO: 269) |
| | | Protein (SEQ ID NO: 270) |
| MERSS | 774 | Nucleic acid (SEQ ID NO: 271) |
| | | Protein (SEQ ID NO: 272) |
| MERSN | 1239 | Nucleic acid (SEQ ID NO: 273) |
| | | Protein (SEQ ID NO: 274) |
| UKS | 1584 | Nucleic acid (SEQ ID NO: 275) |
| | | Protein (SEQ ID NO: 276) |
| SAS | 1593 | Nucleic acid (SEQ ID NO: 277) |
| | | Protein (SEQ ID NO: 278) |
| BRS | 1593 | Nucleic acid (SEQ ID NO: 279) |
| | | Protein (SEQ ID NO: 280) |
| BRbd | 666 | Nucleic acid (SEQ ID NO: 281) |
| | | Protein (SEQ ID NO: 282) |
| WRSS | 1590 | Nucleic acid (SEQ ID NO: 283) |
| | | Protein (SEQ ID NO: 284) |
| UKSbd | 666 | Nucleic acid (SEQ ID NO: 285) |
| | | Protein (SEQ ID NO: 286) |
| SASbd | 666 | Nucleic acid (SEQ ID NO: 287) |
| | | Protein (SEQ ID NO: 288) |
| IDS B.1.617.1 | 1587 | Nucleic acid (SEQ ID NO: 289) |
| | | Protein (SEQ ID NO: 290) |
| IDSbd B.1.617.1 | 663 | Nucleic acid (SEQ ID NO: 291) |
| | | Protein (SEQ ID NO: 292) |

Filovirus sequences that may be used in the system and methods described herein include, but are not limited to, antigenic fragments and/or variants of the following sequences: Bundibugyo ebolavirus VP40 amino acid residues 1-326 (SEQ ID NO: 14), Bundibugyo ebolavirus NP amino acid residues 1-739 (SEQ ID NO: 16), Bundibugyo ebolavirus GP-mucin amino acid residues 313-465 (SEQ ID NO: 18), Tai Forest ebolavirus VP40 amino acid residues 1-326 (SEQ ID NO: 20), Tai Forest ebolavirus NP amino acid residues 1-739 (SEQ ID NO: 22), Tai Forest ebolavirus GP-mucin amino acid residues 313-465 (SEQ ID NO: 24), Reston ebolavirus (Pennsylvania) VP40 amino acid residues 1-331 (SEQ ID NO: 26), Reston ebolavirus (Pennsylvania) NP amino acid residues 1-739 (SEQ ID NO: 28), Reston ebolavirus (Pennsylvania) GP-mucin amino acid residues 314-466 (SEQ ID NO: 30), Sudan ebolavirus (Boniface) VP40 amino acid residues 1-326 SEQ ID NO: 8), Sudan ebolavirus (Boniface) NP amino acid residues 1-738 (SEQ ID NO: 10), Sudan ebolavirus (Boniface) GP-mucin amino acid residues 313-465 (SEQ ID NO: 12), Zaire ebolavirus (Mayinga) VP40 amino acid residues 1-326 (SEQ ID NO: 2), Zaire ebolavirus (Mayinga) NP amino acid residues 1-739 (SEQ ID NO: 4), Zaire ebolavirus (Kikwit) GP-mucin amino acid residues 313-465 (SEQ ID NO: 6), Marburg marburgvirus (Musoke) VP40 amino acid residues 1-303 (SEQ ID NO: 32), Marburg marburgvirus (Musoke) NP amino acid residues 1-695 (SEQ ID NO: 34), and Marburg marburgvirus (Musoke) GP-mucin amino acid residues 289-505 (SEQ ID NO: 36).

Flavivirus sequences that may be used in the system and methods described herein include, but are not limited to, antigenic fragments and/or variants of the following sequences: ZIKV (str.SPH2015) E amino acid residues 291-744 (SEQ ID NO:37), ZIKV (str.SPH2015) NS1 amino acid residues 796-1148 (SEQ ID NO: 40), ZIKV_AS (str.YAP) E amino acid residues 291-744 (SEQ ID NO: 43), ZIKV_AS (str.YAP) E-DIII amino acid residues 591-693 (SEQ ID NO: 46), ZIKV_AS (str.YAP) pM amino acid residues 126-290 (SEQ ID NO: 49), ZIKV_AS (str.YAP) NS1 amino acid residues 796-1148 (SEQ ID NO: 52), ZIKV (str. ArD 41519) E amino acid residues 291-744 (SEQ ID NO: 55), ZIKV (str. ARB7701) E amino acid residues 291-744 (SEQ ID NO: 58), ZIKV_AFR (str.MR-766) E amino acid residues 291-740 (SEQ ID NO: 61), ZIKV_AFR (str.MR-766) E-DIII amino acid residues 587-689 (SEQ ID NO: 64), ZIKV_AFR (str.MR-766) pM amino acid residues 126-290 (SEQ ID NO: 67), ZIKV_AFR (str.MR-766) NS1 amino acid residues 792-1144 (SEQ ID NO: 70), DENV1 E amino acid residues 281-722 (SEQ ID NO: 73), DENV1 E-DIII amino acid residues 579-677 (SEQ ID NO: 76), DENV1 M amino acid residues 206-279 (SEQ ID NO: 79), DENV1 NS1 amino acid residues 776-1126 (SEQ ID NO: 82), DENV1 NS3 amino acid residues 1476-2093 (SEQ ID NO: 85), DENV2 E amino acid residues 281-722 (SEQ ID NO: 88), DENV2 E-DIII amino acid residues 575-675 (SEQ ID NO: 91), DENV2 M amino acid residues 206-279 (SEQ ID NO: 94), DENV2 NS1 amino acid residues 776-1127 (SEQ ID NO: 97), DENV2 NS3 amino acid residues 1476-2092 (SEQ ID NO: 100), DENV3 E amino acid residues 281-722 (SEQ ID NO: 103), DENV 3E-DIII amino acid residues 574-672 (SEQ ID NO: 106), DENV3 M amino acid residues 206-279 (SEQ ID NO: 109), DENV3 NS1 amino acid residues 774-1125 (SEQ ID NO: 112), DENV3 NS3 amino acid residues 1474-2091 (SEQ ID NO: 115), DENV4 E amino acid residues 280-721 (SEQ ID NO: 118), DENV4 E-DIII amino acid residues 574-676 (SEQ ID NO: 121), DENV4 M amino acid residues 205-278 (SEQ ID NO: 124), DENV4 NS1 amino acid residues 775-1125 (SEQ ID NO: 127), DENV4 NS3 amino acid residues 1474-2091 (SEQ ID NO: 130), WNV E amino acid residues 291-741 (SEQ ID NO: 133), WNV E-DIII amino acid residues 586-705 (SEQ ID NO: 136), WNV pM amino acid residues 124-290 (SEQ ID NO: 139), WNV NS1 amino acid residues 792-1143 (SEQ ID NO: 142), YFV E amino acid residues 286-728 (SEQ ID NO: 145), YFV E-DIII amino acid residues 573-683 (SEQ ID NO: 148), YFV pM amino acid residues 122-285 (SEQ ID NO: 151), YFV NS1 amino acid residues 779-1130 (SEQ ID NO: 154), YFV NS3 amino acid residues 1485-2107 (SEQ ID NO: 157), JEV E amino acid residues 299-744 (SEQ ID NO: 160), JEV E-DIII amino acid residues 586-696 (SEQ ID NO: 163), JEV pM amino acid residues 128-294 (SEQ ID NO: 166), JEV NS1 amino acid residues 795-1146 (SEQ ID NO: 169), SLEV E amino acid residues 289-739 (SEQ ID NO: 172), SLEV E-DIII amino acid residues 587-692 (SEQ ID NO: 175), SLEV pM amino acid residues 122-288 (SEQ ID NO: 178), SLEV NS1 amino acid residues 790-1145 (SEQ ID NO: 181), MVEV E amino acid residues 293-743 (SEQ ID NO: 184), MVEV E-DIII amino acid residues 590-698 (SEQ ID NO: 187), MVEV M amino acid residues 218-292 (SEQ ID NO: 190), MVEV NS1 amino acid residues 794-1145 (SEQ ID NO: 193), ROCV E amino acid residue 286-736 (SEQ ID NO: 196), ROCV E-DIII amino acid residues 583-691 (SEQ ID NO: 199), ROCV pM amino acid residues 119-285 (SEQ ID NO: 202), ROCV NS1 amino acid residues 788-1142 (SEQ ID NO: 205), ROCV NS3 amino acid residues 1498-2116 (SEQ ID NO: 208), POWV E amino acid residues 279-725 (SEQ ID NO: 211), POWV E-DIII amino acid residues 578-673 (SEQ ID NO: 214), POWV pM amino acid residues 111-278 (SEQ ID NO: 217), POWV NS1 amino acid residues 776-1128 (SEQ ID NO: 220), TBEV_E (str. SOFJIN-HO) E amino acid residues 281-726 (SEQ ID NO: 223), TBEV (str. SOFJIN-HO) E-DIII amino acid residues 580-675 (SEQ ID NO: 226), TBEV (str. SOFJIN-HO) pM amino acid residues 113-280 (SEQ ID NO: 229), TBEV (str. SOFJIN-HO) NS1 amino acid residues 777-1128 (SEQ ID NO: 232), TBEV_EUR (str.NEUDOERFL) E amino acid residues 281-726 (SEQ ID NO: 235), TBE (str.NEUDOERFL) E-DIII amino acid residues 580-675 (SEQ ID NO: 238), TBE (str.NEUDOERFL) pM amino acid residues 113-280 (SEQ ID NO: 241), and TBE (str.NEUDOERFL) NS1 amino acid residues 777-1128 (SEQ ID NO: 244).

Coronavirus sequences that may be used in the system and methods described herein include, but are not limited to, antigenic fragments and/or variants of the following sequences: SARSS (SEQ ID NO: 248), NL63N (SEQ ID NO: 250), SARS2N (SEQ ID NO: 252), 229EN (SEQ ID NO: 254), SARS2S (SEQ ID NO: 256), HKU1N (SEQ ID NO: 258), OC43N (SEQ ID NO: 260), SARSN (SEQ ID NO: 262), 229ES (SEQ ID NO: 264), NL63S (SEQ ID NO: 266), HKU1S (SEQ ID NO: 268), OC43S (SEQ ID NO: 270), MERSS (SEQ ID NO: 272), MERSN (SEQ ID NO: 274), UKS (SEQ ID NO: 276), SAS (SEQ ID NO: 278), BRS (SEQ ID NO: 280), BRbd (SEQ ID NO: 282), WRSS, (SEQ ID NO: 284), UKSbd (SEQ ID NO: 286), SASbd (SEQ ID NO: 288), IDS B.1.617.1 (SEQ ID NO: 290), and IDSbd B.1.617.1 (SEQ ID NO: 292).

TABLE 3
Recombinant Coronavirus Spike and N antigens
Coronavirus polypeptides expressed in bacteria
Spike RBD protein

| | | | Amino acid | Predicted M.W. |
|---|---|---|---|---|
| | Coronavirus Name | Strains | positions | (with tag) (kD) |
| 1 | SARS-CoV-2 | SARS-2 isolate Wuhan-Hu-1 | 311-550 | 29 |
| 2 | SARS-CoV | SARS coronavirus Urbani | 298-536 | 29 |
| 3 | MERS-CoV | MERS isolate Al-Hasa_1_2013 | 359-615 | 30 |
| 4 | HCoV-OC43 | OC43 strain ATCC VR-759 | 312-636 | 38 |
| 5 | HCov-HKU1 | HKU1 isolate Caen1 | 302-677 | 39 |
| 6 | HCoV-NL63 | NL63 | 456-625 | 23 |
| 7 | HCoV-229E | 229E strain ATCC VR-740 | 274-458 | 23 |

SARS-CoV-2 variant Spike S1 and RBD

| | | | Amino acid | Predicted M.W. |
|---|---|---|---|---|
| | Coronavirus Protein | Variant strain sequence | positions | (kD) |
| 1 | SARS-CoV-2 WRSS | Wuhan reference S1 | 1-531 | 61 |
| 2 | SARS-CoV-2 UKS | B.1.1.7 S1 | 1-531 | 61 |
| 3 | SARS-CoV-2 UKSbd | B.1.1.7 S-RBD | 311-531 | 25 |
| 4 | SARS-CoV-2 SAS | B.1.351/501Y.V2 S1 | 1-531 | 61 |
| 5 | SARS-CoV-2 SASbd | B.1.351/501Y.V2 S-RBD | 311-531 | 25 |
| 6 | SARS-CoV-2 BRS | P.1 S1 | 1-531 | 61 |
| 7 | SARS-CoV-2 BRbd | P.1 S-RBD | 311-531 | 25 |
| 8 | SARS-CoV-2 IDS | B.1.617.1 S1 | 1-532 | 61 |
| 9 | SARS-CoV-2 IDbd | B.1.617.1 S-RBD | 312-532 | 25 |

| | | | |
|---|---|---|---|
| | | Amino acid | Predicted M.W. |
| Coronavirus Name | Strains | positions | (with tag) (kD) |

Nucleoprotein

| | | | | |
|---|---|---|---|---|
| 1 | SARS-CoV-2 | SARS-2 isolate Wuhan-Hu-1 | 1-419 | 47 |
| 2 | SARS-CoV | SARS coronavirus Urbani | 1-422 | 47 |
| 3 | MERS-CoV | MERS isolate Al-Hasa_1_2013 | 1-413 | 46 |
| 4 | HCoV-OC43 | OC43 strain ATCC VR-759 | 1-448 | 51 |
| 5 | HCov-HKU1 | HKU1 isolate Caen1 | 1-441 | 51 |
| 6 | HCoV-NL63 | NL63 | 1-377 | 44 |
| 7 | HCoV-229E | 229E strain ATCC VR-740 | 1-389 | 45 |

Coronavirus polypeptides expressed in mammalian cells
Spike RBD protein

| | | | | |
|---|---|---|---|---|
| 1 | SARS-CoV-2 | SARS-2 isolate Wuhan-Hu-1 | 311-550 | 29 |
| 2 | SARS-CoV | SARS coronavirus Urbani | 298-536 | 29 |
| 3 | MERS-CoV | MERS isolate Al-Hasa_1_2013 | 359-615 | 30 |
| 4 | HCoV-OC43 | OC43 strain ATCC VR-759 | 312-636 | 38 |
| 5 | HCov-HKU1 | HKU1 isolate Caen1 | 302-677 | 39 |
| 6 | HCoV-NL63 | NL63 | 456-625 | 23 |
| 7 | HCoV-229E | 229E strain ATCC VR-740 | 274-458 | 23 |

Nucleoprotein

| | | | | |
|---|---|---|---|---|
| 1 | SARS-CoV-2 | SARS-2 isolate Wuhan-Hu-1 | 1-419 | 47 |
| 2 | SARS-CoV | SARS coronavirus Urbani | 1-422 | 47 |
| 3 | MERS-CoV | MERS isolate Al-Hasa_1_2013 | 1-413 | 46 |
| 4 | HCoV-OC43 | OC43 strain ATCC VR-759 | 1-448 | 51 |
| 5 | HCov-HKU1 | HKU1 isolate Caen1 | 1-441 | 51 |
| 6 | HCoV-NL63 | NL63 | 1-377 | 44 |
| 7 | HCoV-229E | 229E strain ATCC VR-740 | 1-389 | 45 |

Although the invention has been described in some detail by way of illustration and example for purposes of clarity of understanding, it should be understood that certain changes and modifications may be practiced within the scope of the appended claims. Modifications of the above-described modes for carrying out the invention that would be understood in view of the foregoing disclosure or made apparent with routine practice or implementation of the invention to persons of skill in virology, physiology, immunology, and/or related fields are intended to be within the scope of the following claims.

All publications (e.g., Non-Patent Literature), patents, patent application publications, and patent applications mentioned in this specification are indicative of the level of skill of those skilled in the art to which this invention pertains. All such publications (e.g., Non-Patent Literature), patents, patent application publications, and patent applications are herein incorporated by reference to the same extent as if each individual publication, patent, patent application publication, or patent application was specifically and individually indicated to be incorporated by reference.

### EXAMPLES

### Example 1

### Plasma Separation

Plasma separation. PALL Vivid® plasma separation membrane was tested for one step plasma separation from whole blood. The highly asymmetric nature of the membrane allows the cellular components of the blood (red cells, white cells, and platelets) to be captured in the larger pores without lysis, while the plasma flows down into the smaller pores on the downstream side of the membrane. In order to test the plasma separation efficiency, a 3D printed device was designed and printed (FIGS. 1A-1C).

A 9 mm circle was cut and incorporated in the device. A 3D printed crimping ring was included for support. This is to ensure the membrane is crimped and that no blood, leaks around the edges of the membrane. When the filter was filled with blood, the separator allowed plasma to drain to the bottom of the device into a collection trench. The separation was also mediated by capillary action. FIG. 1A shows the top view of the device and the location of the plasma separation membrane filled with blood. FIG. 1B shows the bottom view and the separated plasma in the collection chamber. FIG. 1C shows the array chamber filled with buffer mixed with the plasma. The buffer was injected from the top chamber and it mixed with plasma and delivered to the array chamber through the micro channels. Saturating the membrane completely with 50-60 μl blood yielded the best results, achieving a consistent 4-6 μl volume of plasma separation in 2-3 minutes. The inventors surprisingly discovered that the separated plasma volume (4-6 μl) obtained in these studies is sufficient to complete all assay requirements. This is in contrast to conventional methods that require larger (over 1 mL volume) of sample for a single assay.

### Example 2

### Device Configuration

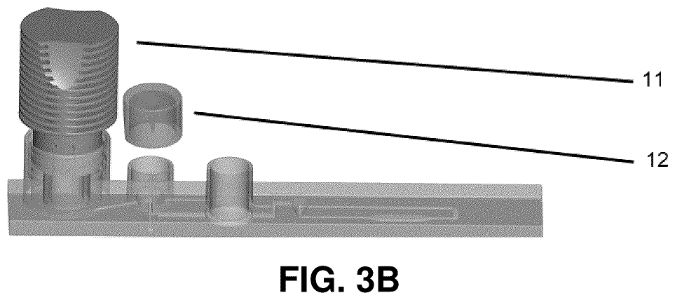

Another 3D printed device was used to test the incorporation of the lyophilized bead of anti-human IgG in the cassette (FIGS. 3A-3B). The device has a conical chamber to house the lyophilized bead in addition to the buffer chamber, plasma separation membrane, and a waste chamber. FIGS. 3A and 3B show, respectively, the top and bottom views of the device. Blood was added to the membrane and after the separation of plasma, a volume of 150 μl buffer was injected. The buffer injection diluted the plasma, dissolved the lyophilized bead, mixed them and delivered to the array chamber successfully. This demonstrates that a very small amount of sample is sufficient to cover the entire assay array area.

### Example 3

### Beads

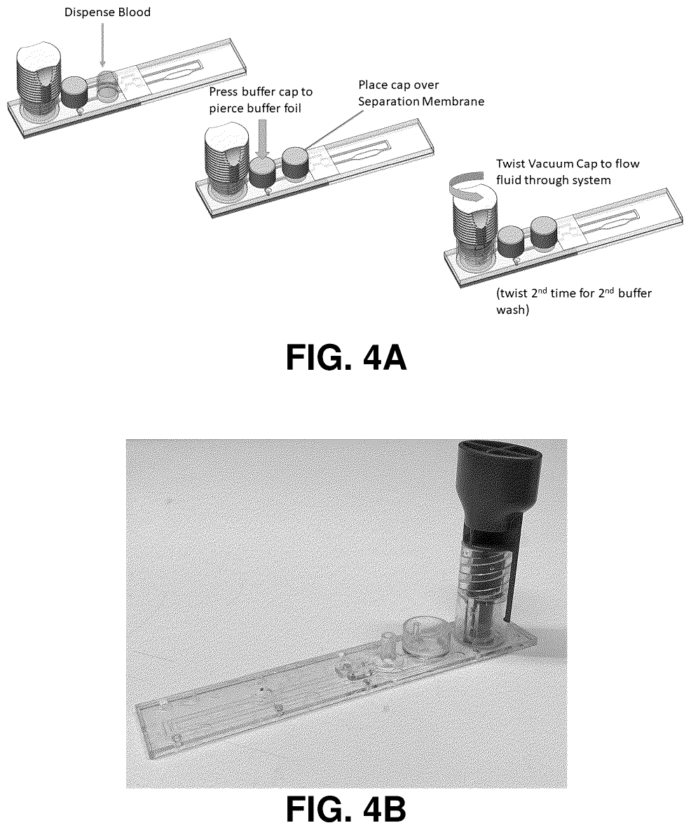

Based on the component testing, prototype units of a semi-integrated cassette with form factor for the final design with on-unit plasma separation were printed with an Object 3D printer and 11122 XC Watershed material. In this design, shown in FIG. 4, the assembly and assay were performed manually. The plasma separation membrane was placed into the well and a compression ring pressed firmly to crimp the membrane to prevent seeping around the edges of the filter. The backside of the cassette has a pressure sensitive adhesive (PSA) backing and after removing it the micro array printed cyclic olefin copolymer (COC) slide was pressed firmly to the exposed adhesive ensuring the correct orientation of the array. The lyophilized bead was placed into the Lyo well and covered with a top PSA. Blood or plasma is applied to the separation chamber, and a volume of 150 μl buffer was delivered by pipette 2 minutes later to mix the plasma with the lyophilized bead. A second buffer wash was used to remove any unbound reagents from the array. The bound antibody was measured using a laser-based confocal fluorescent reader to confirm performance specifications.

The integrated, sealed disposable cassette will enable onboard storage of secondary reagent and a reactive chamber that allows interaction of antibody from plasma with immobilized antigens. The secondary reagent used for this example was anti-human IgG conjugated to Alexa Fluor 488. The assay flow was designed so that the separated plasma will be diluted with buffer first, followed by mixing with the secondary reagent. As an alternative to lyophilization directly in the cassette, the secondary reagent in the form a lyophilized bead was incorporated into the cassette for reconstitution at the initiation of the assay. The bead-format has the best ratio of volume to surface area facilitating fast reconstitution with buffer.

The beads were prepared with a nominal size of about 3 mm in diameter. Trehalose and lactose (10%) were used as excipient stabilizers. A concentration of 2 and 3 μg/ml of the secondary reagent was encapsulated on the bead. The morphology of the bead was spherical and fluorescent imaging was used to study visualization. Leica TCS SP5 broadband confocal microscope was used for producing the fluorescent image with 5× magnification. A single bead was placed on a well of microtiter plate and 150 μl phosphate buffered saline (PBS) was added to the well to measure reagent resuspension. Images taken at every 15 seconds showed that the bead was completely dissolved within 60 seconds (data shown). It is important to note that no mixing was done during this process, whereas the cassette design includes a mixing routine that causes the bead to dissolve in <60 seconds. Negative fluid pressure produced the least amount of air bubbles that could potentially interfere with imaging of results if these move into the microarray chamber (data not shown).

### Example 4

### Covid-19 Peptide Characterization and Production

The multiplexed test can assess infections from the seven described HCoV. The HCoV 229E, NL63, OC43, and HKU1 are globally endemic, and account for up to 30% of upper respiratory tract infections in adults. While infections from the most common HCoV usually result in mild symptoms, the recently emerged SARS (SARS-CoV-1), MERS, and the current SARS-CoV-2/nCoV that causes COVID-19 are associated with higher morbidity and deaths. The hCoV nucleocapsid (N) and spike (S) proteins were selected for test antigens based on high abundance during viral replication and previous immunogenicity data for the nearest-neighbor virus obtained during the SARS outbreak. Trivedi et al. Sci Rep. (2019) 9(1): 1390; Woo et al. J Clin Microbiol (2005) 43(7): 3054-8.

The N protein is more conserved among HCoV and may provide a higher degree of assay sensitivity than S, whereas the S protein, which is more variable and controls receptor interactions, may provide greater specificity than N. Tables 2, 3, and 4 contain the sequences for the coronavirus antigens.

DNA fragments encoding the S glycoprotein receptor binding domain (RBD) and the full-length N protein from the seven selected coronaviruses (Table 4) were synthesized using codons optimized for bacterial expression and cloned into a pET28(+) bacterial expression plasmid in frame and downstream of six histidine residues.

TABLE 4
Source of test antigens for Coronvavirus

| | Coronavirus Name | | Genus | Disease | HCoV strain/Accession |
|---|---|---|---|---|---|
| 1 | Severe acute respiratory | SARS-CoV-2 | beta | COVID-19 | Wuhan-HU-1, NC_045512 |
| | syndrome coronavirus 2 | | | | |
| 2 | Severe acute respiratory | SARS-CoV-1 | beta | SARS | Urbani strain, AAP13441; |
| | syndrome coronavirus 1 | | | | SARS GZ02, AY 390556) |
| 3 | Middle East respiratory | MERS-CoV | beta | MERS | Al-Hasa_1_2013 strain |
| | syndrome coronavirus | | | | AGN70962 |
| 4 | Human coronavirus OC43 | HCoV-OC43 | beta | L/URTI* | OC43/human/USA/008- |
| | | | | | 5/2000, KF530092.1 |
| 5 | Human coronavirus HKU1 | HCov-HKU1 | beta | L/URTI, GI** | HKU1 isolate SI17244, |
| | | | | | MH940245.1 |
| 6 | Human coronavirus NL63 | HCoV-NL63 | alpha | Croup, | NL63/human/USA/904- |
| | | | | L/URTI, GI | 20/1990, KF530104.1 |
| 7 | Human coronavirus 229E | HCoV-229E | alpha | L/URTI | NC_002645 |

*Lower and upper respiratory tract infections
**Gastrointestinal tract infections

The pET28(+) plasmids were transformed into BL21(DE3) E coli competent cells, which were then grown in Luria-Bertani medium (supplemented with 100 μg/mL ampicillin) at 37° C. under vigorous shaking until optical density (600 nm) reaches approximately 0.6.

Recombinant protein expression was induced by IPTG induction (0.5 mM) at 16° C. overnight. Cells were harvested by centrifugation (5000×g, 10 min, 4° C.) and lysed with B-PER Bacterial Protein Extraction Reagent. The His-tagged recombinant S proteins (SSARS-2, SSARS, SMERS, SOC43, SHKU1, SNL63, S229E), N proteins (NSARS-2, NSARS, NMERS, NOC43, NHKU1, NNL63, N229E) were purified from clarified bacterial lysate with immobilized metal affinity chromatography using a Bio-Rad NGC FPLC system. Table 2 lists the sequences used for the coronavirus antigens.

Purified recombinant proteins were analyzed by SDS-PAGE and Western blots to confirm purity and identity. Anti-CoV antibodies can also be used to assess the quality of the antigens produced. Insoluble proteins can be denatured in 6M urea and refolded on a Ni-NTA column by buffer exchange to obtain recombinant proteins that remain soluble for the microarray printing that is performed during the manufacturing process for assay cassettes. Mammalian cell products were also produced. Each of the fourteen (14) plasmids encoding for expressing CoV recombinant spike proteins were transiently transfected into HEK293 cultures for soluble expression of the recombinant proteins.

The proteins were isolated by using nickel column chromatography. The purified protein products were buffer exchanged into phosphate buffered saline, pH 7.4. The final purified proteins were evaluated for protein content by a bicinchonic acid (BCA) assay. Purity was assessed by Coomassie Blue binding by proteins separated by SDS-PAGE, and identity was evaluated by Western Blot analysis using antibodies that target the HIS tag. Anti-CoV antibodies can also be used to assess the quality of the antigens produced. The recombinant N and S proteins can be used in the reaction chamber of a system and method described herein. Further, antigenic fragments from the N and S proteins may also be used.

Spike S-1 and Spike-RBD proteins were synthesized for the following SARS-CoV-2 variants:
(1) The phylogenetic cluster named lineage B.1.1.7 (UK, now called alpha), comprising deletion of residues 69-70 (H,V), and mutation N501Y
(2) B.1.351 (also known as 501Y.V2; now called beta) South Africa, comprising mutations D614G, D80A, D215G, E484K, N501Y, A701V, L18F, R246I and K417N
(3) B.1.617.2 (delta), comprising mutations T19R, (V70F*), T95I, G142D, E156-, F157-, R158G, (A222V*), (W258L*), (K417N*), L452R, T478K, D614G, P681R, D950N
(4) P.1 (Brazil; now called gamma), comprising mutations L18F, T20N, P26S, D138Y, R190S, K417T, E484K, N501Y
   The synthetic gene sequences were cloned into pET-28a(+) vector with an N-terminal His•Tag®/thrombin/T7•Tag® plus an optional C-terminal His•Tag sequence, using NdeI_XhoI cloning site. The first Met codon (M) was deleted to allow N-term His-Tag fusion and a stop codon* was added to eliminate C-term His-Tag.

ACE2 and virus-neutralizing.

### Example 5

### Production of Ebola Virus Disease Peptides

The mammalian host cells HEK293 (293-F, Invitrogen) were cultured and transfected in suspension in serum-free FreeStyle 293 medium (Gibco). Cells were maintained and expanded in vented Erlenmeyer shake flasks (Corning) at 37° C. and 8% CO₂ in a shaking incubator. The mammalian expression vectors containing filoviral protein sequences were transfected into HEK293 cells using 293fectin (Invitrogen). Mock transfections with the pV1JNS vector (CHIKV37997 cassette omitted) will be utilized as negative controls for protein analysis methods. Proteins will be purified from the supernatant. The supernatant may be concentrated and mixed with Ni Sepharose 6 Fast Flow beads (GE Life Sciences) overnight at 4° C. The next day, the beads may be separated by centrifugation and packed into a Bio-Rad Econo column. The column may be washed with PBS, 20% glycerol, 0.2% Tween 20, and 10 mM imidazole. Protein will be eluted with the same buffer containing 500 mM imidazole and dialyzed into PBS containing 10% glycerol, arginine, and glutamic acid. All proteins will be analyzed by SDS-PAGE and western blot for purity, and protein concentrations were determined using the bicinchoninic acid (BCA) assay. Proteins will be aliquoted and stored at −80° C. for long-term storage. Control antigens will be purchased from vendors, and will include human IgG, influenza HA proteins, and bovine serum albumin.

TABLE 5
Ebola Disease Virus Species and Proteins

| | | Amino Acid | GenBank |
|---|---|---|---|
| Species | Gene | Residues | Sequence |
| Bundibugyo | NP | 1-739 | FJ217161.1 |
| ebolavirus | GP-mucin | 313-465 | FJ217161.1 |
| Tai Forest | NP | 1-739 | FJ217162.1 |
| ebolavirus | GP-mucin | 313-465 | FJ217162.1 |
| Reston ebolavirus | NP | 1-739 | AF522874.1 |
| (Pennsylvania) | GP-mucin | 314-466 | AY769362. |
| Sudan ebolavirus | NP | 1-738 | AF173836.1 |
| (Boniface) | GP-mucin | 313-465 | FJ968794.1 |
| Zaire ebolavirus | NP | 1-739 | AF086833.2 |
| (Mayinga) | GP-mucin | 313-465 | JQ352763.1 |
| Marburg marburgvirus | NP | 1-695 | DQ217792.1 |
| (Musoke) | GP-mucin | 289-505 | DQ217792.1 |

Sudan ebolavirus (SUDV) NP Production

SUDV NP was purified from inclusion bodies from E. coli cell lysate. The inclusion bodies were washed, denatured in 6 M guanidine hydrochloride, and loaded onto a HisTrap HP column. The column was washed with 5 column volumes of buffer followed by five column volumes of 6 M Urea in 25 mM HEPES, 0.2M sodium chloride, pH 7.5. The protein was refolded on the column by reducing the urea concentration from 6 M to 0 M in 25 mM HEPES, 0.2M sodium chloride, pH 7.5 over 30 column volumes and eluted with an imidazole gradient. A significant amount of the NP was present in the flow through.

Subsequently the SUDV NP in the column flow through was added to the nickel resin column and packed. The column was washed with 5 column volumes of buffer followed by five column volumes of 6 M Urea in 25 mM HEPES, 0.2 M sodium chloride, pH 7.5. The protein was refolded on the column by reducing the urea concentration from 6 M to 0 M in 25 mM HEPES, 0.2 M sodium chloride, pH 7.5 over 30 column volumes. The SUDV NP bound to the column was eluted with an imidazole step elution. The recovery of SUDV NP was 1.79 mg from the flow through portion). Zaire ebolavirus (EBOV) NP Production

EBOV NP was isolated from the E. coli cells as an inclusion body. The cells were lysed by BPER solution and centrifuged. The inclusion body in the pellet was washed and then denatured in 6 M guanidine hydrochloride and loaded onto a HisTrap HP column. The column was washed with 5 column volumes of the same buffer followed by five column volumes of 6 M Urea in 25 mM HEPES, 0.2 M sodium chloride, pH 7.5. The protein was refolded on the column by reducing the urea concentration from 6 M to 0 M in 25 mM HEPES, 0.2M sodium chloride, pH7.5 over 30 column volumes. The EBOV NP bound to the column was eluted with an imidazole gradient. The initial concentration of NP was 0.244 mg/mL & 0.27 mg/mL from the two fractions with an estimated amount of 10.2 mg. Fractions were pooled and concentrated resulting in approximately 5.2 mg of EBOV NP purified by this method. Recovery was about 50% after concentration.

Bundibugyo ebolavirus (BDBV) NP Production

BDBV NP was similarly isolated from the E. coli cells as an inclusion body as previously described for SUDV and EBOV NP purification method. The BDBV NP bound to the column was eluted with an imidazole gradient. Approximately 5.2 mg at 0.396 mg/mL of BDBV NP was purified.

Taï Forest ebolavirus (TAFV) NP and Reston ebolavirus (RESTV) NP Production

TAFV NP was similarly isolated from the E. coli cells as an inclusion body as previously described other filovirus NP proteins. The TAFV NP bound to the column was eluted with an imidazole gradient. Approximately 6.6 mg of TAFV NP was purified. Similar methods were also used to produce RESTV NP. A total of 3.9 mg RSETV NP was obtained.

In summary, all twelve filovirus proteins GP (6) and six NP (6) proteins to be incorporated into the assay were successfully expressed, purified and concentrated. The surface chemistry to attach the proteins to COC surface and lyophilized bead containing the secondary reagent (anti-Human IgG-Alexa Flour 488) were previously developed.

### Surface Immobilization of Proteins to Assay Cassette Reaction Chamber.

A published chemical modification method (Ref: Raj et al. 2009) may be used to immbolize proteins on COC surfaces. Briefly, COC surfaces are treated with plasma that produces functional groups which facilitates attachment of silane reagent aminipropyl triethoxysilane (APTES) containing amine groups. Proteins that contain carboxyl groups are conjugated to APTES using a cross-linking agent 1,4-Phenlyene diisothiocyanate (PDITC).

### Example 6

### Yellow Fever Virus Peptides

### Assay Protein and Virus Probes

Recombinant E (transmembrane domain deletion), full-length NS1 and pM protein antigens from Yellow Fever Virus (YFV), and similar antigens from 14 other flaviviruses were expressed in E. coli BL21 (DE3) and isolated (Table 1). The NS1 and E proteins were 6×-His tagged (N-terminus), and pM were 6×-His-MBP (N-terminus) fusion proteins. Inclusion body pellets were isolated, solubilized in 1% SDS, and the recombinant proteins were purified by immobilized metal affinity chromatography (IMAC) and FPLC (NGCTM, Bio-Rad). For the production of vaccine strain of YFV, Vero cells (CCL-81™, ATCC) were infected with 17D virus (ATCC #NR-116) and harvested six days after infection. Supernatants containing the virus were filtered, precipitated with polyethylene glycol (PEG 8000, Promega), and resuspended in media.

TABLE 6
Antigens included in the protein microarray to examine serological
immune responses to yellow fever vaccination

| | | | | Antigens^{1, 2} | | | | |
|---|---|---|---|---|---|---|---|---|
| Virus | Isolate | Country | Year | Virus | E | E-DIII | pM/M | NS1 |
| Zika | SV0127/14 | Thailand | 2014 | x | | | | |
| Asian lineage | CPC-0740 | Philippines | 2012 | x | | | | |
| | VABC59 | Puerto Rico | 2015 | x | | | | |
| | SPH2015 | Brazil | 2015 | | x | | | |
| | YAP | Micronesia | 2007 | | x | x | x | x |
| Zika | MR-766 | Uganda | 1947 | x | x | x | x | x |
| African lineage | IBH30656 | Nigeria | 1968 | x | | | | |
| | DAKAR41525 | Senegal | 1984 | x | | | | |
| | DAKAR 41662 | Senegal | 1984 | x | | | | |
| | ARB7701 | Central Africa | 1976 | | x | | | |
| | ArD_41519 | Senegal | 1984 | | x | | | |
| Dengue 1 | HAWAII | USA | 1944 | x | x | x | x | x |
| Dengue 2 | NGC | New Guinea | 1944 | x | x | x | x | x |
| Dengue 3 | H87 | Philippines | 1956 | x | x | x | x | x |
| Dengue 4 | H241 | Philippines | 1956 | x | x | x | x | x |
| West Nile | NY99 | USA | 1999 | | x | x | x | x |
| Yellow fever | 17-D-204 | USA | 1985 | x | x | x | x | x |
| Japanese encephalitis | SA14-14-2 | South Korea | 2006 | | x | x | x | x |
| St. Louis encephalitis | PARTON | USA | 1933 | | x | x | x | x |
| Murray Valley encephalitis | 1-51 | Australia | 1952 | | x | x | x | x |
| Rocio | SPH34675 | Brazil | 1975 | | x | x | x | x |
| Powassan | LB | Canada | 1958 | | x | x | x | x |
| Tick borne encephalitis-E | SOFJIN-HO | Russia | 1937 | | x | x | x | x |
| Tick borne encephalitis - | NEUDOERFL | Austria | 1971 | | x | x | x | x |
| EUR | | | | | | | | |

Control Antigens
Yellow fever virus 17D
IgGs: human, NHP, rabbit, goat, and mouse
IgMs: human, NHP, and rabbit
Anti-human IgG
Influenza hemagglutinin (HA) proteins: Brisbane, California, and Perth strains
E. coli HisMBP
Bovine serum albumin (BSA)
Human cytomegalovirus glycoprotein B (gB)
HEPES print buffer
¹The “x” indicates antigens included and unfilled squares are antigens that were not included.
²Antigen abbreviations: Transmembrane domain truncated envelope protein (E), precursor membrane or membrane protein (pM/M), and non-structural protein 1 (NS1).

### 17D Vaccination Sera and Reagent Antibodies.

Early-immune yellow fever virus antisera from three NHPs (BEI Resources, Manassas, VA), immunized by subcutaneous injection of 0.5 mL of live, attenuated YFV vaccine (strain 17D), were collected 30 days after vaccination. Late-Immune Yellow Fever Virus antisera from the same NHP cohort (BEI Resources), consisted of pooled time-point sera that were collected in approximate 30-day time intervals ranging from 120 to 420 days after vaccination for each animal. Human sera from subjects vaccinated with 17D were obtained from the U.S. Department of Defense Serum Repository (Silver Springs, Maryland). Goat anti-mouse IgG was obtained from Life Technologies, Inc. (Carlsbad, CA). Goat anti-human γ-specific IgG (1:1000) Alexa Fluor 647-conjugated secondary antibody (Southern Biotech) diluted in probe buffer. Mouse anti-sera raised against YFV was kindly provided by Robert Tesh (University of Texas Medical Branch, Galveston, TX), and reference sera from non-human primates (NHP; rhesus macaques) that were vaccinated with YFV strain 17D were obtained from BEI Resources (Manassas, VA).

### Microarray Assembly

Concentrated YFV vaccine strain 17D preparations and recombinant proteins were diluted in microarray printing buffer (50 mM HEPES, 140 mM NaCl, 2 mM DTT, pH 7.3) in 2-fold serial dilutions (1:2-1:32) and concentrations that ranged from 100-1000 μg/mL, respectively, in order to determine optimal binding signal. Two microarray assays were designed. In the first, optimal densities of 17D virus preparations, control antigens, and recombinant proteins from YFV and DENV1-4, were printed for an YFV-focused microarray, while the second microarray included E, pM and NS1 from all 24 species and serotypes of flaviviruses examined. Prepared dilutions of YFV antigens, along with controls, were printed in replicates (n=6) on nitrocellulose-coated (4 or 16 pad) microarray surfaces (ONCYTE® SuperNOVA, Grace Bio-labs, Inc.) using non-contact microarray printers (Scienion sciFLEXARRAYER SX, Berlin, Germany; ArrayJet, Glasgow, UK). Control antigens included IgGs (monkey, human, rabbit, goat, and mouse), IgMs (human, monkey, and rabbit), HisMBP, bovine serum albumin (BSA), three hemagglutinin proteins (HA) from three strains of seasonal influenza, serial dilutions of anti-human IgG, human cytomegalovirus glycoprotein B (CMV-gB), HisMBP-tagged recombinant Jamestown Canyon Virus nucleocapsid (N) protein, and buffer control spots. Table 2 lists the YFV peptide sequences that are preferred for antibody assays and Table 1 lists the preferred antibodies for antigen capture assays.

Proteins were diluted to optimal concentrations in microarray printing buffer with glycerol added to a final concentration of 40%, for final printing. The spotting quality and density was evaluated by protein imaging (SYPRO®Ruby; ThermoFisher, Waltham, MA). The deposited YFV recombinant proteins were also examined by antibody detection of the N-terminal 6×His fusion tag on the microarray surface. The overall mean covariance for all printed probes on the microarray was determined to be ≤14% between all replicates across each of the 16 printed microarrays.

### Protein Microarray Assays

All microarray processing steps were performed at 22° C., protected from light. NHP (1:50) and human (1:150) sera, diluted in probe buffer (1×PBS pH 7.4, 0.1% Tween 20, 1% BSA), were pre-cleared by incubating (1 mg/ml) with E. coli lysate (Promega) with gentle agitation, followed by centrifugation (17,000×g, 5 min) to remove the pelleted immunoprecipitates. Microarrays were blocked with Super G blocking buffer (Grace Bio-labs) for 1.5 h and washed 3 times (5 min each) in wash buffer (1×PBS, 0.2% Tween® 20, 1% BSA). The microarrays were incubated (2 h) with E. coli-cleared serum, washed (5×, 5 min each), and incubated for 1 h with goat anti-human γ-specific IgG (1:1000) diluted in probe buffer for detecting human antibodies or other species specific secondary antibodies to detect binding of primary antibodies for other species. Microarrays were washed 3 times with wash buffer (5 min each) followed by two washes in filtered deionized water to remove any residual salts, and then dried. The microarrays were scanned at 635 nm using a confocal laser scanner (GenePix® 4400A scanner; Molecular Devices) using settings below signal saturation. Antibody binding results were analyzed with GenePix® Pro 7 software. Smith et al mSphere (2018) 3(2). Background-subtracted pixel counts were quantile normalized by using a preprocessCore package in R software (v3.3.3). Bolstad et al. Bioinformatics (2003) 19(2): 185-93. Outliers among data replicates, identified using a modified Z-score (median absolute deviation >3.5), were removed. Pixel counts from replicate spots were averaged to obtain mean fluorescence intensity (MFI) and used for subsequent analyses.

### Data Analysis

Graphs and statistical analyses including: student t-tests (two-tailed) with multiple comparisons, receiver operating characteristic (ROC) curves, linear regression, Pearson's correlation analysis, two-way analysis of variance (ANOVA) analyses with multiple comparison's corrected with Tukey's statistical hypothesis testing, and one-way analysis of variance (ANOVA) with uncorrected multiple comparisons using Kruskal-Wallis non-parametric test were performed using GraphPad Prism v8.3.1. Percent signal change for analysis of cross-reactive antibody responses was calculated as previously described (DENV E ref), where y is the MFI originating from flavivirus proteins and j is the MFI of the infecting virus species (YFV). Hierarchical clustering analyses (average-linkage Euclidean distance (MFI data) or Pearson correlation (relative binding data)) were performed using MeV v4.8.1 within the TM4 software suite. Saeed et al. Biotechniques 2003. 34(2): 374-8.

### Yellow Fever Virus Neutralization Assays

To evaluate YFV-neutralizing antibodies, a previously described flow cytometry-based infectivity assay was used with modifications. de Alwis & de Silva Methods Mol Biol. (2014) 1138: 27-39. The length of infection and the amount of YFV used in neutralization assays were optimized. Vero cells were seeded 24 h prior to infection (38,500 per well) in 24 well plates. Sera were prepared in 4-fold serial dilutions (70 μL volume) in RPMI media containing 1% penicillin/streptomycin with final dilutions ranging from 1:10 to 1:196,830. An aliquot of day 6 harvested YFV was thawed in a 37° C. water bath and then diluted in infection media containing 1% penicillin/streptomycin for a targeted final concentration (1:200) that allowed for the ideal range of 7-30% infection of the cells. Pre-incubation reactions comprised of samples containing an equal volume (70 μL) of both diluted virus and human sera, as well as infection media containing no virus (Vero uninfected control), and diluted YFV alone were incubated (1 h, 37° C., 5% CO₂) in a 96-deep well PCR-clean/Lo-protein binding plate (Eppendorf). The pre-incubation reactions were diluted 1:2 with infection media (140 μL) containing penicillin-streptomycin (1%), 250 μL were added to Vero cells in 24-well plates and the cultures were incubated (1.5 h, 37° C., 5% CO₂). The supernatants were aspirated, the cells rinsed one time with warm media, and 500 μL fresh media (containing 1% penicillin-streptomycin) was added for addition 1 h incubation. The cells (˜48 h) were washed (2×, 500 μL) with PBS (Mediatech, Inc, Manassas, VA) and removed from wells with 300 μL of trypsin-EDTA (Sigma-Aldrich, St. Louis, MO). Trypsinization was inactivated by resuspension of the cells in 1 ml PBS containing 10% FBS, the cells were transferred to 5 ml round-bottom polystyrene tubes (Corning/Falcon®) and incubated on ice for 10 min. Cells were washed in 1×PBS (centrifuged(500×g, 7 min) to remove any remaining trypsin or FBS, and then permeabilized by incubating in 300 μl 1×BD FACS® Permeabilizing Solution 2 (22° C., 15 min). The cells were centrifuged (400×g, 7 min), washed in 1×PBS, then blocked with 1×PBS containing 5% BSA (22° C., 20 min). Following blocking, cells were washed in 1×PBS and resuspended in 50 μL of mouse anti-flavivirus monoclonal antibody (clone D1-4G2-4-15) diluted to 20 μg/mL in 1×PBS prior to incubation (4° C., 1 h). Cells were washed with 1×PBS then incubated for 30 min (4° C.) with (1:1000) goat anti-mouse IgG (H+L) Alexa Fluor 488-conjugated secondary antibody (Life Technologies) diluted in 1×PBS, centrifuged, and stored in PBS containing 2% formaldehyde (ThermoFisher Scientific) at 4° C. prior to flow cytometry. Flow cytometry data was acquired on a BD FACSCalibur™ instrument with BD CellQuest™ Pro software v 5.2.1 and then subsequently analyzed using FlowJo v10.3 software. The data was exported to Excel 2010 (Microsoft Office) and the percent infection values were background subtracted as follows:

$${I}_{S - C} = {I}_{S} - {I}_{C}$$

where I_{s-c} is the percent YFV infection values, Iₛ is the percent infection values obtained from experimental samples, and I_{c} is the percent infection values obtained from the un-infected Vero cell used for initial gating. Following background subtraction, percent neutralization of single and boosted-vaccinated sera antibodies was calculated as follows:

$$\% {N}_{A b} = 100 - [ 100 ( \frac{S}{N {V}_{a v g}} ) ]$$

where S is the percent YFV infection values from the vaccinated patient samples following background subtraction and NV_{avg} the percent YFV infection values obtained from averaging the non-vaccinated control wells.

The serum dilution that neutralized 50% of YFV was calculated by nonlinear, dose-response regression analysis (F constrained to 50) with Prism 8 software (GraphPad Software, Inc., San Diego, CA) following two independent experiments for each patient. Significant analysis of the mean neutralization titers between vaccinated cohorts was calculated by nonparametric Kruskal-Wallis test (GraphPad Prism v8.3.1).

### Animal Use Statements

This research was conducted in compliance with the Animal Welfare Act and other federal statutes and regulations relating to animals and experiments involving animals, and adhered principles stated in the Guide for the Care and Use of Laboratory Animals, National Research Council, 1996, under facilities fully accredited by the Association for the Assessment and Accreditation of Laboratory Animal Care International.

### Human Use Statements

Research on human subjects was conducted in full compliance with DoD, NIH, federal, and state statutes and regulations relating to the protection of human subjects and adheres to principles identified in the Belmont Report (1979). All specimens, data, and human subject research were gathered and conducted for this publication under IRB-approved protocols.

### Results

### Antibody Responses to Primary and Secondary Vaccination Determined by Protein Microarrays.

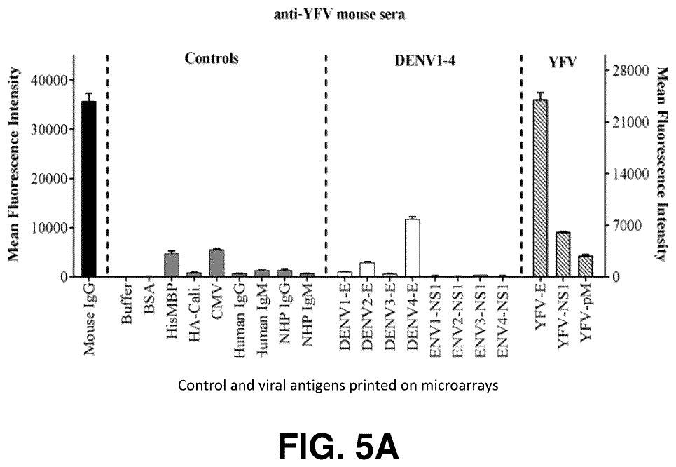

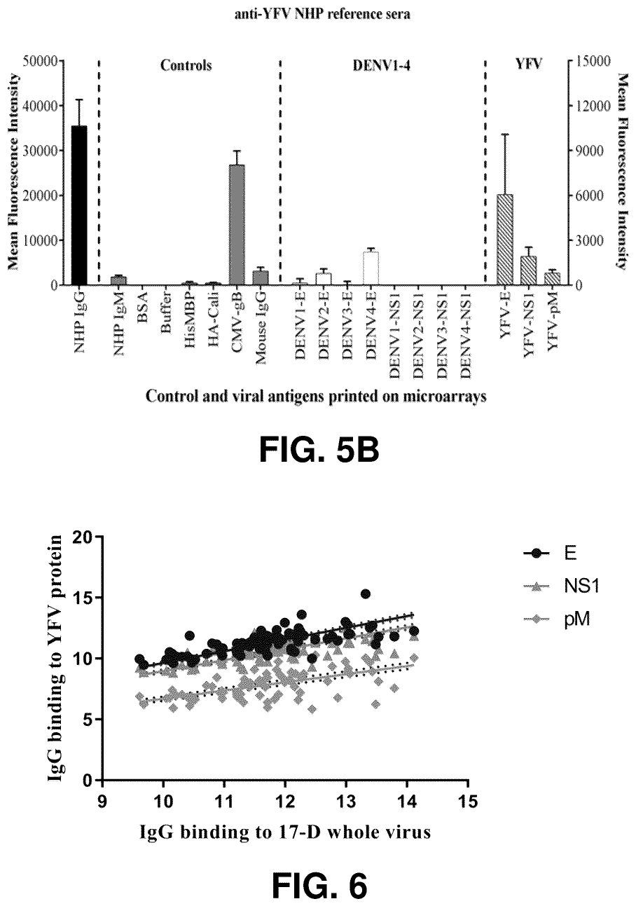

The E (transmembrane domain deleted), domain III of E (E-DIII), NS1, and pM/M from 24 species and strains of flaviviruses that infect humans (Table 1) were prepared and characterized for inclusion in the protein microarray using previously described methods. Keasey et al. Clin Vaccine Immunol. (2017) 24(4). Several control proteins were also printed on the same microarrays and all spot densities (1-10 μg) were calibrated for best signal and lowest background to optimize assay performance. The performance of the YFV antigens was evaluated with sera collected from mice and non-human primates that received the 17D vaccine. These results (FIGS. 5A-B) showed that the E protein from YFV bound the greatest amount of antibody, followed by NS1 and pM for antisera from both animal species. Sera collected from adult human subjects one month to sixteen years from the last day of vaccination with 17D were examined with a focused microarray printed with 17D virus and recombinant proteins (E, pM, NS1) from both YFV and DENV1-4. We limited our results to IgG and did not evaluate IgM, which is detectable for at least four years after vaccination. See, e.g., Gibney et al. Am J Trop Med Hyg. (2012) 87(6): 1112-5.

The results were collated into five specimen collection time intervals to compensate for the variable times from vaccination for each subject (Table 2). For assay performance, the microarray surfaces were incubated with 2 μl of human serum that was diluted with buffer (1:150), antigen-bound antibody was detected with goat anti-human γ-specific IgG conjugated with Alexa Fluor 647, and microarray images were captured by laser scanner for analysis. The total number of seropositive subjects with significantly elevated IgG binding signals (μ+2σ) in comparison to non-vaccinated controls is presented in Table 7. These results demonstrate that the highest level of seroconversions (100%) were captured by E and whole virus assay probes 196 days (median) after primary vaccination, while pM and NS1 were less effective (72% and 54%, respectively). Interestingly, one subject from the 30-90 day interval and two from the time interval of 91-270 days had higher antibody binding to NS1 and pM antigens than YFV-E or whole virus (data not shown). A receiver-operator characteristics (ROC) curve was generated using the microarray antibody binding results of naïve and 17D vaccinated individuals to each YFV antigen in order to determine the strength of the antigen as a classifier of YFV immunity.

TABLE 7
Sera examined from naïve and YFV (17D) immunized subjects.

| Vaccination | Time interval from | | Median day from last | Total |
|---|---|---|---|---|
| cohort | vaccination | | vaccination | subjects |
| Primary³ | 30-90 | days | 51¹ | 19 |
| | 90-270 | days | 196 | 18 |
| | 271-364 | days | 302 | 10 |
| | 1-6 | years | 1106 | 7 |
| | 7-16 | years | 3984 | 8 |
| Boosted | 1-6 | years | 846.5 | 10 |

| | | | |
|---|---|---|---|
| Non-vaccinated | NA⁴ | NA | 11 |

¹Median day after vaccination
²Minimum and maximum range of days after vaccination
³Year after most recent vaccination, days after primary vaccination ranged from 4169-10,098d with 4849d as the median day post-primary vaccination
⁴Not applicable

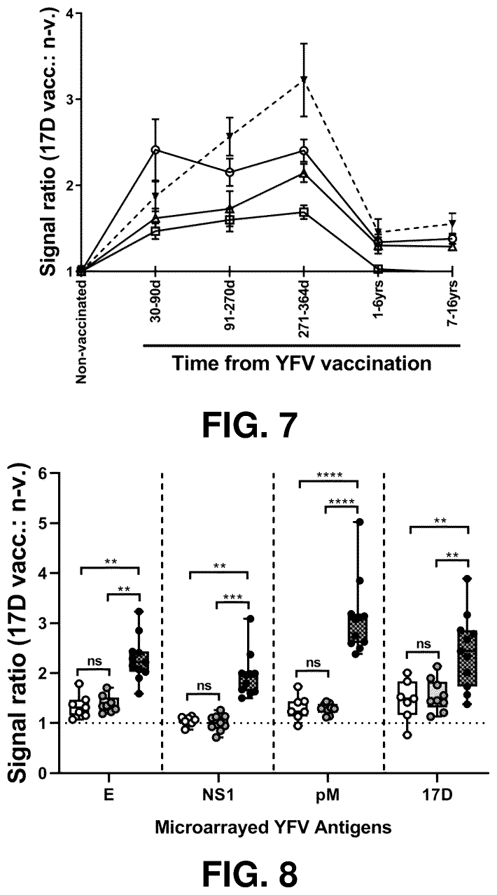

Over a wide range of antigen densities on the microarray, whole virus and E antigens had a greater ability to discriminate between antibody responses of naïve and YFV immune individuals than NS1 and pM, with area under the curve (AUC) values of 0.95 (p 0.0001) and 0.85 (p 0.0052) for virus and E, respectively (Table 3). Furthermore, there was a strong positive correlation (Pearson's r=0.7, p<0.0001) between antibody binding to whole virus and recombinant E (FIG. 6). Taking into consideration serum IgG recognition of both E and whole virus, it was determined that >98% of subjects that received either a single or multiple dose of the 17D vaccine had a significantly elevated (μ+2σ) antibody response to the respective YFV antigen relative to non-vaccinated individuals and the number of seropositive subjects increased from 30-364 days after vaccination. While the maximum number of seroconversions were noted by the median day 196 with any of the YFV antigens, the number of subjects with significant antibody levels declined (FIG. 7) by median day 1106 (˜3 years). In contrast, seroconversions among subjects receiving booster vaccinations with 17D were elevated to levels by median day 846 (˜2.3 years) that equaled or exceeded results obtained from primary vaccination alone. Compared to primary vaccination, antibody responses to all antigens were significantly elevated in boosted subjects (FIG. 8). As noted above, total polyclonal antibody responses to both YFV proteins and whole virus decreased over time from primary vaccination, but were elevated in individuals boosted within approximately the same length of time.

TABLE 8
Receiver operating characteristic curves demonstrate broad dynamic range for
antigen densities to distinguish 17D vaccination from naive antibodies.

| Conc. | AUC values¹ | | | | | |
|---|---|---|---|---|---|---|
| (μg/mL) | E | NS1 | | pM | 17D | Dilution |
| 100 | 0.97 (0.93-1.00) | 0.66 | (0.50-0.82)² | 0.95 (0.87-1.00) | 0.92 (0.85-0.98) | 1:32 |
| 200 | 0.96 (0.92-1.00) | 0.78 | (0.63-0.93) | 0.99 (0.98-1.00) | 0.89 (0.80-0.97) | 1:16 |
| 400 | 0.95 (0.90-1.00) | 0.81 | (0.69-0.93) | 0.95 (0.90-1.00) | 0.94 (0.88-1.00) | 1:8 |
| 800 | 0.98 (0.95-1.00) | 0.85 | (0.76-0.95) | 0.84 (0.74-0.93) | 0.97 (0.94-1.00) | 1:4 |
| 1000 | 0.97 (0.93-1.00) | 0.82 | (0.68-0.96) | 0.85 (0.72-0.99) | 0.97 (0.94-1.00) | 1:2 |

¹Yellow fever antigens with area under the curve (AUC) value >0.80 were used for data analysis.
²Value indicates the area under the curve value (AUC) with the 95% confidence interval values shown in brackets.

### Neutralizing Antibody Responses to Primary and Secondary Vaccination.

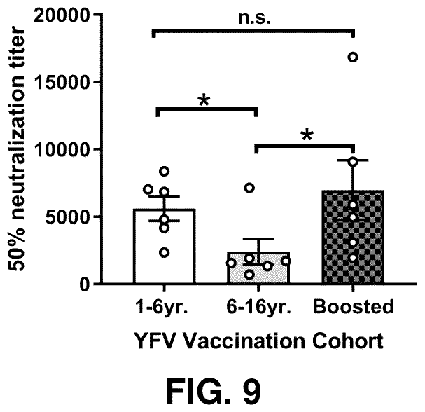

Because virus neutralization by serum antibodies is commonly used to assess functional immune responses to yellow fever vaccination, the levels of neutralizing antibodies for primary and boost vaccines were examined to provide a basis for comparison with the microarray results. Neutralizing antibody titers for a subset of primary (n=6, 1-16 years after vaccination) and secondary (n=6, 1-6 years after final vaccination) 17D-vaccinated subjects were evaluated by a virus-neutralization assay (FIG. 9). These results demonstrated that secondary vaccination significantly increased YFV-neutralizing antibody titers compared to sera collected >6 years from primary vaccination, while PRNT results for boosted subjects were not significantly different from the 1-6 year time interval for primary vaccinations. We also noted a decline in neutralizing antibody titers from 6-16 years compared to 1-6 years, but this did not reach a level of significance.

### Antibody Cross-Reactivity.

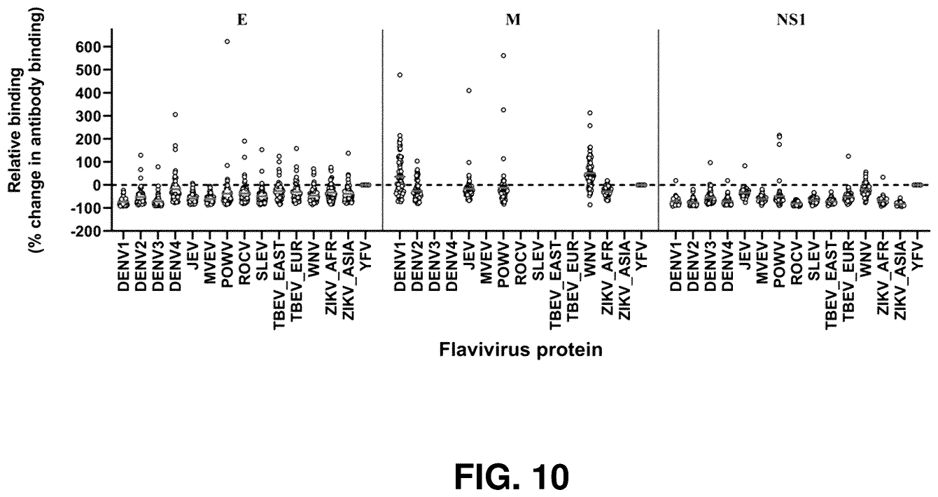

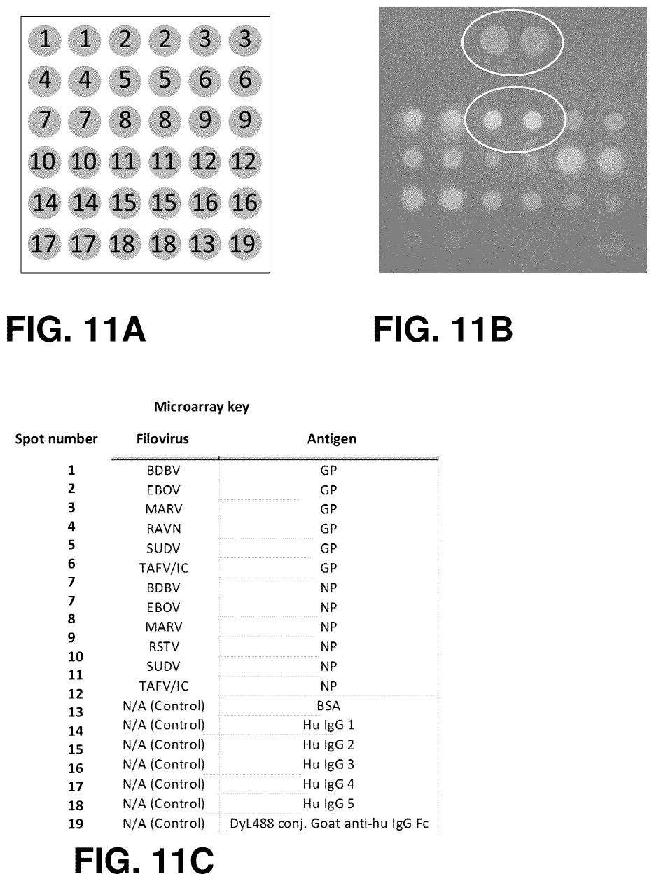

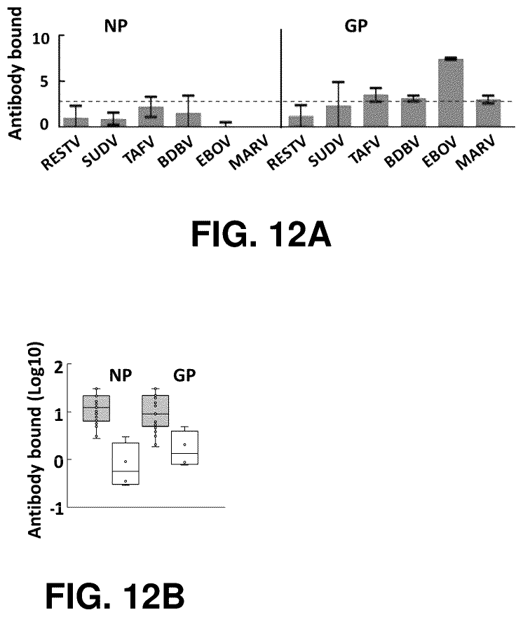

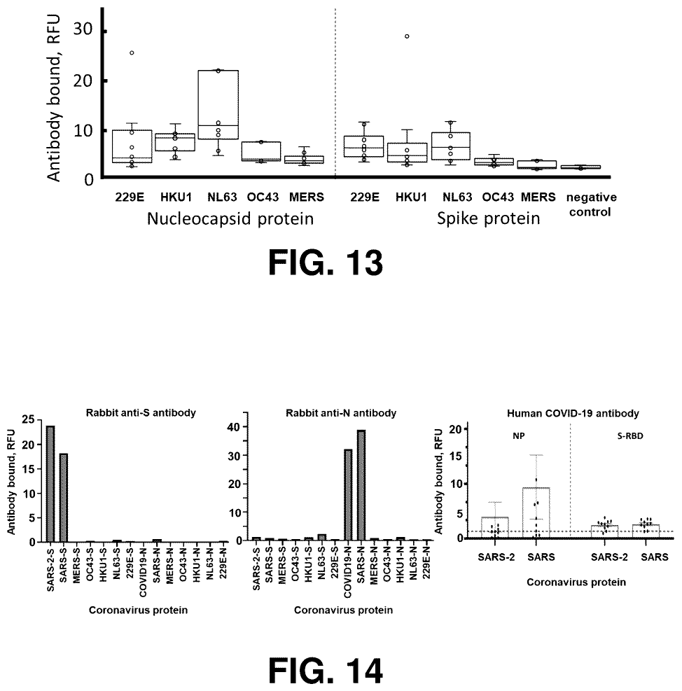

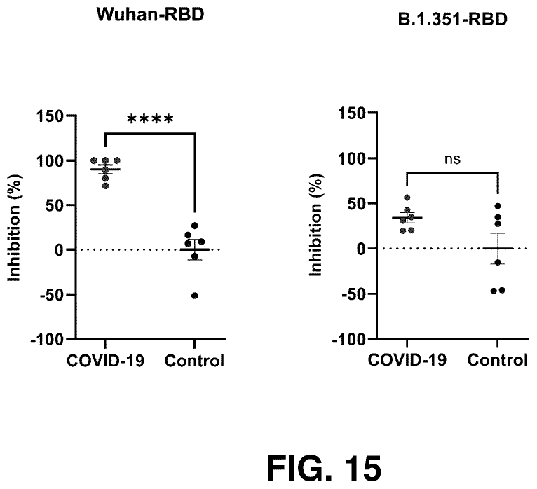

A second microarray was printed that included E, pM and NS1 antigens from 15 species or serotypes of flaviviruses. An examination of sera from primary (n=67) and boosted (n=11) 17D vaccinations showed that antibodies principally recognized E and NS1 from YFV, with a small number of individuals presenting cross-reactive antibodies targeting other flaviviral proteins (FIG. 10). The E antigens of DENV4, TBEV, and ZIKV exhibited modest antibody cross-reactivity with E from YFV, as did the NS1 from WNV and M from DENV1 and WNV. In general, there was <50% relative antibody binding towards all other flavivirus E antigens (FIG. 10) and anti-YFV pM antibodies led to the highest observed level of cross-reactivity.

### Discussion

In the study described here, we examined serological immune responses to 17D vaccination by focusing on individual antigens from YFV and related flaviviruses. Active surveillance of population immunity and disease through laboratory testing is an essential component of the international strategy for control of yellow fever. Eliminate Yellow fever Epidemics (EYE): a global strategy, 2017-2026. Wkly Epidemiol Rec. (2017) 92(16): 193-204.

A reassessment of methods to evaluate immune responses to vaccination will ultimately help to strengthen the management of disease outbreaks. In our study, the frequencies of seropositive subjects assessed by microarray and virus neutralization were greatest up to one year from primary vaccination and declined thereafter. In contrast, specific antibody binding to microarrayed antigens was significantly elevated in boosted subjects compared to primary vaccination, while virus neutralization results for boosted subjects were similar to primary vaccination. The ability to evaluate both primary and boosted vaccinations is important for understanding the duration of immunity.

For 17D vaccination, we observed that antibody cross-reactivities were minimal with recombinant antigens from dengue, Zika, West Nile and other flaviviruses that are pathogenic to human, indicating that specificity could be maximized by use of isolated YFV antigens. Cross-reactive antibodies that result from vaccination have been reported to interfere with virus-neutralization assays for other flaviviruses. Hobson-Peters J Biomed Biotechnol (2012) 2012: 379738; Mansfield et al. J Gen Virol (2011) 92(Pt 12): 2821-9. However, a recent report also found no evidence of interference in the specificity of IgG ELISA results for DENV and ZIKV from YFV-neutralizing antibodies stimulated by the yellow fever vaccine. Souza et al. Int J Infect Dis (2019) 81: 4-5.

Correspondingly, a high prevalence of anti-dengue antibodies in Brazil does not apparently interfere with seroconversion from 17DD vaccination. de Melo et al. (2011) 85(4): 739-47. Further, our results demonstrate that antibody capture by the E antigen produced the highest level of specificity for YFV, followed by pM and NS1 proteins. A greater level of specificity and reproducibility can be achieved by using recombinant test antigens instead of the infected cell lysates that are commonly used for ELISA, as shown here and by the work of others. Basile et al. J Virol Methods (2015) 225: 41-8; Beasley et al. J Clin Microbiol. (2004) 42(6): 2759-65; Kuno Adv Virus Res (2003) 61: 3-65.

It is possible that a test algorithm that includes multiple antigen probes (such as E, pM and NS1) may ultimately be the most robust. However, it may not be possible to distinguish past infection from previous vaccination with the antigenically similar live-attenuated YFV.

Wide-spread and lasting epidemics of yellow fever occurred prior to the implementation of mosquito-control measures and development of the highly effective vaccine. The historical epidemics have been replaced by sporadic outbreaks among immunologically naïve populations.

The results presented here provide a foundation for establishing new high-throughput serological assays that are more amenable to large population-based studies. The printed flavivirus microarray described was covered with a gasket that divided the slide into 16 separate assays. For higher throughput, the gasket wells can be expanded to a practical higher limit that will allow 64 separate sera to be processed on the same slide, using pL of specimen diluted in 20 μL per well. Beyond evaluation of vaccination, more detailed data for antibody responses in mild and asymptomatic infections in comparison to life-threatening cases of yellow fever will be important to establish better correlates of protective immunity. Table 2 lists the preferred sequences for the YFV and Table 1 lists the preferred antibodies. The E, prM/M and NS1 peptides are preferred, and a configuration of six columns by six rows is preferred for arrays to include duplicate antigens from the other flaviviruses that infect humans. The remaining spots can be used for negative or positive controls, which can include HA proteins from seasonal influenza viruses, a capture reagent that will bind human antibodies, buffer only, or other control proteins and substances. The antigen array can also consist of YFV peptides replicated in 2-6 spots along with negative and positive control spots.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

1. A system for detecting the presence of a virus in a sample comprising a platform comprising a buffer chamber in fluid communication with a sample receiver, the sample receiver comprising a sample chamber and a membrane and is in fluid communication with a secondary agent depot, the secondary agent depot comprising a secondary agent and is in fluid communication with a reaction chamber, the reaction chamber comprising an array comprising at least one viral peptide and an optical window and is in fluid communication with a waste chamber.

2. The system of claim 1, wherein the virus is a coronavirus, preferably SARS-CoV-1, MERS-CoV, SARS-CoV-2 (COVID-19), HCoV-OC43, HCoV-HKU1, HCoV-NL63, HCoV-229E or a combination thereof.

3. The system of claim 1, wherein the viral peptide is a coronavirus peptide hemmaglutinin esterase (He), membrane protein (M), envelope small membrane protein (E), nucleocapsid (N), spike (S), or a combination thereof.

4. The system of claim 1, wherein the viral peptide is a SARS-CoV peptide, MERS-CoV peptide, SARS-CoV-2 (COVID-19) peptide, HCoV-OC43 peptide, HCoV-HKU1 peptide, HCoV-NL63 peptide, HCoV-229E peptide, an antigenic fragment thereof, or a combination thereof.

5. The system of claim 3, wherein the viral peptide is a SARS-CoV-2 (COVID-19) peptide, an antigenic fragment thereof, or a combination thereof.

6. The system of claim 1, wherein the S protein is SSARS-2, SSARS, SMERS, SOC43, SHKU1, SNL63, S229E, antigenic fragments thereof, or a combination thereof.

7. The system of claim 1, wherein the N protein is NSARS-2, NSARS, NMERS, NOC43, NHKU1, NNL63, N229E, antigenic fragments thereof, or a combination thereof.

8. (canceled)

9. The system of claim 1, wherein the coronavirus viral peptide comprises a sequence selected from the group consisting of an amino acid sequence with at least about 90% sequence homology to the amino acid sequences of 248, 250, 252, 254, 256, 258, 260, 262, 264, 266, 268, 270, 272, 274, antigenic fragments, epitopes contained therein, or combinations thereof.

10. (canceled)

11. The system of claim 1, wherein the virus is a filovirus, optionally a Zaire ebolavirus (Ebola Virus), Sudan ebolavirus (Sudan virus), Taï Forest ebolavirus (Cote d'Ivoire ebolavirus) (Taï Forest virus), Bundibugyo ebolavirus (Bundibugyo virus), Reston ebolavirus (Reston virus), Bombali ebolavirus (Bombali virus), Marburg marburgvirus (Marburg virus), or a combination thereof.

12. (canceled)

13. The system of claim 11, wherein the Ebola virus peptide is a Zaire ebolavirus (Ebola Virus) peptide, Sudan ebolavirus (Sudan virus) peptide, Taï Forest ebolavirus (Côte d'Ivoire ebolavirus)(Taï Forest virus) peptide, Bundibugyo ebolavirus (Bundibugyo virus) peptide, Reston ebolavirus (Reston virus) peptide, Bombali ebolavirus (Bombali virus) peptide, Marburg marburgvirus (Marburg virus) an antigenic fragment thereof; or a combination thereof.

14. The system of claim 11, wherein the Filoviral peptide is glycoprotein (GP), nucleocapsid protein (NP), minor nucleoprotein (VP30), polymerase complex protein (VP35), matrix (VP40), VP24, an antigenic fragment thereof, or a combination thereof.

15. The system of claim 11, wherein the Filoviral peptide is glycoprotein, nucleocapsid protein (NP), an antigenic fragment thereof, or a combination thereof.

16. (canceled)

17. The system of claim 11, wherein the Filoviral peptide comprises a sequence selected from the group consisting of the amino acid sequences of SEQ ID NOs: 2, 4, 6, 8, 10, 12, 16, 18, 20, 22, 24, 26, 28, 30, 32, 34, 36, an antigenic fragment thereof, an epitope therein, or a combination thereof.

18. The system of claim 11, wherein the Filoviral peptide comprises a NP sequence selected from the group consisting of an amino acid sequence with at least about 90% sequence homology to the amino acid sequences of SEQ ID NOs: 4, 10, 16, 22, 28, 34, an antigenic fragment thereof, an epitope therein, or a combination thereof.

19. The system of claim 11, wherein the Filoviral peptide comprises a VP sequence selected from the group consisting of the amino acid sequences of SEQ ID NOs: 4, 10, 16, 22, 28, 34, an antigenic fragment thereof, an epitope therein, or a combination thereof.

20. (canceled)

21. (canceled)

22. The system of claim 1, wherein the viral peptide is a structural protein from the Yellow Fever virus, optionally Capsid, pM, E, or a combination thereof.

23. The system of claim 1, wherein the viral peptide is a non-structural protein from the Yellow Fever virus, optionally NS1, NS2a, NS2b, NS3, NS4a, NS4b, NS5, or a combination thereof.

24. (canceled)

25. The system of claim 1, wherein the viral peptide is a flaviviral peptide and comprises an amino acid sequence with at least 90% sequence homology to the amino acid sequence of SEQ ID NO: 37, 40, 43, 46, 49, 52, 55, 58, 61, 64, 67, 70, 73, 76, 79, 82, 88, 91, 94, 97, 100, 103, 106, 109, 112, 115, 118, 121, 124, 127, 130, 133, 136, 139, 142, 145, 148, 151, 154, 157, 160, 163, 166, 169, 172, 175, 178, 181, 184, 187, 190, 193, 196, 199, 202, 205, 208, 211, 214, 217, 220, 223, 226, 229, 232, 235, 238, 241, 244, antigenic fragments thereof, eptiopes contained therein, or a combination thereof.

26. (canceled)

27. The system of claim 1, wherein the reaction chamber further comprises a viral peptide selected from the group consisting of influenza A virus peptides, influenza B virus peptides, influenza C peptides, enterovirus peptides, respiratory syncytial virus (RSV) peptides, parainfluenza peptides, adenovirus peptides, or a combination thereof.

28. The system of claim 27, wherein the influenza A virus is H1N1, H1N2, H3N2, H5N1. H1N2, H7N9, or a combination thereof.

29. A system for detecting the presence of a coronavirus in a sample comprising a platform comprising a buffer chamber in fluid communication with a sample receiver, the sample receiver comprising a sample chamber and a membrane and is in fluid communication with a secondary agent depot, the secondary agent depot comprising a secondary agent and is in fluid communication with a reaction chamber, the reaction chamber comprising an array comprising at least one anti-viral antibody or antigen binding fragment thereof and an optical window and is in fluid communication with a waste chamber.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. A method for detecting the presence of a virus in a sample comprising using the system of claim 1.

66. (canceled)

67. (canceled)

68. (canceled)

69. (canceled)

70. (canceled)

71. (canceled)

72. (canceled)

73. (canceled)
